(19) United States
(12) Patent Application Publication
(10) Pub. No.: US 2024/0350650 A1
(43) Pub. Date: Oct. 24, 2024

(54) LINKER AND CONJUGATE THEREFOR

(71) Applicant: BEIJING HOPE PHARMACEUTICAL CO.LTD., Beijing (CN)

(72) Inventors: Haining LV, Beijing (CN); Yingzhao WANG, Beijing (CN); Changru LIU, Beijing (CN); Yanling LIU, Beijing (CN); Sheng TANG, Beijing (CN); Chunxia LI, Beijing (CN); Mingsheng ZHAI, Beijing (CN); Jiuquan YANG, Beijing (CN); Bojing LIU, Beijing (CN); Hongyun LI, Beijing (CN)

(73) Assignee: BEIJING HOPE PHARMACEUTICAL CO.LTD., Beijing (CN)

(21) Appl. No.: 18/570,945

(22) PCT Filed: May 19, 2022
(86) PCT No.: PCT/CN2022/093808

§ 371 Date: Dec. 15, 2023

(30) Foreign Application Priority Data
Jun. 18, 2021 (CN) 202110677212.3

(51) Int. Cl. A61K 47/68

(52) U.S. Cl. CPC A61K 47/68

(57) ABSTRACT

A linker, comprising an Lb structure fragment, the Lb structure being selected from the following formula, wherein subscript q is selected from any integer from 1 to 20, preferably from 3 to 10, and most preferably from 5 to 8. The present application also relates to a use of the linker in the preparation of a linker-drug and a ligand-drug conjugate.

### FIELD OF THE INVENTION

The present application relates to the field of biological medicine, and in particular, relates to a linker and a conjugate thereof.

### BACKGROUND OF THE INVENTION

At present, antibody-drug conjugates (ADCs) allow the linkage of monoclonal antibodies or antibody fragments with bioactive cytotoxins via stable chemical linker compounds, which makes full use of the binding specificity of the antibodies to normal cells and to tumor cell surface antigens as well as the high efficiency of cytotoxic substances, while avoiding the defects of the former's low efficacy and the latter's excessive toxic and side effects. This means that the antibody-drug conjugates are able to bind to tumor cells more precisely and reduce the impact on normal cells as compared to conventional chemotherapy drugs. However, only a few classes of drugs have been demonstrated to have sufficient activity as antibody-drug conjugates, and to have both suitable toxicity and other pharmacological properties in order to guarantee clinical development. Camptothecin (CPT) is one class of drugs of interest.

It is a family of water-soluble cytotoxic quinoline alkaloids isolated from Camptotheca acuminata (Camptotheca, Happy tree), which is a kind of tree native to Tibet, China. Camptothecin inhibits DNase topoisomerase I. Topoisomerase I is an endonuclear enzyme non-covalently bound to a torsionally stretched supercoiled double-stranded DNA to produce instantaneous single-strand breaks (named “cleavable complexes”) in a DNA molecule. This allows intact complementary DNA strands to pass through during replication, transcription, recombination, and other DNA functions. These enzyme-bridged DNA breaks, called cleavage complexes, are subsequently resealed by the topoisomerase I enzyme. The dissociation of the enzyme restores the intact new relaxed DNA double helix. The application of camptothecin derivatives in antibody-drug conjugates (ADCs) have been reported in documents such as WO2014057687, WO2019195665, and WO2019236954.

The design of chemical linkers for covalently binding antibodies to drugs to form ADCs also plays a role in the development of ADCs. For example, the linkers should keep stable in the blood so as to limit damage to healthy tissues. The decomposition or decay of the ADC allows release of cytotoxic drugs before the ADC is delivered to a site of interest. Once the ADC reaches the site of interest, the ADC must efficiently release the cytotoxic drug in an active form. The balance between plasma stability and effective drug release at target cells remains to be explored and may depend on the linker design. The linker technology affects the efficacy, specificity, and safety of ADCs. There is a need for a linker for ADCs to provide serum stability and increased solubility to allow effective conjugation and intracellular delivery of hydrophobic drugs.

Compared with highly active drugs used in other ADCs, CPT, as an anti-tumor clinical drug, shows the advantage in terms of toxic and side effects, and provides a reasonable choice for further improving the therapeutic effect of ADCs and increasing the drug load of ADCs to increase the targeted drug release amount of ADCs in vivo. However, camptothecin and most of other ADC-conjugated drugs are lipophilic molecules, and would lead to instability as well as aggregation and settlement of ADCs when a traditional linker is used. In addition, excessively high lipophilicity would lead to rapid metabolic elimination in the liver and other metabolic organs in the body, resulting in poor PK properties. Therefore, it is necessary to explore, from the structure of the linker, how to ensure a high drug load while optimizing the physical and chemical properties of ADCs to achieve a good activity in vivo and in vivo. Hence, there is still a need for further development of ADC drugs with better efficacy.

### SUMMARY OF THE INVENTION

An object of the present application is to provide a novel linker, a method for preparing the linker, and uses of the linker in preparation of a linker-drug conjugate and an antibody-drug conjugate. The linker of the present application can increase the drug load to thereby improve the efficiency of drug delivery, and at the same time, a ligand-drug conjugate prepared using the linker is not prone to aggregation and settlement and shows good anti-tumor activity in vitro and in vitro, thereby indirectly revealing good in vivo stability and PK properties of the ligand-drug conjugate prepared using the linker.

In an aspect, the present application provides a linker comprising an Lb structure fragment, wherein an Lb structure is selected from the following formula:

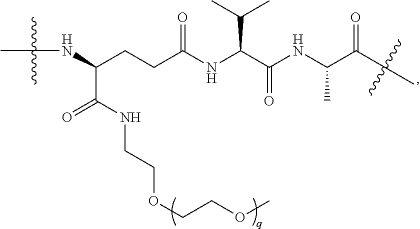

wherein a subscript q is any integer selected from 1-20, preferably 3-10, and most preferably 5-8.

In some embodiments, the linker comprises an Lb-Lc structure fragment, the Lb structure being linked to the Lc fragment via carbonyl, wherein Lc is a releasable assembly unit and is capable of linking a drug unit.

In some embodiments, the Lc structure is selected from the following formulas:

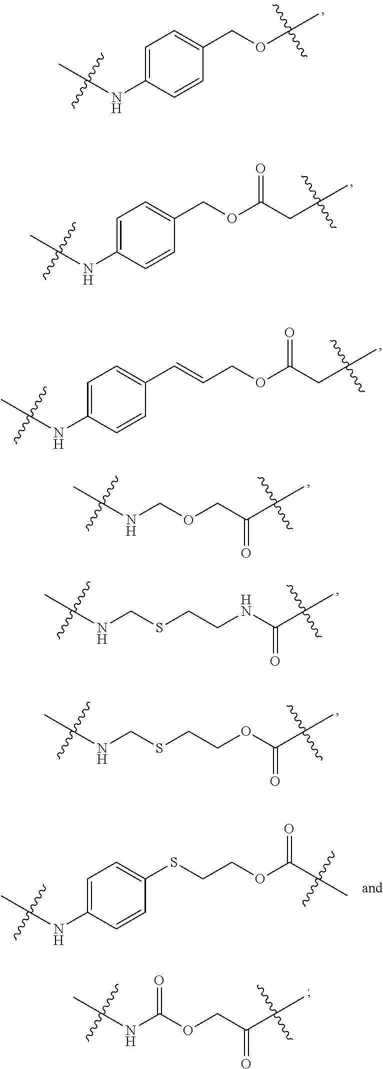

optionally, the Lc structure is linked to carbonyl of the Lb structure via amino; and Lc is capable of linking the drug unit via carbonyl.

In some embodiments, the linker has a structure shown in Formula (I):

La—Bₚ [symbol] -Lb-Lc)ₘ Formula (I),
wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

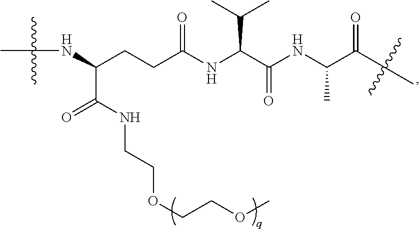

wherein a subscript q is any integer selected from 1-20, preferably 3-10, and most preferably 5-8, and the Lb structure is linked to an La or B structure via amino and to the Lc fragment via carbonyl;
Lc is a releasable assembly unit, the Lc structure is linked to carbonyl of the Lb structure via amino, and Lc is capable of linking the drug unit via carbonyl.

In some embodiments, the B structure is selected from the following formula:

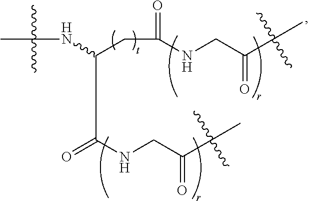

wherein a subscript t is any integer independently selected from 1-4, each subscript r is any integer independently selected from 1-4, and the B structure is linked to the La structure via amino and to the Lb structure fragment via carbonyl.

In some embodiments, the B structure is selected from the following formula:

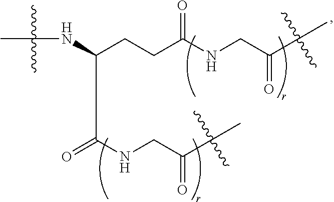

wherein each subscript r is any integer independently selected from 1-4, and the B structure is linked to the La structure via amino and to the Lb structure fragment via carbonyl.

In some embodiments, the La structure comprises a maleimide type linker fragment.

In some embodiments, the La structure has the following structure:

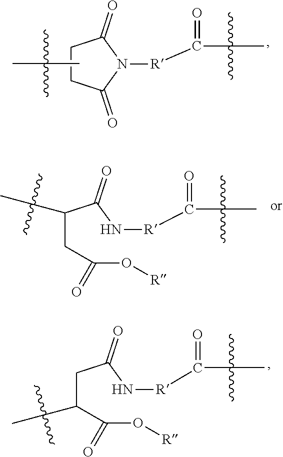

and is linked to the Lb or B structure fragment via carbonyl, and is capable of linking the ligand unit via position(s) 3 and/or 4 of succinamide; and R′ is selected from the group consisting of: optionally substituted C₁-C₁₀ alkyl, C₁-C₁₀ alkoxy, C₁-C₁₀ aminoalkyl and C₁-C₁₀ alkyl-aryl,

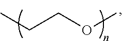

R″ is selected from: optionally substituted C₁-C₁₀ alkyl; wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl; and wherein a subscript n is any integer selected from 1-10, preferably 1-5, and most preferably 1-3.

In some embodiments, the La structure is selected from the following formulas:

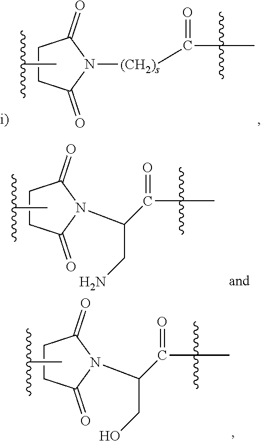

wherein the La structure is linked to the Lb or B structure fragment via carbonyl, and is capable of linking the ligand unit via position(s) 3 and/or 4 of succinimide;

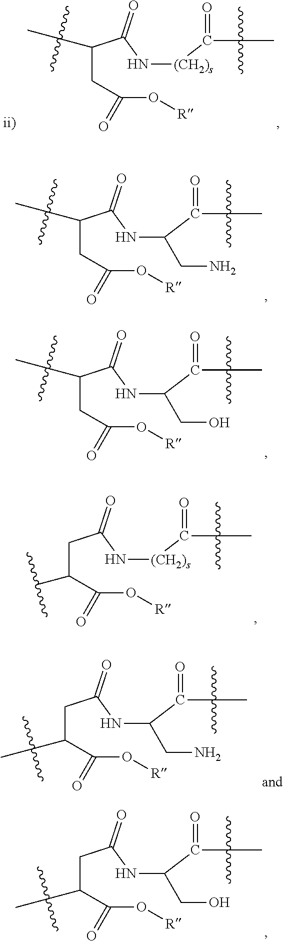

wherein the La structure is linked to the Lb or B structure fragment via carbonyl, and is capable of linking the ligand unit via position(s) 3 and/or 4 of succinamide; and R″ is selected from: methyl, ethyl or propyl;
wherein each subscript s is any integer independently selected from 1-10, preferably 1-8, and more preferably 1-5.

In some embodiments, the La structure is selected from the following formula:

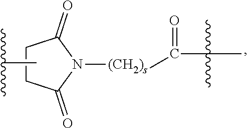

wherein the La structure is linked to the Lb or B structure fragment via carbonyl, and is capable of linking to the ligand unit via position(s) 3 and/or 4 of succinimide; and wherein the subscript s is any integer selected from 1-10, preferably 1-8, and more preferably 1-5.

In some embodiments, the La structure is selected from the following formula:

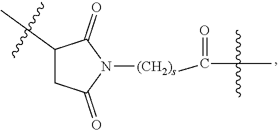

wherein a subscript s is any integer selected from 1-10, preferably 1-8, and more preferably 1-5, and the La structure is linked to the B or Lb structure fragment via carbonyl, and is capable of linking the ligand unit via position 3 of succinimide/succinamide.

In some embodiments, the La structure is selected from the following formula:

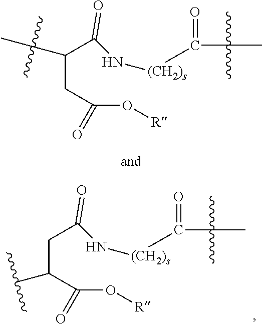

wherein R″ is selected from the group consisting of methyl, ethyl and propyl, a subscript s is any integer selected from 1-10, preferably 1-8, and more preferably 1-5, and the La structure is linked to the B or Lb structure fragment via carbonyl.

In some embodiments, the linker has a structure shown in Formula (Ia) or Formula (Ib):

La-Lb-Lc, Formula (Ia),

La—B [symbol] Lb-Lc)₂, Formula (Ib),
wherein,
La is an extension unit capable of linking a ligand unit;
B is a branching unit having a structure selected from the following formula:

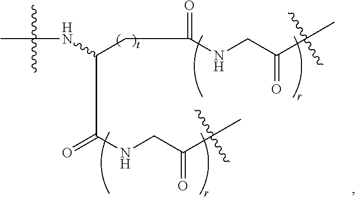

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

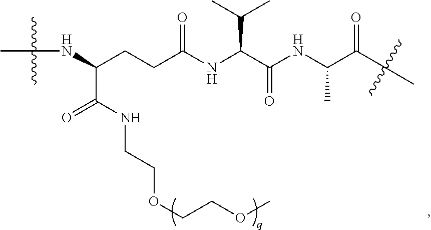

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit capable of linking a drug unit.

In some embodiments, the linker is selected from the following structures:

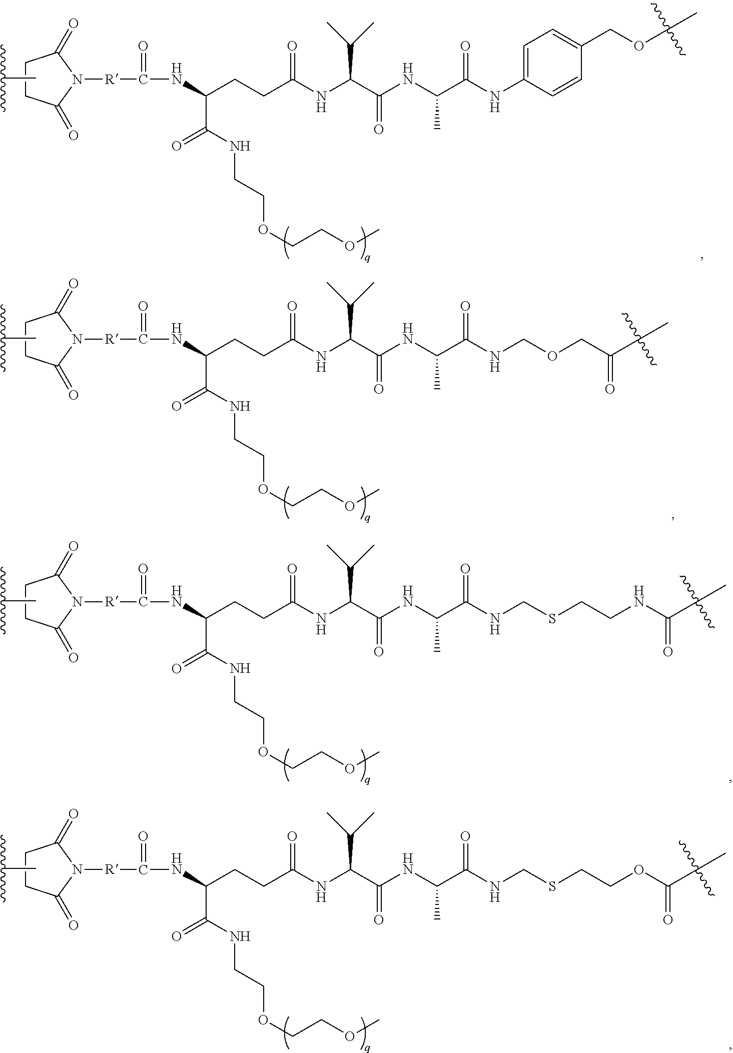

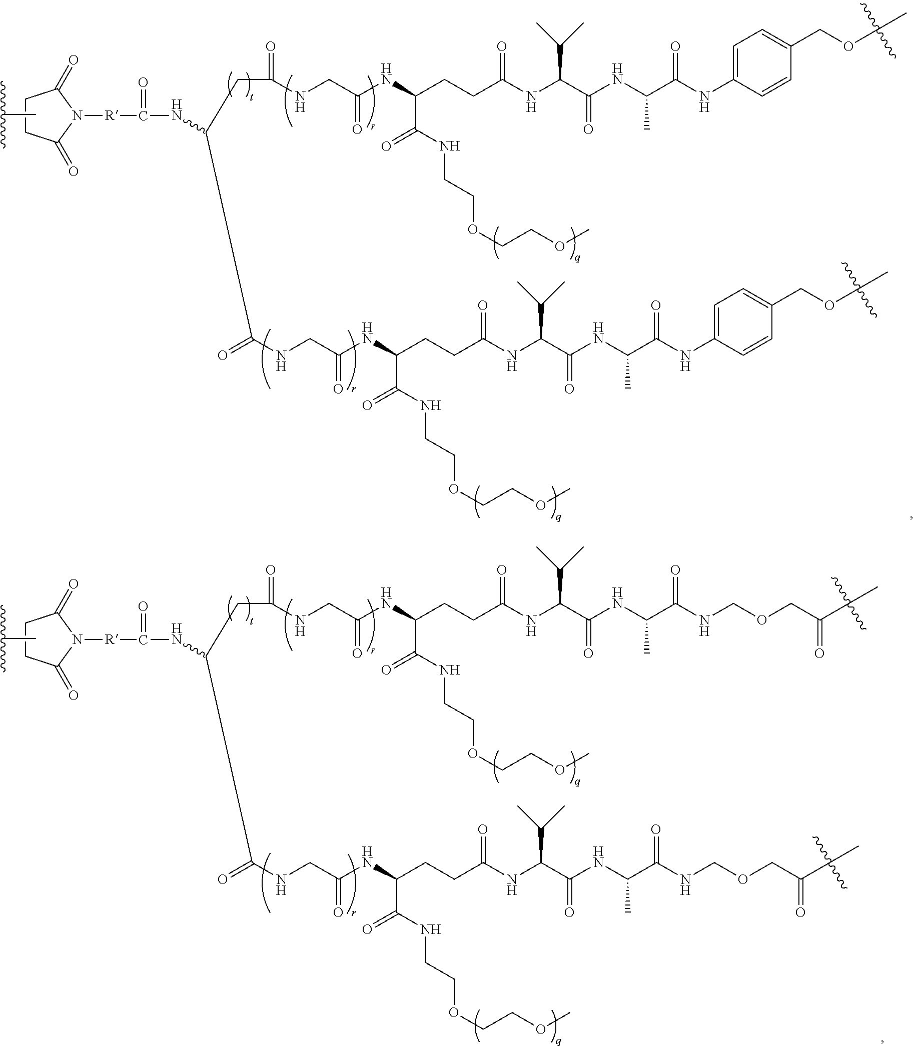

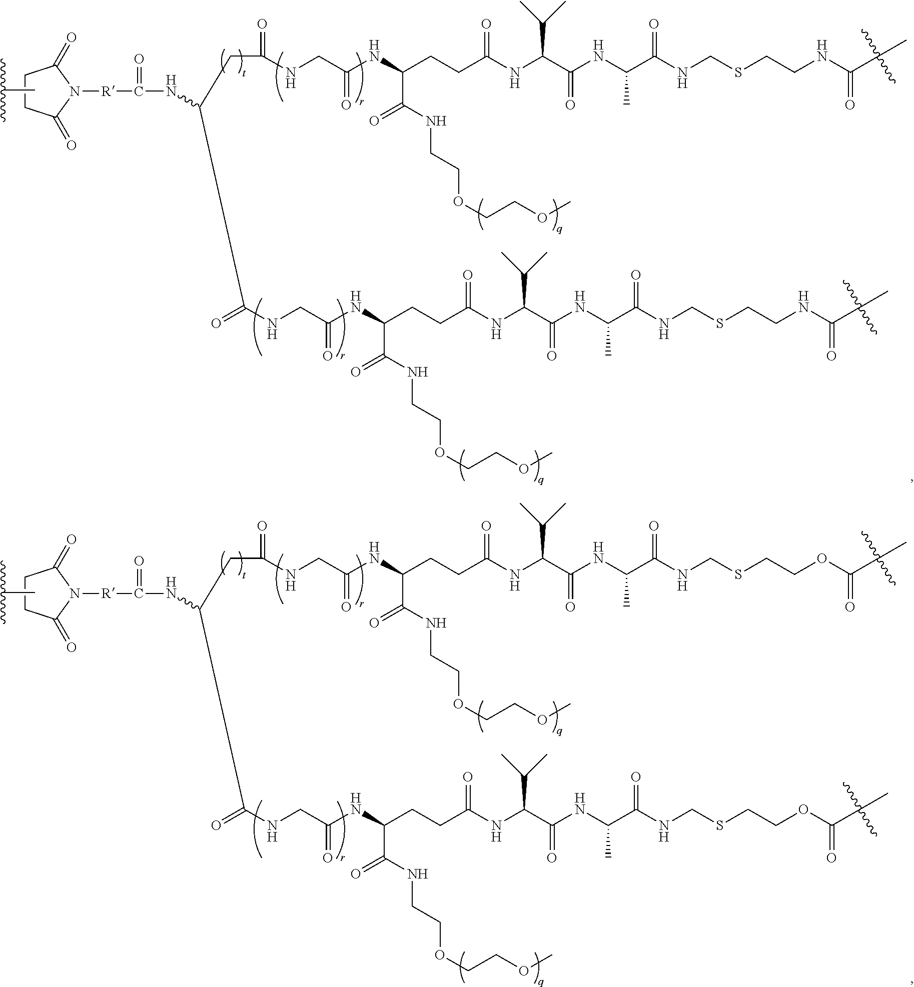

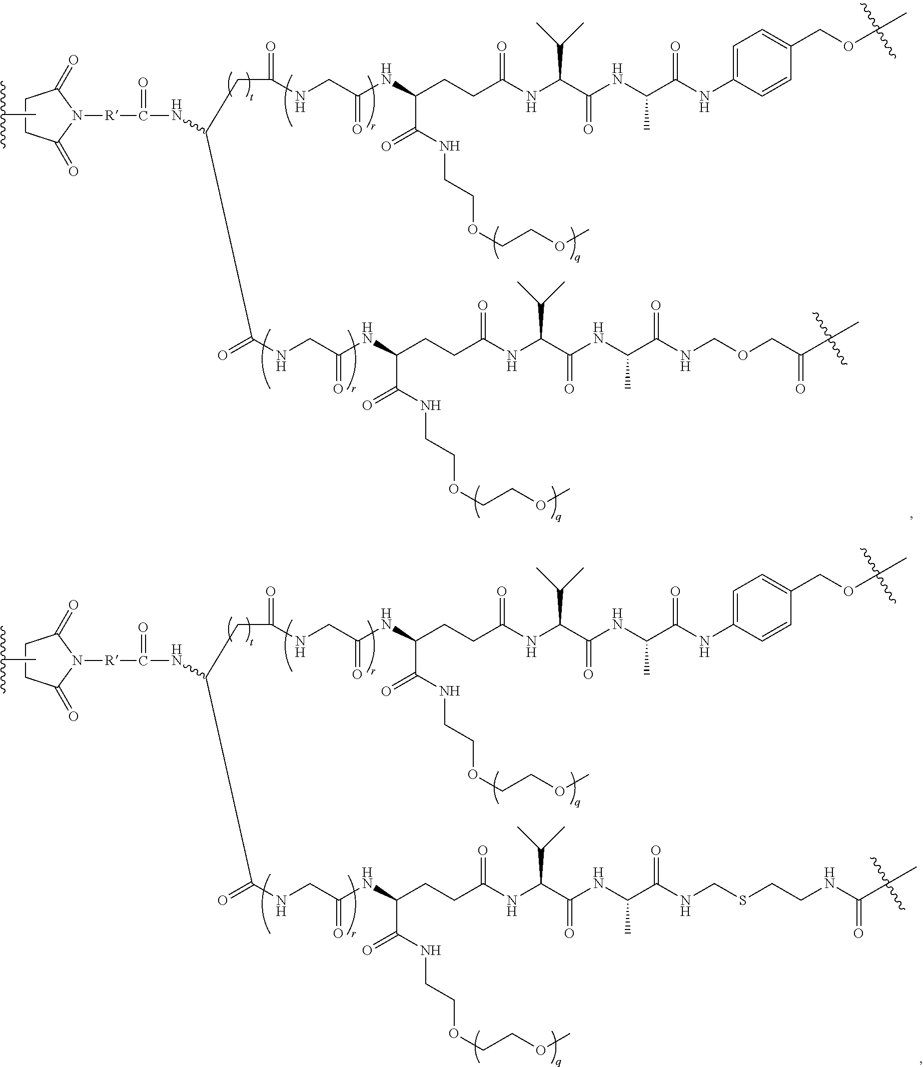

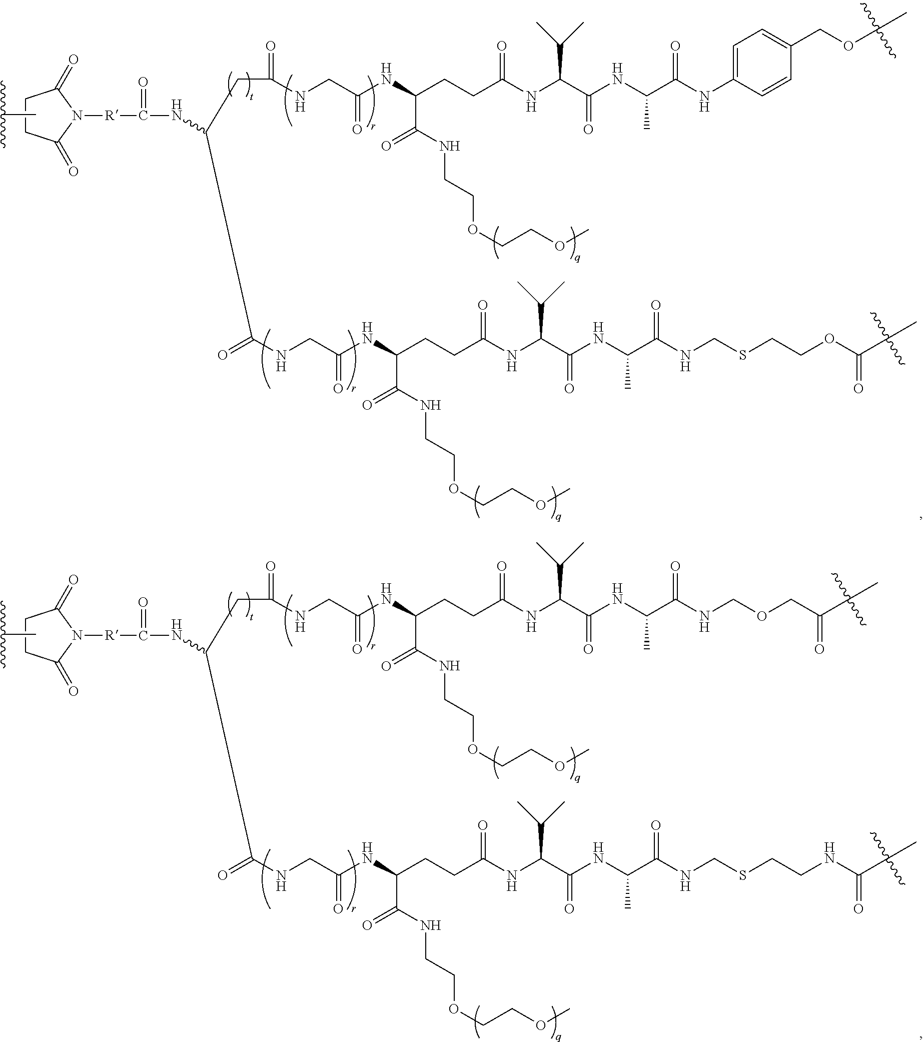

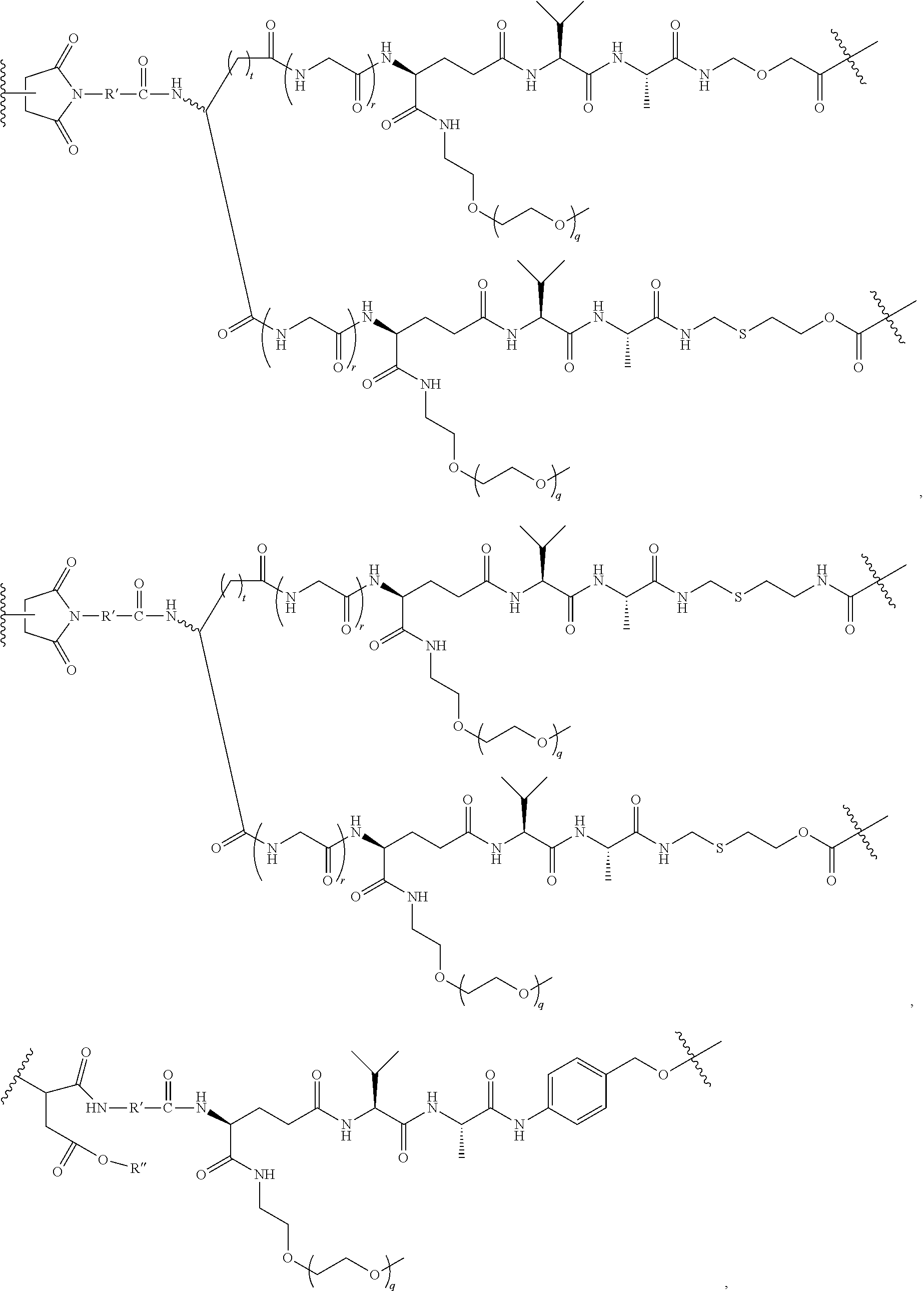

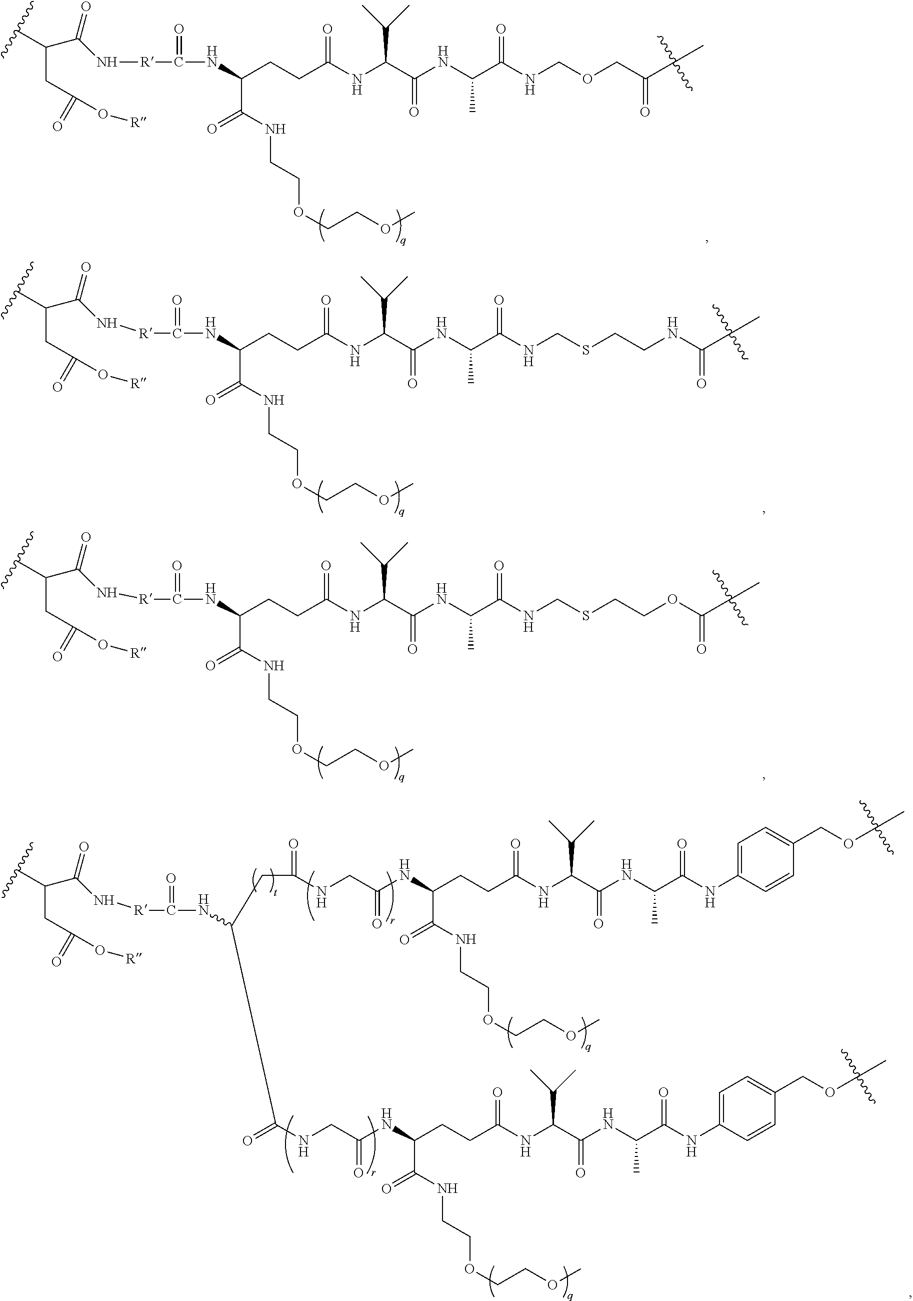

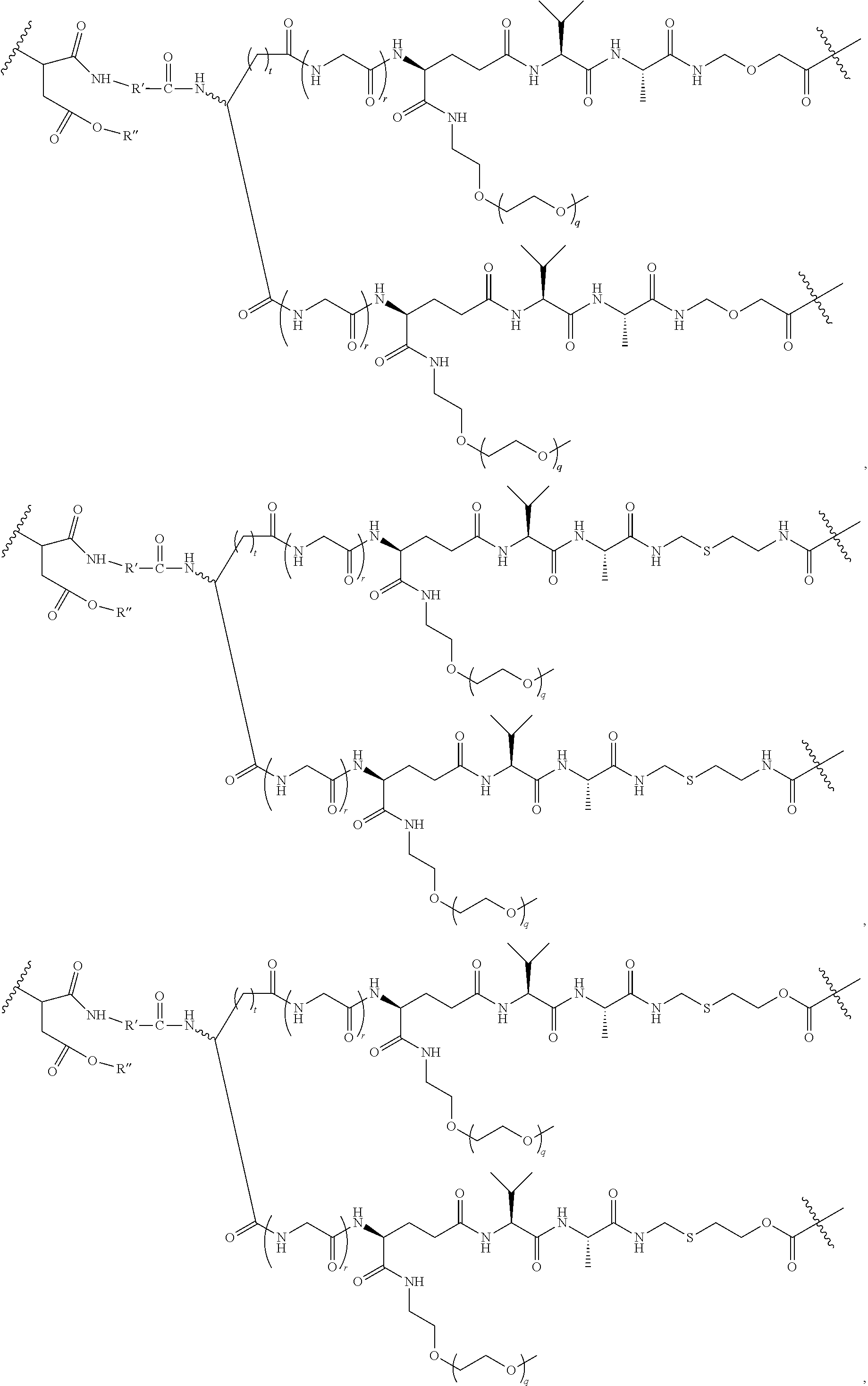

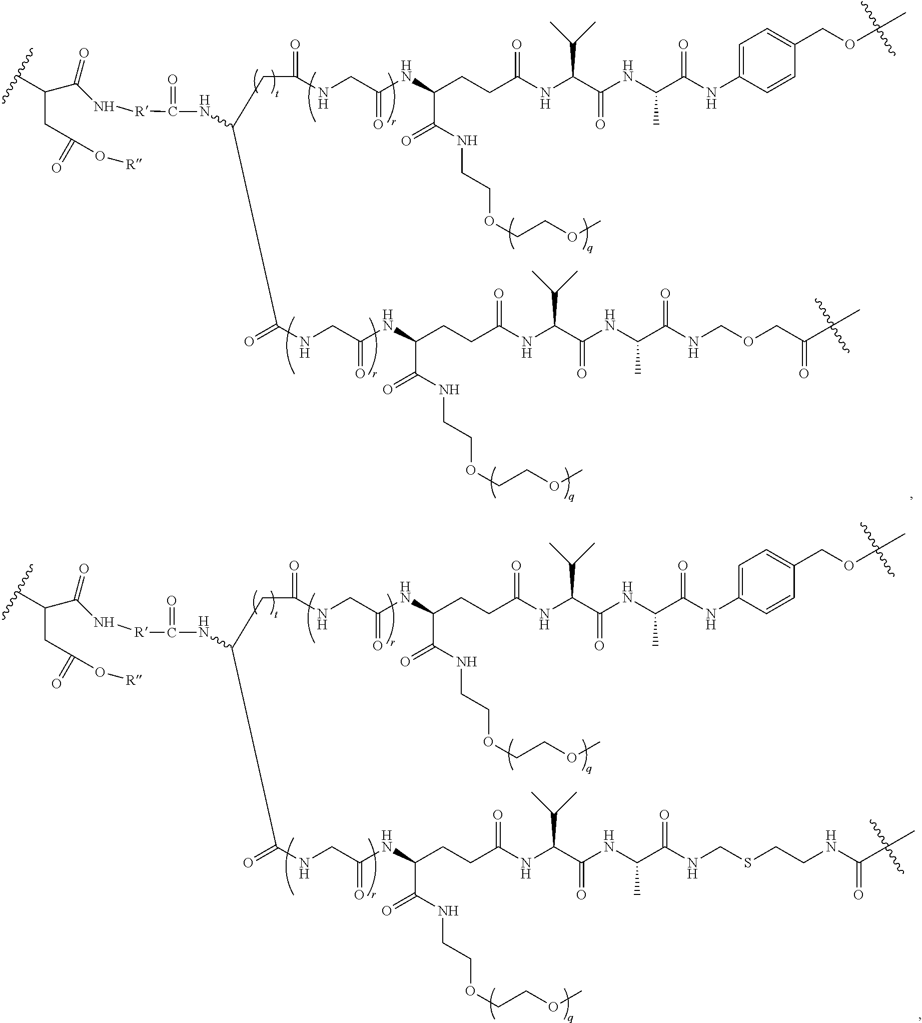

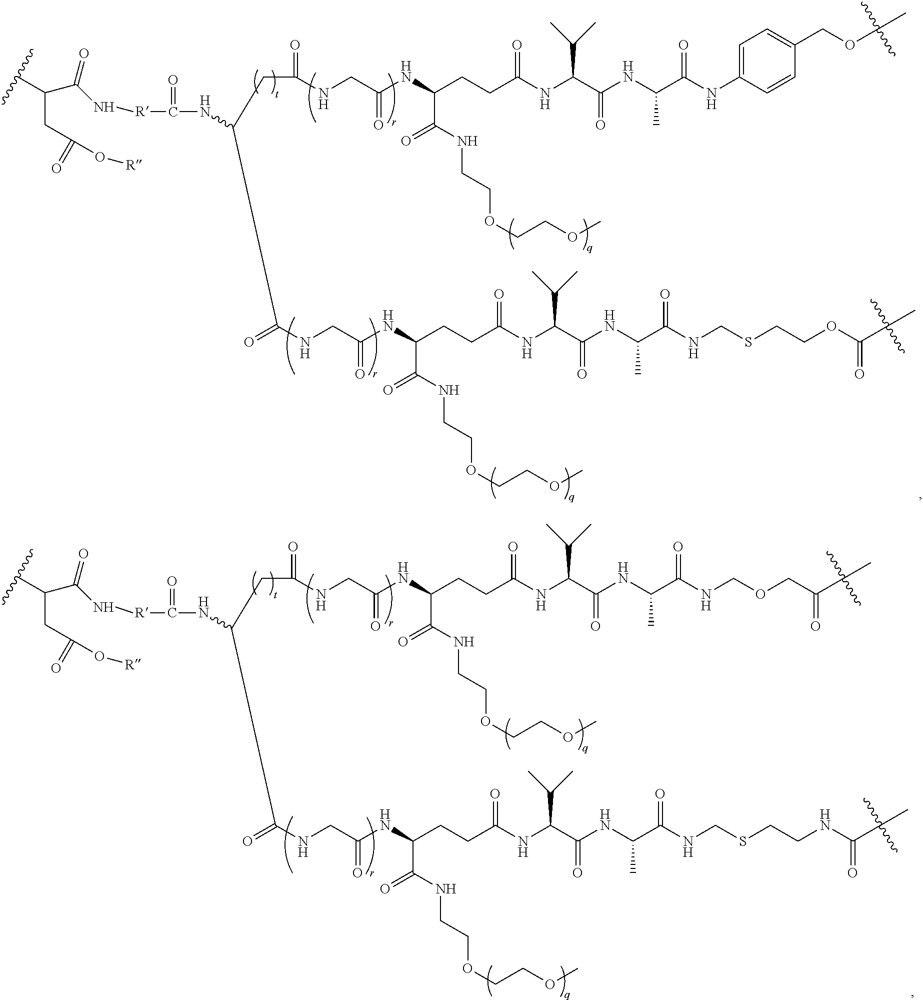

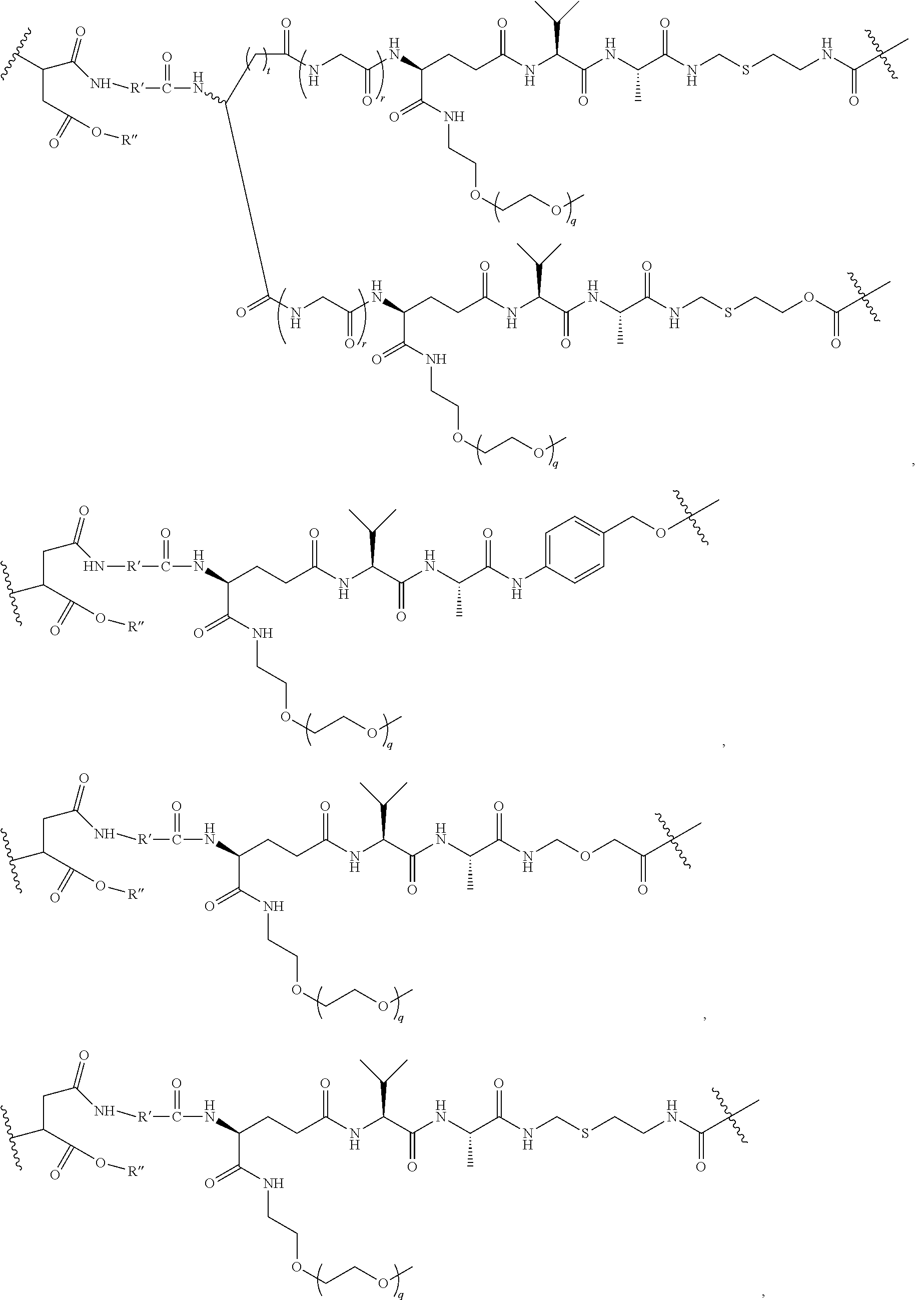

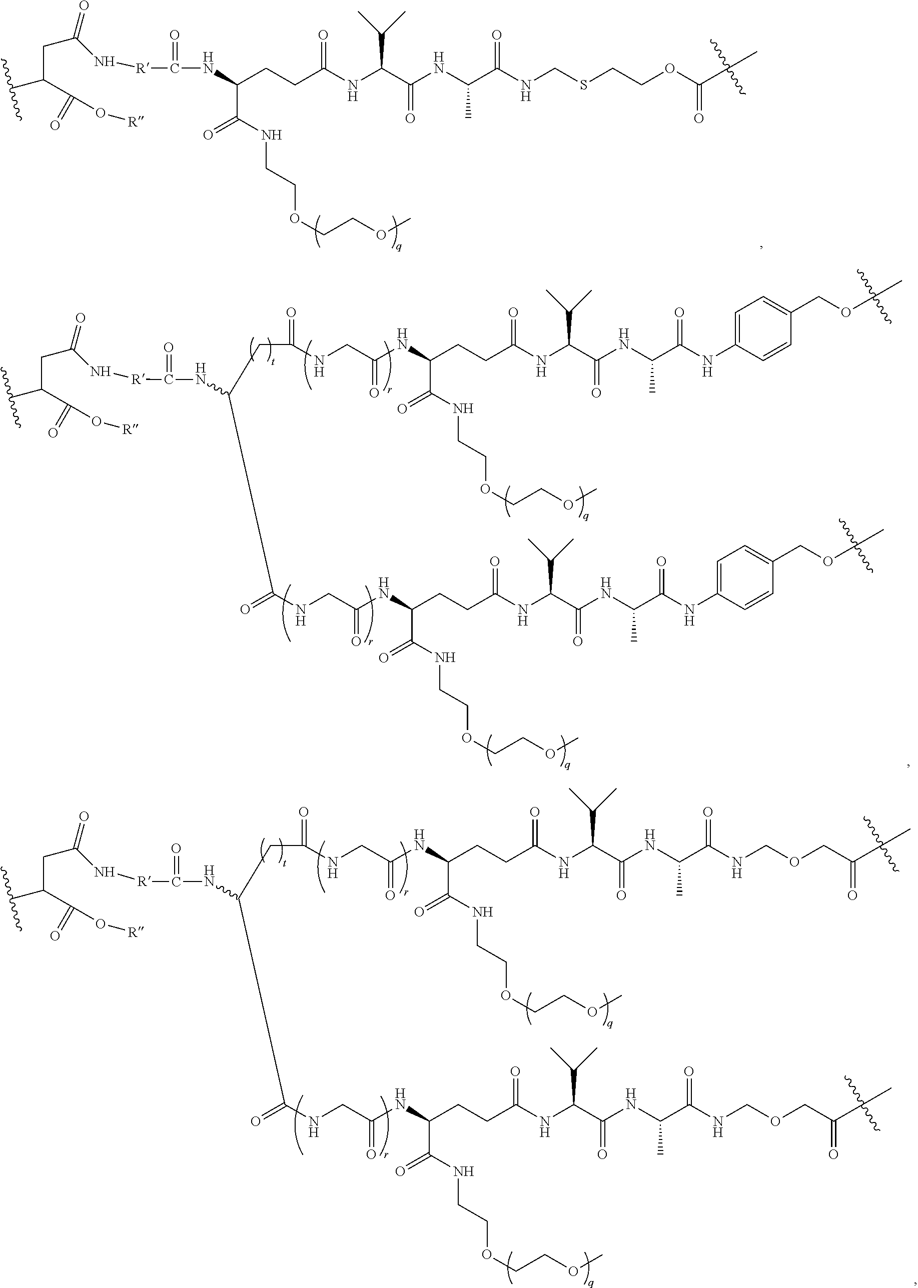

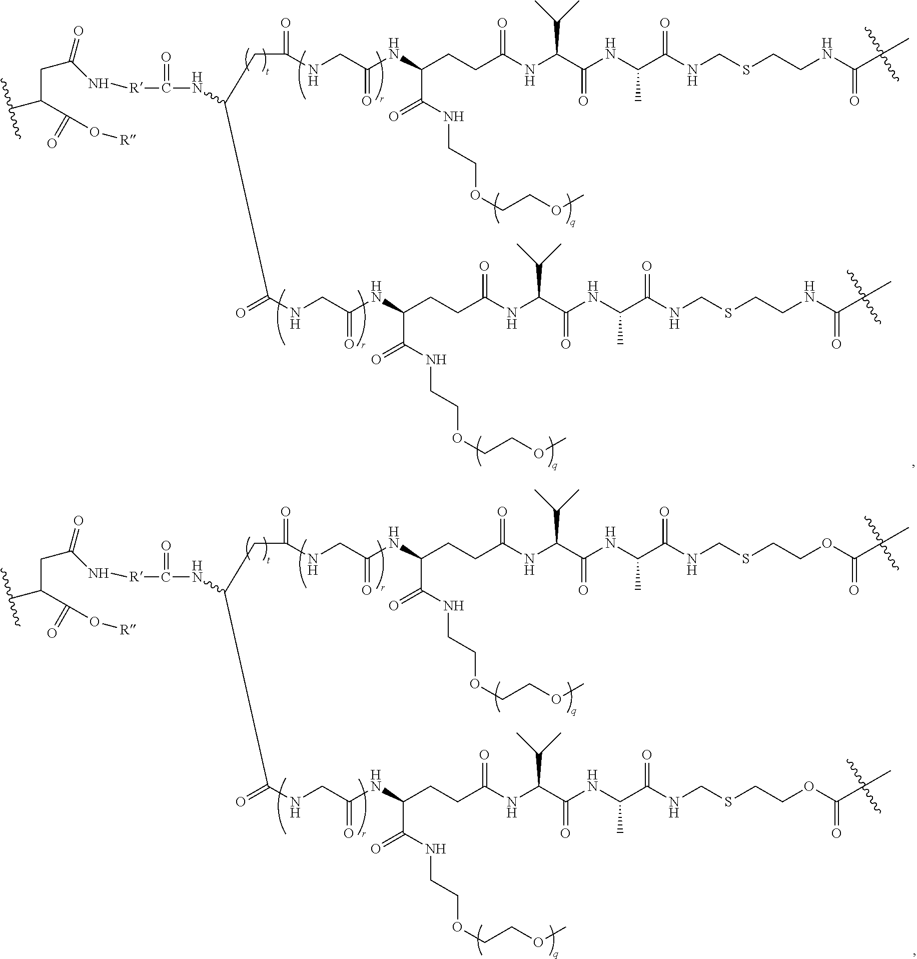

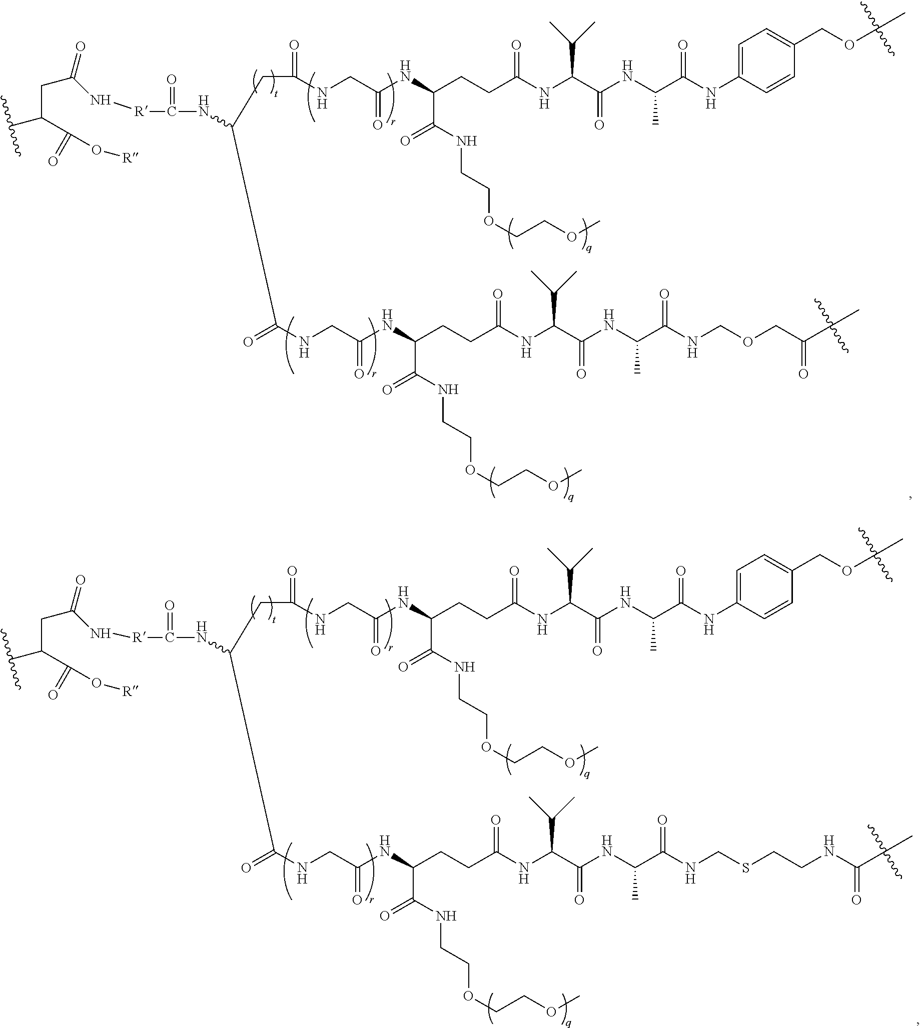

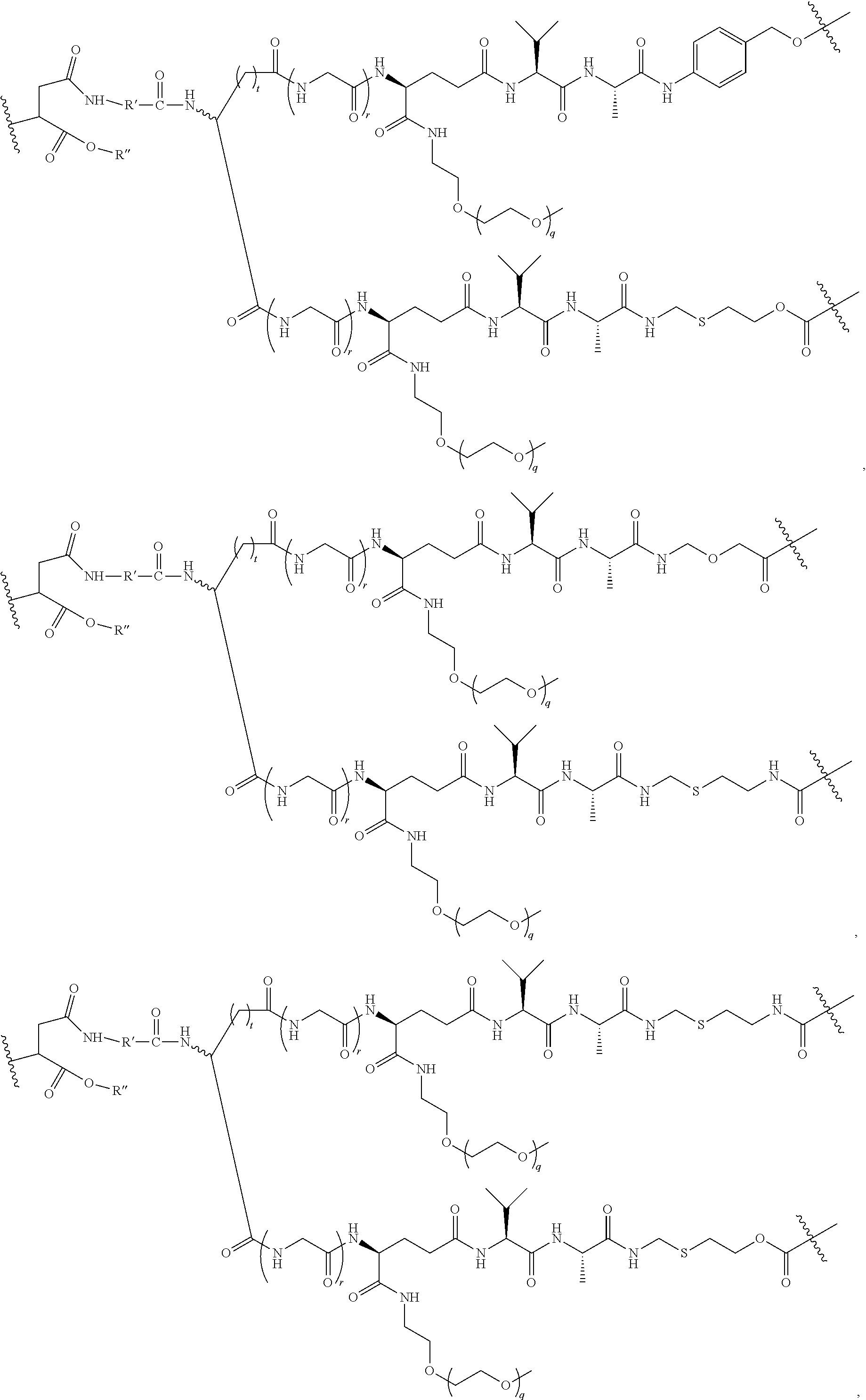

wherein,
R′ is selected from: optionally substituted C₁-C₁₀ alkyl, C₁-C₁₀ alkoxy, C₁-C₁₀ aminoalkyl and C₁-C₁₀ alkyl-aryl,

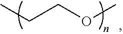

is optionally substituted C₁-C₁₀ alkyl; wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl; and wherein a subscript n is any integer selected from 1-10, preferably 1-5, and most preferably 1-3; and
each subscript r is any integer independently selected from 1-4, each subscript t is any integer independently selected from 1-4, and each subscript q is any integer independently selected from 1-20, preferably 3-10, and most preferably 5-8.

In some embodiments, the linker is selected from the following structures:

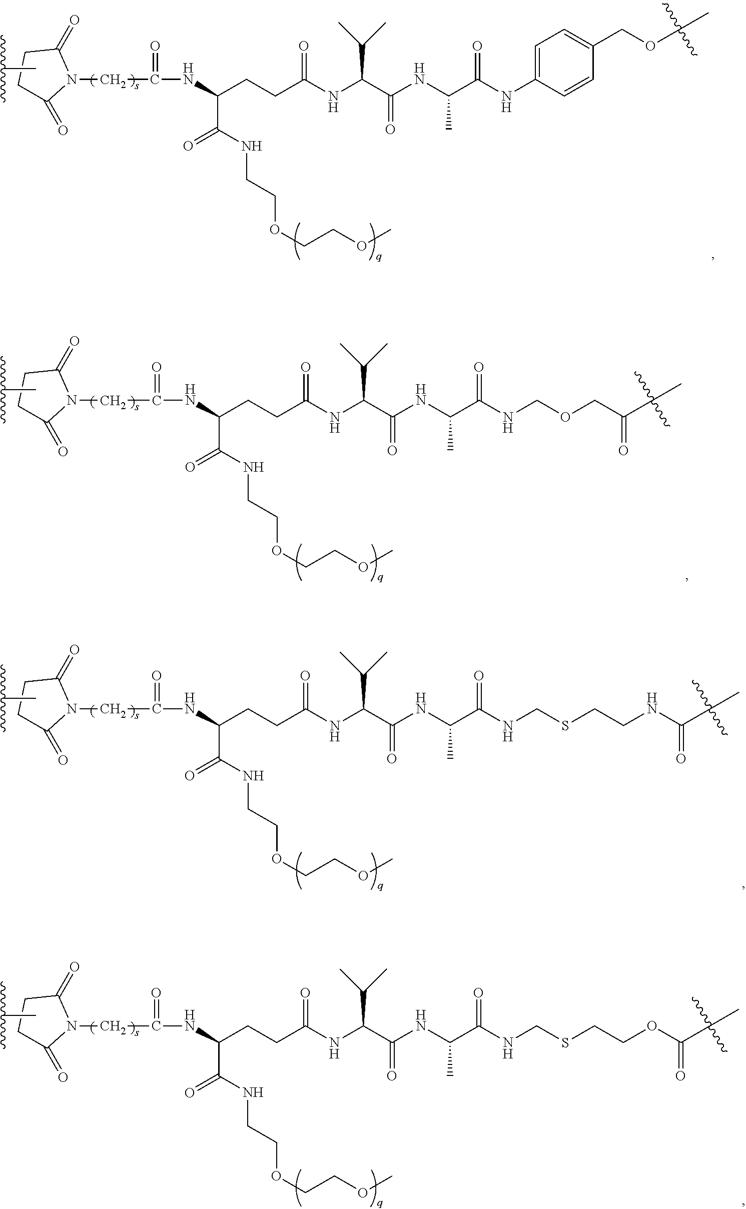

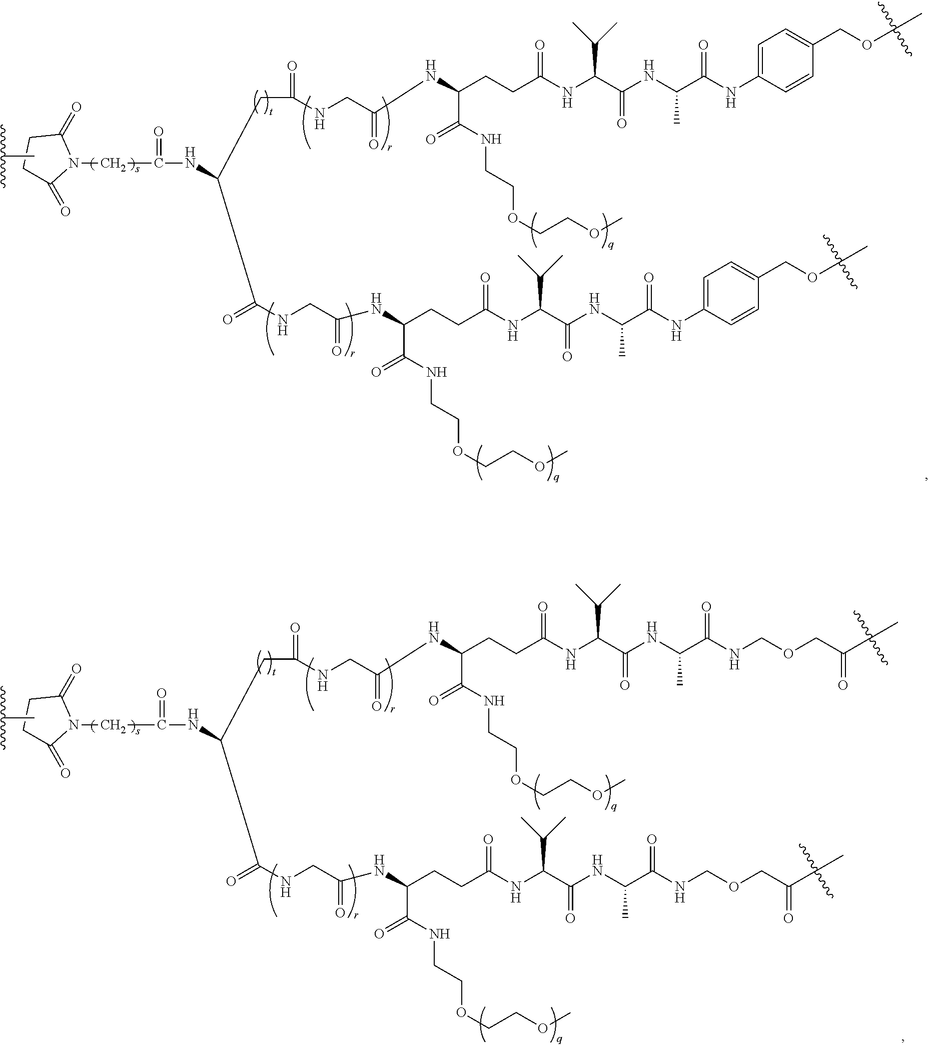

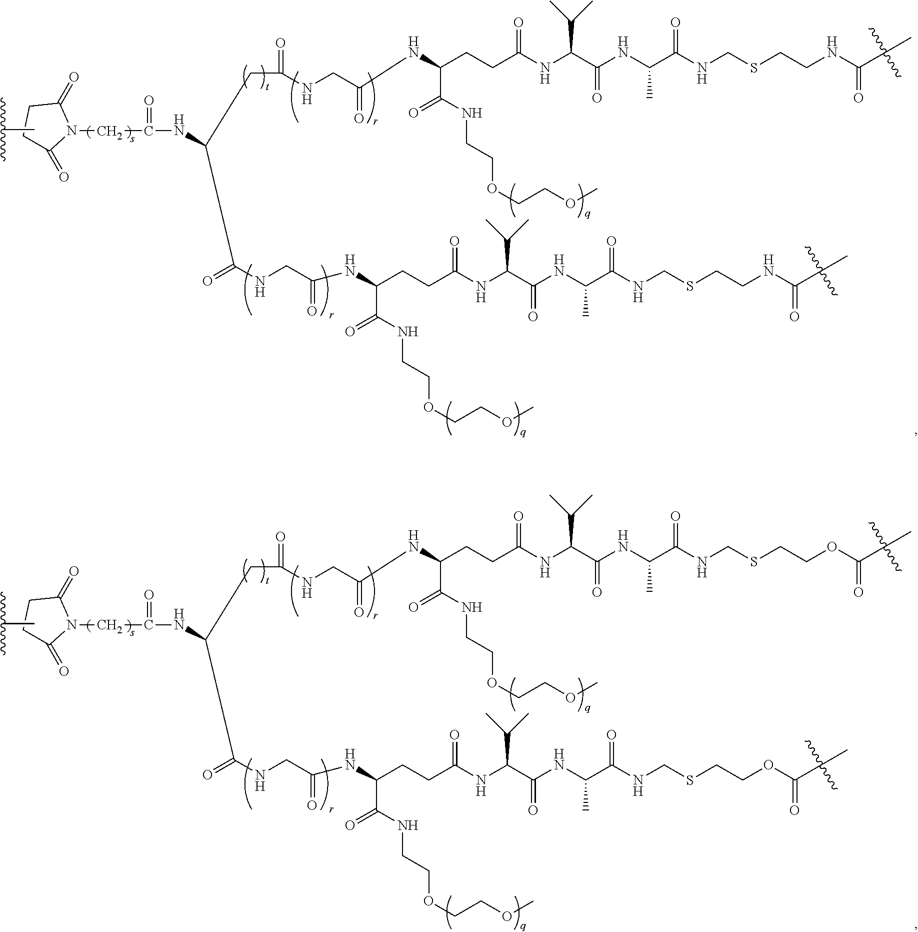

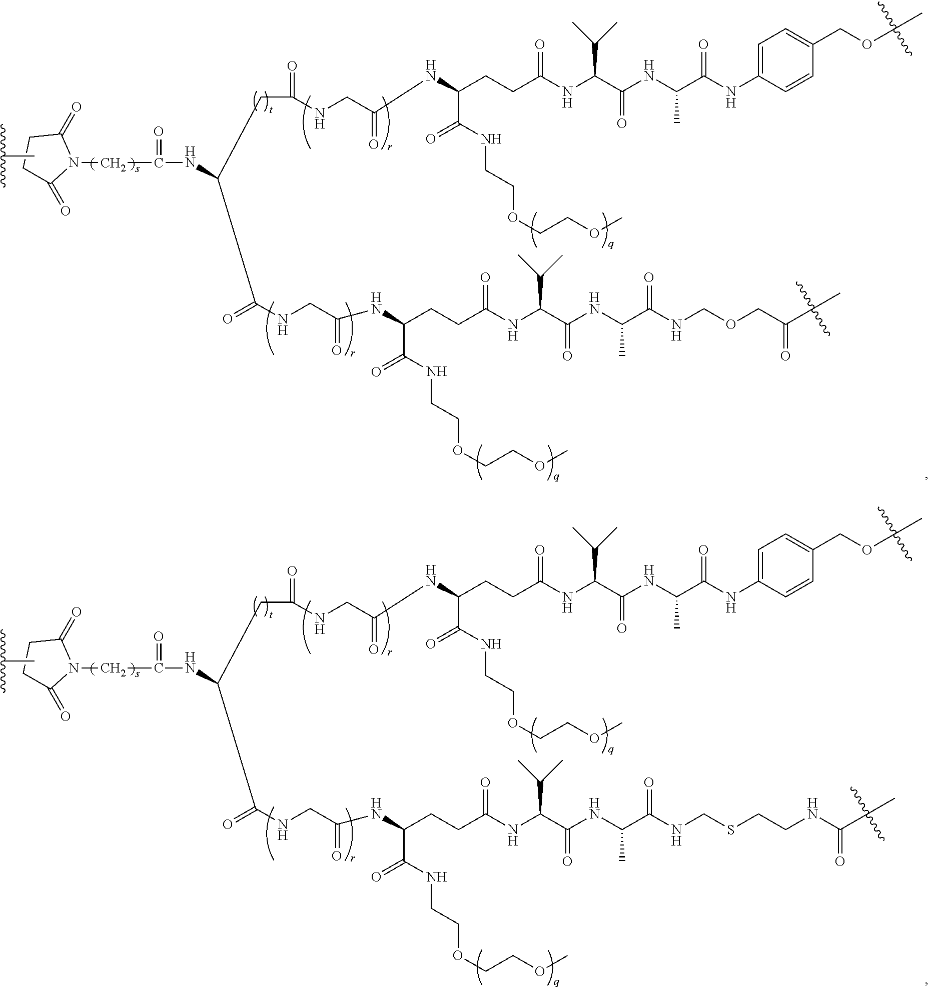

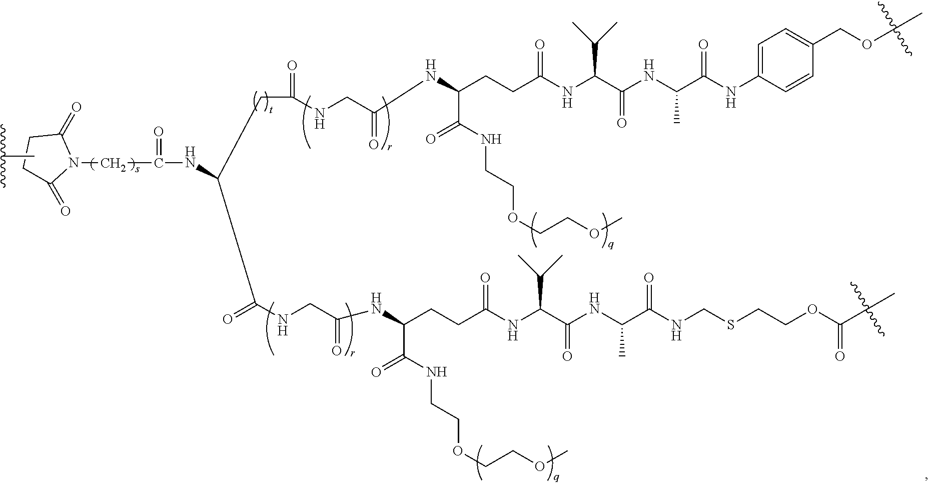

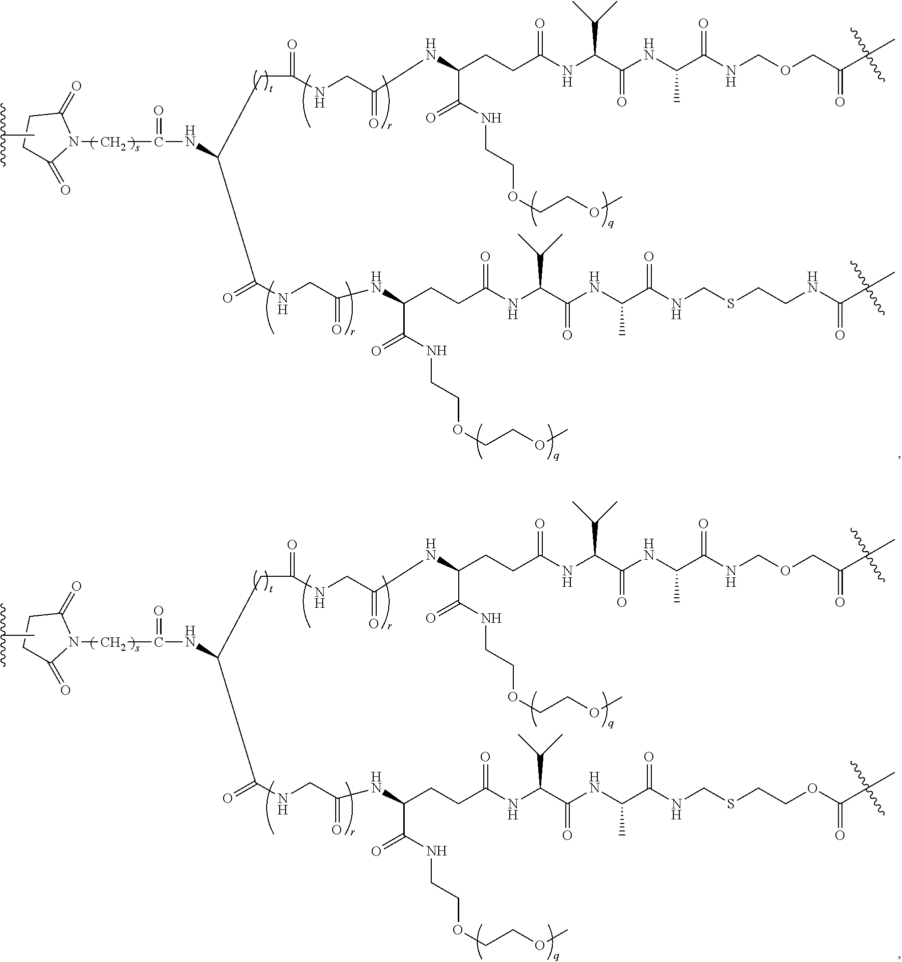

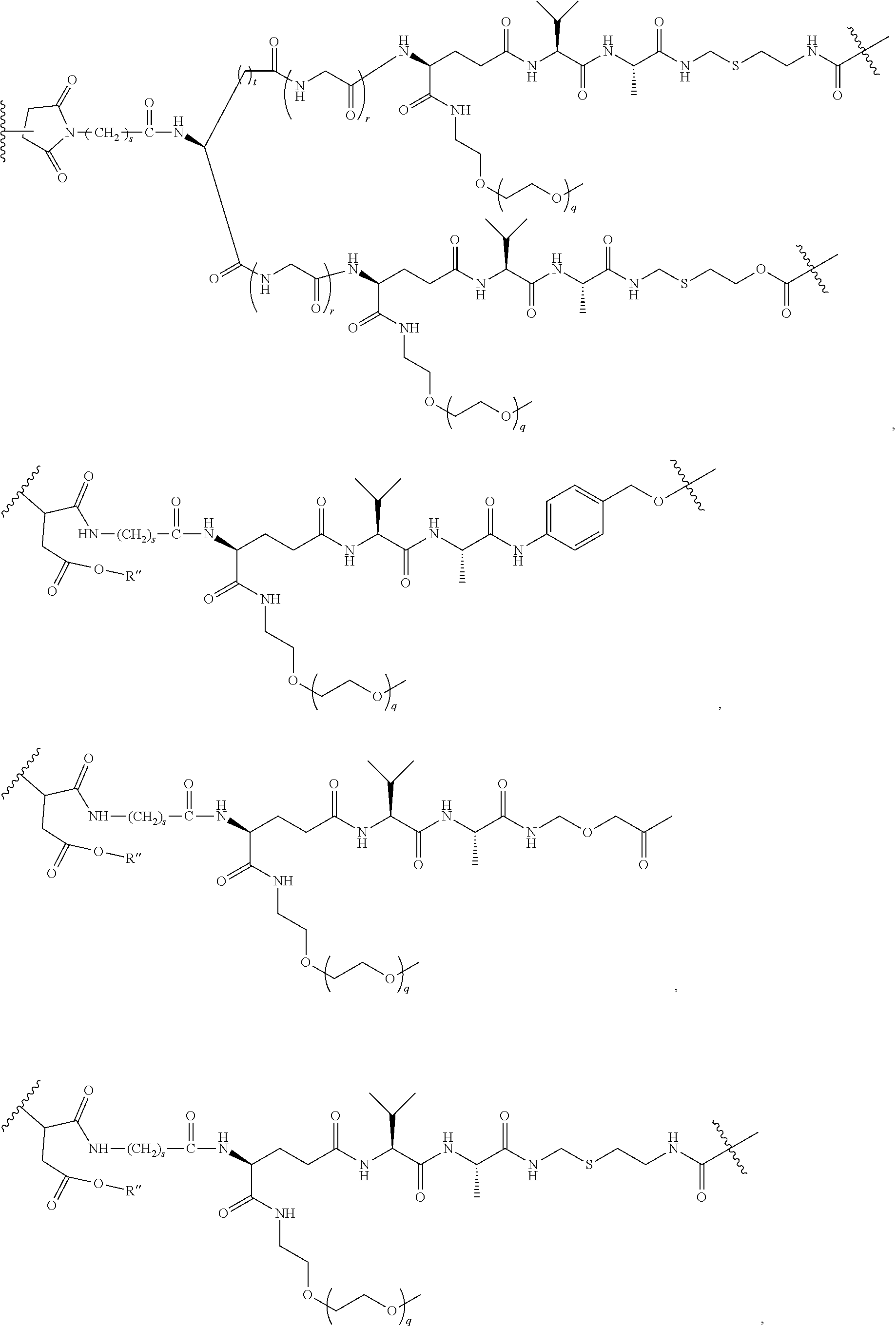

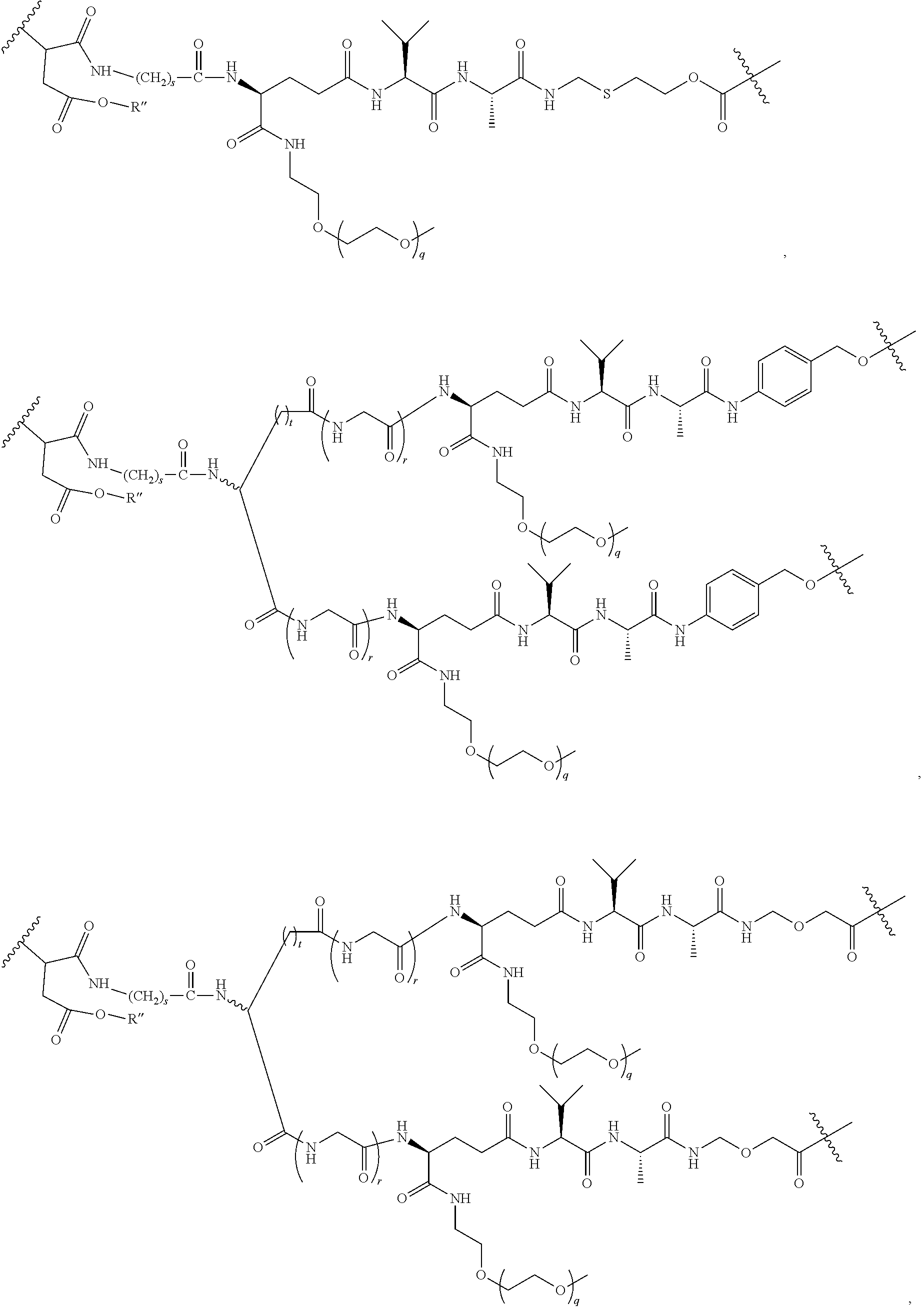

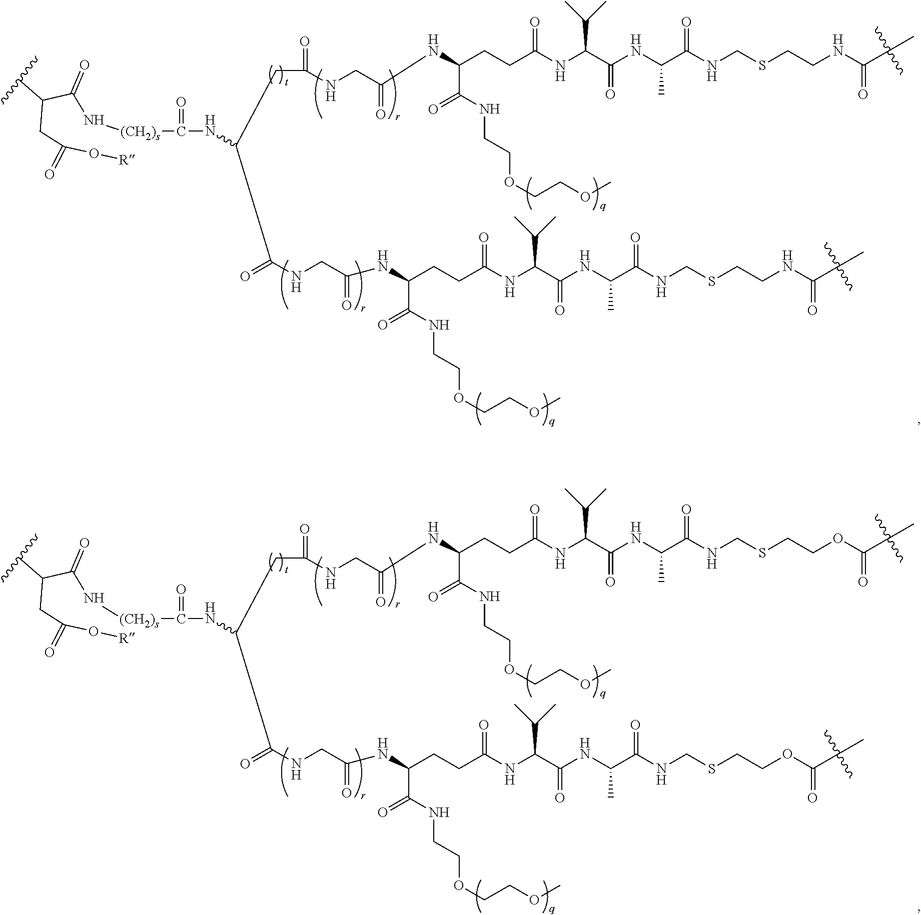

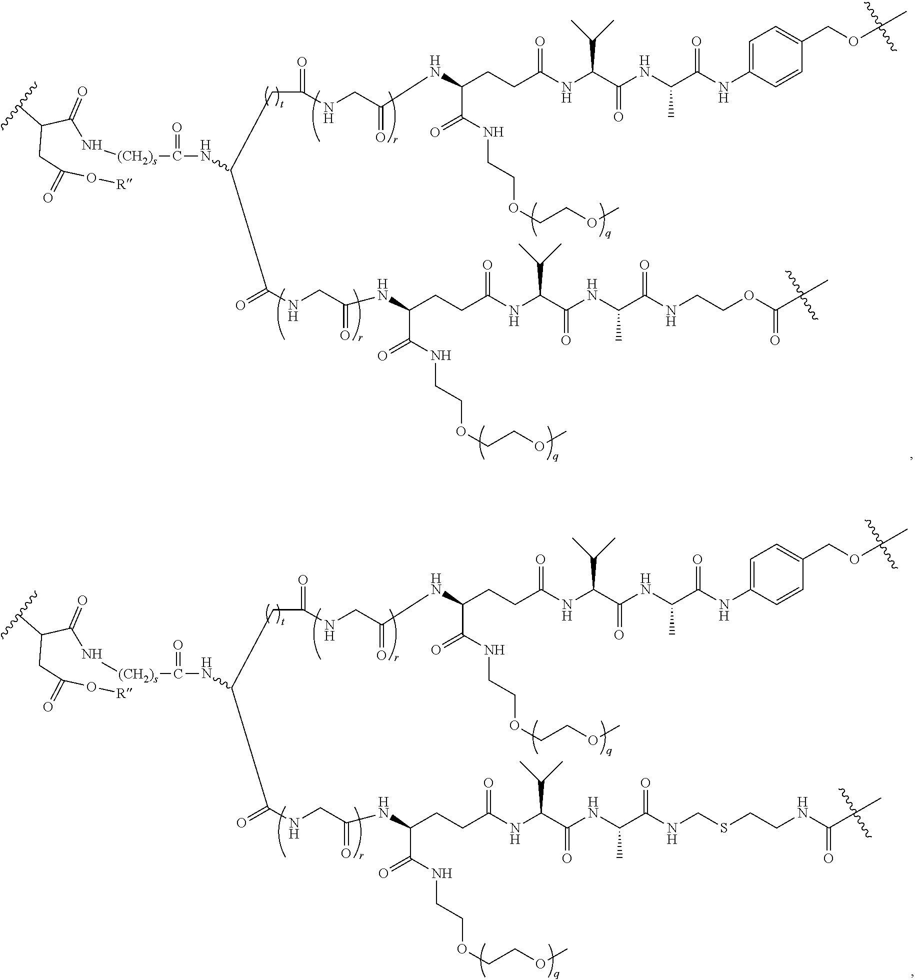

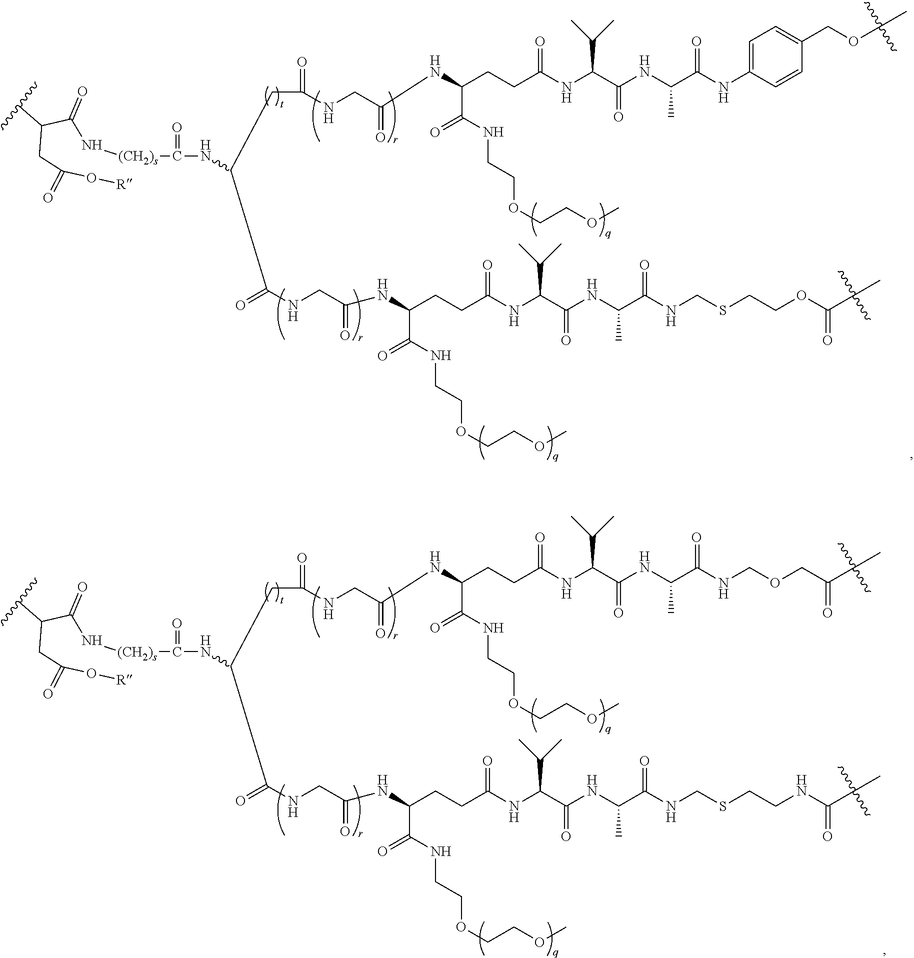

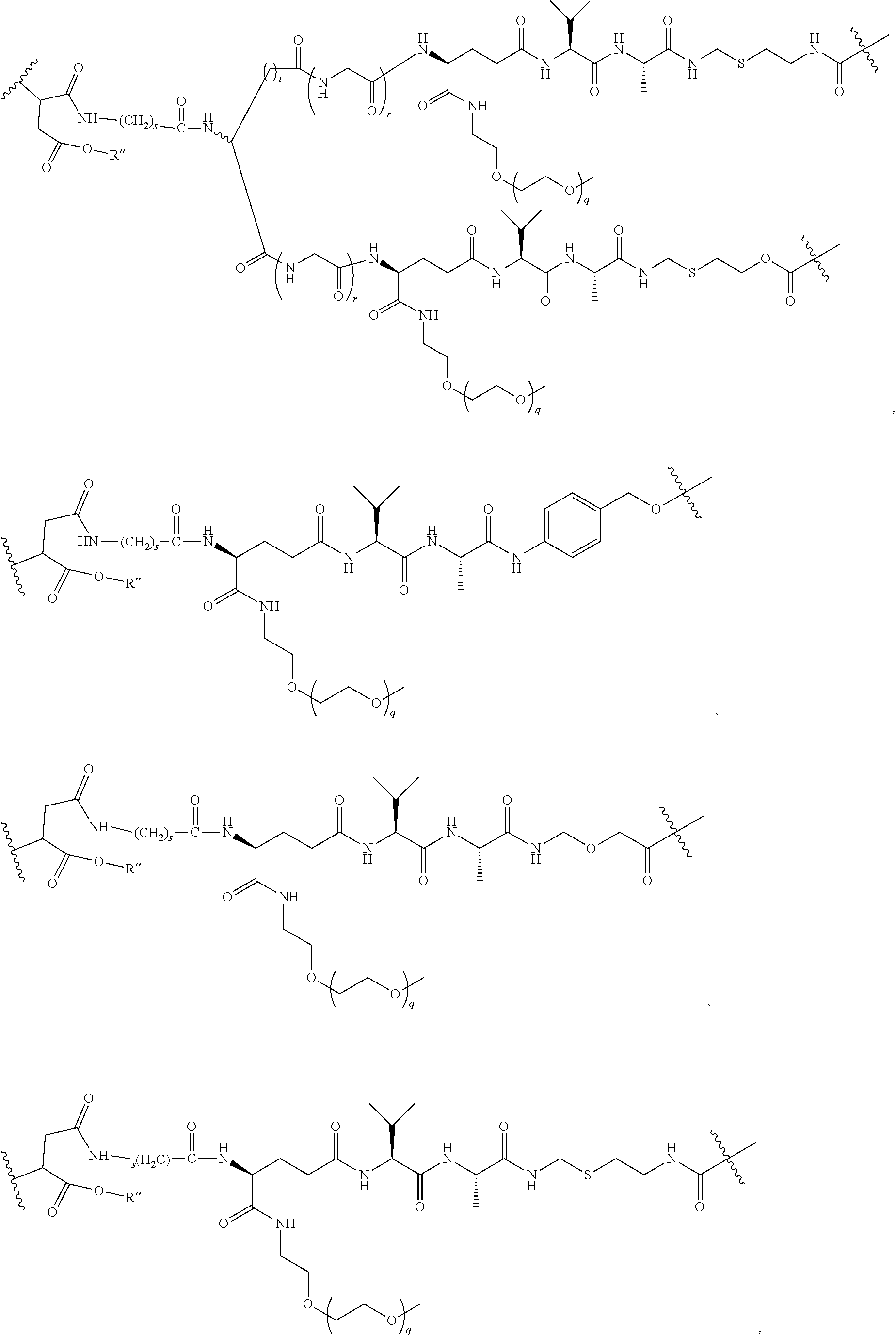

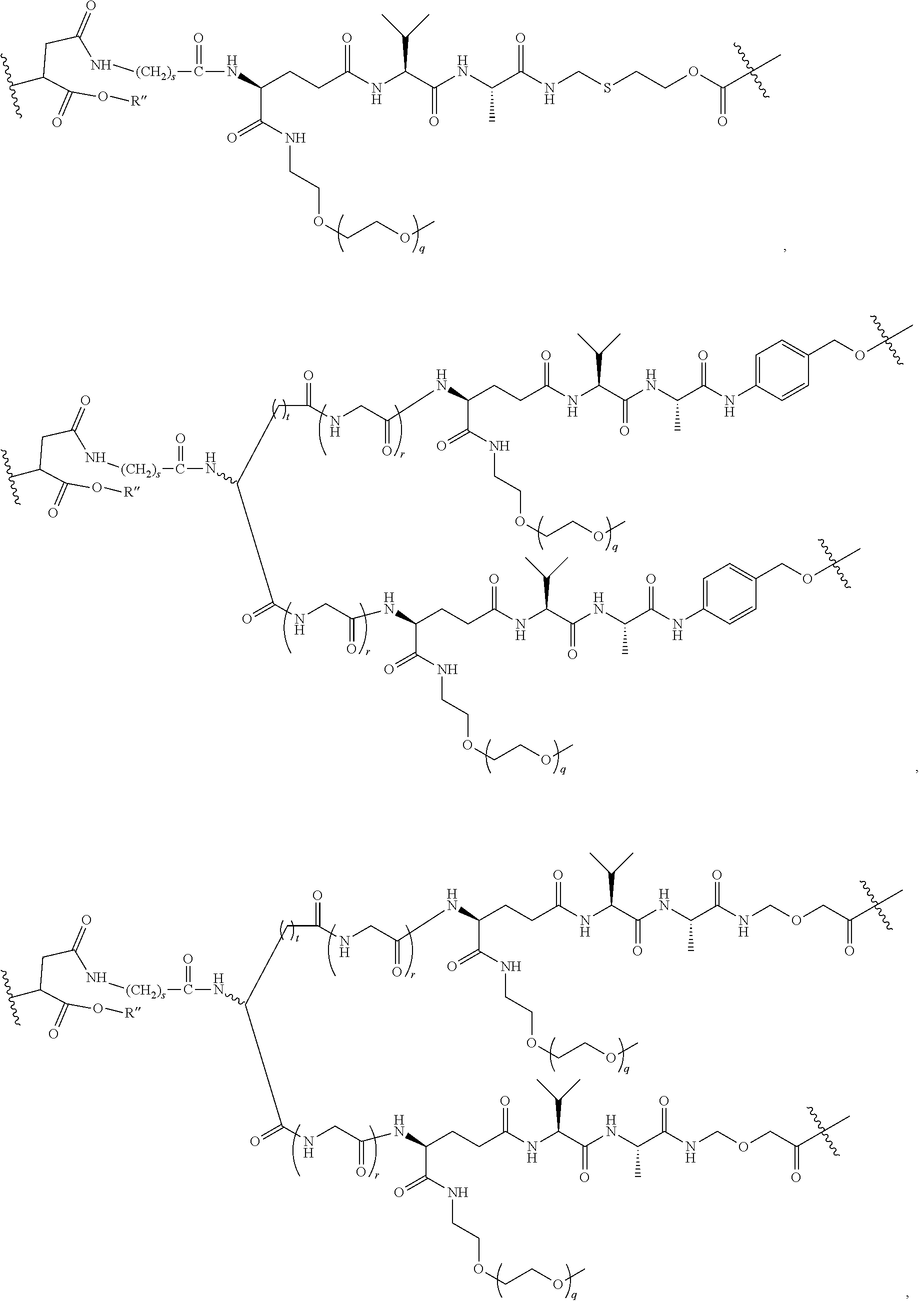

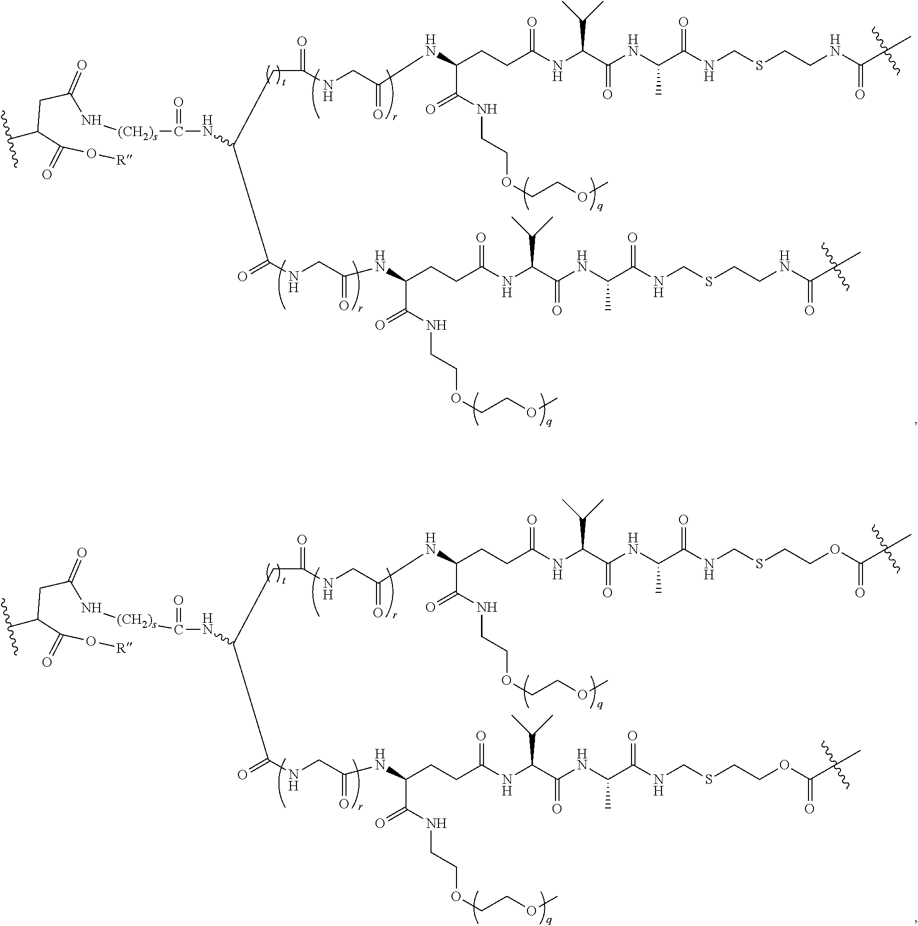

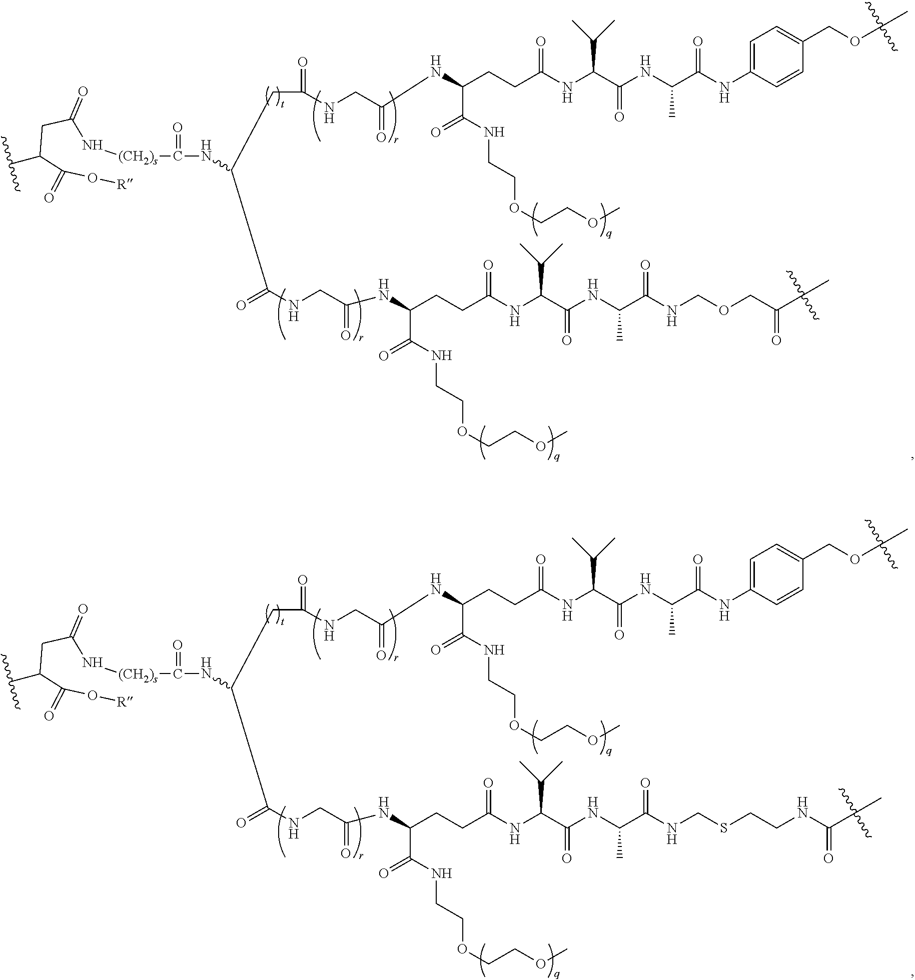

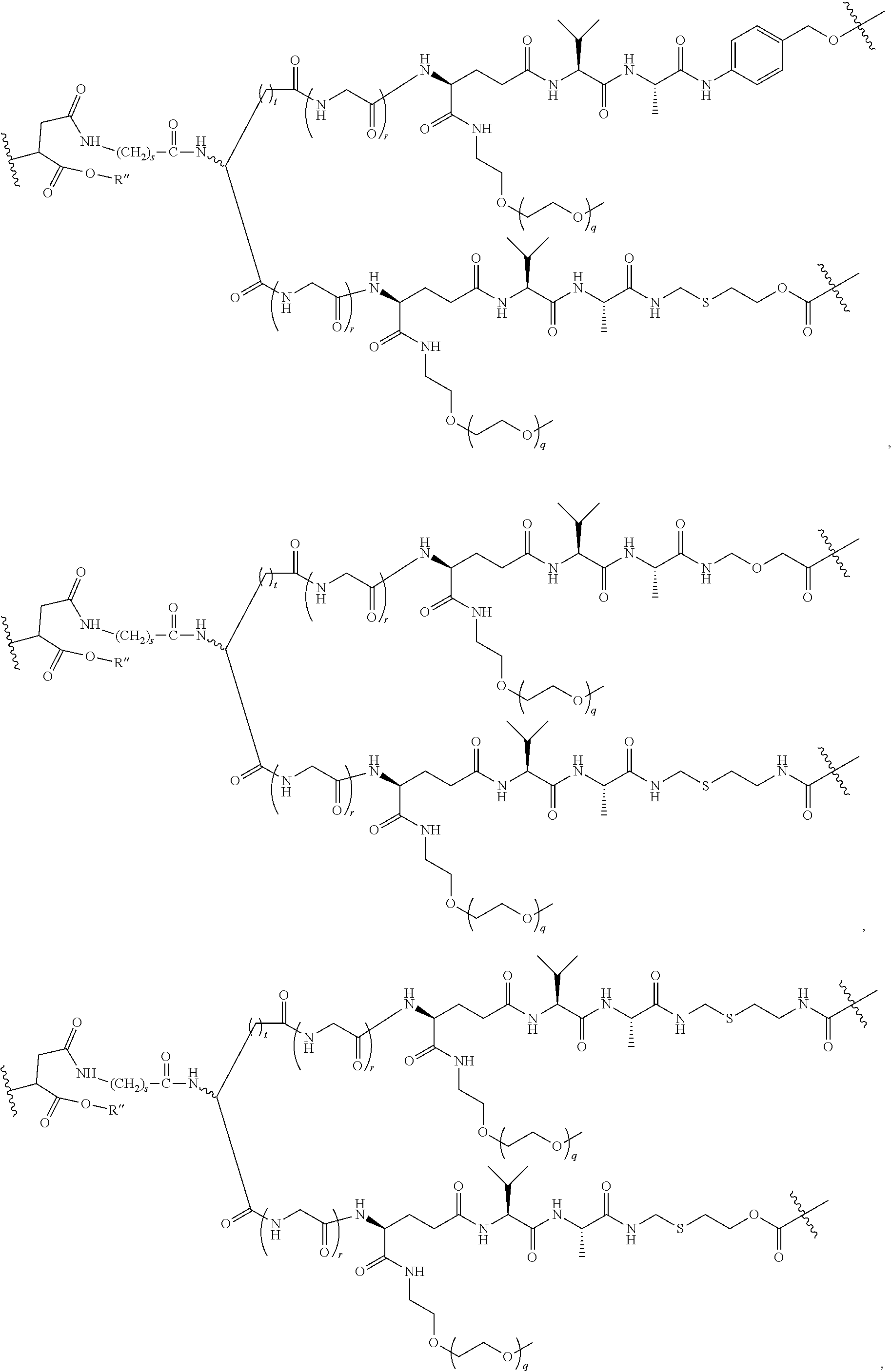

wherein R″ is selected from the group consisting of methyl, ethyl and isopropyl, each subscript s is any integer independently selected from 1-10, each subscript r is any integer independently selected from 1-4, each subscript t is any integer independently selected from 1-4, and each subscript q is any integer independently selected from 1-20, preferably 3-10, and most preferably 5-8.

In some embodiments, the linker is selected from the following structures:

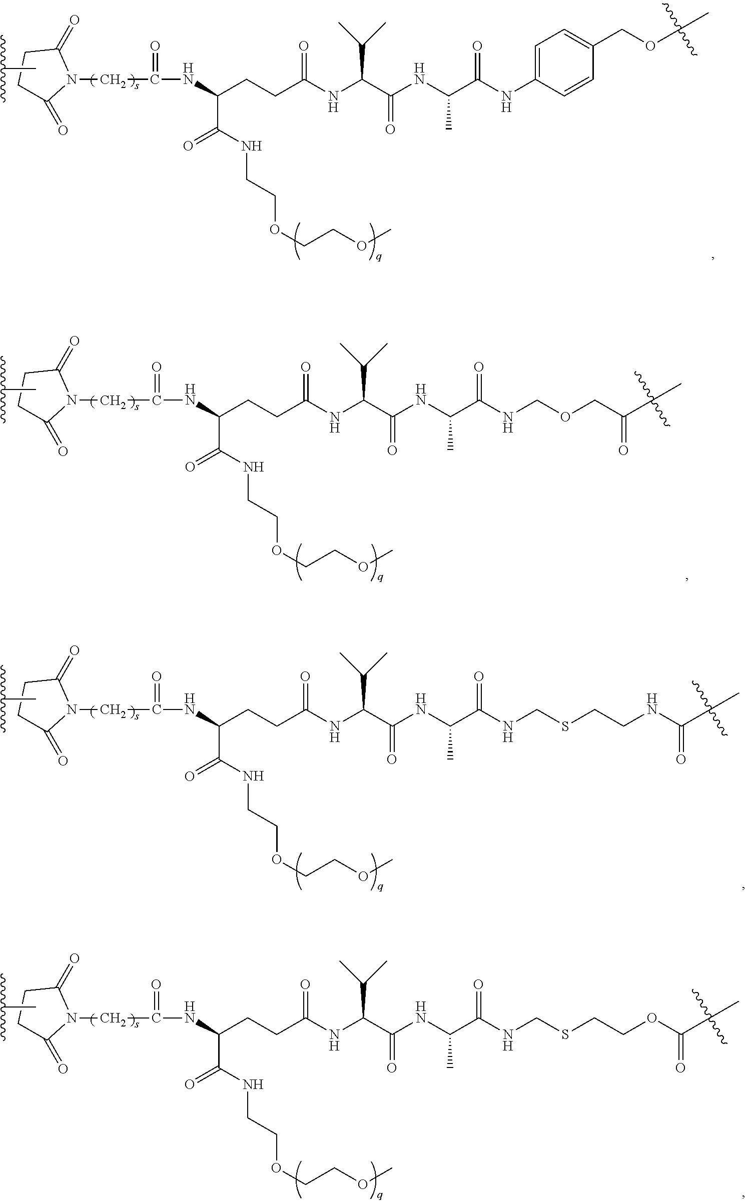

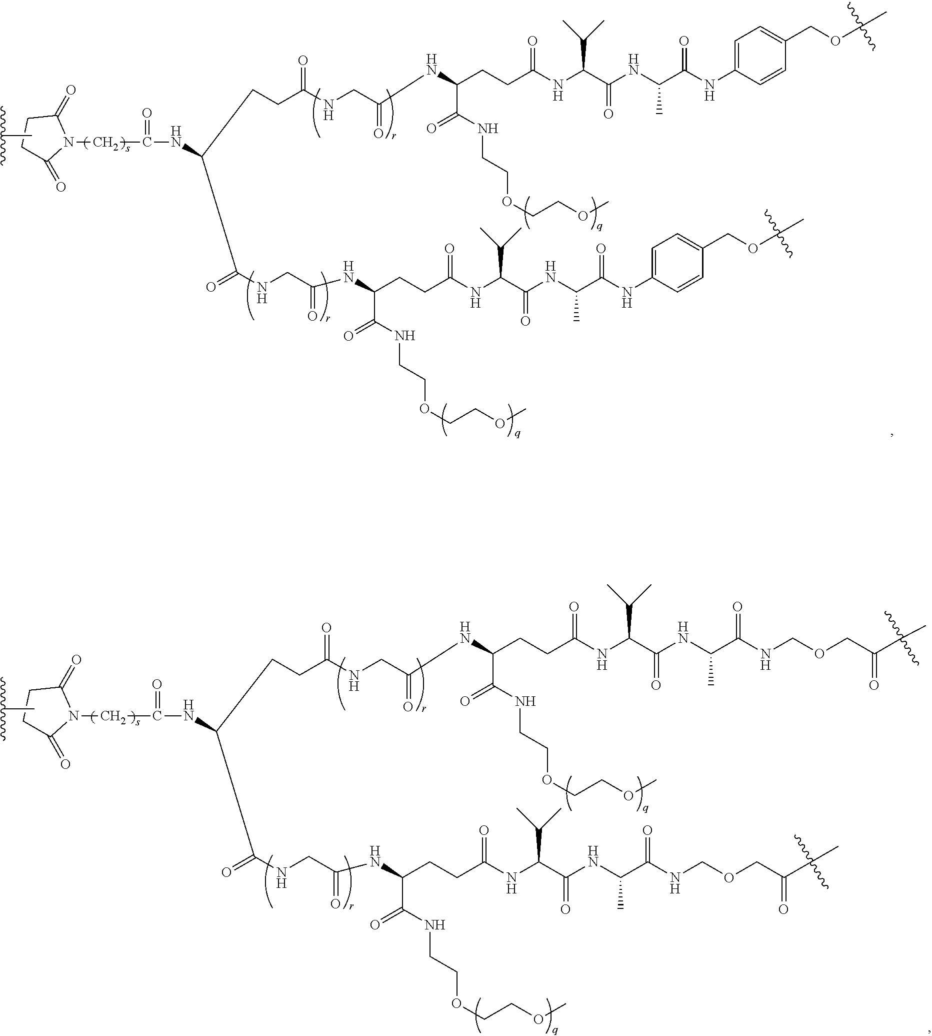

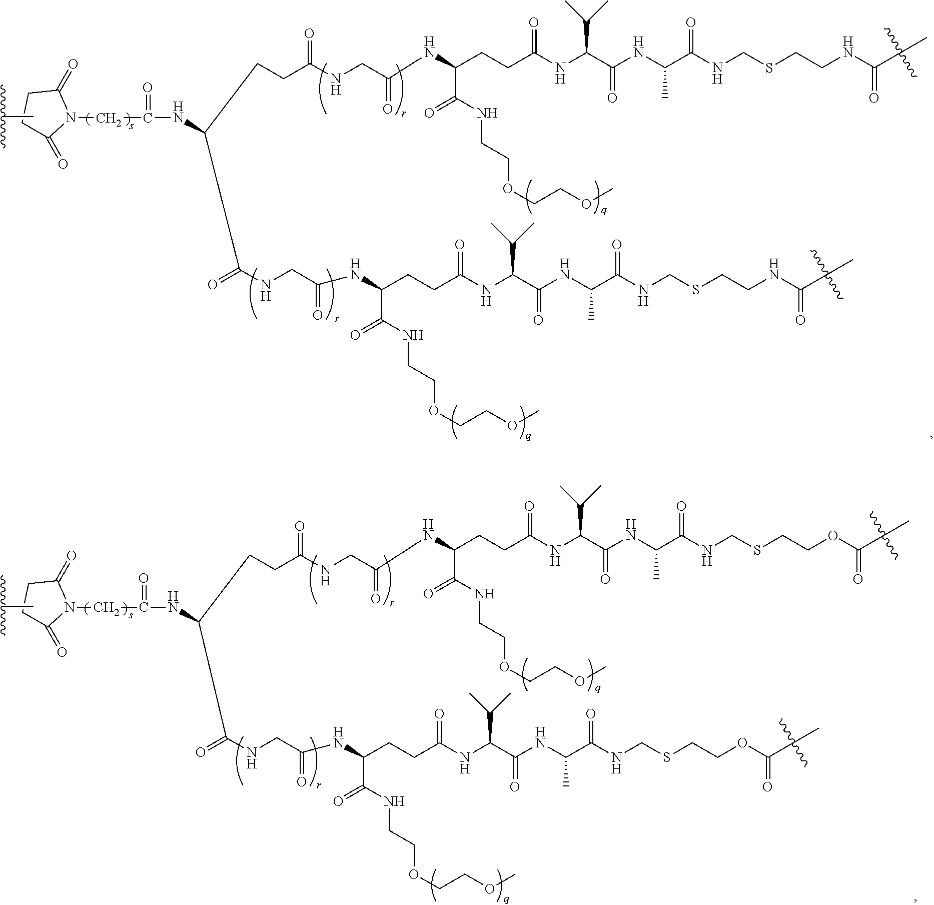

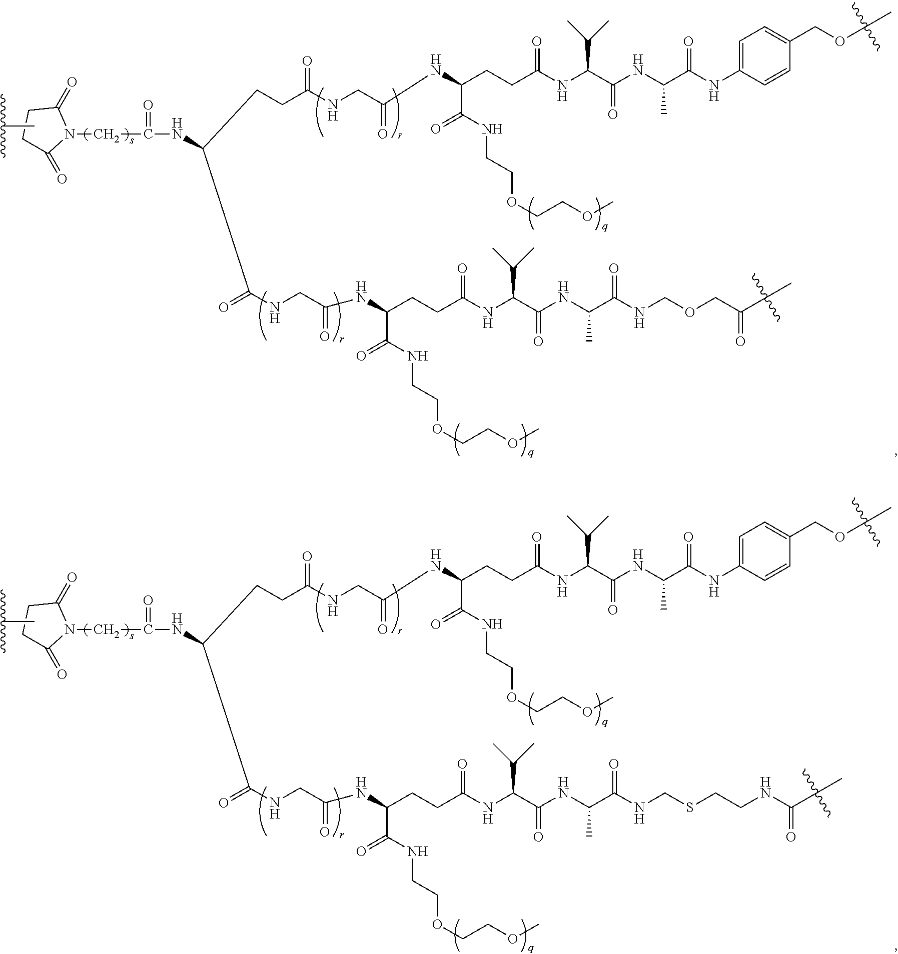

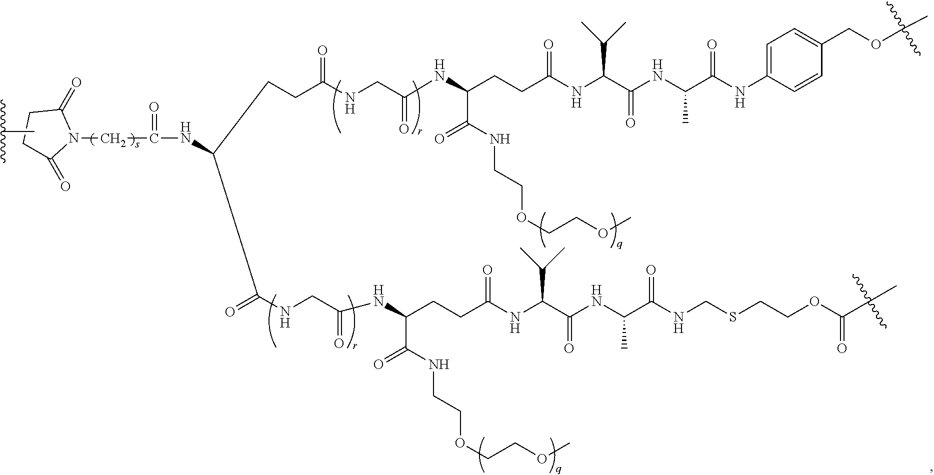

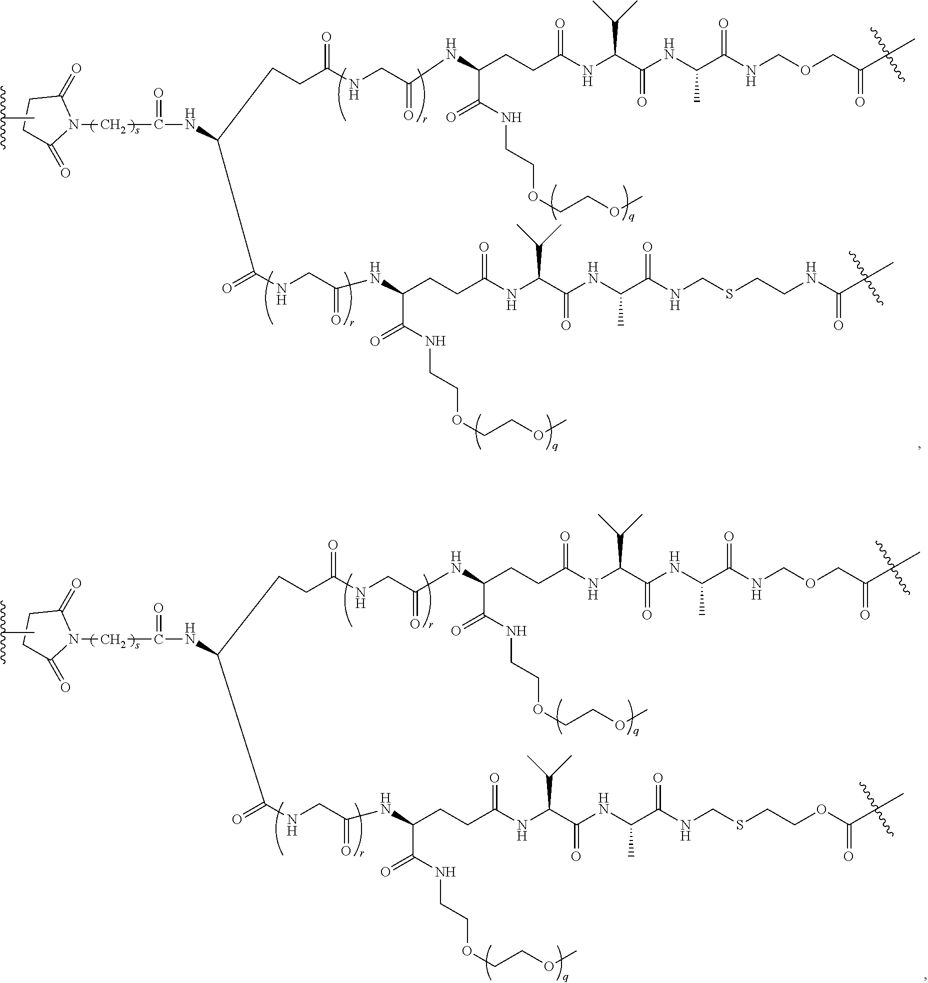

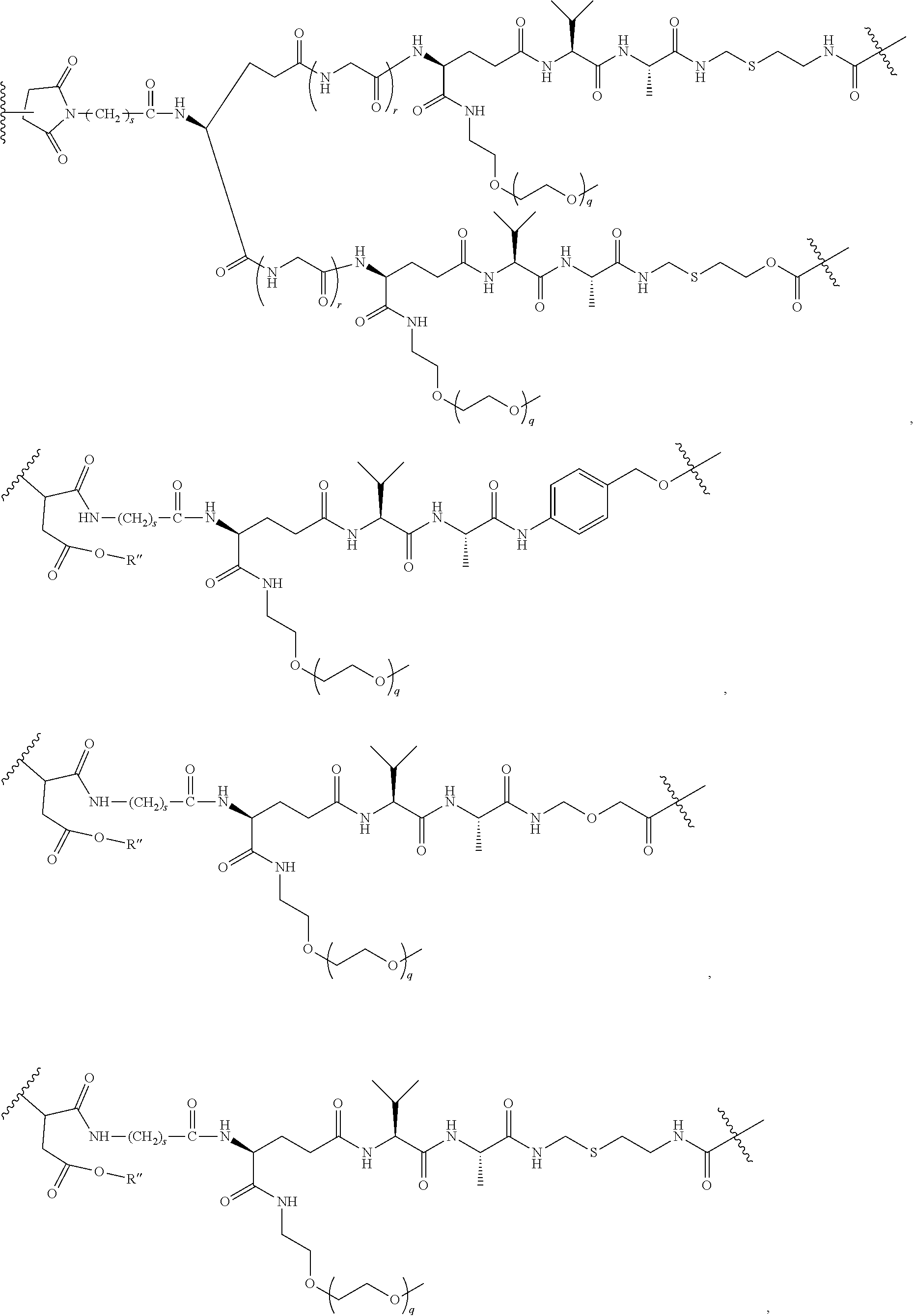

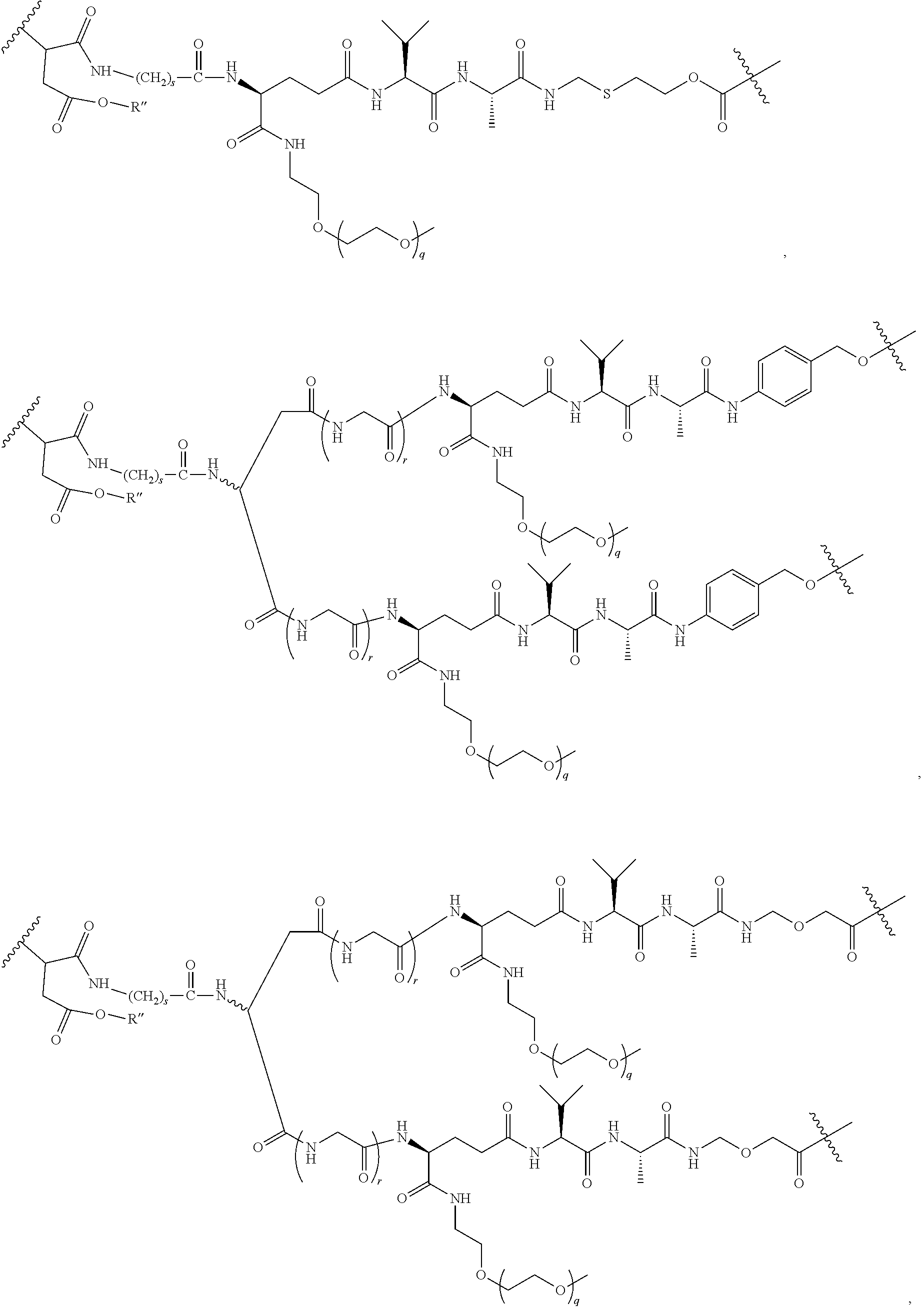

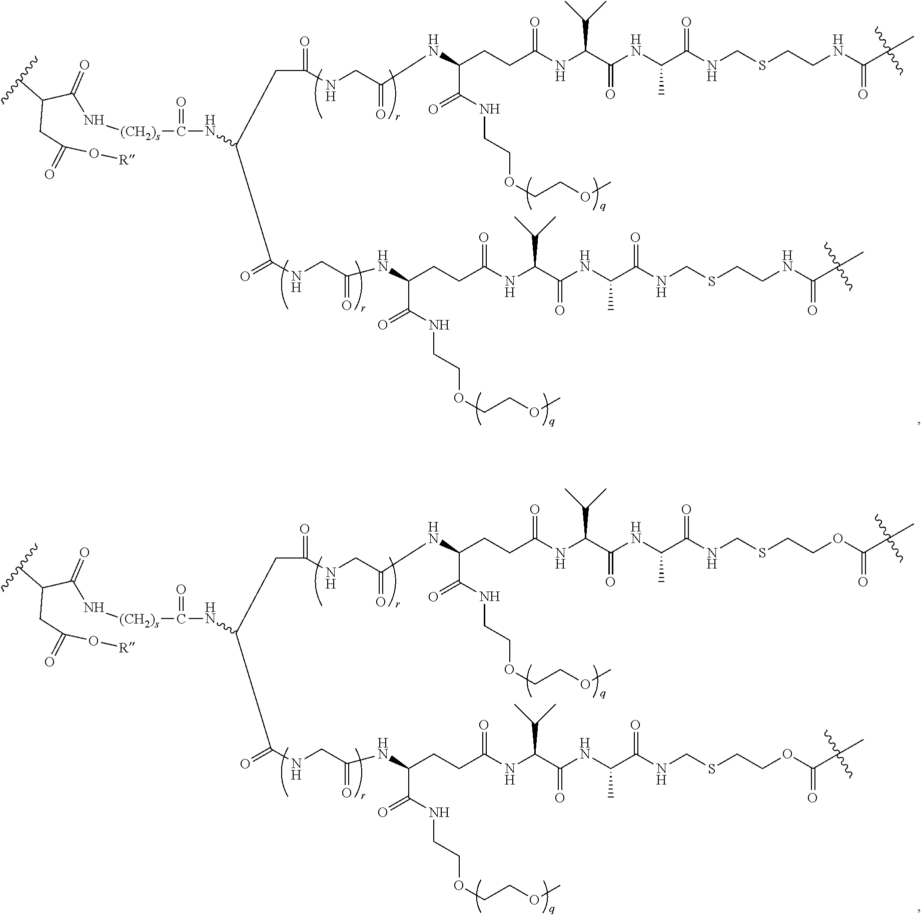

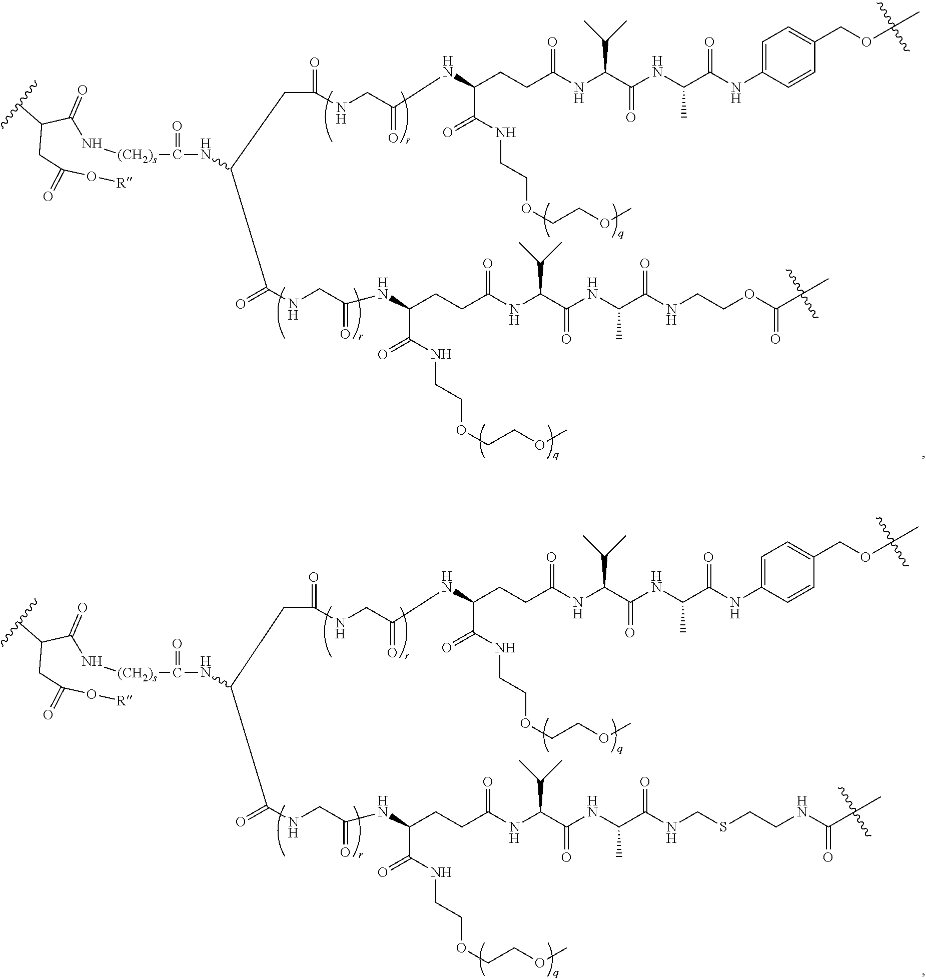

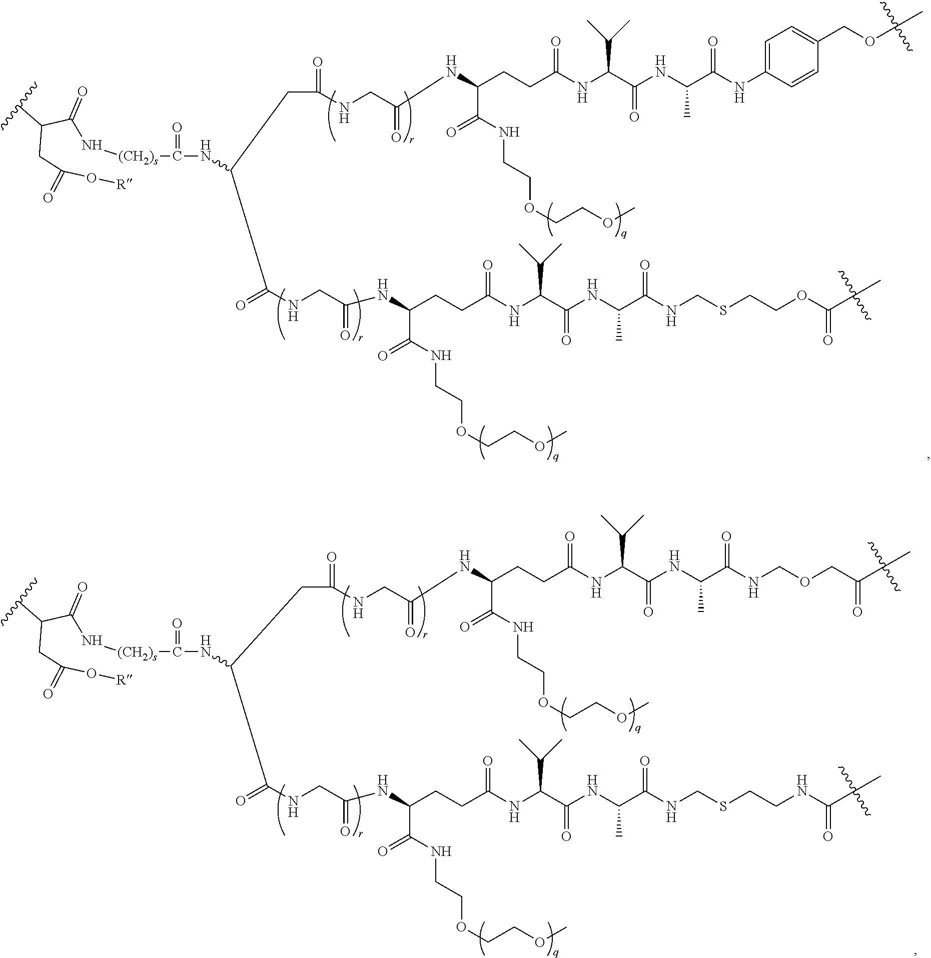

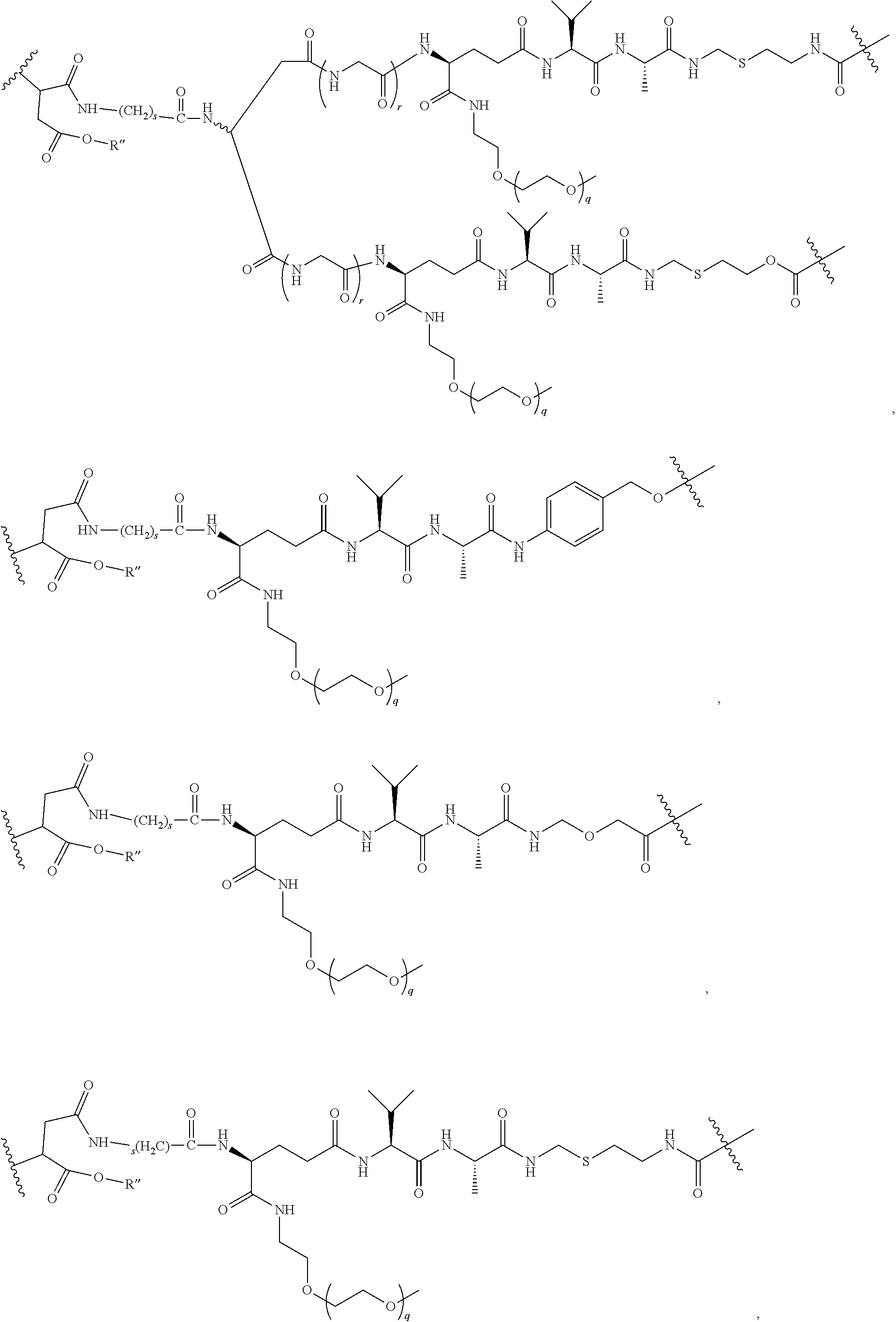

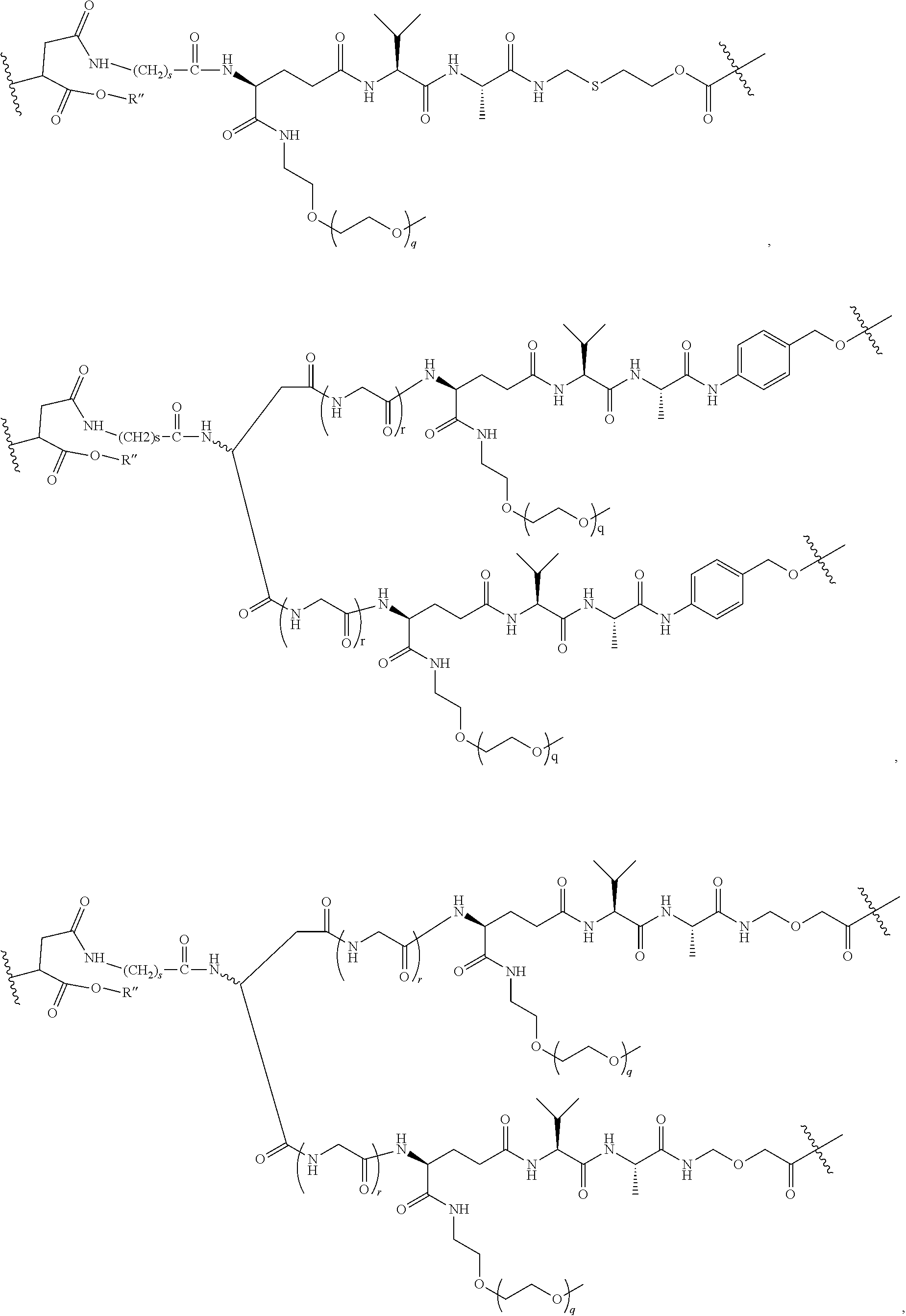

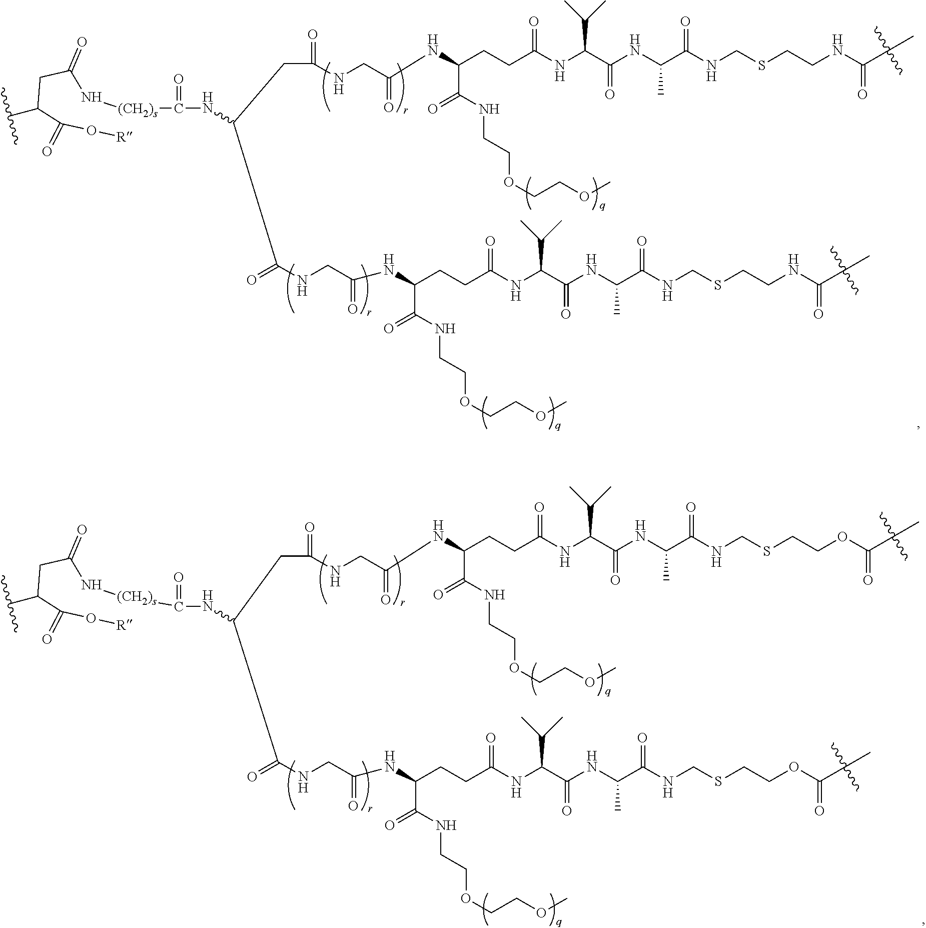

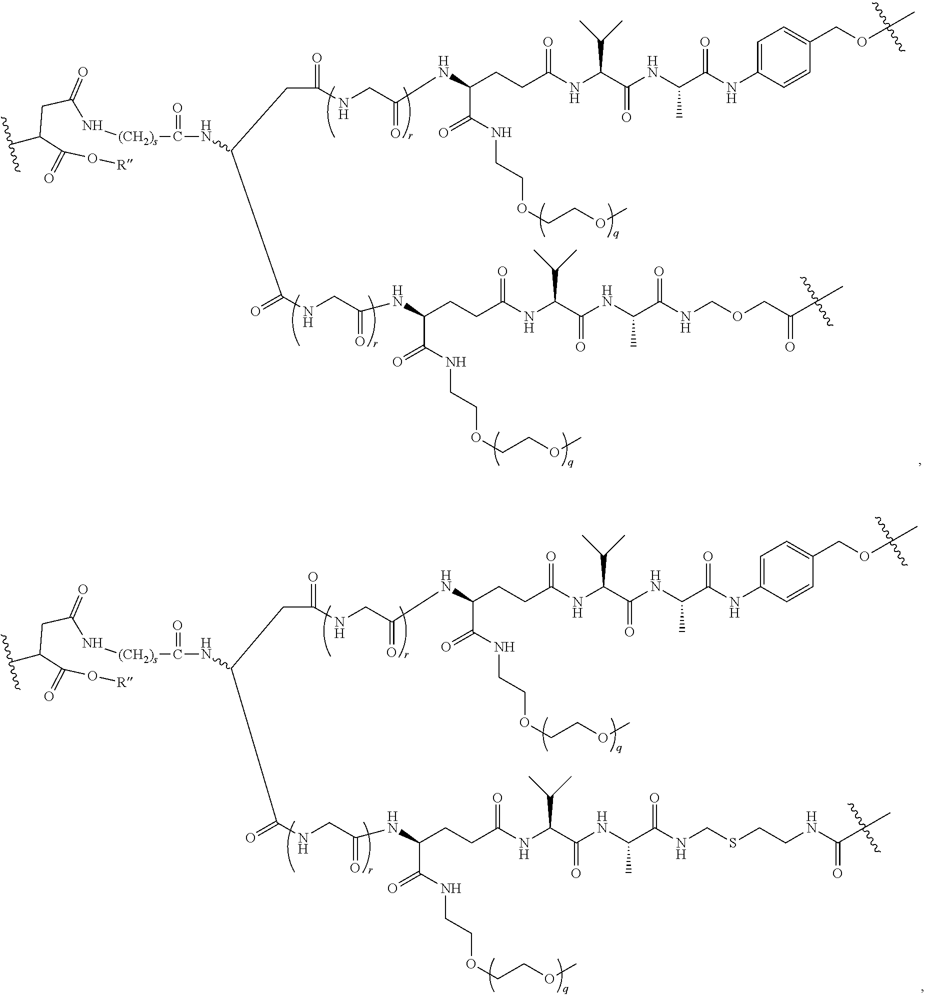

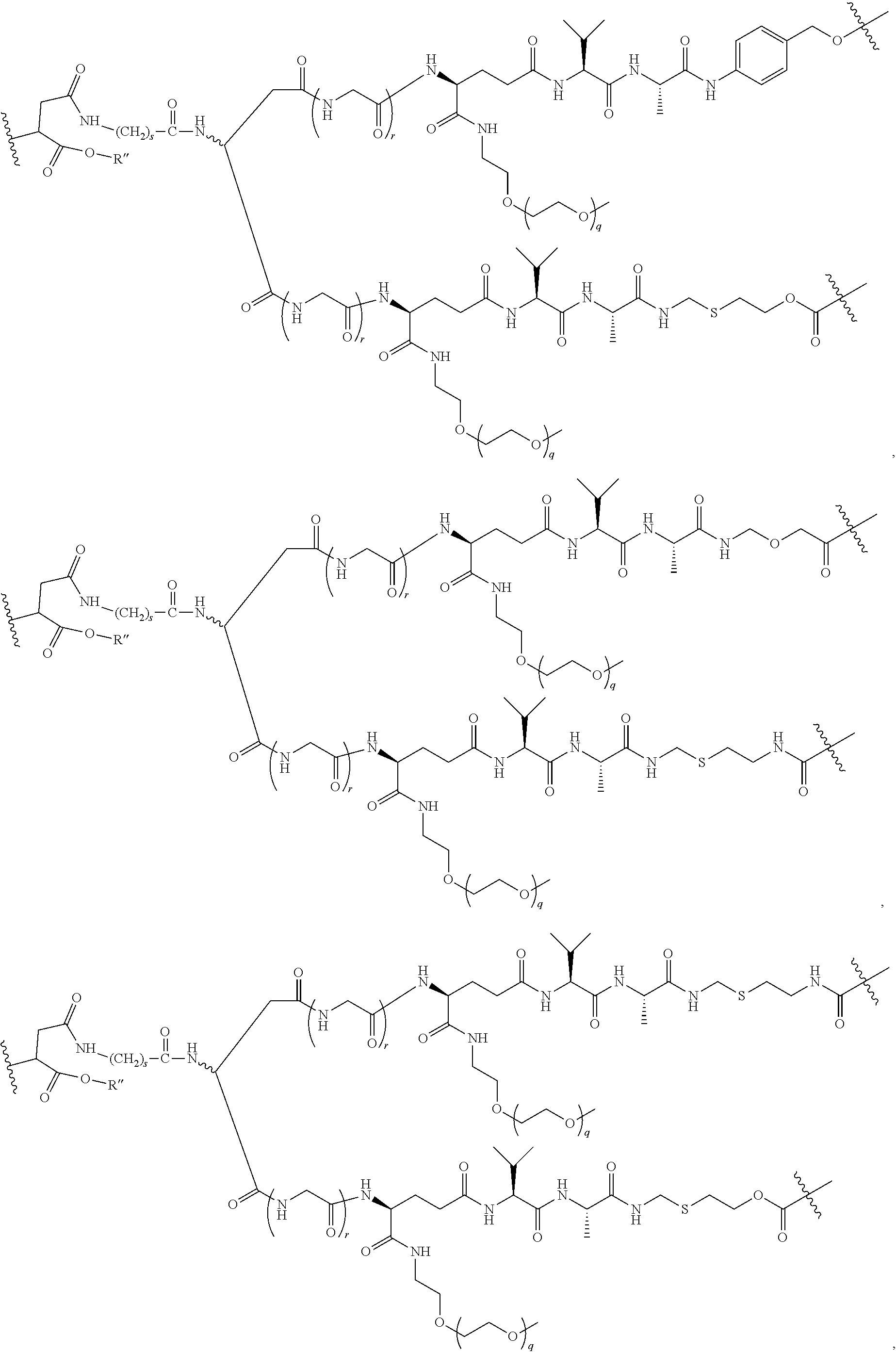

wherein R′ is selected from the group consisting of methyl, ethyl and isopropyl, each subscript s is any integer independently selected from 1-10, each subscript r is any integer independently selected from 1-4, and each subscript q is any integer independently selected from 1-20, preferably 3-10, and most preferably 5-8.

In another aspect, the present application provides use of the previously defined linker in preparation of a drug, wherein the drug comprises a linker-drug conjugate or a ligand-drug conjugate.

In another aspect, the present application provides a linker-drug conjugate comprising the previously defined linker and having a structure shown in Formula (II):

La—Bₚ [symbol] Lb-Lc-D)ₘ Formula (II),
or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

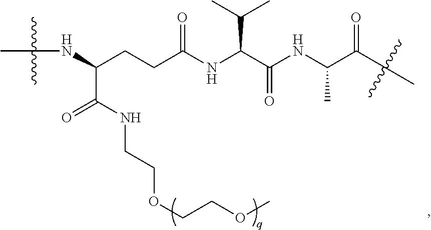

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the linker-drug conjugate has a structure shown in Formula (IIa) or (IIb):

La-Lb-Lc-D Formula (IIa),

La—B [symbol] Lb-Lc-D)₂, Formula (IIb),
or a pharmaceutically acceptable salt thereof, wherein,
La is an extension unit capable of linking a ligand unit;
B is a branching unit having a structure selected from the following formula:

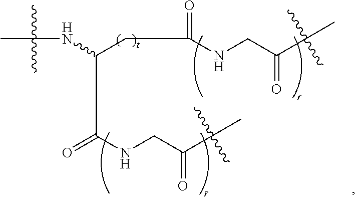

wherein a subscript t is any integer independently selected from 1-4, each subscript r is any integer independently selected from 1-4, and the B structure is linked to the La structure via amino and to the Lb structure fragment via carbonyl;
Lc is a releasable assembly unit; and
the Lb structure is selected from the following formula:

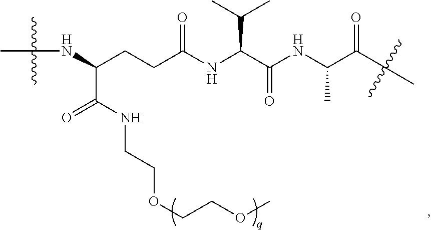

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the drug unit D comprises a detectable label, a drug, a cytokine, an enzyme, or their combinations.

In some embodiments, the drug unit D comprises a cytotoxic agent and/or cytostatics.

In some embodiments, the drug unit D comprises a DNase topoisomerase I (topology I) inhibitor.

In some embodiments, the drug unit D comprises camptothecin and a derivative thereof.

In some embodiments, the linker-drug conjugate has a structure shown in Formula (III):

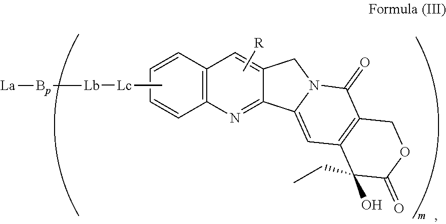

or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

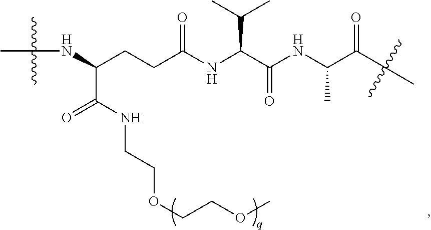

wherein a subscript q is any integer selected from 1-20, preferably 3-10, and most preferably 5-8, and the Lb structure is linked to an La or B structure via amino and to the Lc fragment via carbonyl; and

Lc is a releasable assembly unit; and

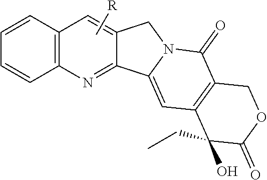

represents camptothecin or a derivative thereof.

In some embodiments, the linker-drug conjugate has a structure shown in Formula (IIIa) or (IIIb):

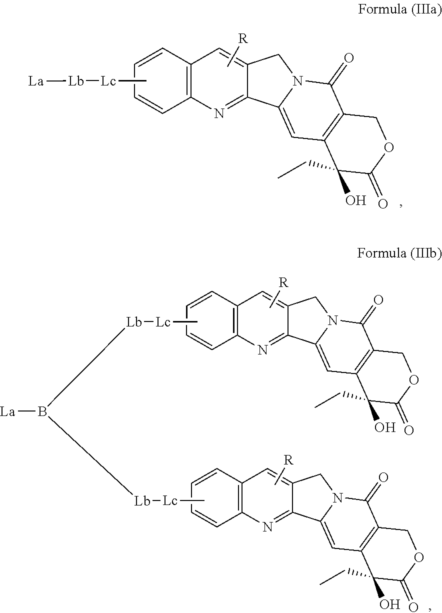

or a pharmaceutically acceptable salt thereof, wherein
La is an extension unit capable of linking a ligand unit;
B is a branching unit having a structure selected from the following formula:

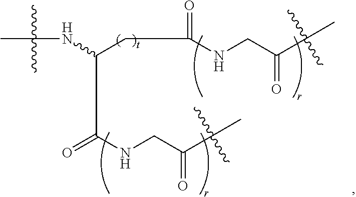

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

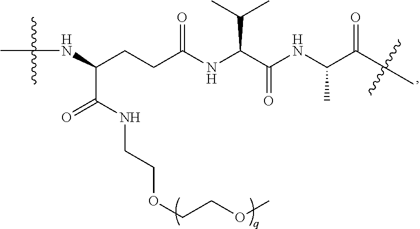

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

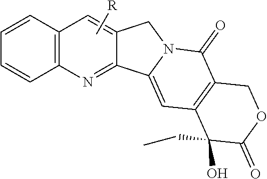

represents camptothecin or a derivative thereof.

In some embodiments, the camptothecin and the derivative thereof comprise 10-difluoromethyl camptothecin compound and a derivative thereof.

In some embodiments, the 10-difluoromethyl camptothecin compound has the following structure:

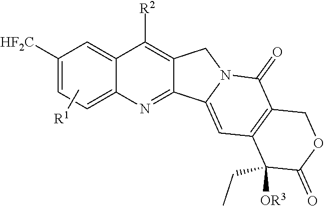

in which R¹ is selected from the group consisting of hydrogen, optionally substituted amino and optionally substituted C₁-C₆ alkyl, and a binding site of R¹ is any one of three unsubstituted sites on a benzene ring; R² is selected from the group consisting of hydrogen, optionally substituted amino and optionally substituted C₁-C₆ alkyl; and R³ is selected from the group consisting of hydrogen, acyl and optionally substituted C₁-C₆ alkyl.

In some embodiments, the 10-difluoromethyl camptothecin compound has the following structure:

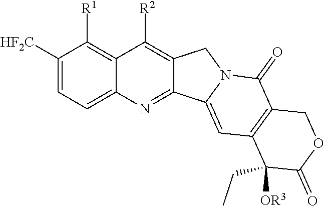

in which R¹ is selected from the group consisting of hydrogen, optionally substituted amino and optionally substituted C₁-C₆ alkyl; R² is selected from the group consisting of hydrogen, optionally substituted amino and optionally substituted C₁-C₆ alkyl; and R³ is selected from the group consisting of hydrogen, acyl and optionally substituted C₁-C₆ alkyl.

In some embodiments, R₁ is selected from the group consisting of hydrogen, optionally substituted amino and optionally substituted C₁-C₄ alkyl.

In some embodiments, R¹ is selected from the group consisting of hydrogen, amino and

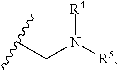

and R⁴ and R⁵ are each selected from the group consisting of hydrogen, optionally substituted C¹-C⁶ alkyl, and optionally substituted aryl.

In some embodiments, R² is selected from the group consisting of hydrogen, optionally substituted amino and optionally substituted C₁-C₄ alkyl.

In some embodiments, R² is selected from the group consisting of ethyl,

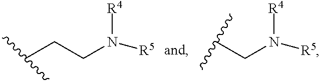

wherein R⁴ and R⁵ are each selected from the group consisting of hydrogen, optionally substituted C₁-C₆ alkyl, and optionally substituted aryl.

In some embodiments, R³ is selected from the group consisting of hydrogen, acyl and optionally substituted C₁-C₄ alkyl.

In some embodiments, R³ is selected from the group consisting of hydrogen,

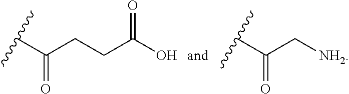 [SMILES: *C(=O)CCC(=O)O ; *C(=O)CN]

In some embodiments, the 10-difluoromethyl camptothecin compound has the following structure:

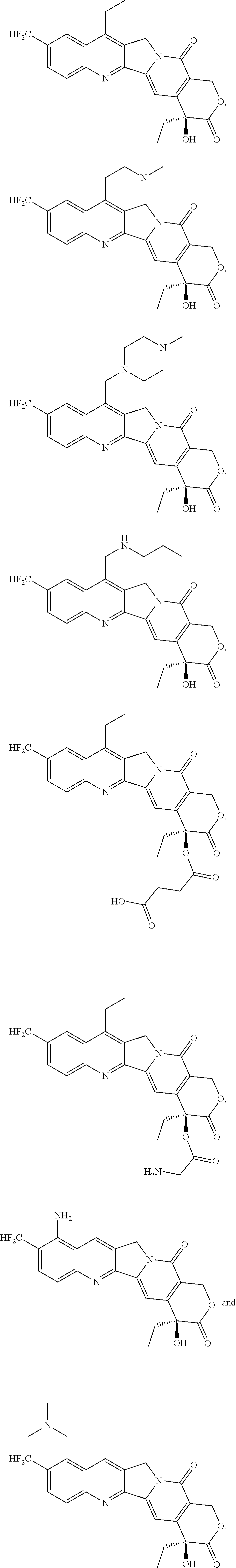

In some embodiments, the camptothecin and the derivative thereof are selected from the group consisting of: camptothecin, exatecan, topotecan, irinotecan, belotecan, lurtotecan GG-211, CKD-602, gimatecan ST1481, karenitecin BNP-1350, BN-80915, hydroxycamptothecin HCPT, 9-aminocamptothecin, 9-nitrocamptothecin, 7-ethyl-10-hydroxycamptothecin SN38, 7-ethyl-10-difluoromethylcamptothecin and their derivatives.

In some embodiments, the camptothecin and the derivative thereof are selected from the group consisting of camptothecin, exatecan, 7-ethyl-10-hydroxycamptothecin, 7-ethyl-10-difluoromethylcamptothecin, and their solvates, hydrates, stereoisomers, tautomers, isotope labels, metabolites, prodrugs, and pharmaceutically acceptable salts.

In some embodiments, the linker-drug conjugate has a structure shown in Formula (IVa), (IVb), (IVc), or (IVd):

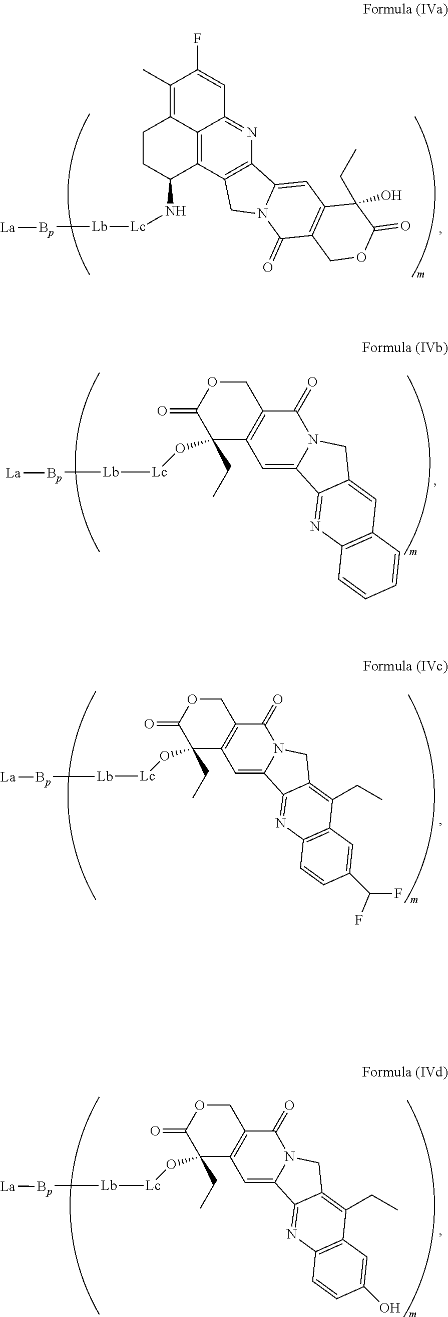

or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

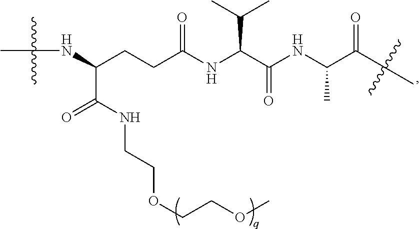

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

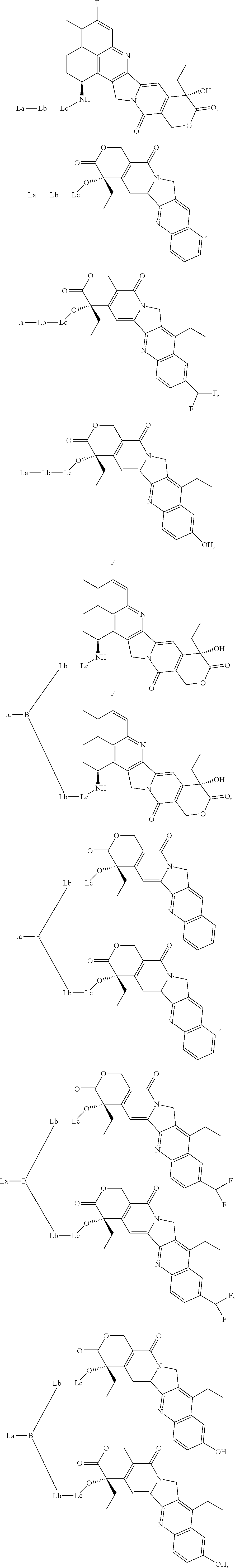

or a pharmaceutically acceptable salt thereof, wherein,
La is an extension unit;
B is a branching unit having a structure selected from the following formula:

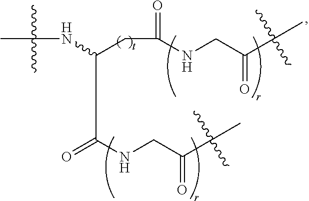

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

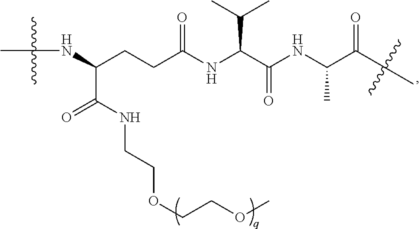

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In another aspect, the present application provides a ligand-drug conjugate comprising the previously defined linker or the previously defined linker-drug conjugate and having a structure shown in Formula (V):

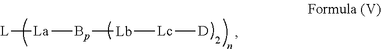

or a pharmaceutically acceptable salt thereof, wherein,
L is a ligand unit, wherein a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

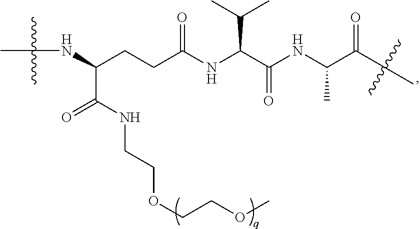

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (Va) or (Vb):

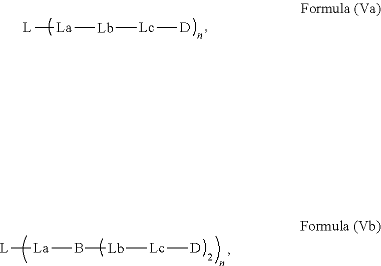

or a pharmaceutically acceptable salt thereof, wherein,
L is a ligand unit, wherein a subscript n is any integer selected from 1-8, and preferably 4-8;
La is an extension unit;
B is a branching unit having a structure selected from the following formula:

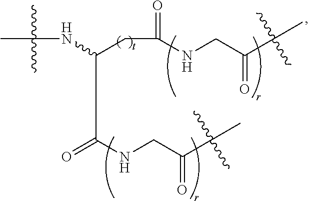

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

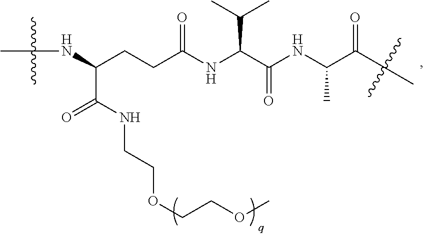

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the drug unit D comprises a cytotoxic agent and/or cytostatics.

In some embodiments, the drug unit D comprises a cytotoxic agent for cancer treatment.

In some embodiments, the drug unit D comprises a DNase topoisomerase I (topology I) inhibitor.

In some embodiments, the drug unit D comprises camptothecin and a derivative thereof.

In some embodiments, the ligand-drug conjugate has a structure shown in Formula (VI):

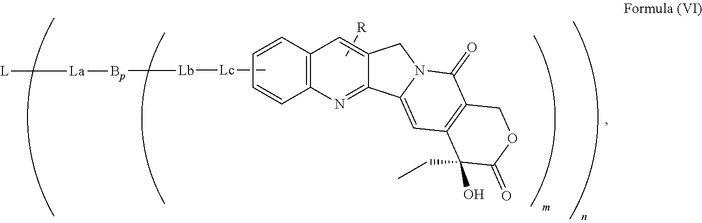

or a pharmaceutically acceptable salt thereof, wherein,
L is a ligand unit, and a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

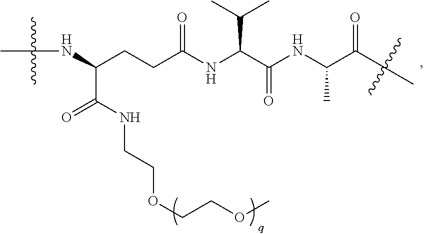

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

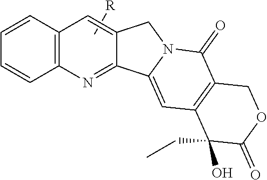

represents camptothecin or a derivative thereof.

In some embodiments, the ligand-drug conjugate has a structure shown in Formula (VIa) or

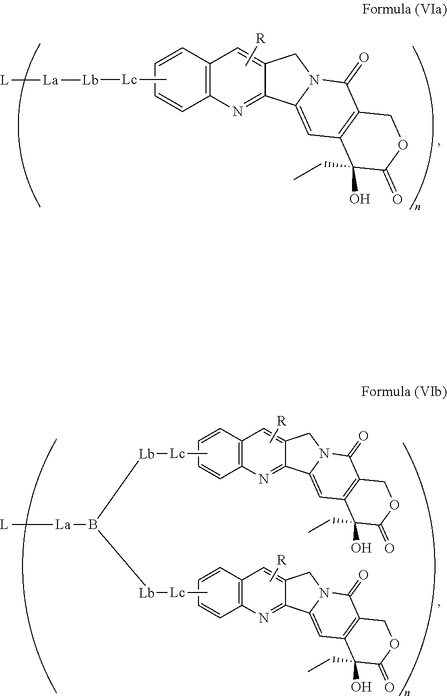

or a pharmaceutically acceptable salt thereof, wherein,
L is a ligand unit, wherein a subscript n is any integer selected from 1-8, and preferably 4-8;
La is an extension unit;
B is a branching unit having a structure selected from the following formula:

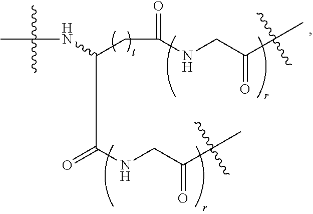

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

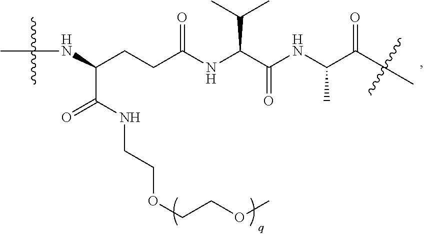

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

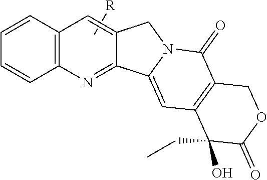

represents camptothecin or a derivative thereof.

In some embodiments, the camptothecin and the derivative thereof comprise 10-difluoromethyl camptothecin compound and a derivative thereof.

In some embodiments, the 10-difluoromethyl camptothecin compound has the following structure:

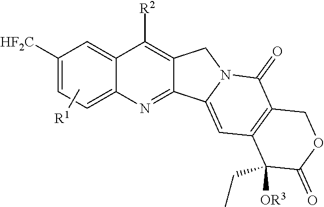

wherein,
R¹ is selected from the group consisting of hydrogen, optionally substituted amino and optionally substituted C₁-C₆ alkyl, and a binding site of R¹ is any one of three unsubstituted sites on a benzene ring;
R² is selected from the group consisting of hydrogen, optionally substituted amino and optionally substituted C₁-C₆ alkyl; and
R³ is selected from the group consisting of hydrogen, acyl and optionally substituted C₁-C₆ alkyl.

In some embodiments, the 10-difluoromethyl camptothecin compound has the following

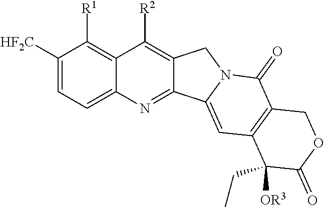

wherein,
R¹ is selected from the group consisting of hydrogen, optionally substituted amino and optionally substituted C₁-C₆ alkyl; and
R² is selected from the group consisting of hydrogen, optionally substituted amino and optionally substituted C₁-C₆ alkyl; and
R³ is selected from the group consisting of hydrogen, acyl and optionally substituted C₁-C₆ alkyl.

In some embodiments, R₁ is selected from the group consisting of hydrogen, optionally substituted amino and optionally substituted C₁-C₄ alkyl.

In some embodiments, R¹ is selected from the group consisting of hydrogen, amino and

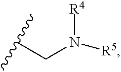

and R⁴ and R⁵ are each selected from the group consisting of hydrogen, optionally substituted C₁-C₆ alkyl, and optionally substituted aryl.

In some embodiments, R² is selected from the group consisting of hydrogen, optionally substituted amino and optionally substituted C₁-C₄ alkyl.

In some embodiments, R² is selected from the group consisting of ethyl,

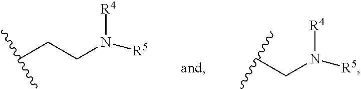

wherein R⁴ and R⁵ are each selected from the group consisting of hydrogen, optionally substituted C₁-C₆ alkyl, and optionally substituted aryl.

In some embodiments, R³ is selected from the group consisting of hydrogen, acyl and optionally substituted C₁-C₄ alkyl.

In some embodiments, R³ is selected from the group consisting of hydrogen,

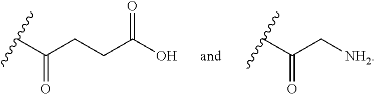 [SMILES: *C(=O)CCC(=O)O ; "and" ; *C(=O)CN]

In some embodiments, the 10-difluoromethyl camptothecin compound has the following

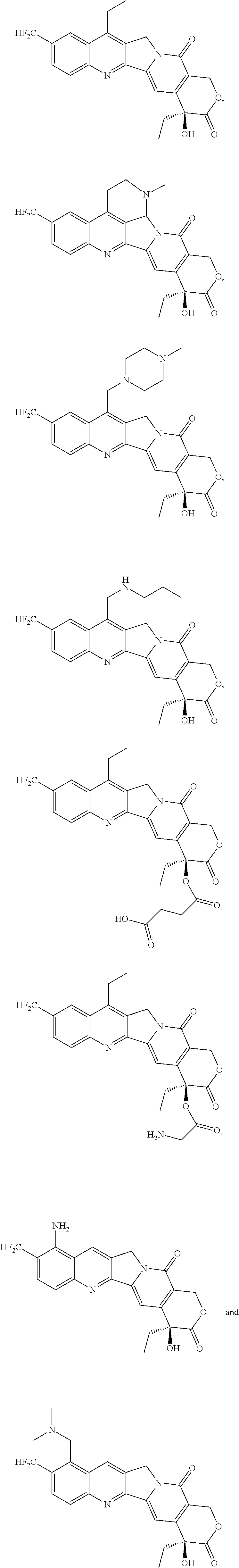

In some embodiments, the camptothecin and the derivative thereof are selected from the group consisting of: camptothecin, exatecan, topotecan, irinotecan, belotecan, lurtotecan GG-211, CKD-602, gimatecan ST1481, karenitecin BNP-1350, BN-80915, hydroxycamptothecin HCPT, 9-aminocamptothecin, 9-nitrocamptothecin, 7-ethyl-10-hydroxycamptothecin SN38, 7-ethyl-10-difluoromethylcamptothecin and their derivatives.

In some embodiments, the camptothecin and the derivative thereof are selected from the group consisting of camptothecin, exatecan, 7-ethyl-10-hydroxycamptothecin, 7-ethyl-10-difluoromethylcamptothecin, and their solvates, hydrates, stereoisomers, tautomers, isotope labels, metabolites, prodrugs, and pharmaceutically acceptable salts.

In some embodiments, the ligand-drug conjugate has a structure shown in Formula (VIIa), (VIIb), (VIIc) or (VIId):

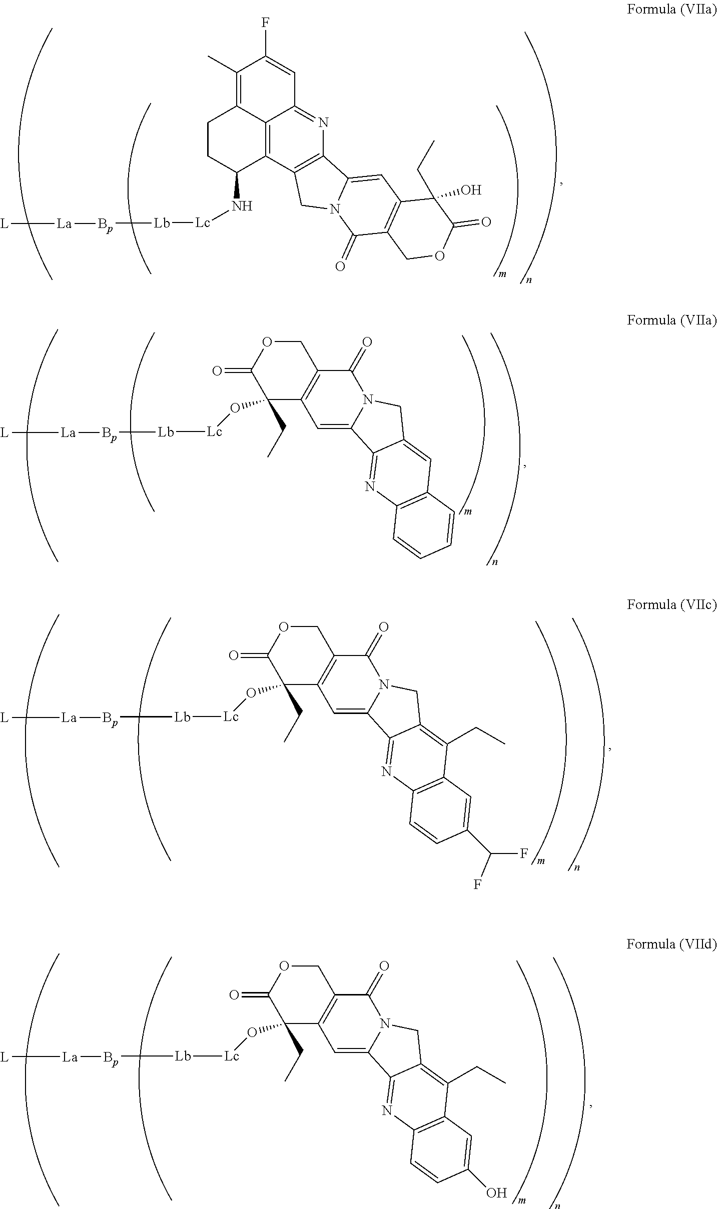

or a pharmaceutically acceptable salt thereof, wherein,
L is a ligand unit, and a subscript n is any integer selected from 1-8, and preferably 4-8;
La is an extension unit;
B is an optional branching unit, a subscript p is 0 or 1, and wherein m is 1 when p is 0, and m is 2, 3, or 4 when p is 1;
the Lb structure is selected from the following formula:

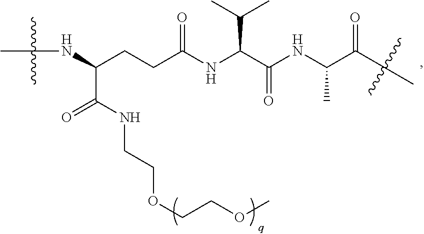

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

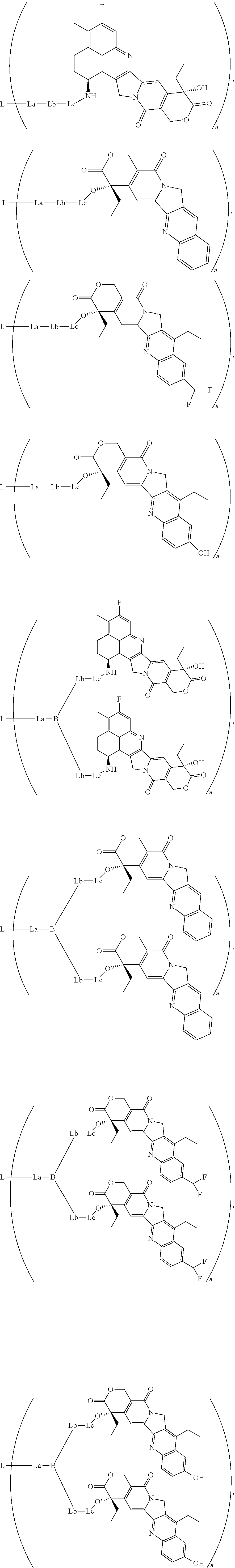

or a pharmaceutically acceptable salt thereof, wherein,
L is a ligand unit, and a subscript n is any integer selected from 1-8, and preferably 4-8;
La is an extension unit;
B is a branching unit having a structure selected from the following formula:

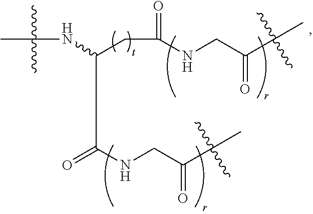

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

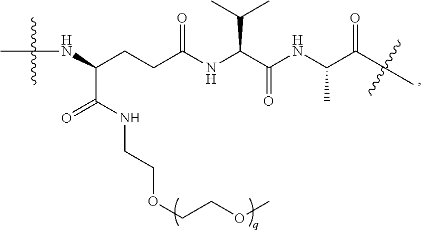

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand comprises an antibody.

In some embodiments, the antibody comprises a monoclonal antibody, a polyclonal antibody, a dimer, a multimer, a polyspecific antibody, a complete antibody, an antibody fragment, a human antibody, a humanized antibody, a chimeric antibody, or an antibody from another species.

In some embodiments, the antibody fragment comprises: Fab, Fab′, F(ab′)2, a Fv fragment, a scFv antibody fragment, a linear antibody, a single-domain antibody such as sdAB, a cameliaceae VHH domain, or a polyspecific antibody formed from an antibody fragment.

In some embodiments, the antibody comprises modified or unmodified analogues and derivatives, and the antibody is allowed to retain antigen-binding immune specificity thereof.

In some embodiments, the antibody or antibody fragment has a number of linkage points necessary for the drug-linker.

In some embodiments, the ligand-drug conjugate has a structure shown in Formula (VIII):

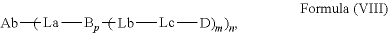

or a pharmaceutically acceptable salt thereof, wherein,
Ab represents an antibody, wherein a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

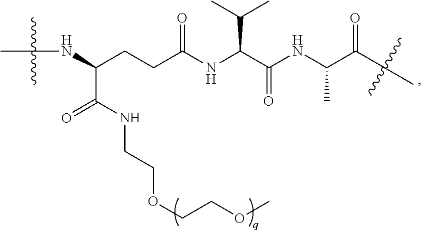

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (VIIIa) or (VIIIb):

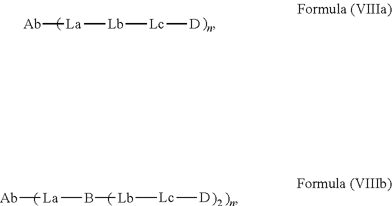

or a pharmaceutically acceptable salt thereof, wherein,
Ab represents an antibody, and a subscript n is any integer selected from 1-8, and preferably 4-8;
La is an extension unit;
B is a branching unit having a structure selected from the following formula:

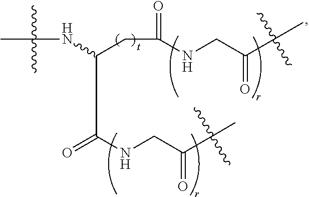

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

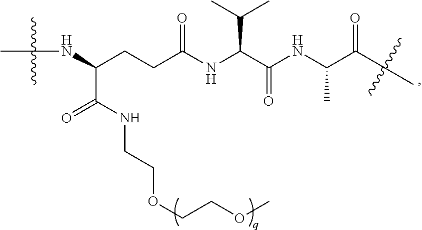

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (IX):

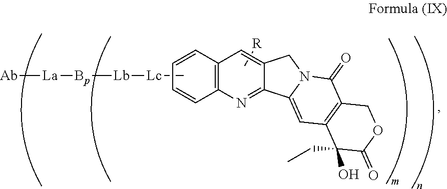

or a pharmaceutically acceptable salt thereof, wherein,
Ab represents an antibody, and a subscript n is any integer selected from 1-8, and preferably 4-8;
La is an extension unit,
B is an optional branching unit, a subscript p is 0 or 1, and wherein m is 1 when p is 0, and m is 2, 3, or 4 when p is 1;
the Lb structure is selected from the following formula:

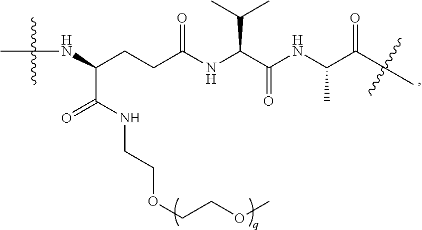

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

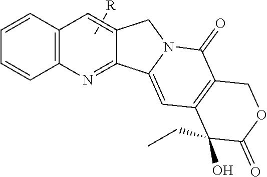

represents camptothecin or a derivative thereof.

In some embodiments, the ligand-drug conjugate has a structure shown in Formula (IXa) or (IXb):

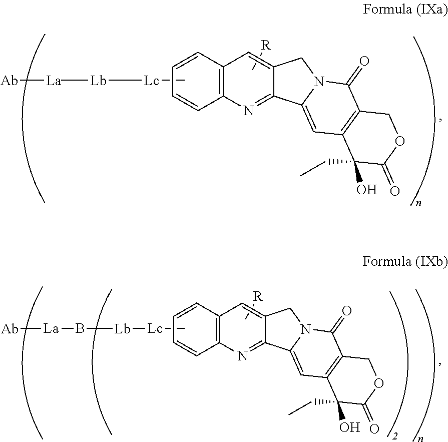

or a pharmaceutically acceptable salt thereof, wherein,
Ab represents an antibody, and a subscript n is any integer selected from 1-8, and preferably 4-8;
La is an extension unit;
B is a branching unit having a structure selected from the following formula:

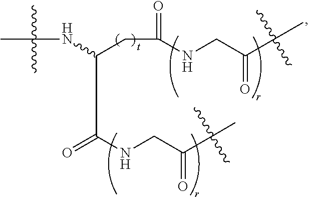

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

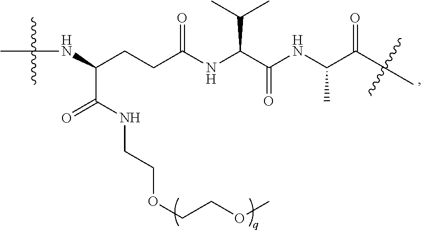

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

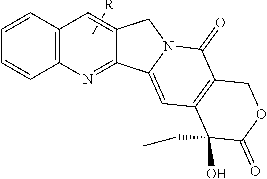

represents camptothecin or a derivative thereof.

In some embodiments, the ligand-drug conjugate has a structure shown in Formula (Xa), (Xb), (Xc) or (Xd):

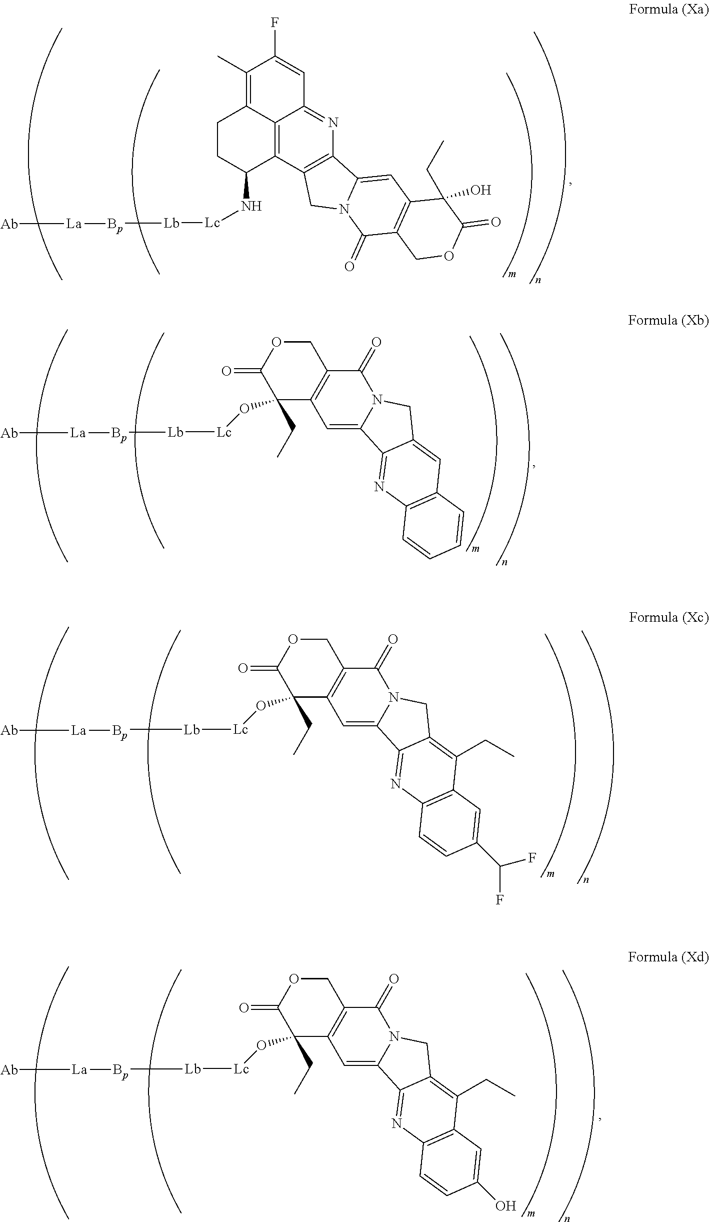

or a pharmaceutically acceptable salt thereof, wherein,
Ab represents an antibody, and a subscript n is any integer selected from 1-8, and preferably 4-8;
La is an extension unit;
B is an optional branching unit, a subscript p is 0 or 1, and wherein m is 1 when p is 0, and m is 2, 3, or 4 when p is 1;
the Lb structure is selected from the following formula:

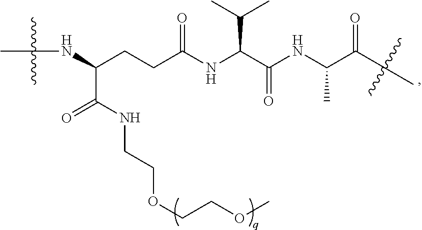

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

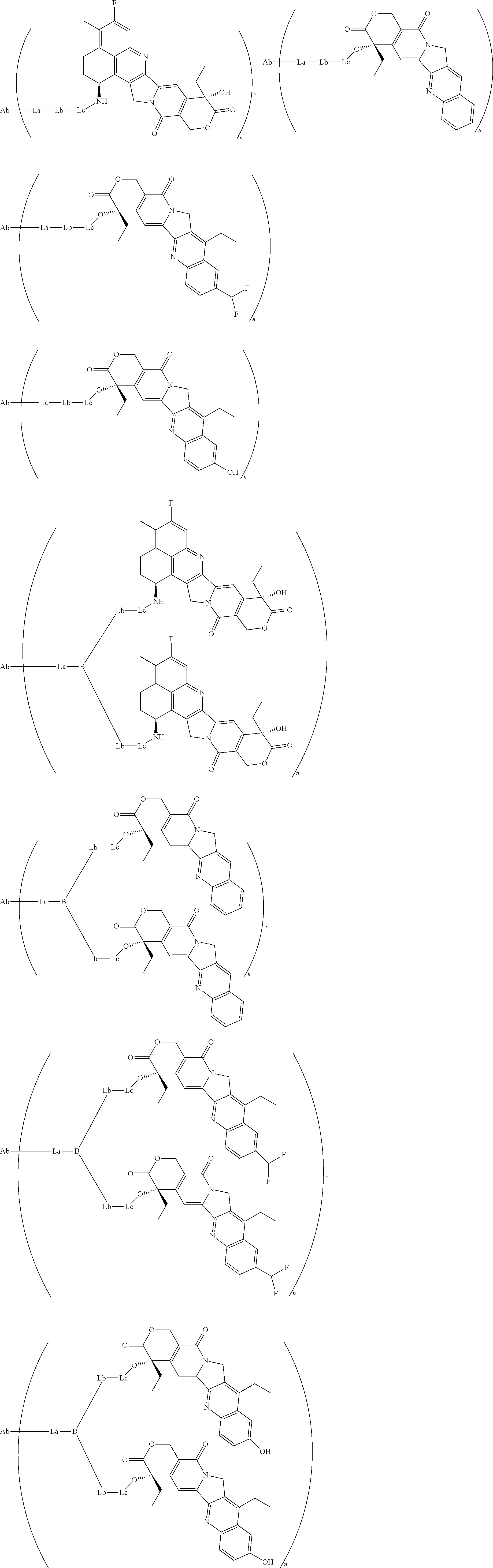

or a pharmaceutically acceptable salt thereof, wherein,
Ab represents an antibody, and a subscript n is any integer selected from 1-8, and preferably 4-8;
La is an extension unit;
B is a branching unit having a structure selected from the following formula:

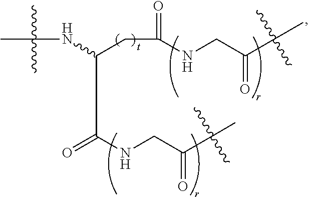

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

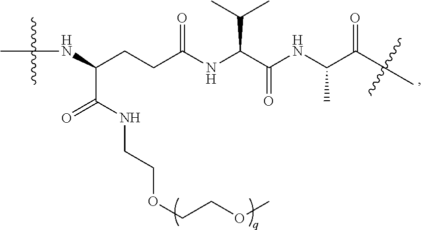

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

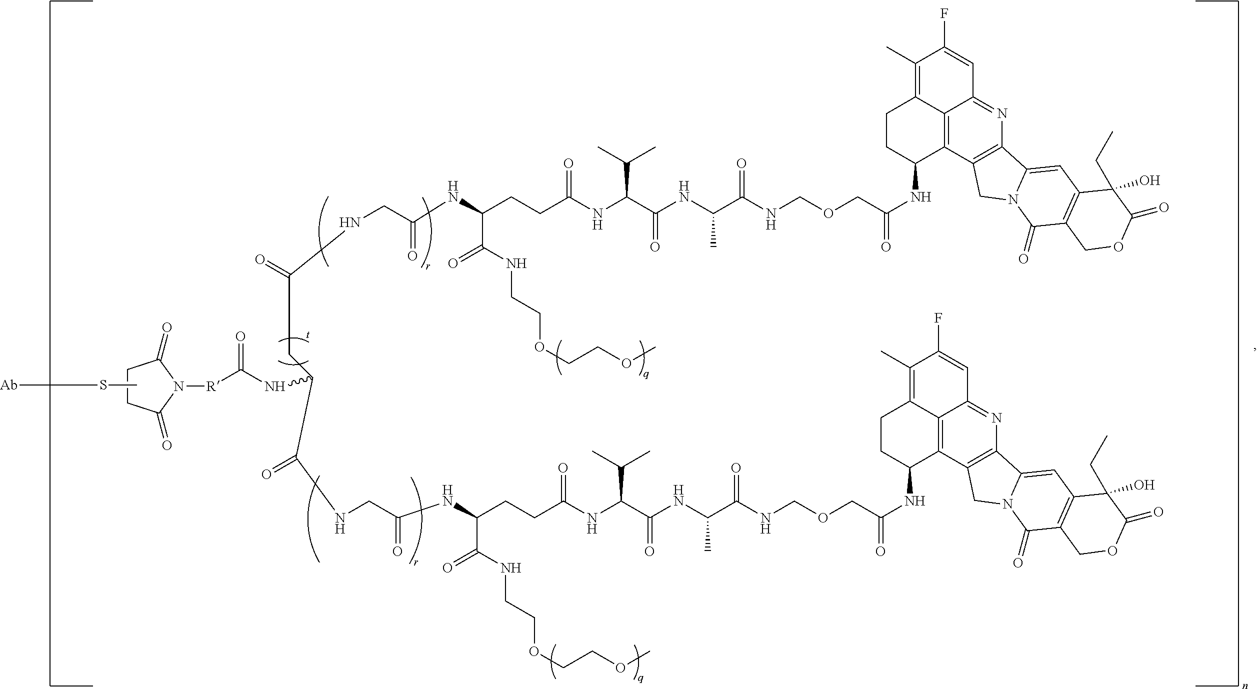

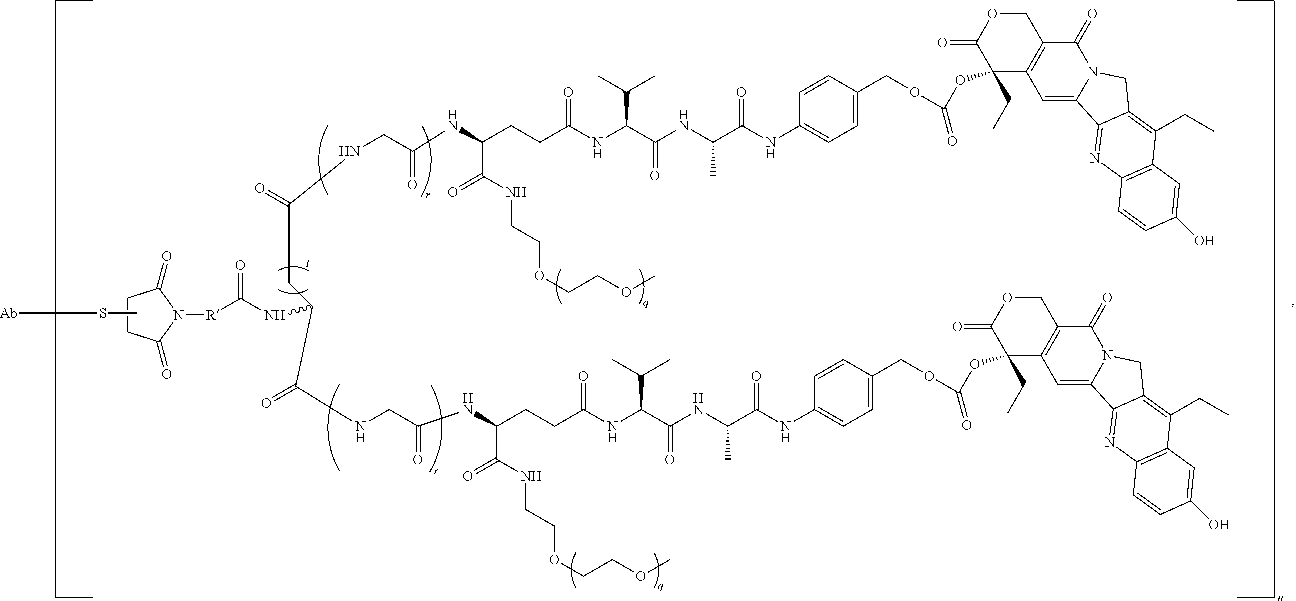

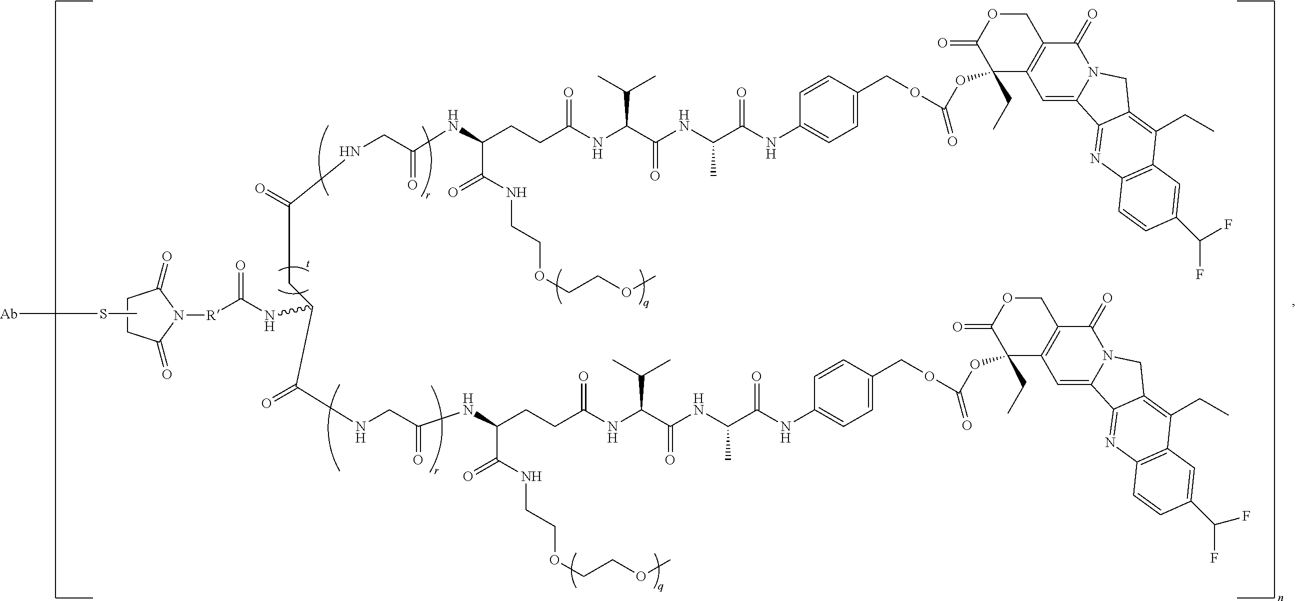

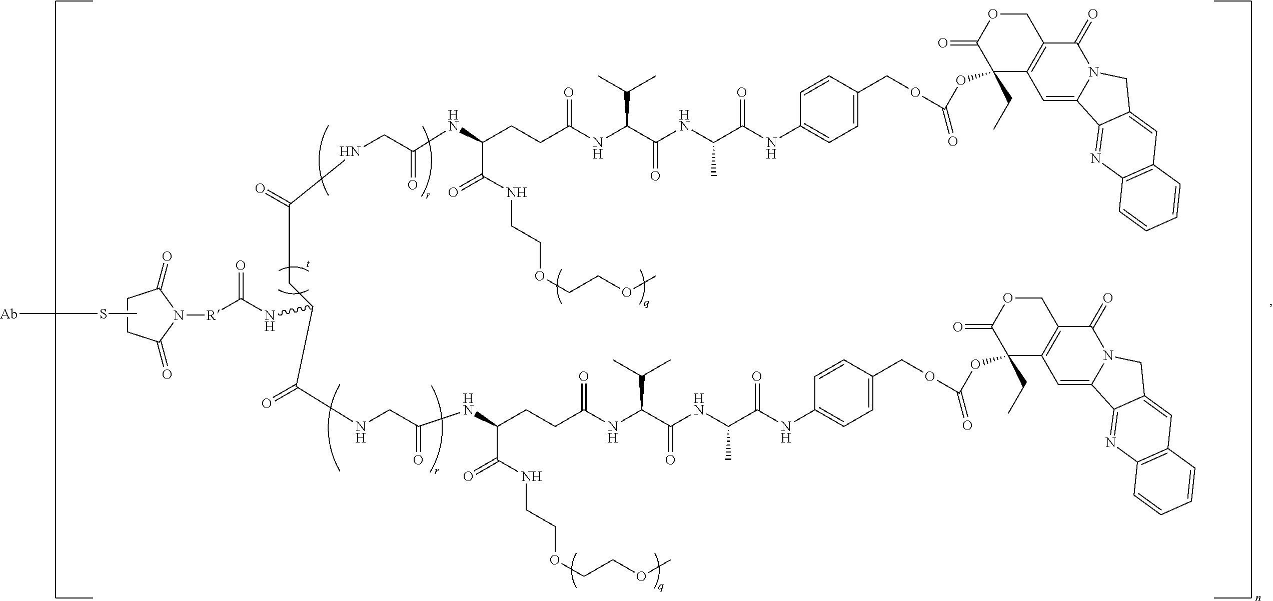

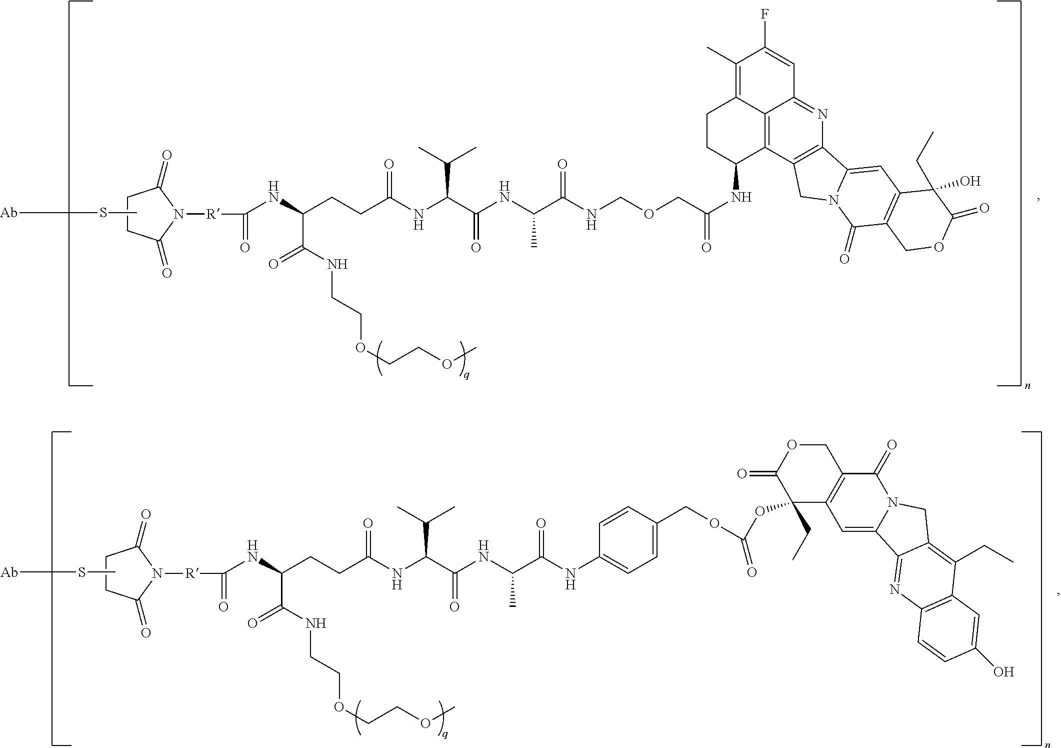

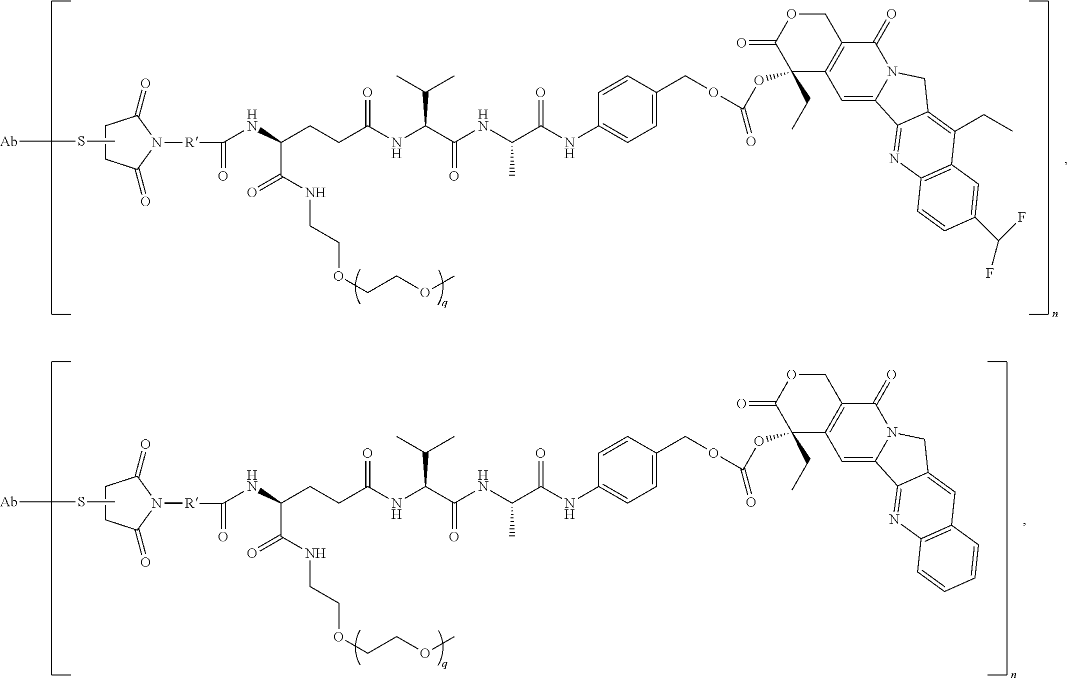

or a pharmaceutically acceptable salt thereof, wherein,
Ab represents an antibody,
R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl;
each subscript r is any integer independently selected from 1-4,
each subscript t is any integer independently selected from 1-4, and
each subscript q is any integer independently selected from 1-20, preferably 3-10, and most preferably 5-8;
each subscript n is any integer selected from 1-8, and preferably 4-8;

In some embodiments, the ligand-drug conjugate is selected from the following structure:

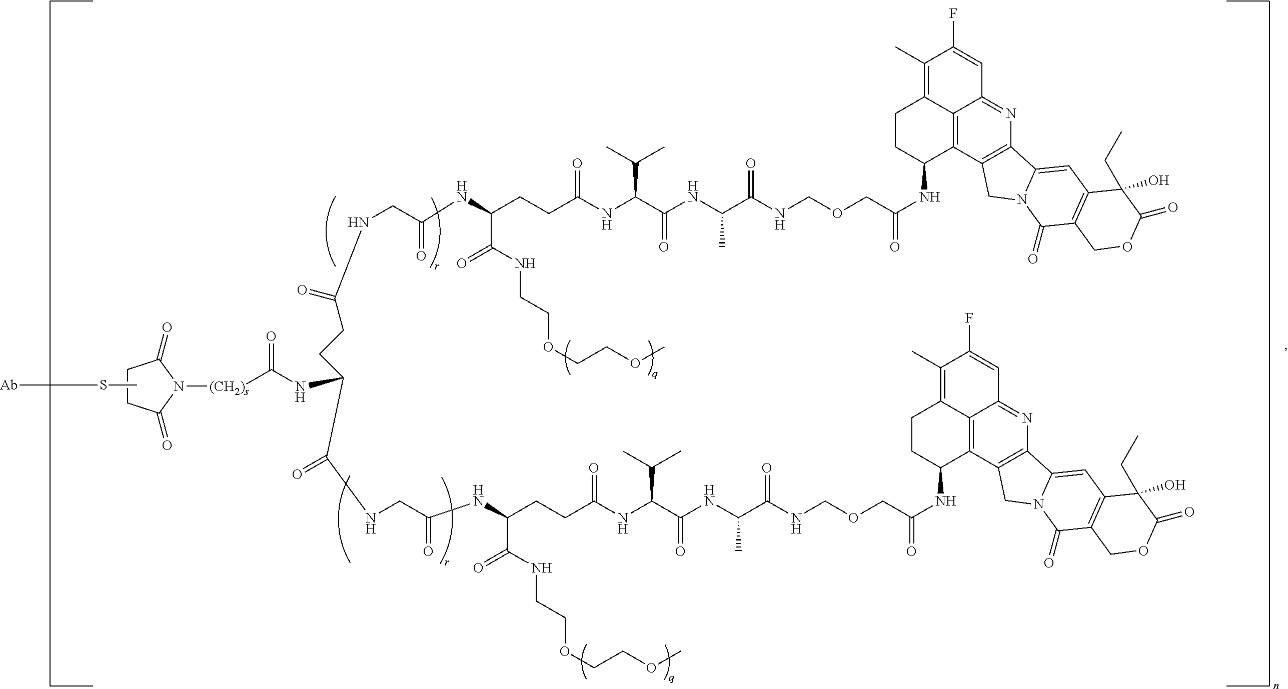

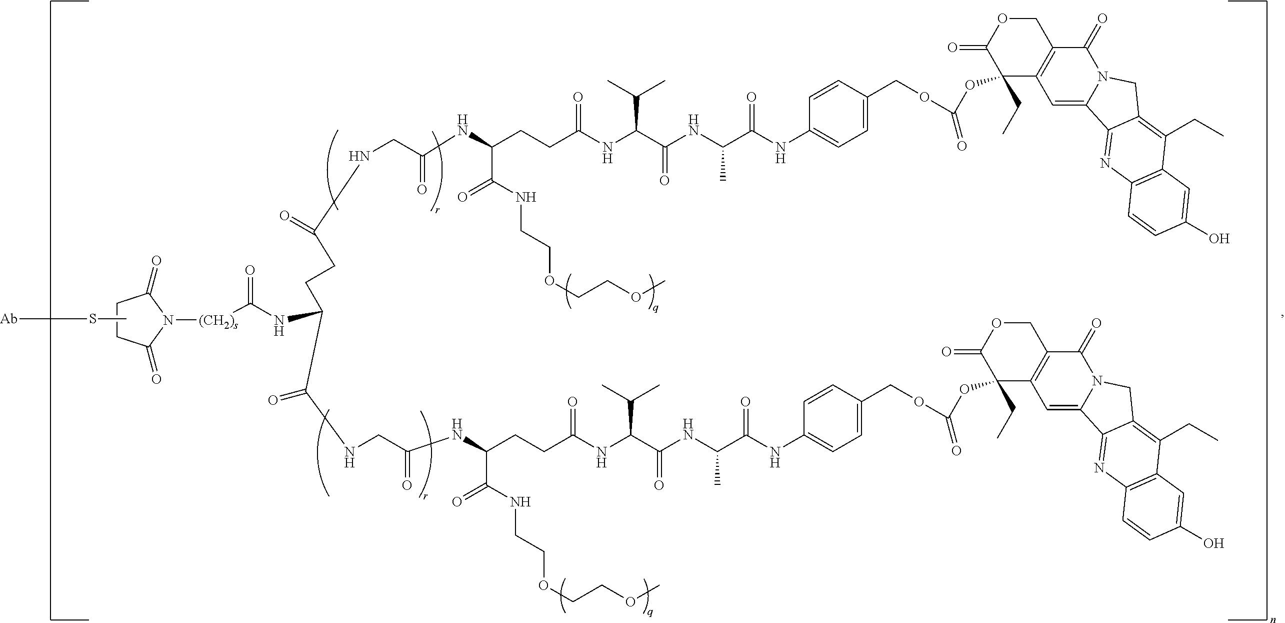

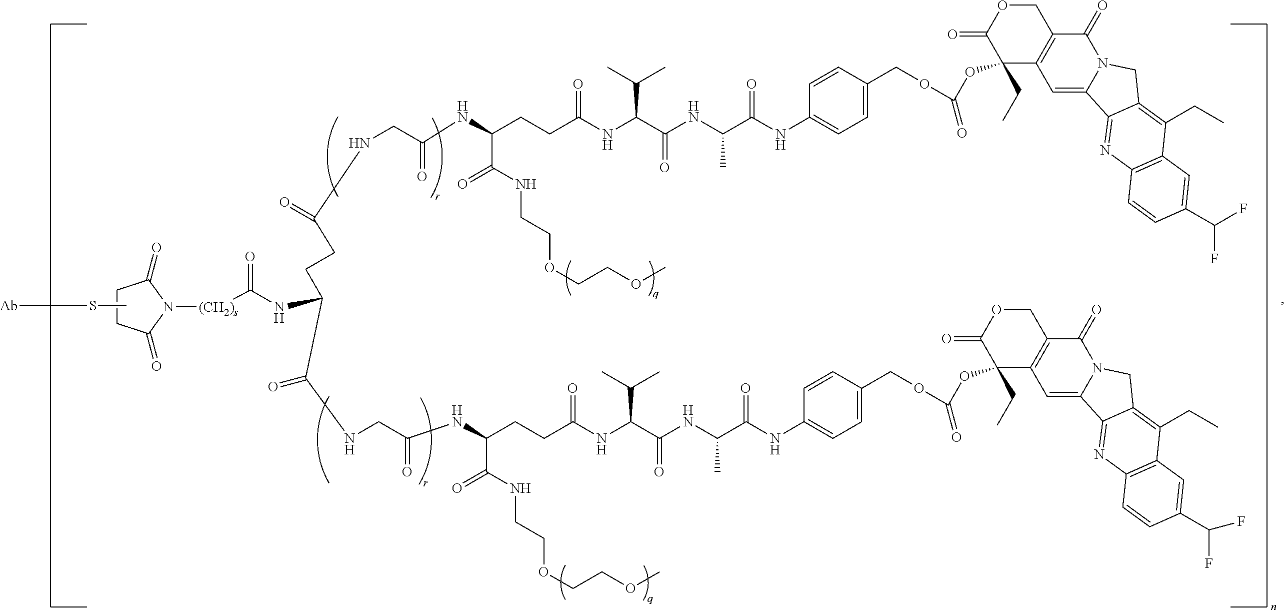

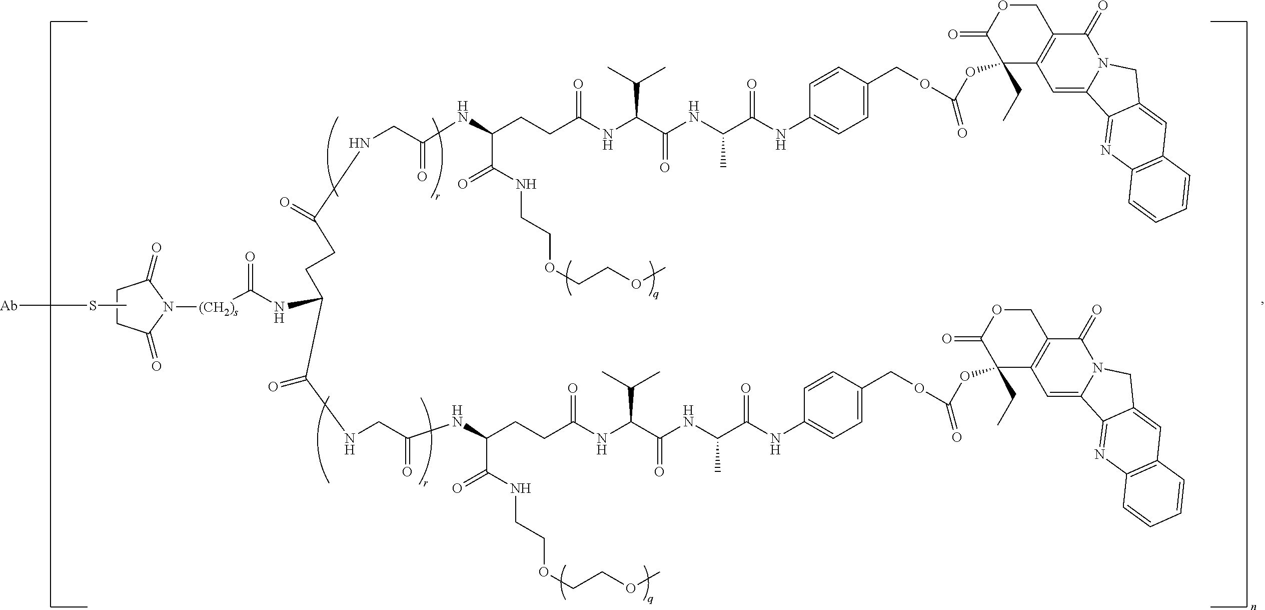

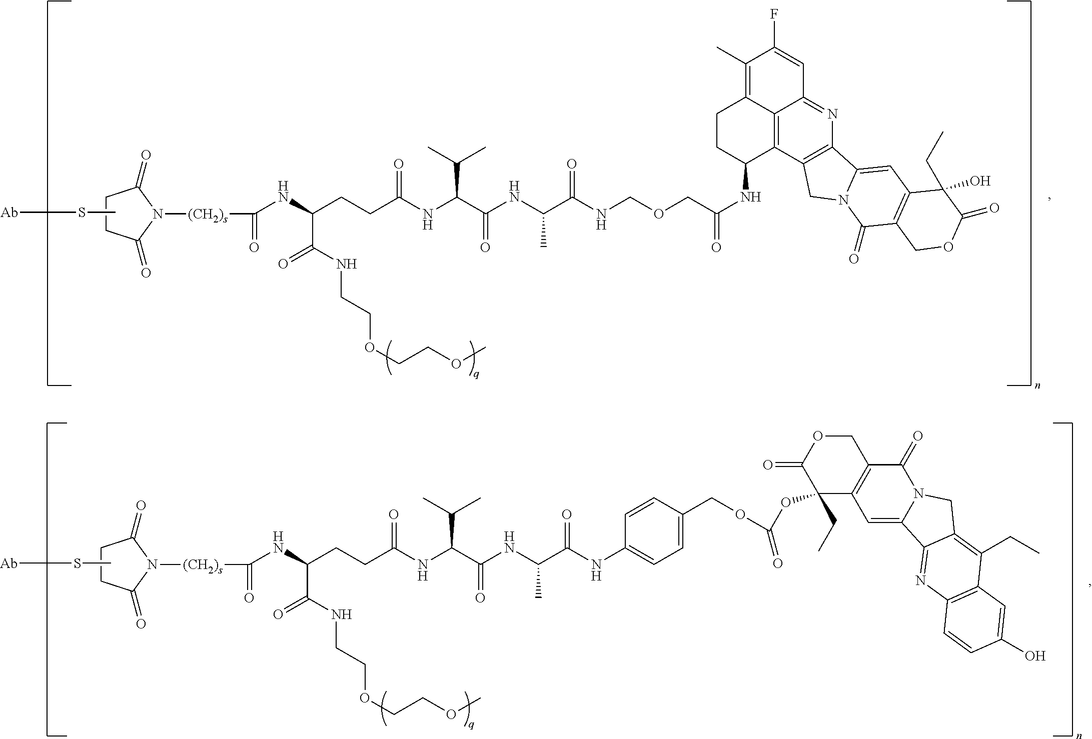

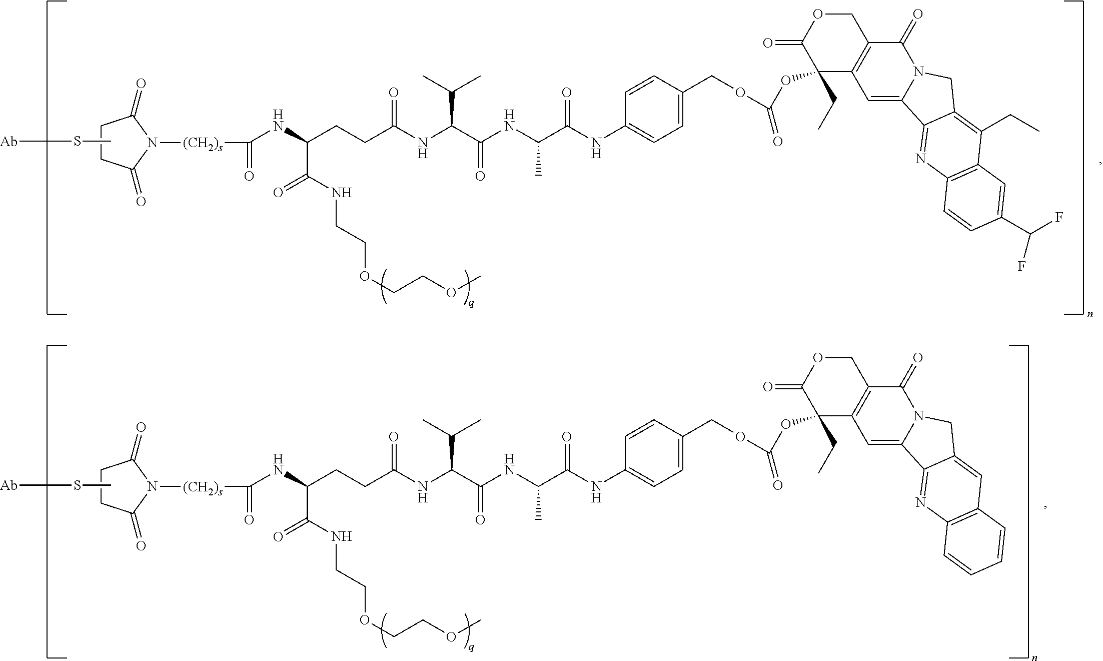

or a pharmaceutically acceptable salt thereof, wherein,
Ab represents an antibody;
each subscript s is any integer independently selected from 1-10, and preferably 1-5;
each subscript q is any integer selected from 1-20, preferably 3-10, and most preferably 5-8;
each subscript r is any integer selected from 1-4; and
each subscript n is any integer selected from 1-8, and preferably 4-8;

In some embodiments, the subscript s is 2.

In some embodiments, the subscript r is 2.

In some embodiments, the subscript q is 7.

In some embodiments, the La structure is linked to an antibody via a position 3 of succinimide.

In some embodiments, the ligand comprises a monoclonal antibody mAb or an antigen-binding fragment thereof.

In some embodiments, the ligand-drug conjugate is selected from the following structure:

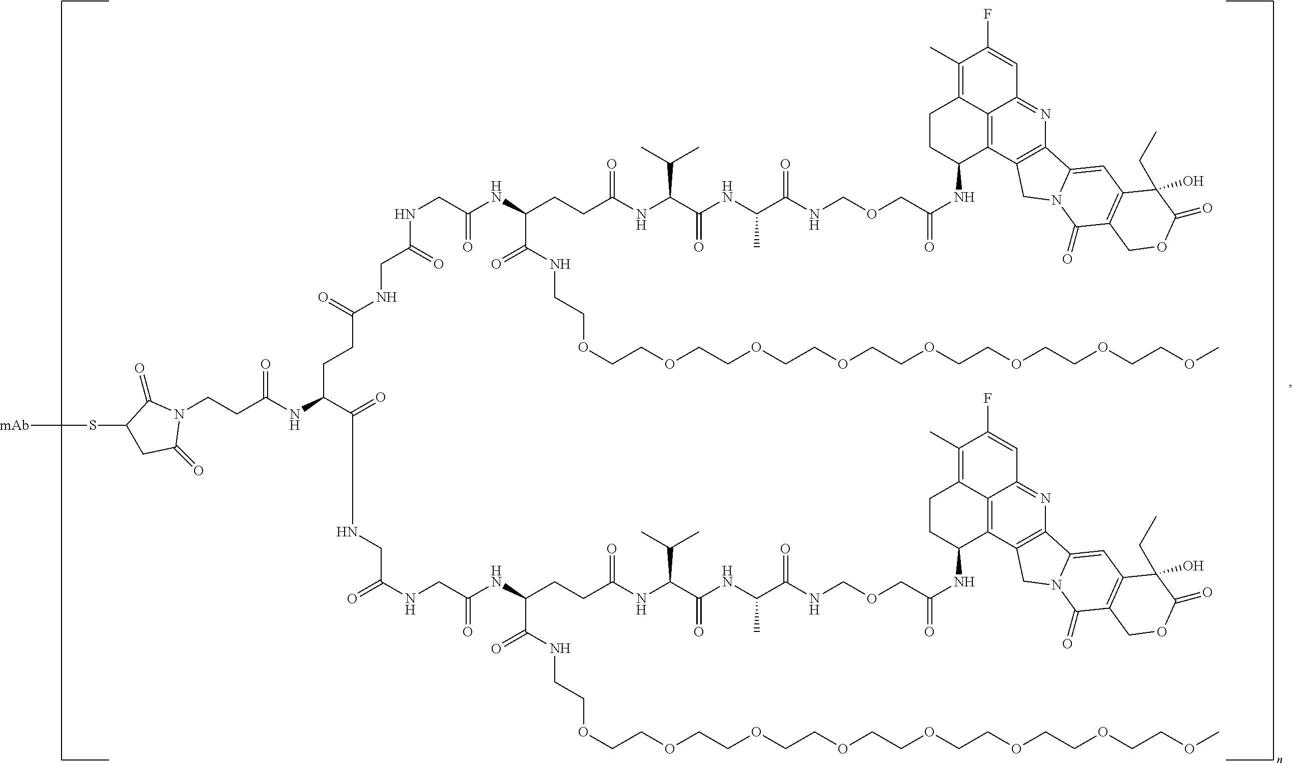

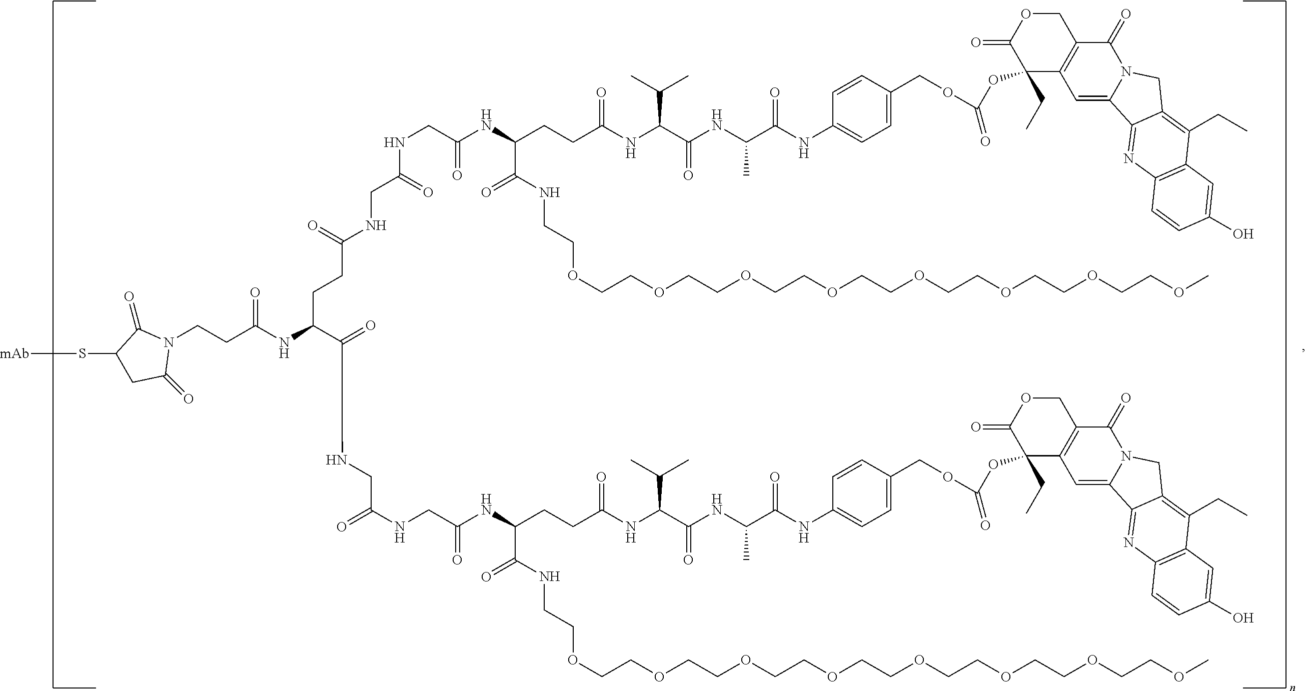

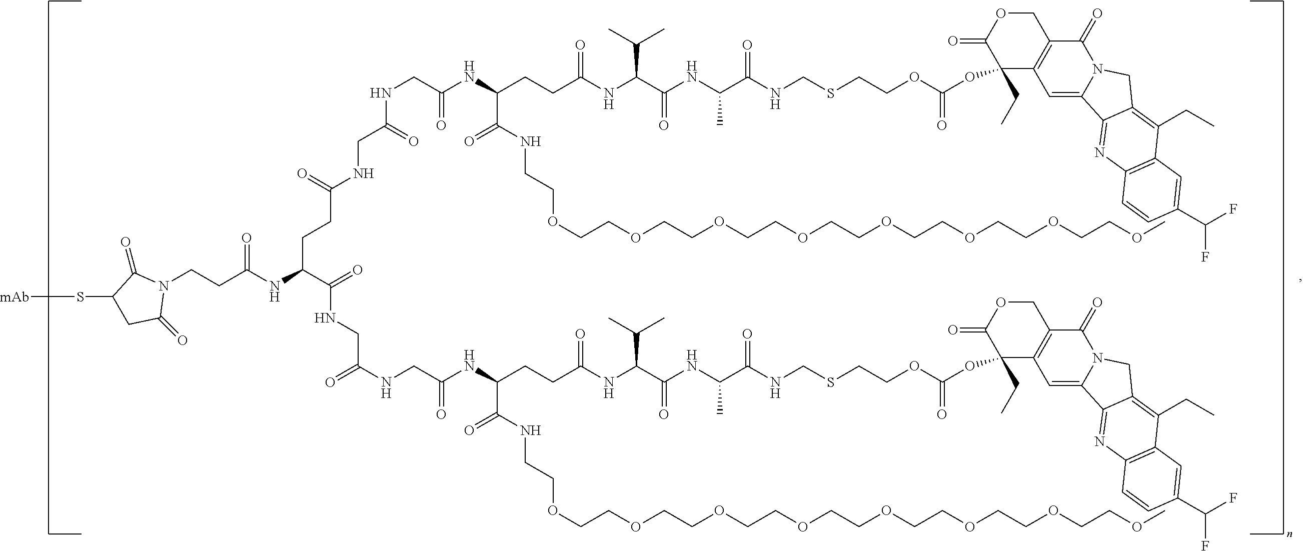

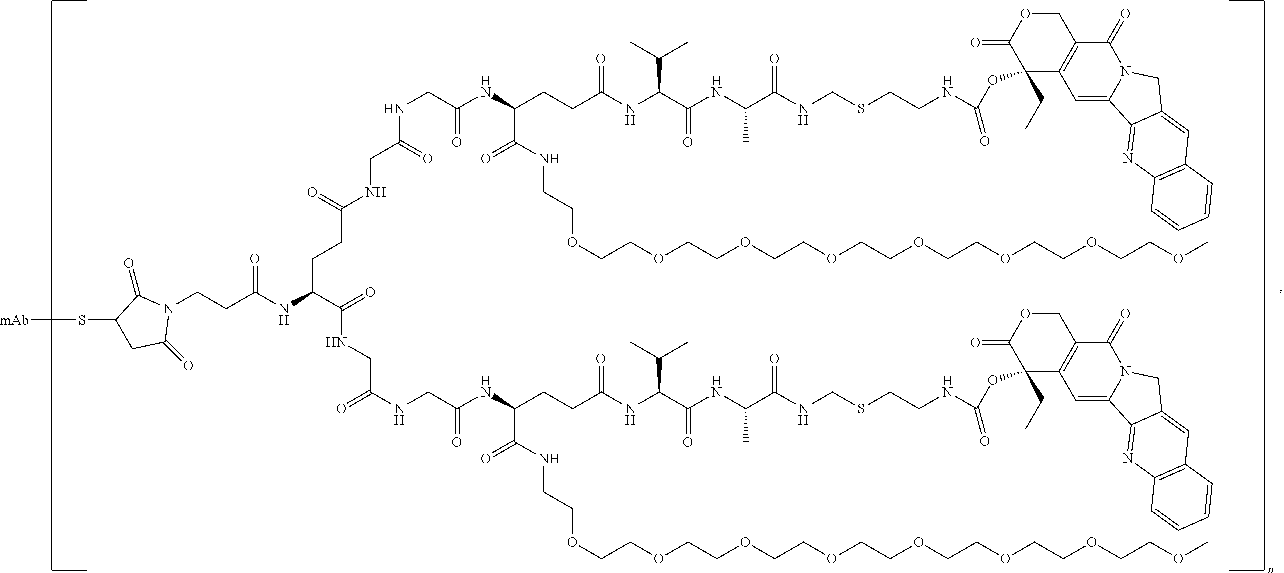

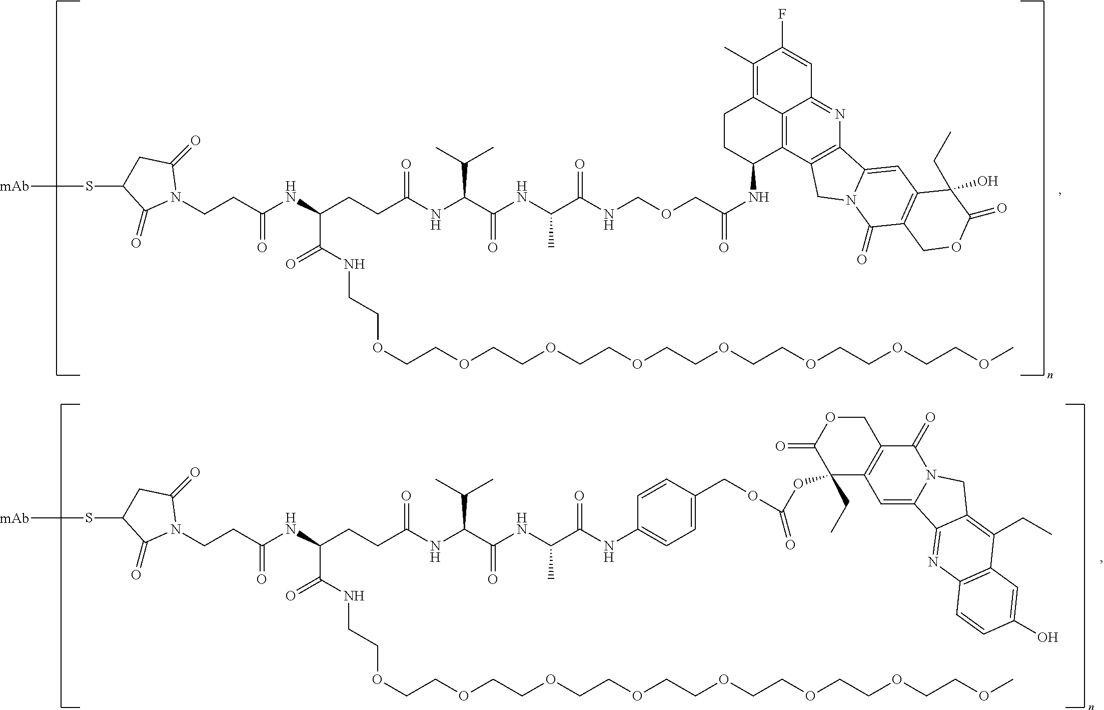

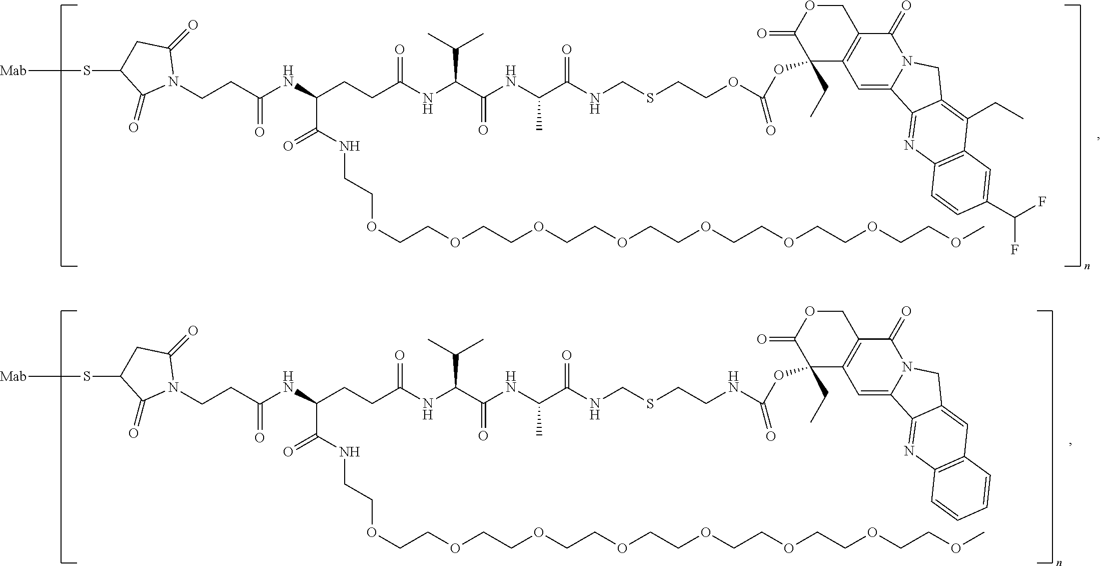

or a pharmaceutically acceptable salt thereof, wherein mAb represents a monoclonal antibody.

In some embodiments, the ligand targets and binds to a tumor antigen.

In some embodiments, in the ligand-drug conjugate, the ligand targets and binds to a receptor tyrosine-protein kinase ERBB2 (HER2), an epidermal growth factor receptor (EGFR), TROP2 and/or FGFR2b.

In some embodiments, the ligand comprises an anti-HER2 antibody, an anti-EGFR antibody, an anti-TROP2 antibody and an anti-FGFR2b antibody.

In some embodiments, the anti-HER2 antibody comprises trastuzumab, pertuzumab or inetetamab.

In some embodiments, the anti-TROP2 antibody comprises sacituzumab.

In some embodiments, the anti-FGFR2b antibody comprises bemarituzumab.

In some embodiments, the anti-EGFR antibody comprises cetuximab, panitumumab, necitumumab or nimotuzumab.

In another aspect, the present application provides a method for preparing the defined ligand-drug conjugate, comprising conjugating an antibody to the previously defined linker or the previously defined linker-drug conjugate.

In another aspect, the present application provides use of the previously defined linker-drug conjugate or the previously defined ligand-drug conjugate in preparation of a drug for treatment of diseases with overexpressed tumor antigens.

In some embodiments, the tumor antigens comprise HER2, EGFR, TROP2 and/or FGFR2b.

In some embodiments, the diseases with overexpressed tumor antigens comprise tumors.

In some embodiments, the diseases with overexpressed tumor antigens comprise tumors expressing HER2, EGFR, TROP2 and/or FGFR2b.

In another aspect, the present application provides use of the previously defined linker-drug conjugate or the previously defined ligand-drug conjugate in preparation of a drug for cancers.

In some embodiments, the cancers comprise lymphoma, leukemia or solid tumors.

In some embodiments, the cancers comprise cancers with upregulated expression and/or activity of HER2.

In some embodiments, the cancers comprise cancers with upregulated expression and/or activity of EGFR.

In some embodiments, the cancers comprise cancers with upregulated expression and/or activity of TROP2.

In some embodiments, the cancers comprise cancers with upregulated expression and/or activity of FGFR2b.

In some embodiments, the cancers comprise a lung cancer, a breast cancer, a prostate cancer, a urothelial cancer, a gastric cancer, a colorectal cancer, an esophageal cancer, a salivary gland cancer, gastroesophageal junction adenocarcinoma, a biliary tract cancer, Paget's disease, a pancreatic cancer, an ovarian cancer or uterine carcinosarcoma.

In another aspect, the present application provides use of the previously defined linker-drug conjugate or the previously defined ligand-drug conjugate in treatment of cancers.

In another aspect, the present application provides a pharmaceutical composition, comprising the previously defined linker-drug conjugate or the previously defined ligand-drug conjugate, and a pharmaceutically acceptable carrier.

In another aspect, the present application provides a method for treating diseases or disorders, comprising administering an effective amount of the previously defined linker-drug conjugate, the previously defined ligand-drug conjugate, or the previously defined pharmaceutical composition to a subject in need thereof.

In some embodiments, the diseases or disorders comprise cancers.

In another aspect, the present application provides a method for inhibiting cancer cell activity or killing cancer cells, comprising contacting cancer cells with an effective amount of the previously defined linker-drug conjugate, the previously defined ligand-drug conjugate, or the previously defined pharmaceutical composition.

In another aspect, the present application provides a drug cassette comprising the previously defined linker-drug conjugate, the previously defined ligand-drug conjugate, or the previously defined pharmaceutical composition, and optionally, an additional therapeutic agent.

Other aspects and advantages of the present application may be readily perceived by those skilled in the art from the detailed description below. The detailed description below only illustrates and describes the exemplary embodiments of the present application. As would be appreciated by those skilled in the art, the content of the present application allows those killed in the art to change the specific embodiments disclosed without departing from the spirit and scope involved in the present application. Accordingly, the accompanying drawings and the description in the specification of the present application are merely for an exemplary but not restrictive purpose.

### BRIEF DESCRIPTION OF THE DRAWINGS

The specific features of the invention involved in the present application are listed in the appended claims. The characteristics and advantages of the invention involved in the present application may be better understood by referring to the exemplary embodiments and the accompanying drawings described in detail below. The accompanying drawings are briefly illustrated as follows.

FIG. 1A to FIG. 1B show the SEC detection results of the antibody-drug conjugate Tr-1b of the present application;

FIG. 2 shows the RPLC detection results of the antibody-drug conjugate Tr-1b of the present application;

FIG. 3 shows the effect curves of the antibody-drug conjugate Tr-1b of the present application on SK-BR-3 cells;

FIG. 4 shows the effect curves of the antibody-drug conjugate Tr-1b of the present application on NCI-N87 cells;

FIG. 5 shows the effect curves of the antibody-drug conjugate Sa-1b of the present application on MDA-MB-468 cells;

FIG. 6 shows the effect curves of the antibody-drug conjugate Sa-1b of the present application on SNU216 cells;

FIG. 7 shows the effect curves of the antibody-drug conjugate Sa-1b of the present application on NCI-H1650 cells;

FIG. 8 shows the effect curves of the antibody-drug conjugate Sa-1b of the present application on NCI-H596 cells;

FIG. 9 shows the effect curves of the antibody-drug conjugate Be-1b of the present application on SNU16 cells;

FIG. 10 shows the effect curves of the antibody-drug conjugate Be-1b of the present application on OCUM-2M cells;

FIG. 11 shows the tumor inhibition effect of the antibody-drug conjugate Tr-1b of the present application against a human gastric cancer cell NCI-N87 model; and

FIG. 12 shows the tumor inhibition effect of the antibody-drug conjugate Tr-1b of the present application against a human breast cancer cell BT474 model.

### DETAILED DESCRIPTION OF THE PREFERRED EMBODIMENTS

The embodiments of the invention of the present application will be illustrated by specific examples below. Those familiar with this technology may easily understand other advantages and effects of the invention of the present application from the content disclosed in the specification.

### Terms & Definitions

In the present application, the term “antibody” is generally used in the broadest sense and specifically covers monoclonal antibodies, polyclonal antibodies, dimers, multimers, polyspecific antibodies (for example, bispecific antibodies), and antibody fragments, provided that they show the desirable biological activity (Milleretal (2003), Jour. Of Immunology, 170: 4854-4861). The naturally occurring form of an antibody is generally a tetramer and consists of two identical immunoglobulin chain pairs, each with a light chain and a heavy chain. In each pair, light-chain and heavy-chain variable regions (VL and VH) together are primarily responsible for binding to antigens. Light-chain and heavy-chain variable domains each consist of framework regions interrupted by three highly variable regions, also known as “complementary determining regions” or “CDRs”. Constant zones can be recognized by and interact with the immune system. (See, for example, Janeway et al., 2001, Immunol. Biology, 5th edition, Garland Publishing, New York). The antibody can be of any type (for example, IgG, IgE, IgM, IgD, and IgA), any class (for example, IgG1, IgG2, IgG3, IgG4, IgA1, and IgA2), or any subclass. The antibody can be derived from any suitable species. In some embodiments, the antibody is of a human or mouse origin. The antibody can be, for example, a human, humanized, or chimeric antibody.

In the present application, the term “monoclonal antibody” generally refers to an antibody obtained from a substantially homogeneous antibody population (that is, except for a few potentially naturally occurring mutation, the antibodies constituting the population are identical). The monoclonal antibody is highly specific for a single antigen site. The modifier “monoclonal” indicates that the antibody acquires this feature from the substantially homogeneous antibody population, and should not be understood to mean that the antibody need to be produced by any particular method.

In the present application, the “intact antibody” is generally an antibody comprising an antigen-binding variable region, a light-chain constant domain (CL) and heavy-chain constant domains CH1, CH2, CH3 and CH4. The constant domains can be natural sequence constant domains (for example, human natural sequence constant domains) or their amino acid sequence variants.

In the present application, the “antibody fragment” generally comprises a portion of a complete antibody, including its antigen-binding or variable region. Examples of the antibody fragment include Fab, Fab′, F(ab′)2 and Fv fragments, double-stranded antibodies, three-stranded antibodies, four-stranded antibodies, linear antibodies, single-chain antibody molecules, scFv, scFv-Fc, polyspecific antibody fragments formed from one or more antibody fragments, one or more fragments generated by an Fab expression library, or epitope-binding fragments of any of the above, with the epitope-binding fragments immunospecifically binding to target antigens (for example, cancer cell antigens, viral antigens, or microbial antigens).

In the present application, the term “antigen” generally refers to an entity specifically binding to an antibody.

In the present application, the terms “specific binding” and “specifically binding” refer to the highly selective binding of the antibody or antibody derivative to the corresponding epitope of its target antigen, instead of a plurality of other antigens. Generally, the antibody or antibody derivative conducts binding with an affinity of at least about 1×10⁻⁷ M, preferably 10⁻⁸ M to 10⁻⁹ M, 10⁻¹⁰ M, 10⁻¹¹ M or 10⁻¹² M, and it binds to a predetermined antigen with an affinity at least twice as great as its binding affinity for a non-specific antigen other than the intended antigen or closely related antigen (for example, BSA, casein).

In the present application, the term “inhibition” generally refers to the reduction by detectable amount, or complete prevention.

In the present application, the term “tumor” generally refers to all neoplastic cell growth and proliferation, both malignant and benign, as well as all pre-cancerous and cancerous cells and tissues. In the present application, the tumor may include a solid tumor and/or a hematologic tumor. The terms “cancer”, “cancerous”, “cytoproliferative condition”, “proliferative condition” and “tumor” are not mutually exclusive when mentioned herein. In some embodiments, the tumor may refer to a mass of flesh containing a majority of cancer cells, for example, cells displaying the characteristics of any cancer described herein. Examples of the tumor may include primary tumors of any of the above types of cancers, or metastatic tumors at second sites derived from any of these types of cancers.

In the present application, the term “tumor antigen” generally includes the meaning known in the art, including any molecule expressed on a tumor cell (or associated with tumor cell development), and known or recognized as having an effect on the tumorigenic properties of tumor cells. Many tumor antigens are known in the art. Whether a molecule is a tumor antigen may also be determined according to techniques and assays familiar to those skilled in the art, for example, clonogenic assay, transformation assay, in vitro or in vivo tumor formation assay, gel migration assay, gene knockout analysis or the like. The term “tumor antigen” can refer to a human transmembrane protein, i.e., a cell membrane protein anchored in a cell lipid bilayer. The human transmembrane protein used herein generally comprise an “extracellular domain” that binds to a ligand, a lipophilic transmembrane domain, a conserved intracellular domain (for example, a tyrosine kinase domain), and a carboxyl-terminal signaling domain having several tyrosine residues that can be phosphorylated. The tumor antigen includes molecules such as EGFR, HER2, HER3, HER4, EpCAM, CEA, TRAIL, a TRAIL receptor 1, a TRAIL receptor 2, a lymphotoxin β receptor, CCR4, CD19, CD20, CD22, CD28, CD33, CD40, CD80, CSF-1R, CTLA-4, a fibroblast activating protein (FAP), hepsin, melanoma-associated chondroitin sulfate proteoglycan (MCSP), a prostate-specific membrane antigen (PSMA), a VEGF receptor 1, a VEGF receptor 2, IGF1-R, TSLP-R, TIE-1, TIE-2, TNF-α, a TNF-like apoptosis weak inducer (TWEAK), or IL-1R.

In the present application, the “HER receptor” generally refers to receptor protein tyrosine kinase belonging to the HER receptor family, including EGFR, HER2, HER3 and HER4 receptors and other members of this family to be identified in the future. The HER receptor will generally comprise an extracellular domain, and can bind to a HER ligand; a lipophilic transmembrane domain; a conserved intracellular tyrosine kinase domain; and a carboxyl-terminal signaling domain containing several tyrosine residues that can be phosphorylated. In some embodiments, the HER receptor is a natural sequence human HER receptor.

The terms “ErbB1”, “HER1”, “epidermal growth factor receptor”, and “EGFR” are used interchangeably herein, and refer to, for example, the EGFR disclosed in Carpenter et al., Ann. Rev. Biochem. 56: 881-914 (1987), including its naturally occurring mutant form (for example, mutant EGFRs deleted in Humphrey et al., PNAS(USA), 87: 4207-4211 (1990)).

The terms “ErbB2” and “HER2” are used interchangeably herein, and generally refer to, for example, the human HER2 protein (Genebank number X03363) described in Semba et al., PNAS(USA), 82: 6497-6501 (1985) and Yamamoto et al., Nature, 319: 230-234 (1986). The extracellular domain of “HER2” generally comprises four domains, namely, a domain I (amino acid residues approximately at positions 1-195), a domain II (amino acid residues approximately at positions 196-319), a domain III (amino acid residues approximately at positions 320-488), and a domain IV (amino acid residues approximately at positions 489-630) (exclusion of signal peptides from the residue numbers). See Garrett et al., Mol. Cell. 11: 495-505 (2003); Cho et al., Nature. 421: 756-760 (2003); Franklin et al., Cancer Cell. 5: 317-328 (2004); or Plowman et al., Proc. Natl. Acad. Sci. 90:1746-1750 (1993).

In the present application, the terms “trastuzumab”, “pertuzumab”, “inetetamab”, “cetuximab”, “panitumumab”, “necitumumab”, “matuzumab” and “nimotuzumab” are used in their general and ordinary meanings as understood in the art.

In the present application, the term “cytotoxic agent” or “cytotoxin agent” generally refers to a substance that has a cytotoxic activity and causes cellular destruction. The term is intended to include radioisotopes, chemotherapeutic agents, and toxins (for example, small molecule toxins or enzymatically active toxins of a bacterial, fungal, plant or animal origin), including analogues and derivatives of their synthesis. The term “cytotoxic activity” generally refers to the cell-killing effect of intracellular metabolites of drugs (such as camptothecin conjugates). The cytotoxic activity can be expressed in IC50 value, which indicates the unit volume concentration (mole or mass) allowing survival of half of the cells.

In the present application, the term “cell growth inhibitor” or “cytostatics” generally refers to a substance with a cell growth inhibition activity, including a substance that inhibits cell growth or proliferation. The cytostatics include inhibitors such as protein inhibitors, for example enzyme inhibitors. The cytostatics have cytostatic activity. The term “cytostatic activity” generally refers to the antiproliferative effect of intracellular metabolites of drugs (such as camptothecin conjugates).

In the present application, the term “camptothecin” generally refers to a pyrroloquinoline cytotoxic alkaloid, which consists of a quinoline ring AB, a pyrrole ring C, a pyridone ring D and a α-hydroxylactone ring E, wherein the 20-position is of an S configuration, with the structure as follows:

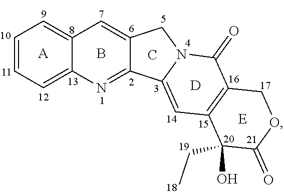

The lactone ring (E ring), the pyridone ring (D ring) and the C20 hydroxyl are considered to be groups necessary for CPT to exert the inhibitory effect of topoisomerase I to achieve an anti-tumor effect.

In the present application, the term “10-difluoromethyl camptothecin compound” generally refers to a camptothecin derivative with difluoromethyl substitution present at position 10 of camptothecin as disclosed in CN201810497805, which is incorporated into the present application in its entirety by reference.

In the present application, the terms “camptothecin”, “exatecan”, “topotecan”, “irinotecan”, “belotecan”, “lurtotecan”, “CKD-602”, “gimatecan”, “karenitecin”, BN-80915, “hydroxycamptothecin”, “9-aminocamptothecin”, “9-nitrocamptothecin”, “7-ethyl-10-hydroxycamptothecin”, and “7-ethyl-10-difluoromethylcamptothecin” are used according to their general and ordinary meaning as understood in the art.

In the present application, the term “pharmaceutically acceptable” ingredient generally refers to a substance that is suitable for humans and/or animals without excessive adverse side effects (such as toxic, irritating and allergic responses), i.e., with a reasonable benefit/risk ratio.

In the present application, the term “solvate” generally refers to an association or complex of one or more solvent molecules and the compound of the present application. Non-limiting examples of the solvent for producing the solvate include, but are not limited to, water, isopropanol, ethanol, methanol, DMSO, ethyl acetate, acetic acid, and ethanolamine. The term “hydrate” generally refers to a complex with water as a solvent molecule.

In the present application, the term “optical isomers” or “stereoisomers” generally refers to that fact that compounds have the same chemical composition but different spatial arrangement of atoms or group, including any of various stereoisomeric configurations (including geometric isomers) that may exist. It should be understood that a substituent can be linked with the chiral center of a carbon atom. The term “chiral” refers to the fact that molecules have non-overlapping characteristics with their mirror pair of molecules, while the term “non-chiral” refers to the fact that molecules can overlap with their mirror pair of molecules. Accordingly, the present invention comprises the enantiomers, diastereoisomers or racemes of a compound. The “enantiomers” refer to a pair of stereoisomers that do not overlap in mirror image. The mixture of a pair of enantiomers at 1:1 is a “racemic” mixture. In due time, the term is used to name the racemic mixture. The “diastereoisomers” are stereoisomers having at least two asymmetric atoms, but they are not mirror images of each other. The absolute stereochemistry is described according to the Cahn-Ingold-PrelogR-S system. When compounds are pure enantiomers, the stereochemistry of each chiral carbon can be designated as R or S. Depending on the direction (dextrorotation or levogyration) in which the plane-polarized light is rotated at the wavelength of the sodium D-line, the resolved compounds with unknown absolute configurations can be designated as (+) or (−). Certain compounds described herein contain one or more asymmetric centers or axes, and thus may give rise to enantiomers, diastereomers and other stereoisomeric forms, which can be designated as (R)- or (S)- according to the absolute stereochemistry.

In the present application, the term “tautomers” generally refer to structural isomers, which have different energies and can be mutually transformed by a low energy barrier. For example, proton tautomers (also known as tautomeric isomers of protons) include protolysis-based mutual transformation, for example, ketone-enol and imino-enamine isomerization. Valence tautomers involve the mutual transformation based on recombination of some bonded electrons.

In the present application, the term “isotopes” generally include atoms having the same atomic number but different mass number. Examples of isotopes that can be incorporated into the compound of the present application and pharmaceutically acceptable salts thereof include isotopes of hydrogen, carbon, nitrogen, oxygen, phosphorus, sulfur, fluorine, iodine and chlorine, for example, ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I. The subject matter disclosed in the present application may further include the isotope-labeled forms of the compounds of the present application.

In the present application, the term “metabolite” generally refers to a product produced by in vivo metabolism of a particular compound or its salt. The metabolites of compounds can be identified using conventional techniques known in the art, and their activity may be determined using tests as described in the present application. Such products may be produced by, for example, oxidation, hydroxylation, reduction, hydrolysis, amidation, deamidation, esterification, deesterification, enzymatic cleavage or the like of the compound administered. Accordingly, the present application may further comprise the metabolites of the compounds of the present application, including compounds produced by a method comprising contacting the compounds of the present application with mammals for a period of time sufficient to produce its metabolites.

In the present application, the term “prodrug” or “precursor drug” generally refers to a drug precursor compound, which, when administered to a subject, undergoes chemical transformation via a metabolic or chemical process to obtain the compounds of the present application or salts thereof. The content of prodrugs is well known in the art (see, for example, Berge et al. (1977) “Pharmaceutical Salts”, J. Pharm. Sci. 66: 1-19).

In the present application, the term “pharmaceutically acceptable salt” means a salt capable of maintaining the biological effects and properties of the compounds of the present application, and the salt generally has no biological or other disadvantage. It includes pharmaceutically acceptable organic or inorganic salts, examples of which include, but are not limited to, sulfates, citrates, acetates, oxalates, chlorides, bromides, iodides, nitrates, bisulfates, phosphates, acid phosphates, isonicotinates, lactates, salicylates, acid citrates, tartrates, tannins, pantothenates, bitartrates, ascorbate, succinates, maleates, gentisates, fumarates, gluconates, glucuronates, saccharates, formates, benzoates, glutamates, methanesulfonates, ethanesulfonates, benzenesulfonates, p-toluenesulfonates, bis-hydroxynaphphates (i.e., 1,1′-methylene-bis-(2-hydroxy-3-naphthoic acid) salt), alkali metal (for example, sodium and potassium) salts, alkaline earth metal (for example, magnesium) salts, and ammonium salts. The pharmaceutically acceptable salts may involve the inclusion of an additional molecule, which is, for example, an acetate ion, a succinate ion or other countering ions. The counterion may be any organic or inorganic fraction for stabilizing the charge on the parent compound. In addition, the pharmaceutically acceptable salts may have more than one charged atom in their structure. In instances where a plurality of charged atoms are part of a pharmaceutically acceptable salt, the salt may have a plurality of counter ions. Thus, the pharmaceutically acceptable salt may have one or more charged atoms and/or one or more counter ions.

In the present application, the term “linker” or “linker unit” generally refers to a bifunctional portion linking a drug (for example, camptothecin) to a ligand unit in a drug-ligand conjugate. The linker unit of the present application has a plurality of components (for example, in some embodiments, which have extension units of alkaline units; branching units that may or may not exist; spacer unit; and releasable assembly units).

In the present application, the term “PEG”, “PEG unit” or “polyethylene glycol” is an organic portion consisting of repeated ethylene-oxy subunits, and may be polydisperse, monodisperse or discrete (i.e., having a discrete number of ethylene-oxy subunits). Polydisperse PEGs are mixtures heterogeneous in size and molecular weight, while monodisperse PEGs are generally purified from the heterogeneous mixtures and thus have a single chain length and molecular weight. The PEG unit of the present application may comprise one or more polyethylene glycol chains, each of which is composed of one or more ethylene-oxy units that are mutually covalently linked. The polyethylene glycol chains may be linked together in a linear, branching or star configuration, for example. Generally, in each polyethylene glycol chain, a terminal ethylene-oxy subunit that is not covalently linked to the rest of the linker unit is modified by a PEG capping unit, which is generally an optionally substituted alkyl such as —CH₃, CH₂CH₃ or CH₂CH₂CO₂H. In some embodiments, the PEG unit has a single polyethylene glycol chain with 1 to 20 —CH₂CH₂O-subunits, which are covalently linked in series and terminated by a PEG capping unit at one end.

In the present application, the term “optionally substituted” indicates that the group involved may be substituted or not substituted. During substitution, the substituent of an “optionally substituted” group may include, but is not limited to, one or more substituents independently selected from the following groups alone or in combination or a specially designated group of groups: alkyl, alkenyl, alkynyl, cycloalkyl, aryl, heteroaryl, heterocycloalkyl, hydroxyl, alkoxy, sulfydryl, cyano, halogen, carbonyl, thiocarbonyl, isocyanate, thiocyanate, isothiocyanate, nitro, per-haloalkyl and amino including monosubstituted and disubstituted amino groups, and their protected derivatives. Non-limiting examples of the optional substituent include halogen, —CN, ═O, ═N—OH, ═N—OR, ═N—R, —OR, —C(O)R, —C(O)OR, —OC(O)R, —OC(O)OR, —C(O)NHR, —C(O)NR₂, —OC(O)NHR, —OC(O)NR₂, —SR—, —S(O)R, —S(O)₂R, —NHR, —N(R)₂, —NHC(O)R, —NRC(O)R, —NHC(O)OR, —NRC(O)OR, S(O)₂NHR, —S(O)₂N(R)₂, —NHS(O)₂NR₂, —NRS(O)₂NR₂, —NHS(O)₂R, —NRS(O)₂R, C₁₋₆ alkyl, C₁₋₆ alkoxy, aryl, heteroaryl, cycloalkyl, heterocycloalkyl, C₁₋₆ haloalkyl, and C₁₋₆ haloalkoxy, wherein each R is independently selected from H, halogen, C₁₋₆ alkyl, C₁₋₆ alkoxy, aryl, heteroaryl, cycloalkyl, heterocycloalkyl, C₁₋₆ haloalkyl, and C₁₋₆ haloalkoxy. The position and number of such substituents are determined according to the well-known valence state limits of each group. For example, ═O is a suitable substituent for alkyl but not for aryl. Two substituents may be linked together to form a five-, six- or seven-membered aromatic or non-aromatic carbon ring or heterocyclic ring containing one to three heteroatoms, for example, to form methylenedioxy or ethylenedioxy.

When it comes to specific nomenclature, the substituent is generally placed before the substituted group, for example, “C₁₋₃ alkoxy C₃₋₈ cycloalkyl C₁₋₆ alkyl” refers to C₁₋₆ alkyl substituted by C₃₋₈ cycloalkyl which is in turn substituted by C₁₋₃ alkoxy. For instance, methoxycyclobutylmethyl has a structural formula as follows:

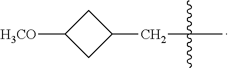 [SMILES: *CC1CC(OC)C1]

In the present application, the number of carbon atoms is generally indicated by the prefix “Cₓ-C_{y}” or “C_{x-y}”, wherein x is the minimum number of carbon atoms in the substituent and y is the maximum number. For example, “C₁-C₆ alkyl” or “C₁₋₆ alkyl” refers to an alkyl substituent containing 1 to 6 carbon atoms. To further illustrate, C₃-C₆ cycloalkyl or C₃₋₆ cycloalkyl refers to saturated cycloalkyl containing 3 to 6 carbon ring atoms.

In the present application, the term “alkyl” generally refers to a straight-chain, branching-chain or cyclic saturated substituent consisting of carbon and hydrogen. Non-limiting examples of alkyl include: methyl, ethyl, propyl (including n-propyl and isopropyl), butyl (including n-butyl, isobutyl, sec-butyl and tert-butyl), amyl, isoamyl, hexyl, and the like. Depending on the case, alkyl may be optionally substituted on each carbon as defined by the claims. Typical substituents include, but are not limited to: fluorine, chlorine, OH, cyano, alkyl (optionally substituted), cycloalkyl, and the like.

In the present application, the term “cycloalkyl” generally refers to a saturated monocyclic cyclohydrocarbon group. A single ring typically consists of 3 to 10 carbon atoms. Non-limiting examples of cycloalkyl include cyclopropyl, cyclobutyl, cyclopentyl, cyclopentenyl, cyclohexyl, cyclohexenyl, cyclohexadienyl, cycloheptyl and the like;

In the present application, the term “heterocyclyl” or “heterocycle” generally refers to the inclusion of at least one atom on the structure of the ring, specifically, for example, the inclusion of one or more non-aromatic heterocyclyl, bicyclic heterocyclyl and polycyclic heterocyclyl of the same or different heteroatoms arbitrarily selected from O, S and N.

The term “aryl” generally refers to a hydrocarbon monocyclic or bicyclic aromatic ring system, where such a ring can be fused. If the ring is a fused ring, one of the rings must be a fully unsaturated ring, and the fused ring can be a fully saturated, partially unsaturated, or completely unsaturated ring. The term “fused” means that the second ring exists (i.e., is linked or formed) by sharing two adjacent atoms with the first ring. The term “aryl” includes aromatic groups, for example, phenyl, naphthonyl, tetrahydronaphthyl, indanyl, biphenyl, 4-(pyridin-3-yl)phenyl, 2,3-dihydro-1H indanyl and 1,2,3,4-tetrahydronaphthoyl.

The term “heteroaryl” generally refers to an aromatic group containing at least one heteroatom (for example, oxygen, sulfur, nitrogen or their combinations) in a 5- to 10-membered aromatic ring system (for example, pyrrole, pyridinyl, pyrazole, indole, indrazol, thienyl, furan, benzofuran, oxazolyl, imidazolyl, tetrazolyl, triazinyl, pyrimidyl, pyrazinyl, thiazolyl, purinyl, benzimidazolyl, quinolyl, isoquinolyl, benzothiophenyl, benzooxazolyl, 1H-benzo[d][1,2,3]triazolyl, etc.). The heteroaromatic groups may consist of monocyclic or fused ring systems. A typical monoheteroaryl ring is a 5- to 6-membered ring containing 1 to 3 heteroatoms independently selected from oxygen, sulfur and nitrogen, and a typical fused heteroaryl ring system is a 9- to 10-membered ring system containing 1 to 4 heteroatoms independently selected from oxygen, sulfur and nitrogen. The fused heteroaryl ring system may consist of two heteroaryl rings fused together, or heteroaryl fused to aryl (for example, phenyl).

In the present application, the wedge solid line key ( [symbol] ) and the wedge dashed key ( [symbol] ) are typically used to represent the absolute configuration of a stereocenter, and the straight solid line key ( [symbol] ) and the straight dashed key ( [symbol] ) are used to represent the relative configuration of the stereocenter. Unless otherwise specified, all compounds present in the present invention are intended to include all possible optical isomers, such as monochiral compounds, or mixtures (i.e., racemates) of various chiral compounds. Among all compounds of the present invention, each chiral carbon atom may optionally be of an R configuration or S configuration, or a mixture of R configuration and S configuration.

In the present application, the term “carrier” may comprise a pharmaceutically acceptable carrier, excipient, or stabilizer, which is nontoxic for the cells or mammals that are exposed to it at the dose and concentration used. Generally, the physiologically acceptable carrier is a PH buffered aqueous solution. Non-limiting examples of the physiologically acceptable carrier may comprise: buffers, for example phosphate, citrate, and other organic acids; antioxidants, including ascorbic acid; low-molecular-weight (less than about 10 residues) polypeptides, and proteins, for example serum albumin, gelatin, or immunoglobulin; hydrophilic polymers, for example polyvinylpyrrolidone; amino acids, such as glycine, glutamine, asparagine, arginine, or lysine; monosaccharides, disaccharides, and other carbohydrates, including glucose, mannose, or dextrin; chelating agents, for example EDTA; sugar alcohols, for example mannitol or sorbitol; salt-forming counterions, for example sodium; and/or nonionic surfactants, for example TWEEN™, polyethylene glycol (PEG), and PLURONICS™. In some embodiments, the pharmaceutically acceptable carrier is a naturally-occurring pharmaceutically acceptable carrier.

In the present application, the term “abnormal” generally refers to deviations from the standard, for example, an ordinary healthy object or cell and/or a group of ordinary healthy objects or cells. As used herein, the term “abnormal expression” refers to the abnormal expression of gene products (for example, RNAs, proteins, polypeptides, or peptides) of a cell or object as compared to a normal healthy cell or object and/or a group of normal healthy cells or objects. This abnormal expression may be caused by gene amplification or inhibited gene expression. In some embodiments, the “abnormal expression” associated with DNase topoisomerase I refers to the elevated, decreased or inappropriate expression of DNase topoisomerase I. In a specific embodiment, the term “abnormal activity” refers to the deviation of DNase topoisomerase I from its normal activity in a healthy cell or object and/or a group of normal healthy cells or objects.

In the present application, the term “upregulated expression” generally refers to an increase in the expression of nucleic acid mRNA levels or an increase in the expression of peptide levels. The term may also refer to post-translational modifications required for increased peptide activity and/or function, for example, addition of sugar moieties, phosphorylation or the like.

In the present application, the term “inhibitor” generally refers to a compound/substance that is known in the art and capable of completely or partially preventing or reducing the physiological function (activity) of one or more specific proteins (for example, topoisomerase I). Inhibitors are also called “antagonists.” In the context of the present invention, the inhibitors of topoisomerase I may prevent or reduce or inhibit or inactivate the physiological activity of topoisomerase I.

In the present application, examples of the term “topoisomerase I inhibitor” include, but are not limited to: topotecan, gimatecan, irinotecan, camptothecin and its analogues, 9-nitrocamptothecin and macromolecular camptothecin conjugate PNU-166148 (Compound A1 in WO 99/17804); 10-hydroxycamptothecin acetate; etoposide; idabicin hydrochloride; irinotecan hydrochloride; teniposide; topotecan, topotecan hydrochloride; doxorubicin; epirubicin, epirubicin hydrochloride; 4′-epirubicin, mitoxantrone, mitoxantrone hydrochloride; daunorubicin, daunorubicin hydrochloride, valrubicin, and dasatinib (BMS-354825).

In the present application, the term “treat” or “treatment” generally refers to therapeutic treatment and prophylactic treatment for the purpose of inhibiting or slowing down (mitigating) undesirable physiological changes or conditions, such as the development or spread of cancer. For the purposes of the present invention, beneficial or desired clinical results include, but are not limited to, alleviated symptoms, weakened disease level, stabilized disease status (i.e., no deterioration), delayed or slowed disease progression, improved or mitigated disease status, and remission (whether partial or total), whether detectable or non-detectable. Compared to the expected survival term without treatment, “treatment” may also mean a longer survival term. Those in need of treatment include those who already have the disease or condition and those who are prone to the disease or condition. Within the context of cancer, the term “treatment” may also include any or all of the following: killing tumor cells; inhibiting the growth of tumor cells, cancer cells or tumors; inhibiting the replication of tumor cells or cancer cells; reducing the overall tumor burden or reducing the number of cancer cells; and improving one or more symptoms of concomitant diseases.

The term “dosing” or “administration” includes a route for introduction of the defined compound into a subject to achieve its intended function. Non-restrictive examples of available routes of dosing include injection (subcutaneous, intravenous, parenteral, intraperitoneal, or intrathecal injection), topical administration, oral administration, inhalation, rectal administration, and transcutaneous administration.

The term “contact” generally refers to contacting two or more different types of substances together in any order, in any way, and for any length of time. When applied to cells, “contact” means a method by which the compound of the present application is delivered, in vitro or in vivo, to a target cell or placed directly close to the target cell.

In the present application, the term “effective amount” generally includes an amount necessary for effectively achieving the desired result at a necessary dose and in a necessary time period. The effective amount of a compound can vary depending on factors such as the disease status, age, and weight of a subject, and the capability of the compound to elicit the desired response in the subject. A dose regimen may be adjusted to provide the optimal response to treatment.

In the present application, the term “therapeutically effective amount” generally refers to the amount of a conjugate for effective treatment of diseases or disorders in mammals. In the case of cancer, the therapeutically effective amount of conjugate may reduce the number of cancer cells; reduce tumor size; inhibition (i.e., delay to a certain extent, preferably terminate) the infiltration of cancer cells into peripheral organs; inhibit (i.e., delay to a certain extent, preferably terminate) tumor metastasis; inhibit tumor growth to a certain extent; and/or alleviate one or more symptoms associated with the cancer to some extent. In terms of the extent to which a drug can inhibit cancer cell growth and/or kill existing cancer cells, it may involve inhibiting cell growth and/or cytotoxicity. In terms of cancer treatment, the efficacy may be measured, for example, by assessing the time to disease progression (TTP) and/or determining the response rate (RR).

In the present application, the term “subject” or “patient” refers to an animal, for example, a mammal, including but not limited to primates (for example, humans), cattle, sheep, goats, horses, dogs, cats, rabbits, rats, mice or the like. In some embodiments, the subject is a human.

### DETAILED DESCRIPTION OF THE INVENTION

### Linker

In an aspect, the present application provides a linker comprising an Lb structure fragment, wherein an Lb structure is selected from the following formula:

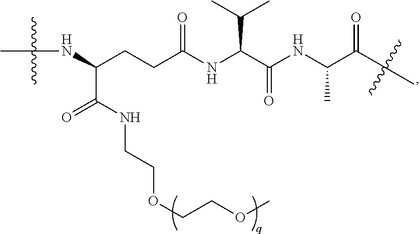

wherein a subscript q is any integer selected from 1-20, preferably 3-10, and most preferably 5-8. For example, the subscript q is selected from 1, 2, 3, 4, 5, 6, 7, 8, 9, 10, 11, 12, 13, 14, 15, 16, 17, 18, 19 and 20.

In some embodiments, the linker comprises an Lb-Lc structure fragment, the Lb structure being linked to an Lc fragment via carbonyl, wherein Lc is a releasable assembly unit and is capable of linking a drug unit.

In some embodiments, the Lc structure is selected from the following formulas:

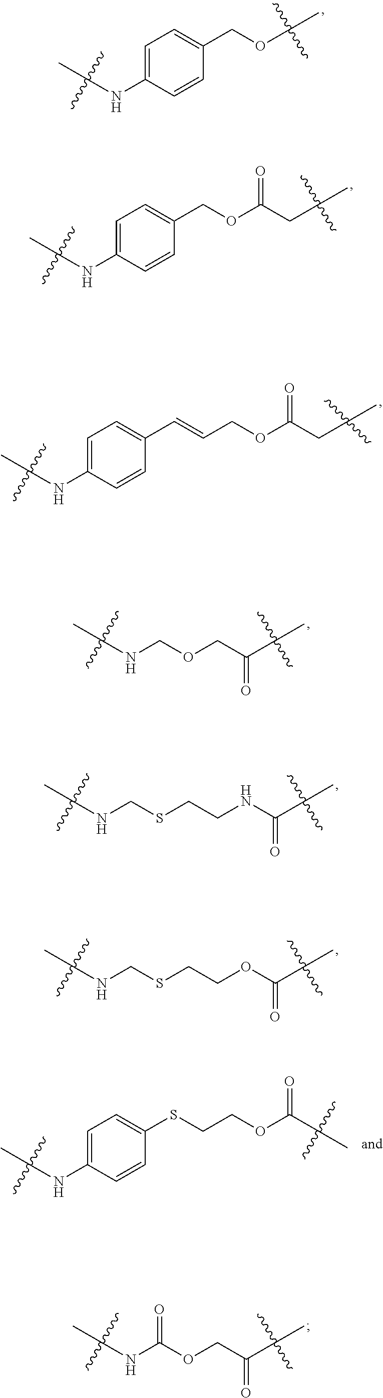

optionally, the Lc structure is linked to carbonyl of the Lb structure via amino; and Lc is capable of linking the drug unit via carbonyl.

For example, the Lb-Lc structure fragment may be selected from the following formulas:

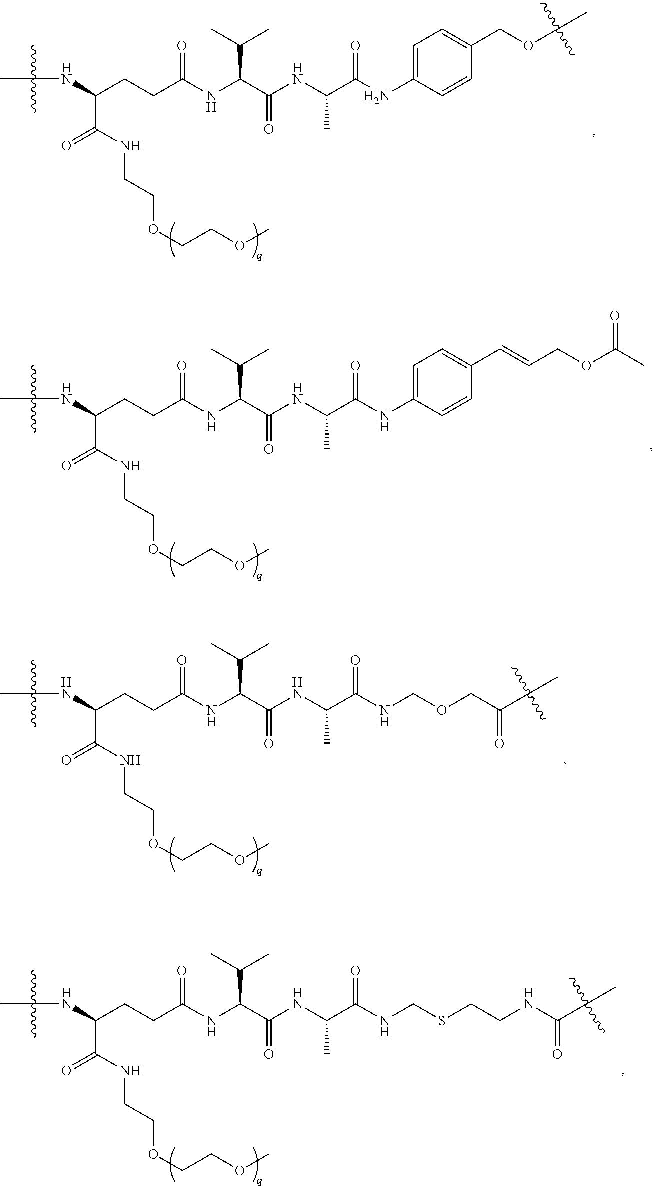

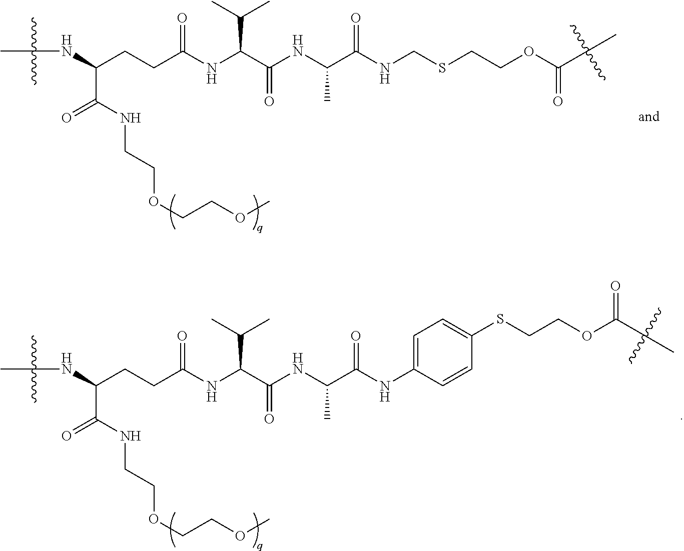

In some embodiments, the linker has a structure shown in Formula (I):

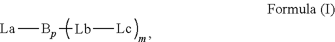

wherein,
La is an extension unit capable of linking a ligand unit;
B is an optional branching unit, wherein a subscript p is 0 or 1, and wherein m is I when p is 0, and m is 2, 3, or 4 when p is 1;
the Lb structure is selected from the following formula:

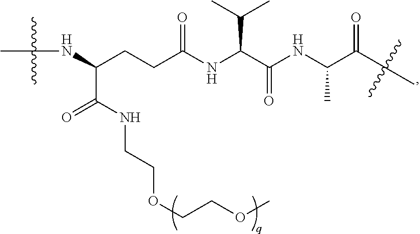

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit capable of linking a drug unit.

In some embodiments, the linker has a structure shown in Formula (Ia), (Ib), (Ic), or (Id):

La-Lb-Lc, Formula (Ia),

La—B [symbol] Lb-Lc)₂, Formula (Ib),

La—B [symbol] Lb-Lc)₃ or Formula (Ic),

La—B [symbol] Lb-Lc)₄, Formula (Id),

In some embodiments, the B structure is selected from the following formula:

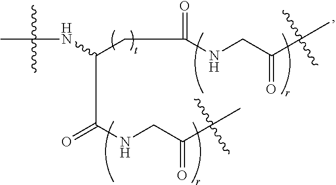

wherein a subscript t is any integer independently selected from 1-4, each subscript r is any integer independently selected from 1-4, and the B structure is linked to the La structure via amino and to the Lb structure fragment via carbonyl.

In some embodiments, the B structure is selected from the following formula:

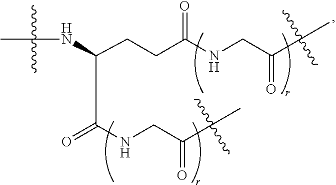

wherein each subscript r is any integer independently selected from 1-4, and the B structure is linked to the La structure via amino and to the Lb structure fragment via carbonyl.

In some embodiments, the La structure comprises a maleimide type linker fragment.

In some embodiments, the La structure has the following structure:

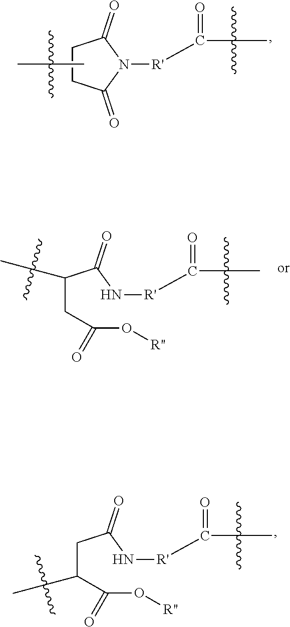

and the La structure is linked to the Lb or B structure fragment via carbonyl, and is capable of linking the ligand unit via position(s) 3 and/or 4 of succinimide/succinamide; and R′ is selected from the group consisting of: optionally substituted C₁-C₁₀ alkyl, C₁-C₁₀ alkoxy, C₁-C₁₀ aminoalkyl and C₁-C₁₀ alkyl-aryl,

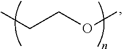

R″ is selected from: optionally substituted C₁-C₁₀ alkyl; wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl; and wherein a subscript n is any integer selected from 1-10, preferably 1-5, and most preferably 1-3.

In some embodiments, R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, hydroxyl, C₁-C₁₀ alkyl, C₁-C₁₀ alkoxy, C₁-C₁₀ aminoalkyl, and C₁-C₁₀ haloalkyl.

In some embodiments, R′ is selected from the group consisting of: optionally substituted C₁-C₁₀ alkyl, C₁-C₁₀ alkoxy, C₁-C₁₀ aminoalkyl and C₁-C₁₀ alkyl-aryl.

In some embodiments, the La structure is selected from the following formulas:

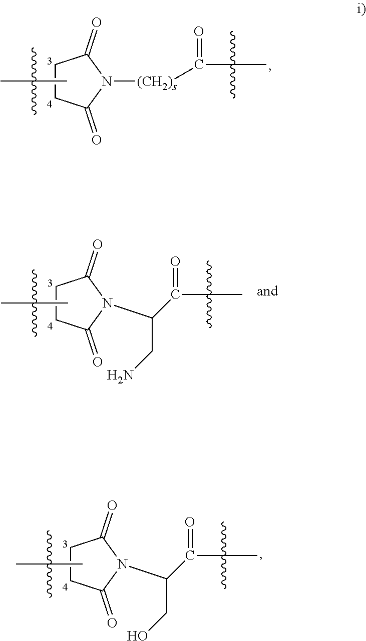

wherein the La structure is linked to the Lb or B structure fragment via carbonyl, and is capable of linking the ligand unit via position(s) 3 and/or 4 of succinimide;

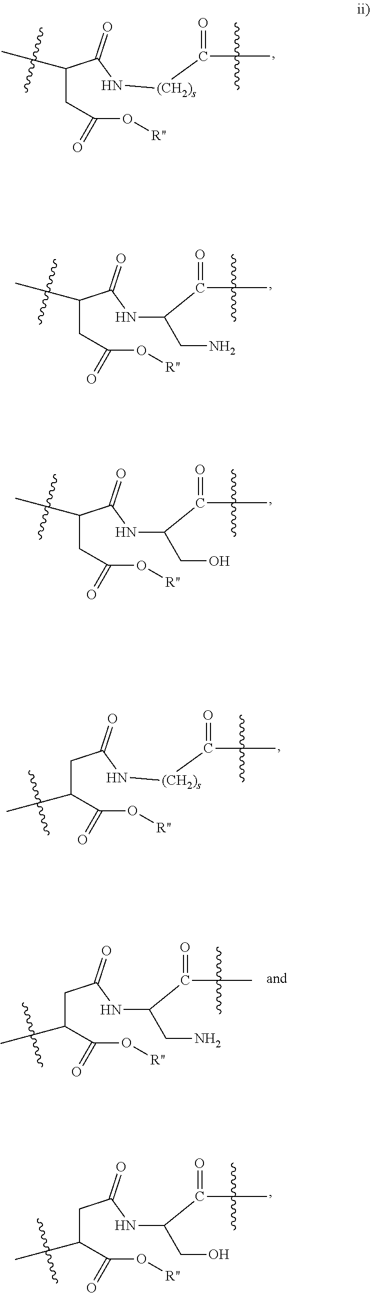

wherein the La structure is linked to the Lb or B structure fragment via carbonyl; and R″ is selected from the group consisting of: methyl, ethyl and isopropyl;
wherein each subscript s is any integer independently selected from 1-10, preferably 1-8, and more preferably 1-5.

In some embodiments, the La structure is selected from the following formula:

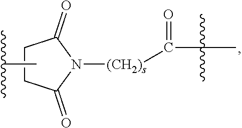

wherein the La structure is linked to the Lb or B structure fragment via carbonyl, and is capable of linking the ligand unit via position(s) 3 and/or 4 of succinimide; and wherein the subscript s is any integer selected from 1-10, preferably 1-8, and more preferably 1-5.

In some embodiments, the La structure is selected from the following formula:

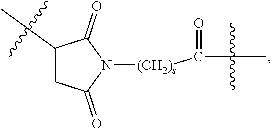

wherein a subscript s is any integer selected from 1-10, preferably 1-8, and more preferably 1-5, and the La structure is linked to the B or Lb structure fragment via carbonyl, and is capable of linking the ligand unit via position 3 of succinimide.

In some embodiments, the La structure is selected from the following formula:

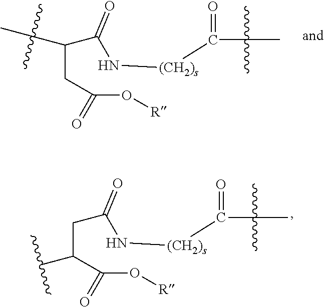

wherein R″ is selected from the group consisting of methyl, ethyl and propyl, a subscript s is any integer selected from 1-10, preferably 1-8, and more preferably 1-5, and the La structure is linked to the B or Lb structure fragment via carbonyl.

For example, the La structure may be selected from the following formulas:

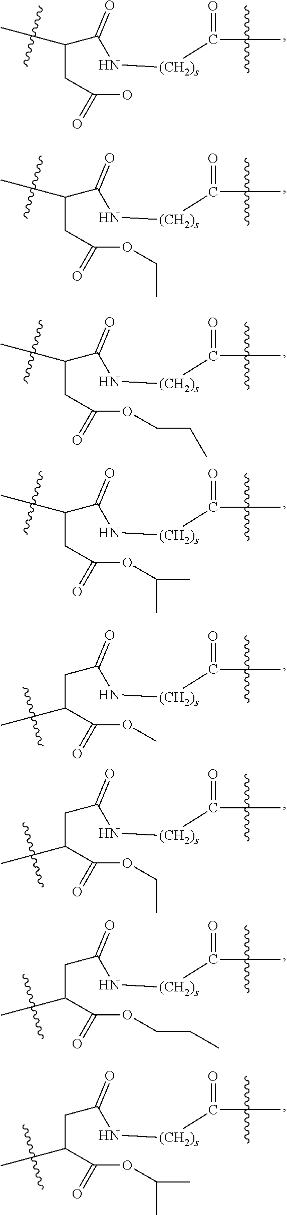

a subscript s is any integer selected from 1-10, preferably 1-8, and more preferably 1-5, and the La structure is linked to the B or Lb structure fragment via carbonyl.

In some embodiments, the linker has a structure shown in Formula (Ia) or Formula (Ib):

La-Lb-Lc, Formula (Ia),

La—B [symbol] Lb-Lc)₂, Formula (Ib),
wherein,
La is an extension unit capable of linking a ligand unit;
B is a branching unit having a structure selected from the following formula:

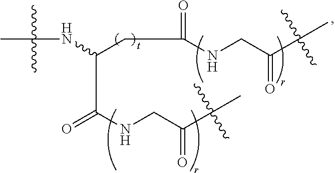

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

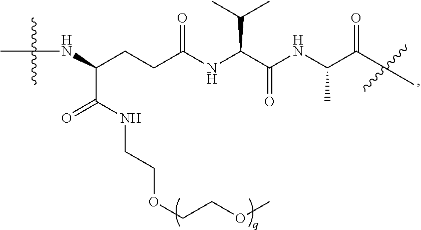

wherein a subscript q is any integer selected from 1-20, preferably 3-10, and most preferably 5-8, and the Lb structure is linked to an La or B structure via amino and to the Lc fragment via carbonyl; and Lc is a releasable assembly unit and is capable of linking a drug unit.

In some embodiments, the linker is selected from the following structures:

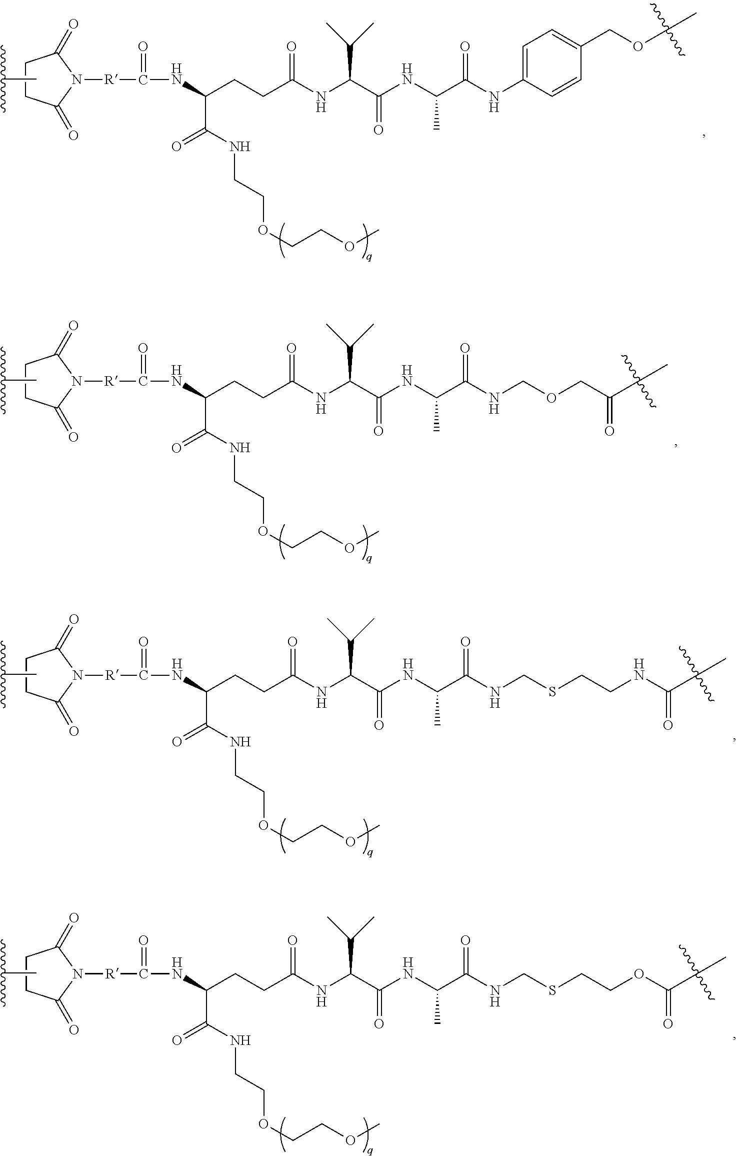

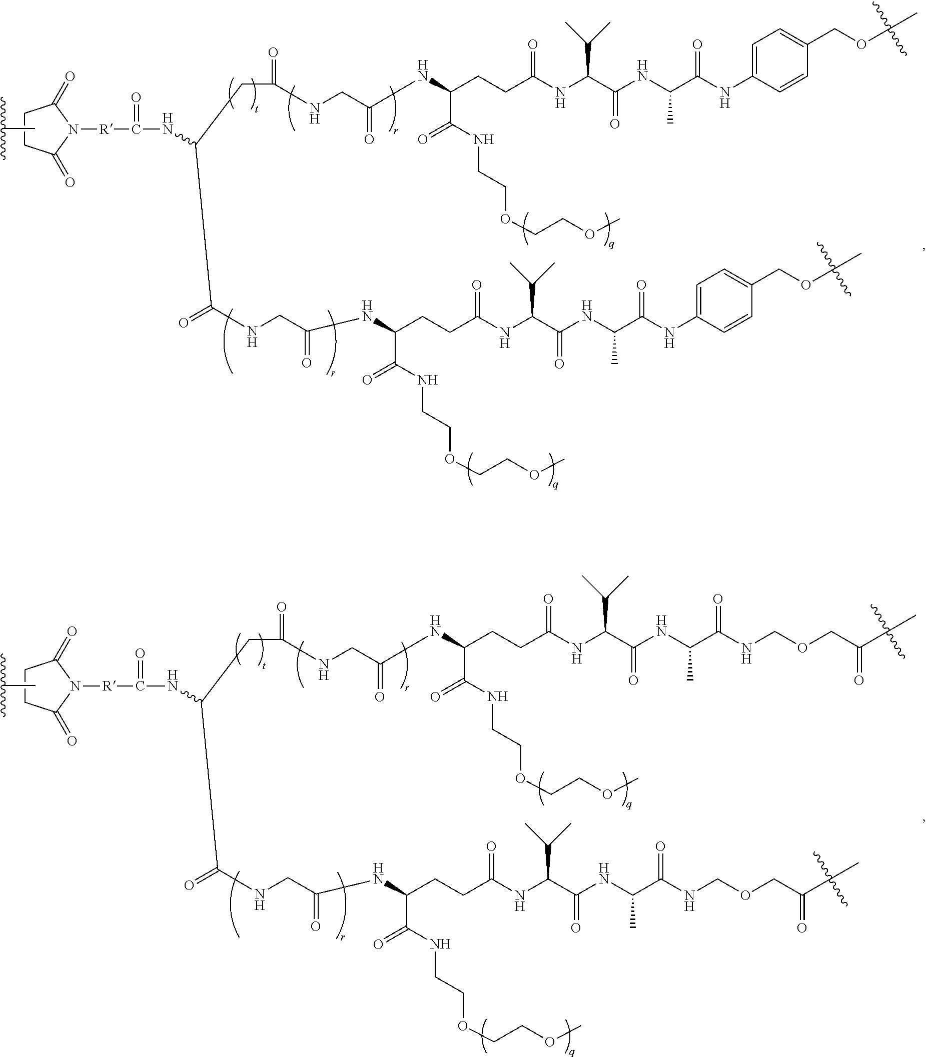

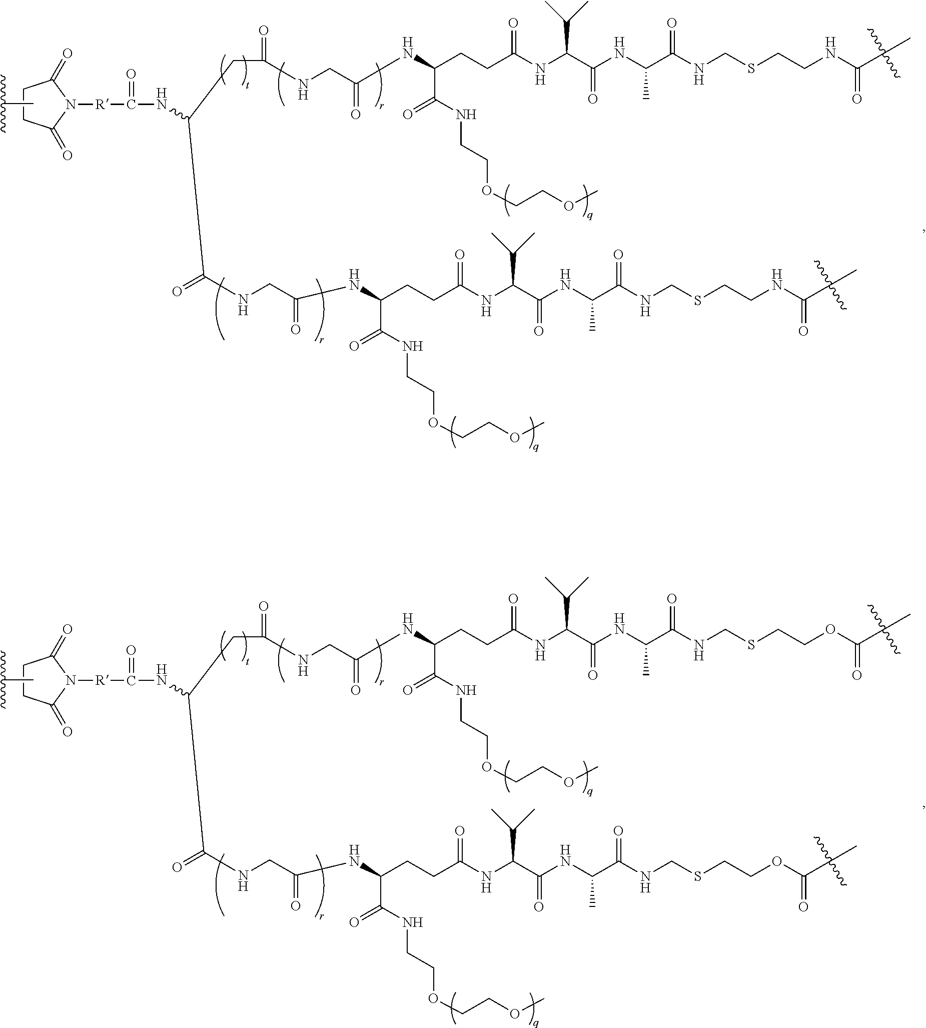

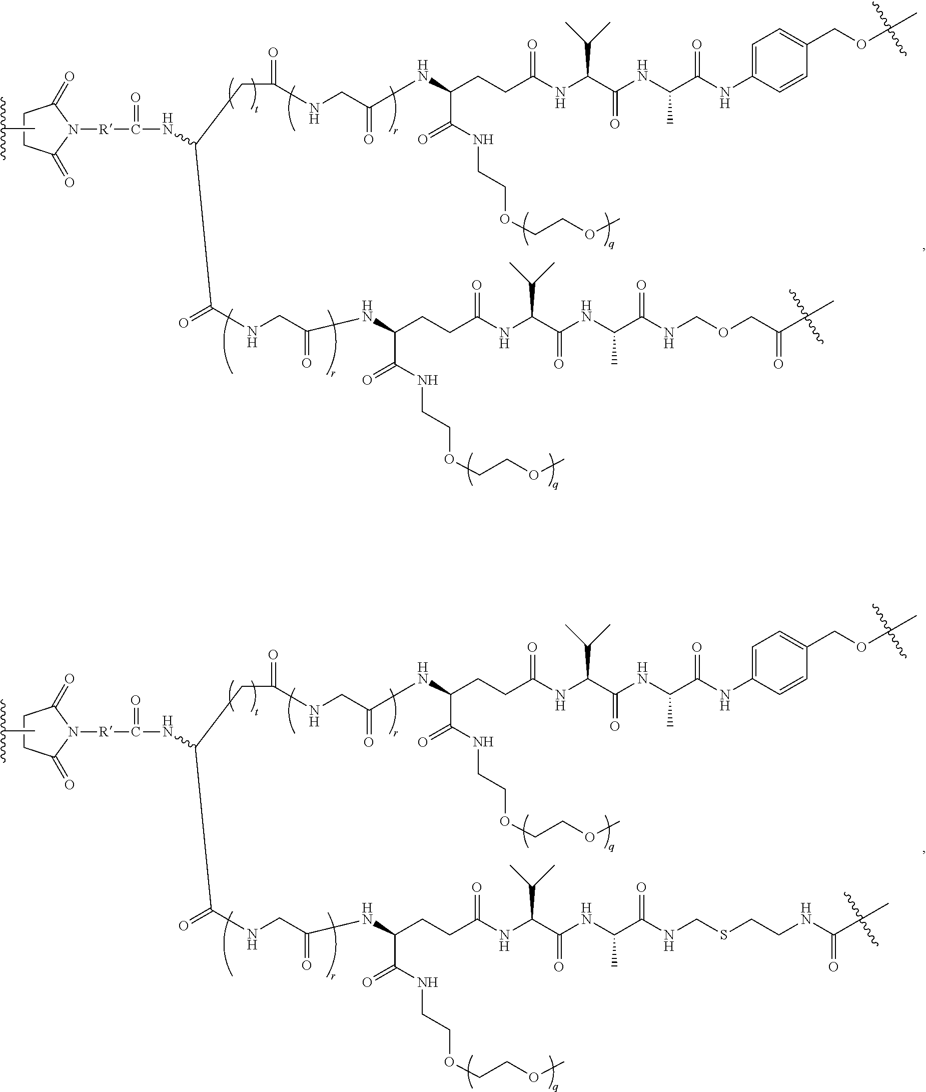

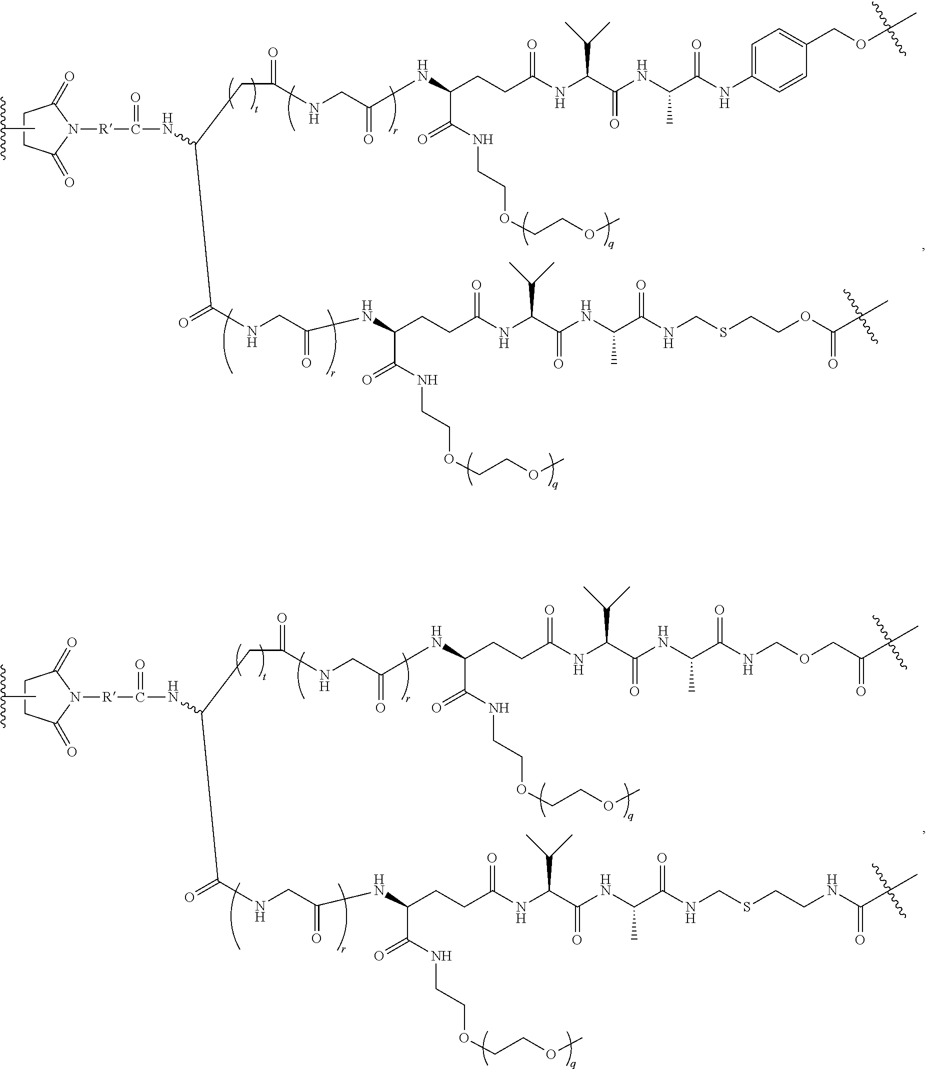

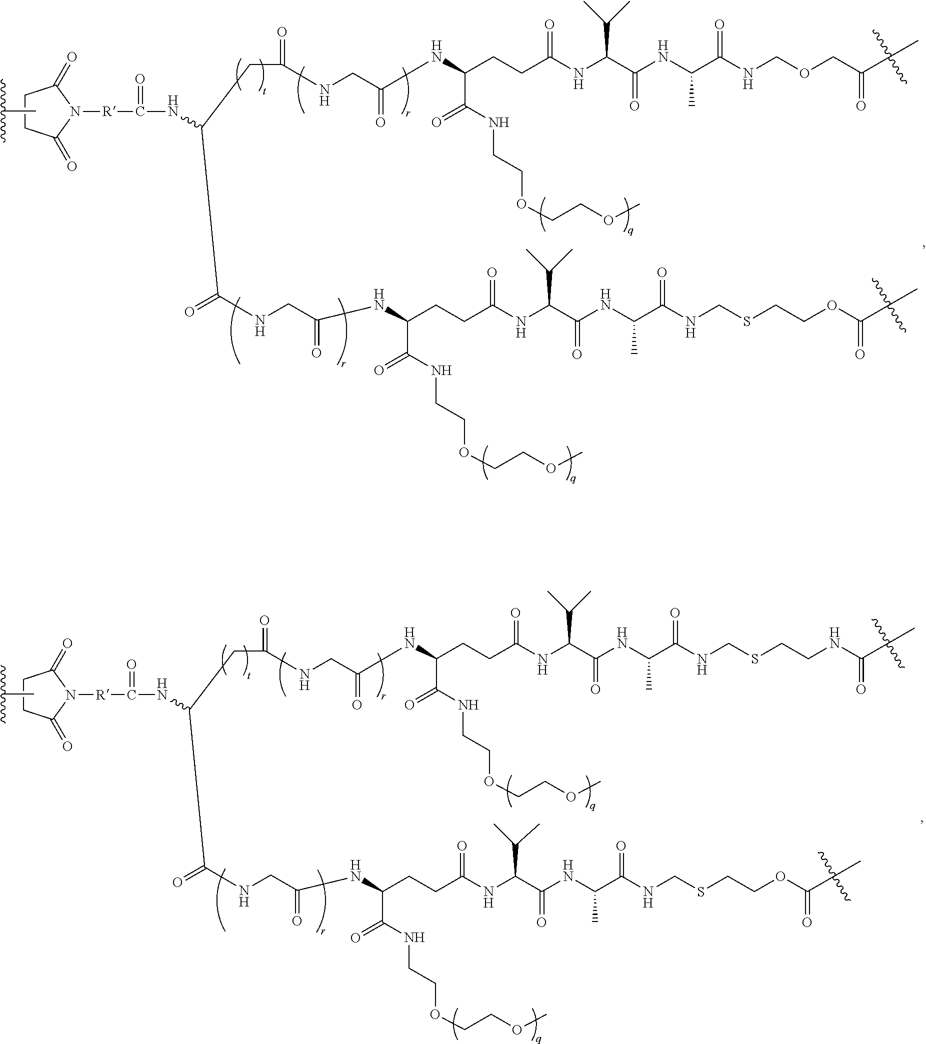

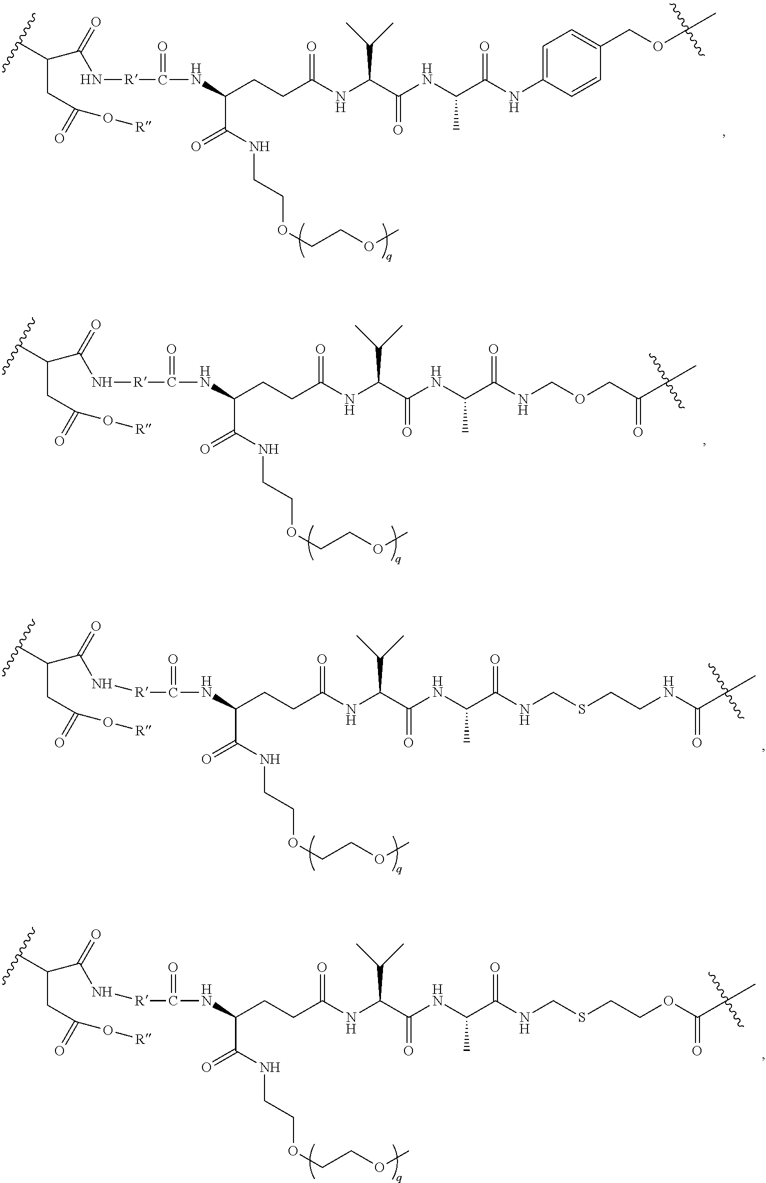

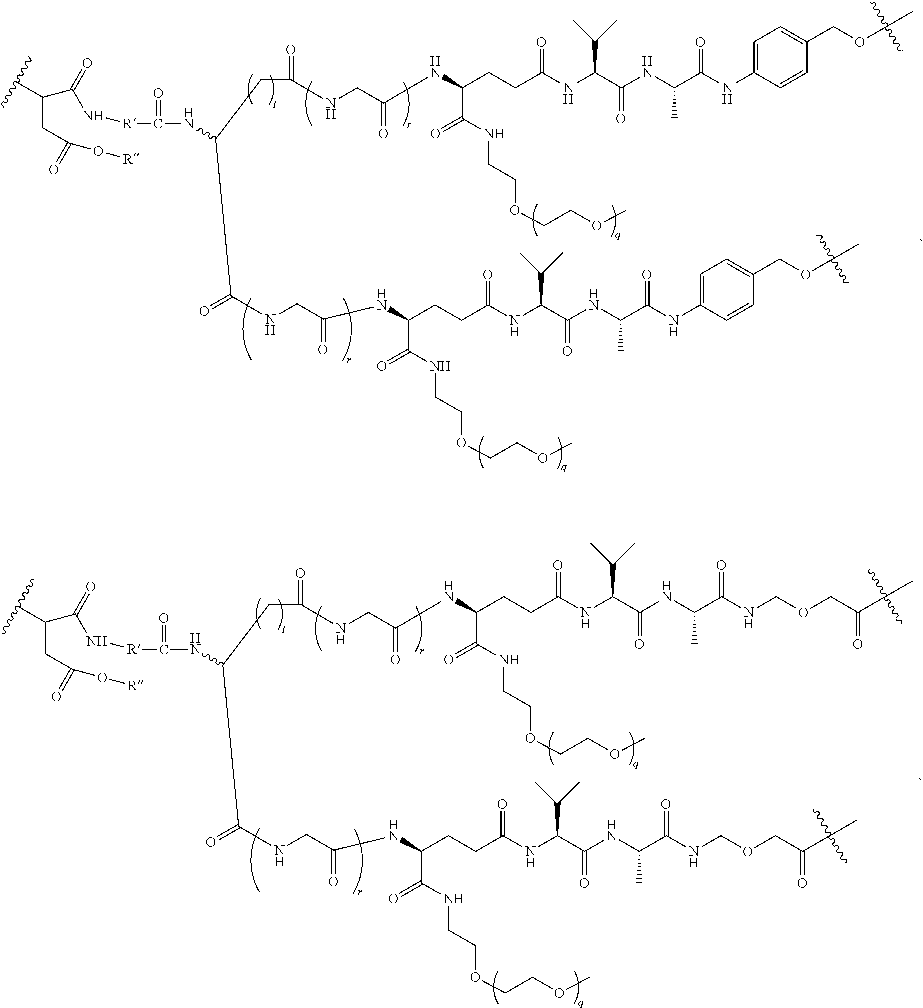

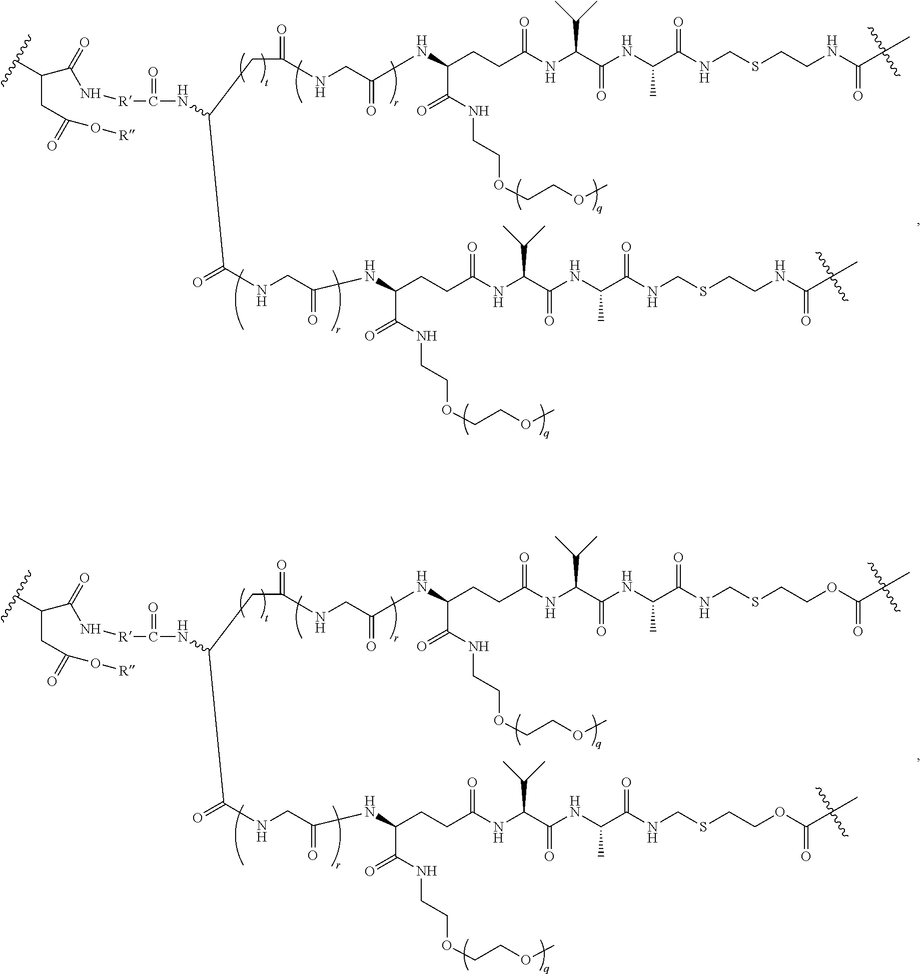

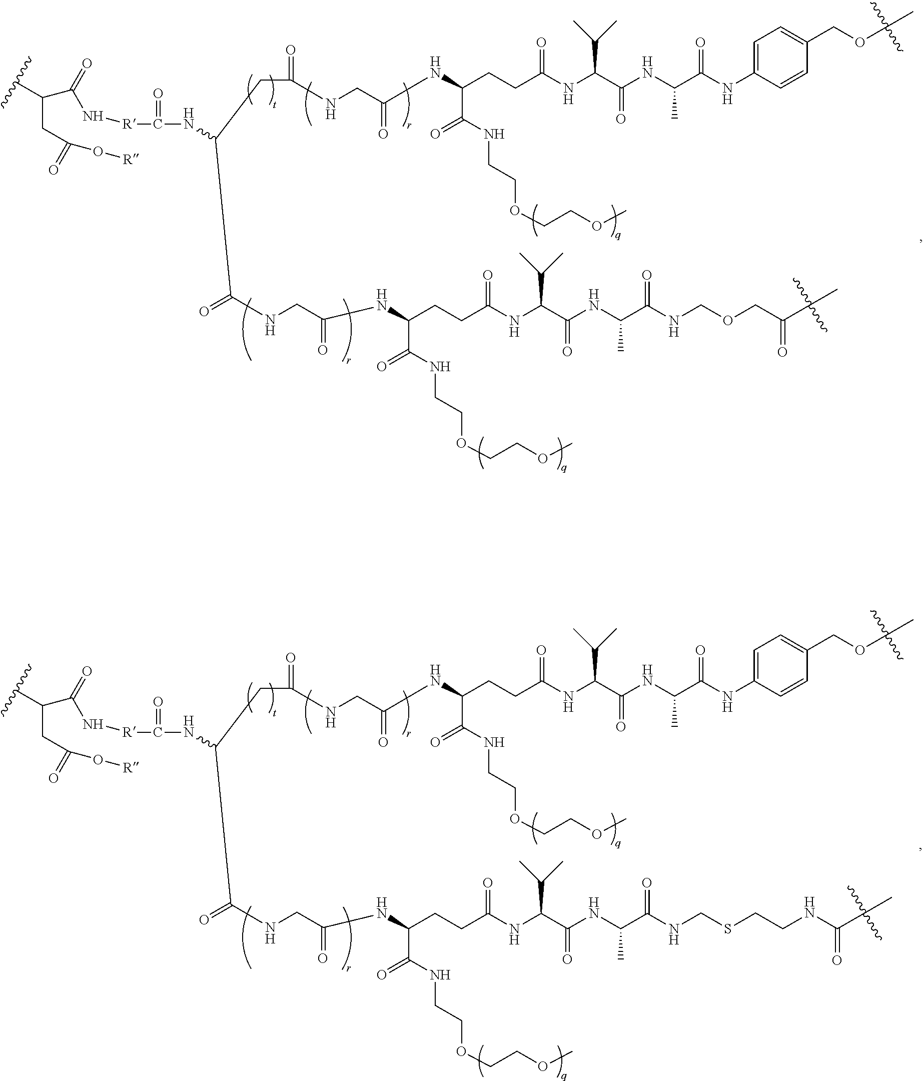

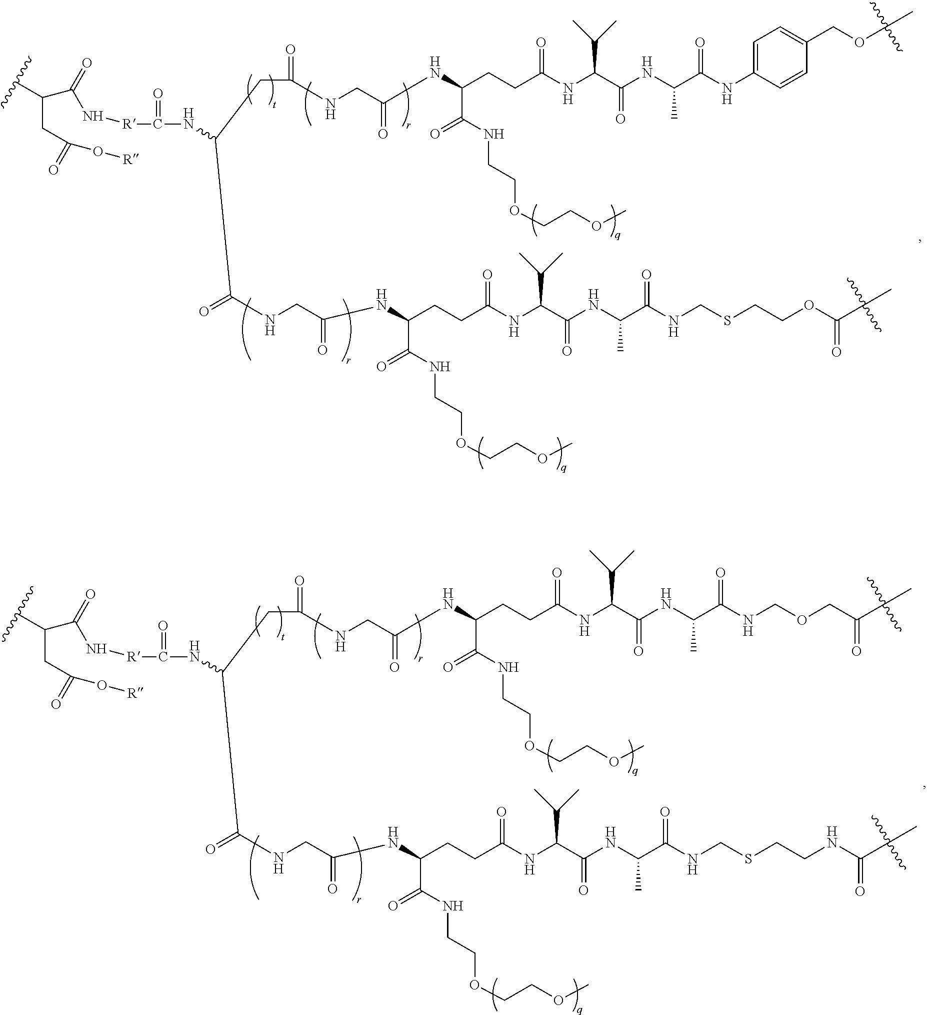

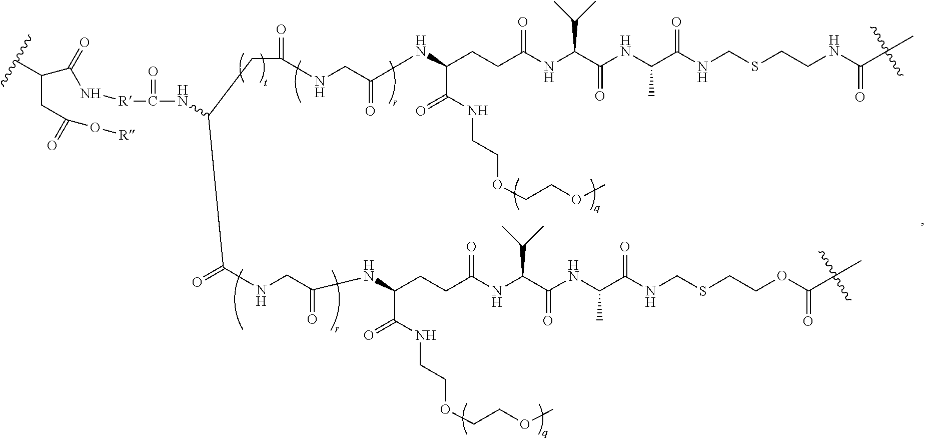

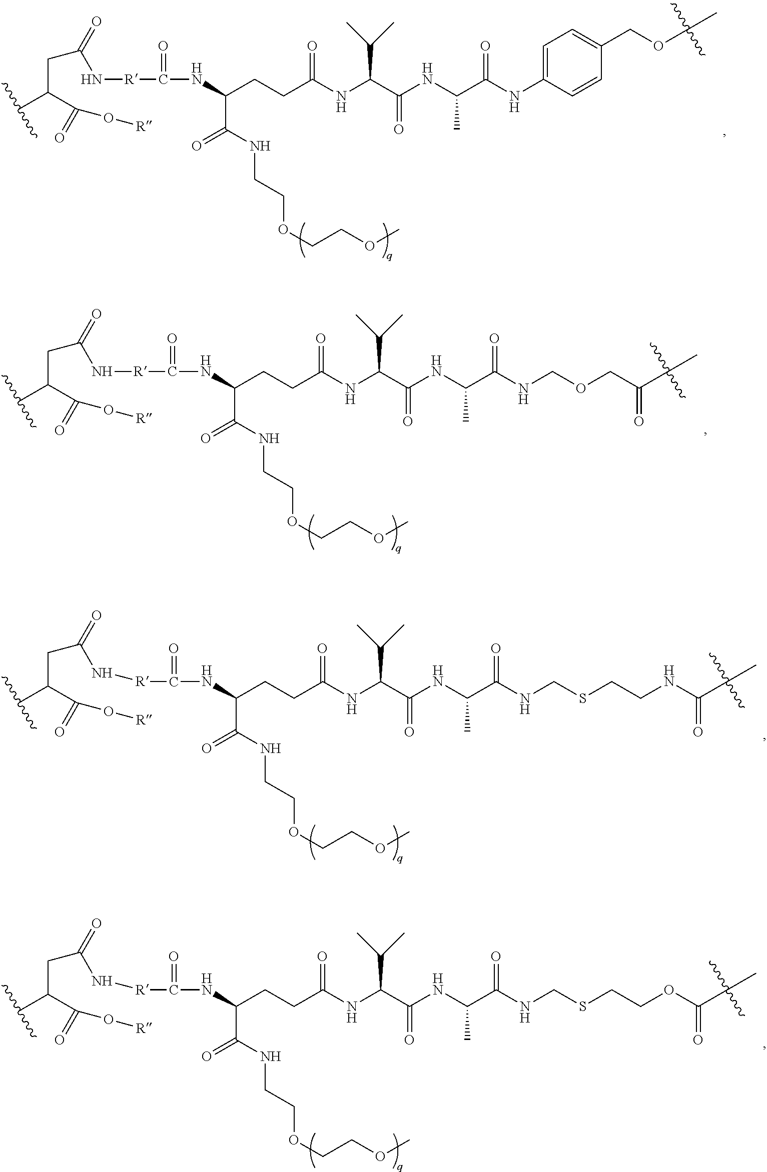

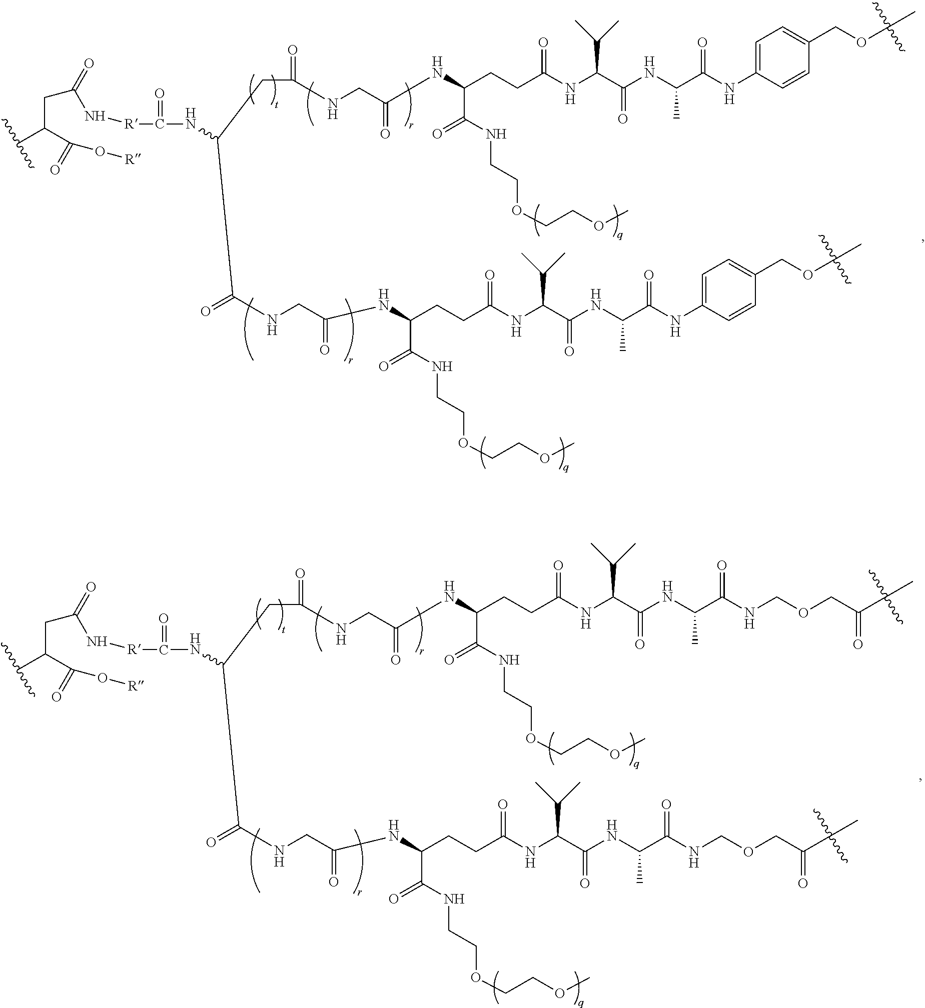

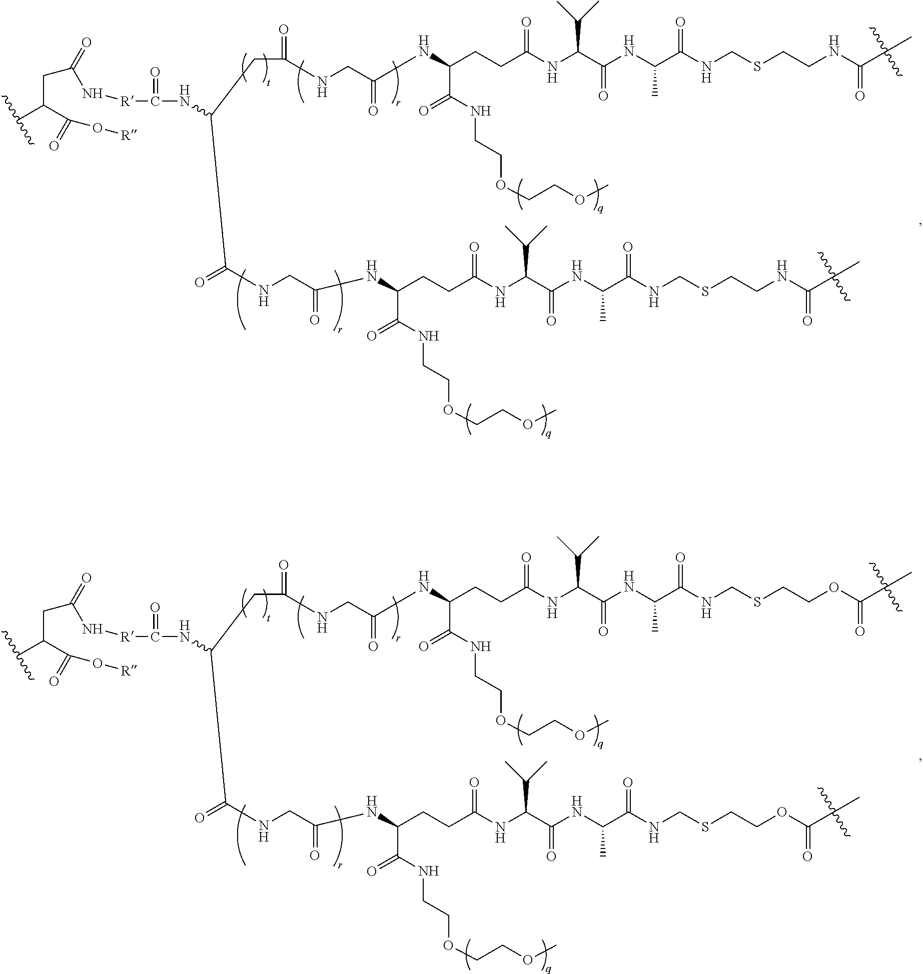

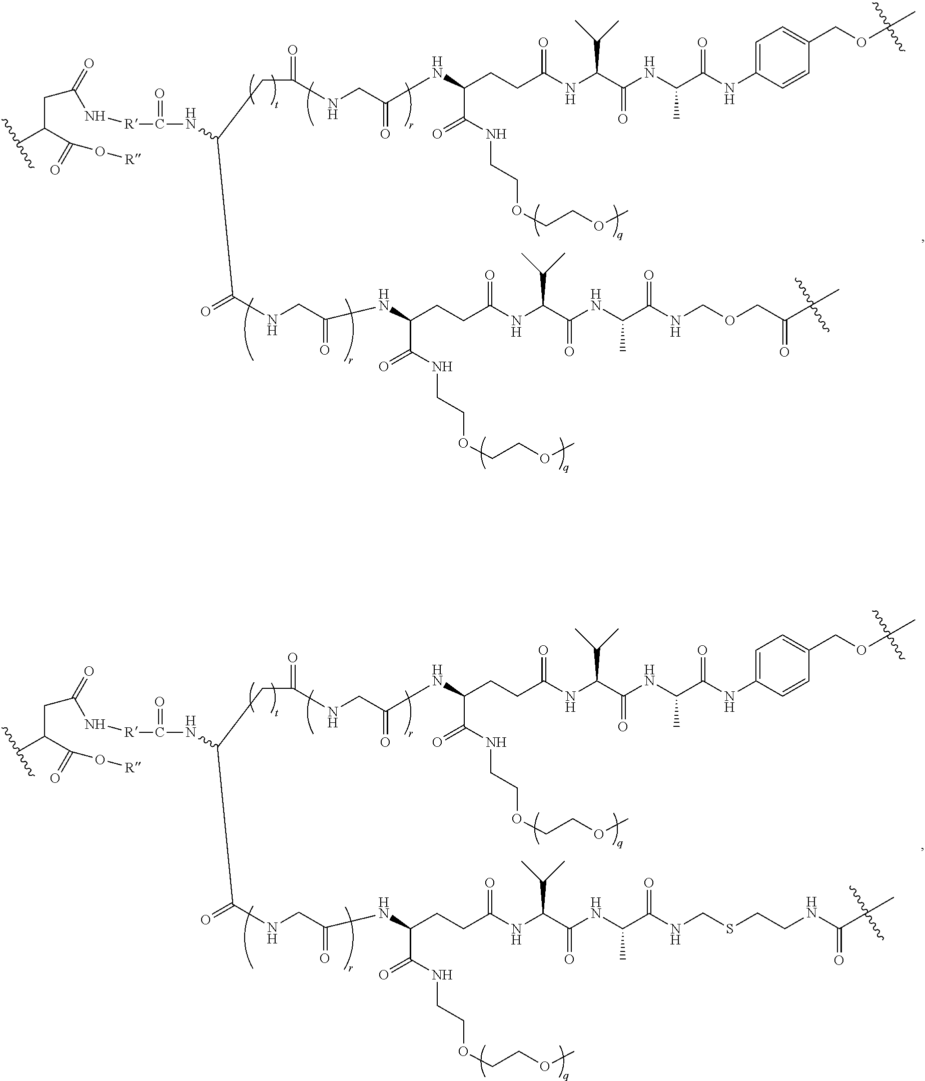

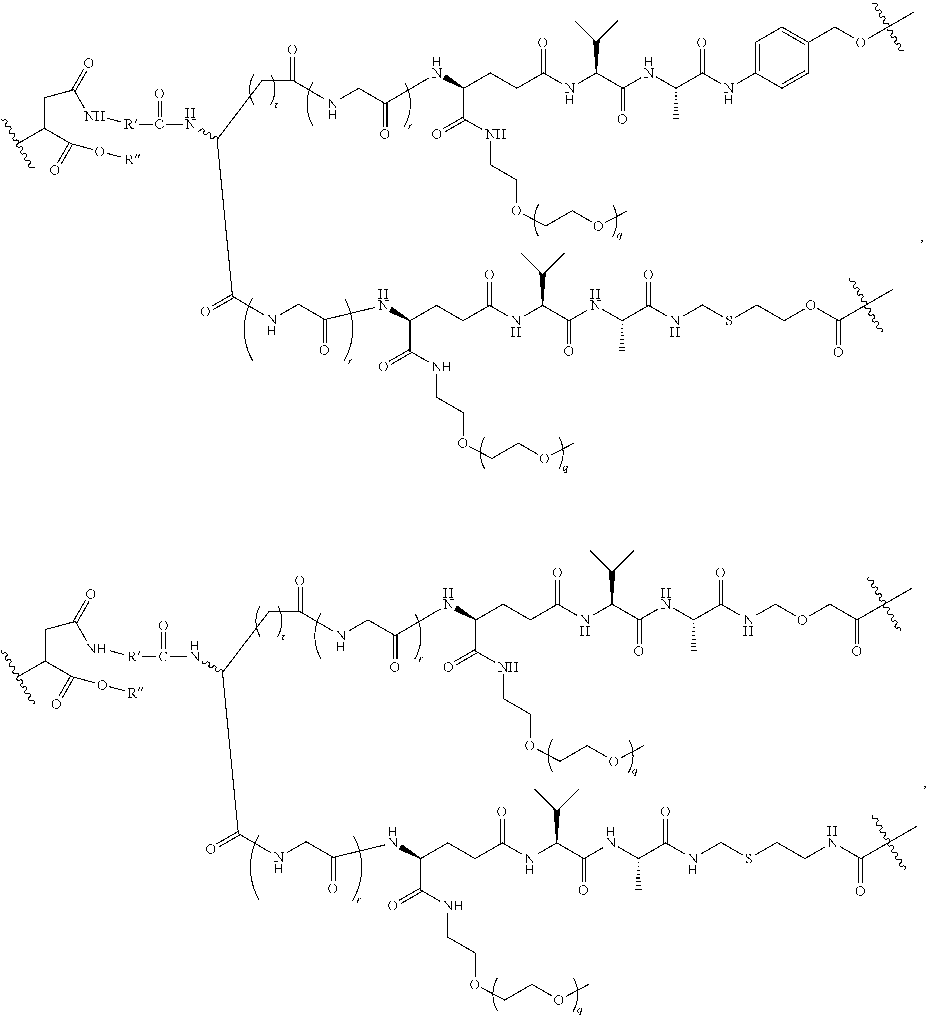

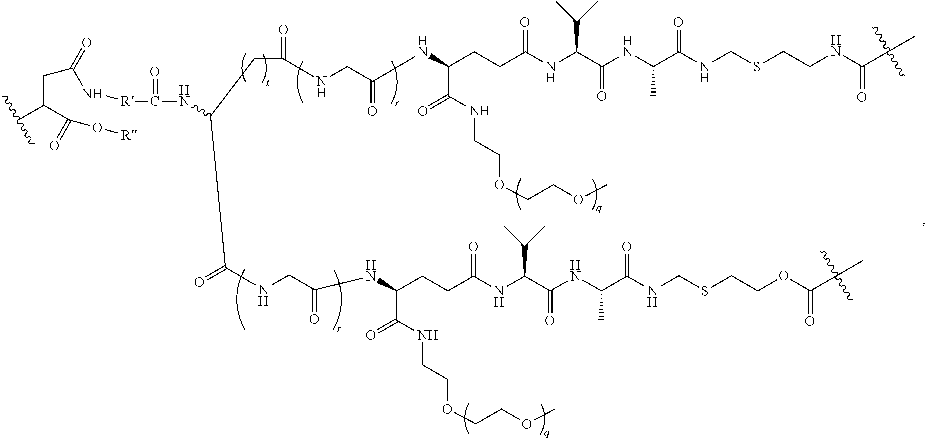

wherein,
R′ is selected from: optionally substituted C₁-C₁₀ alkyl, C₁-C₁₀ alkoxy, C₁-C₁₀ aminoalkyl and C₁-C₁₀ alkyl-aryl,

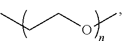

R″ is optionally substituted C₁-C₁₀ alkyl; wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl; and wherein a subscript n is any integer selected from 1-10, preferably 1-5, and most preferably 1-3; and
each subscript r is any integer independently selected from 1-4, each subscript t is any integer independently selected from 1-4, and each subscript q is any integer independently selected from 1-20, preferably 3-10, and most preferably 5-8.

In some embodiments, the linker is selected from the following structures:

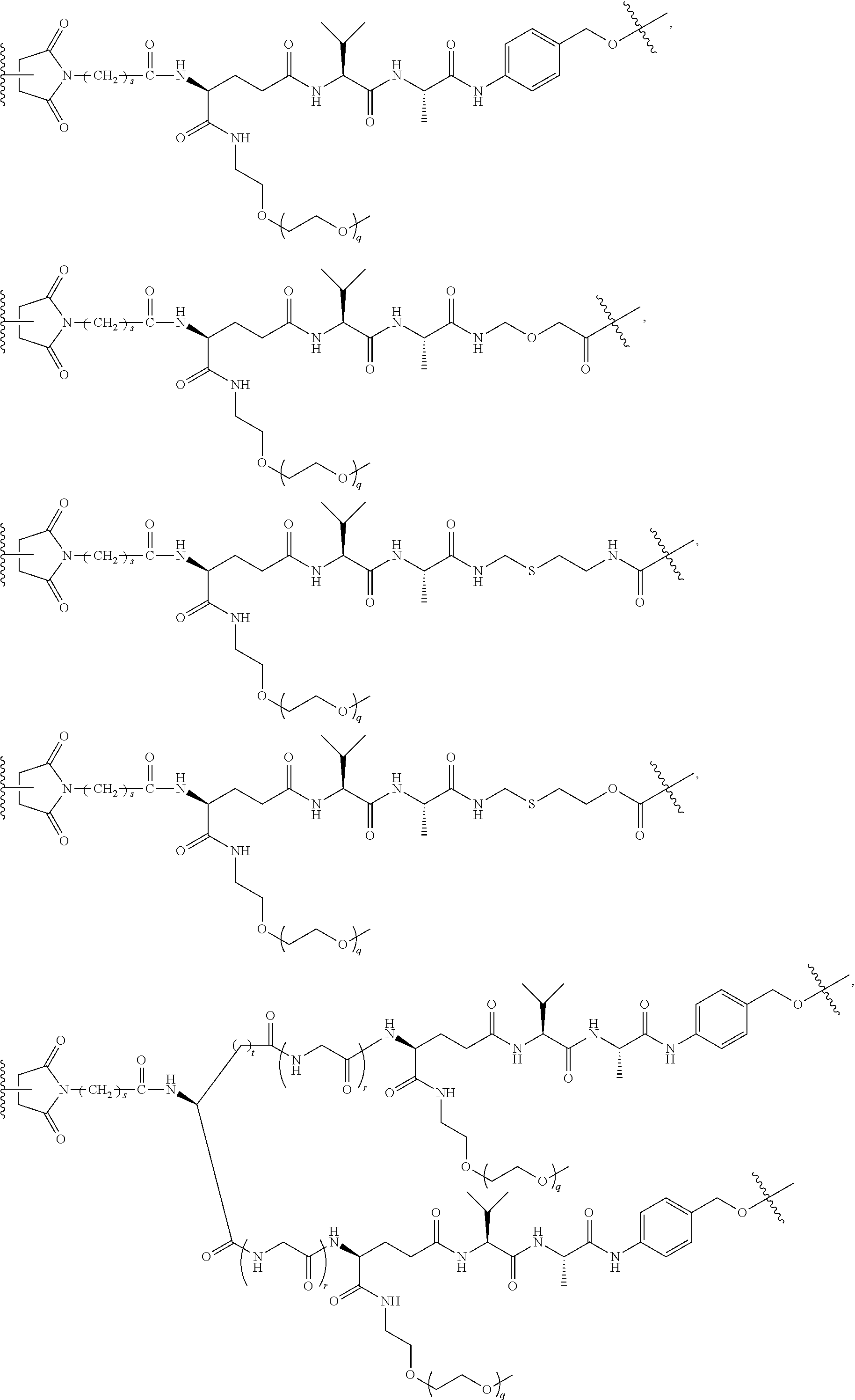

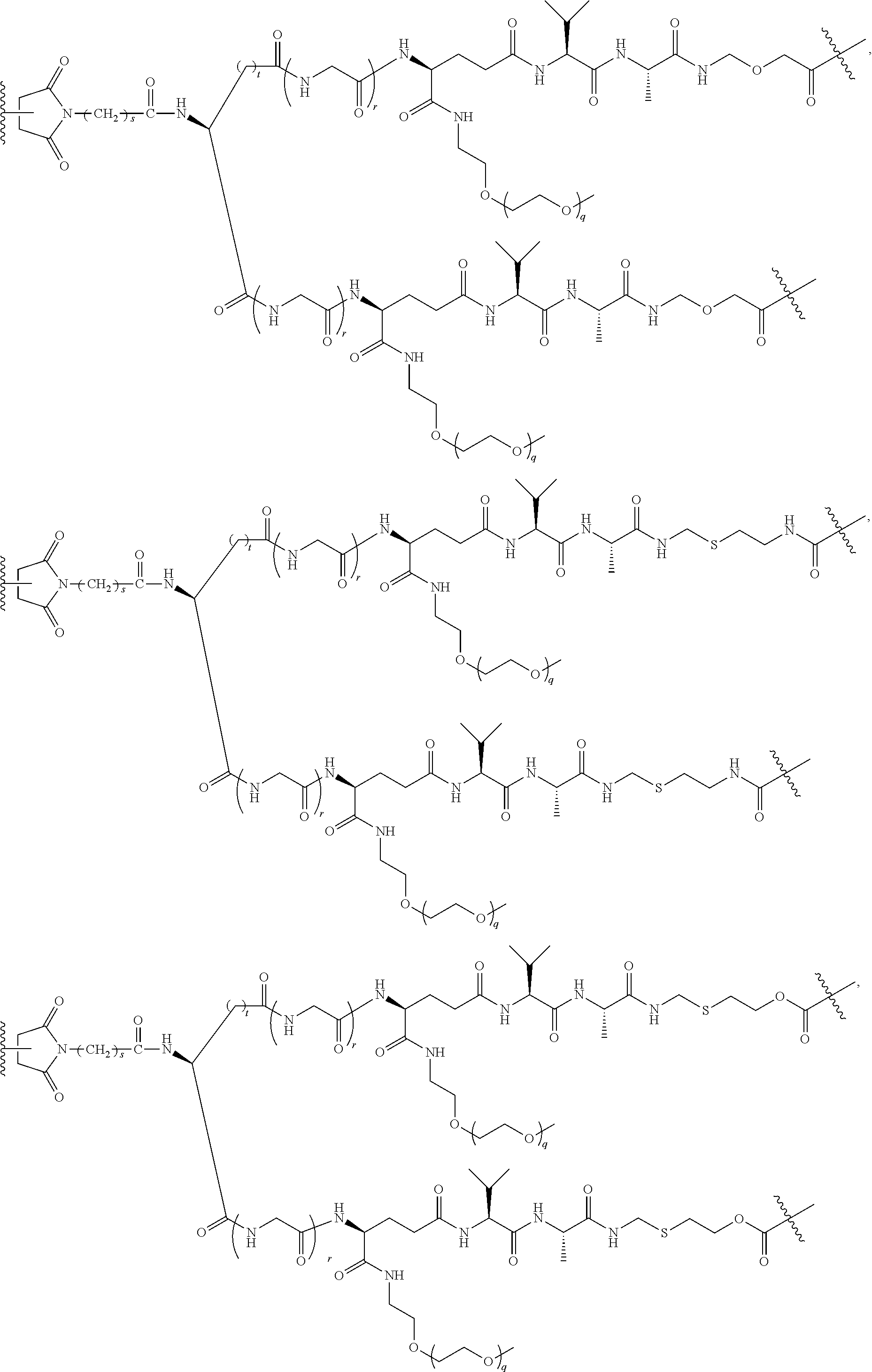

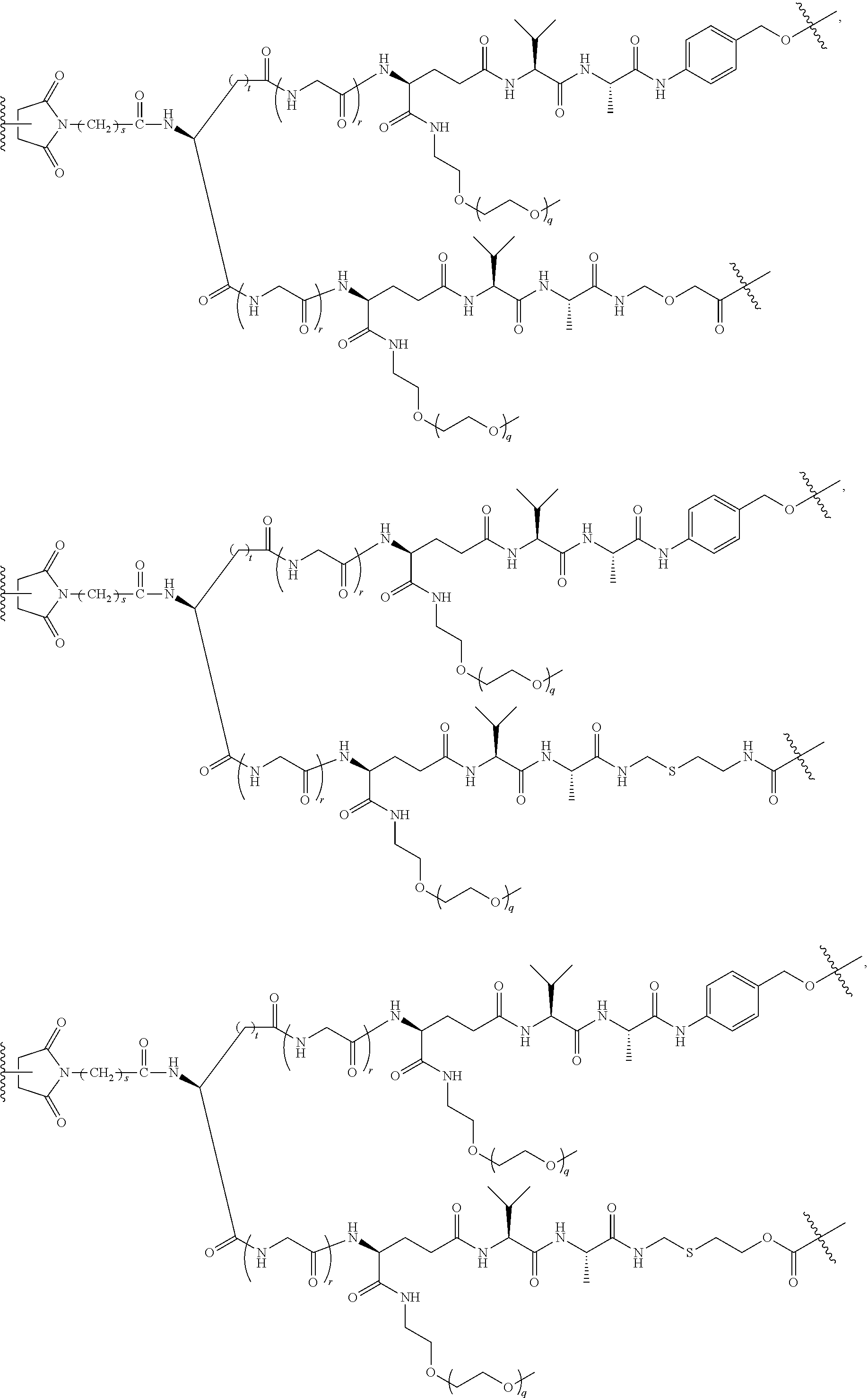

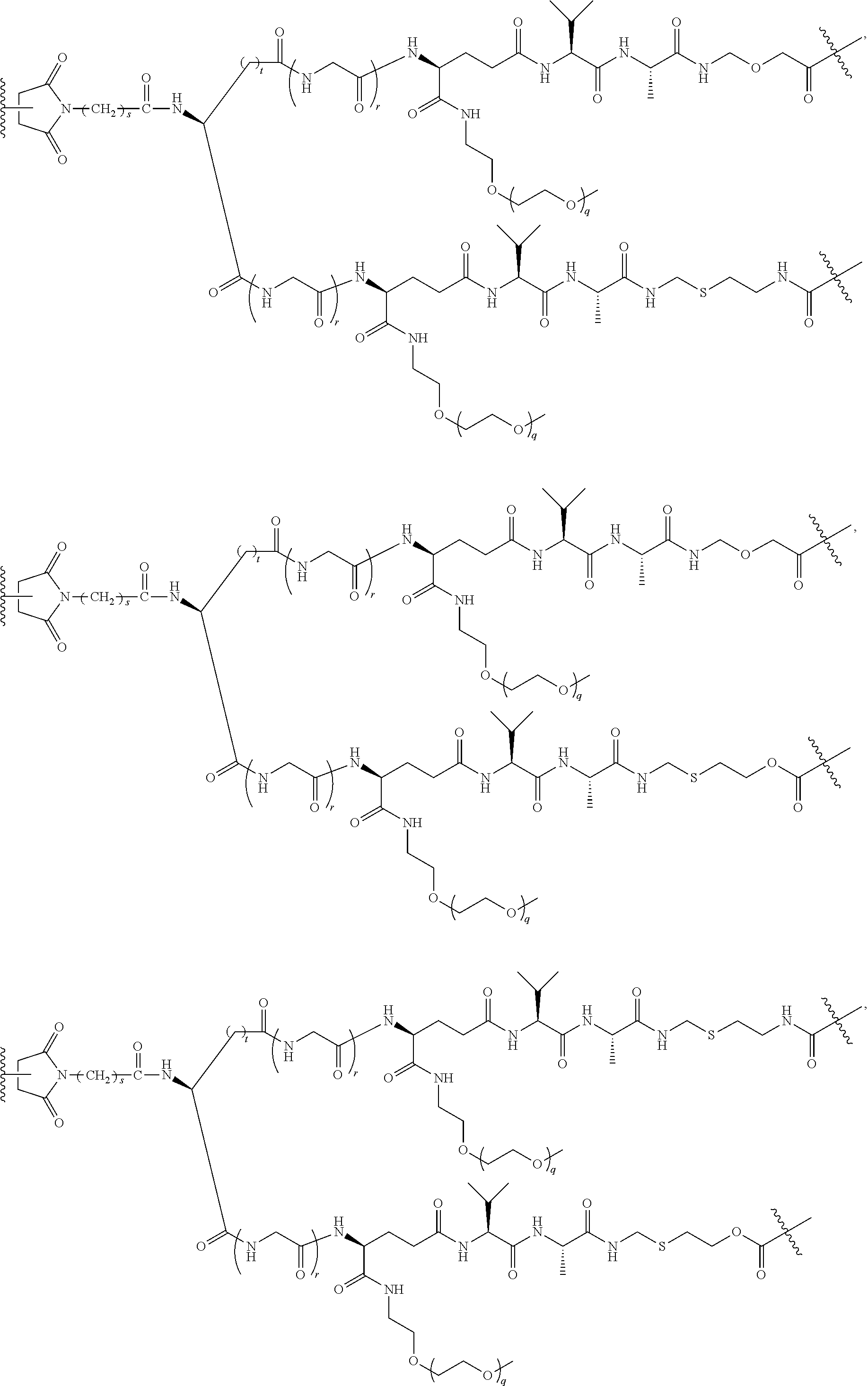

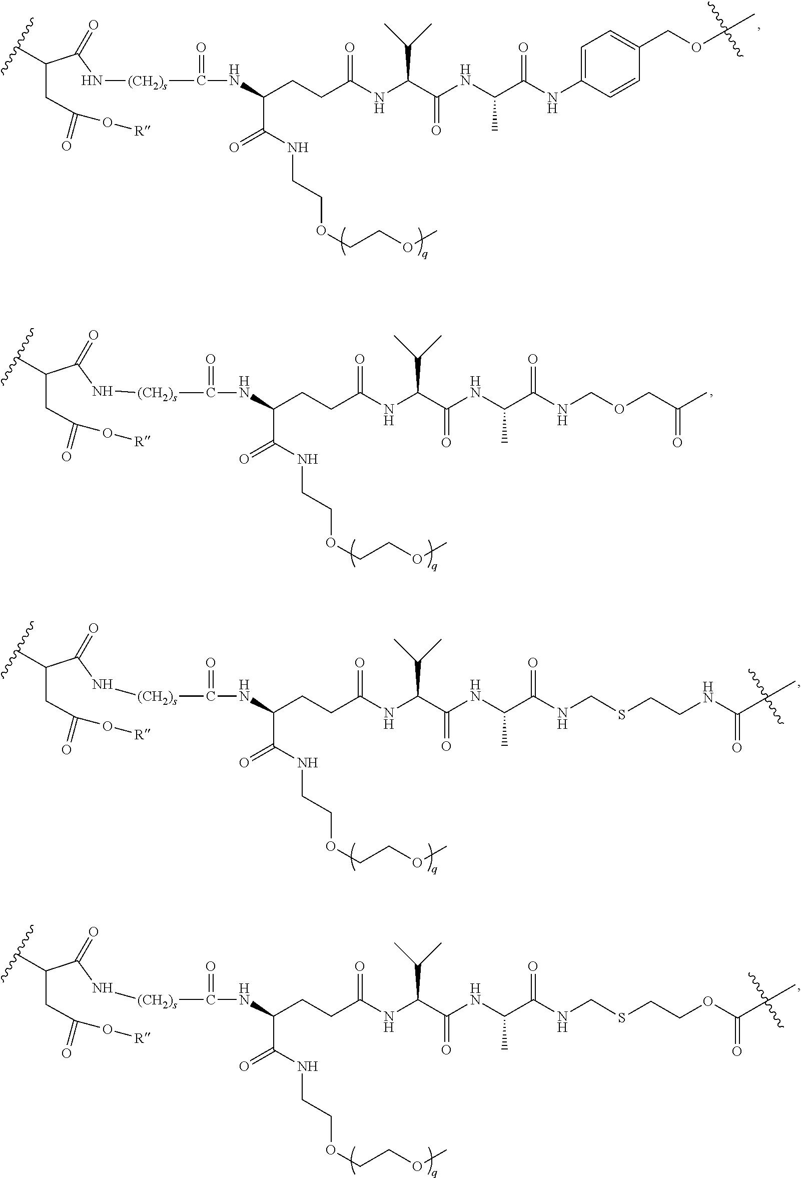

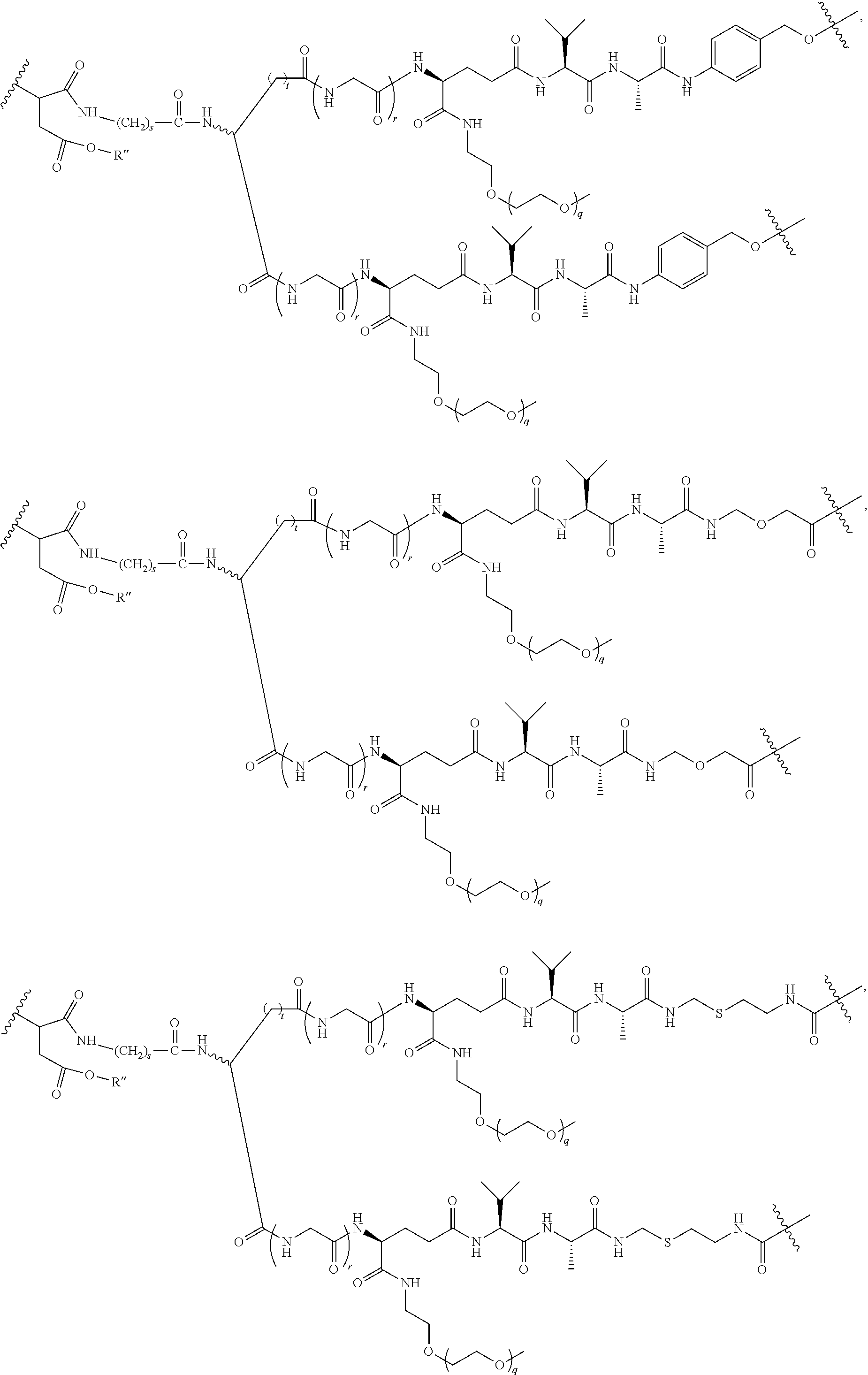

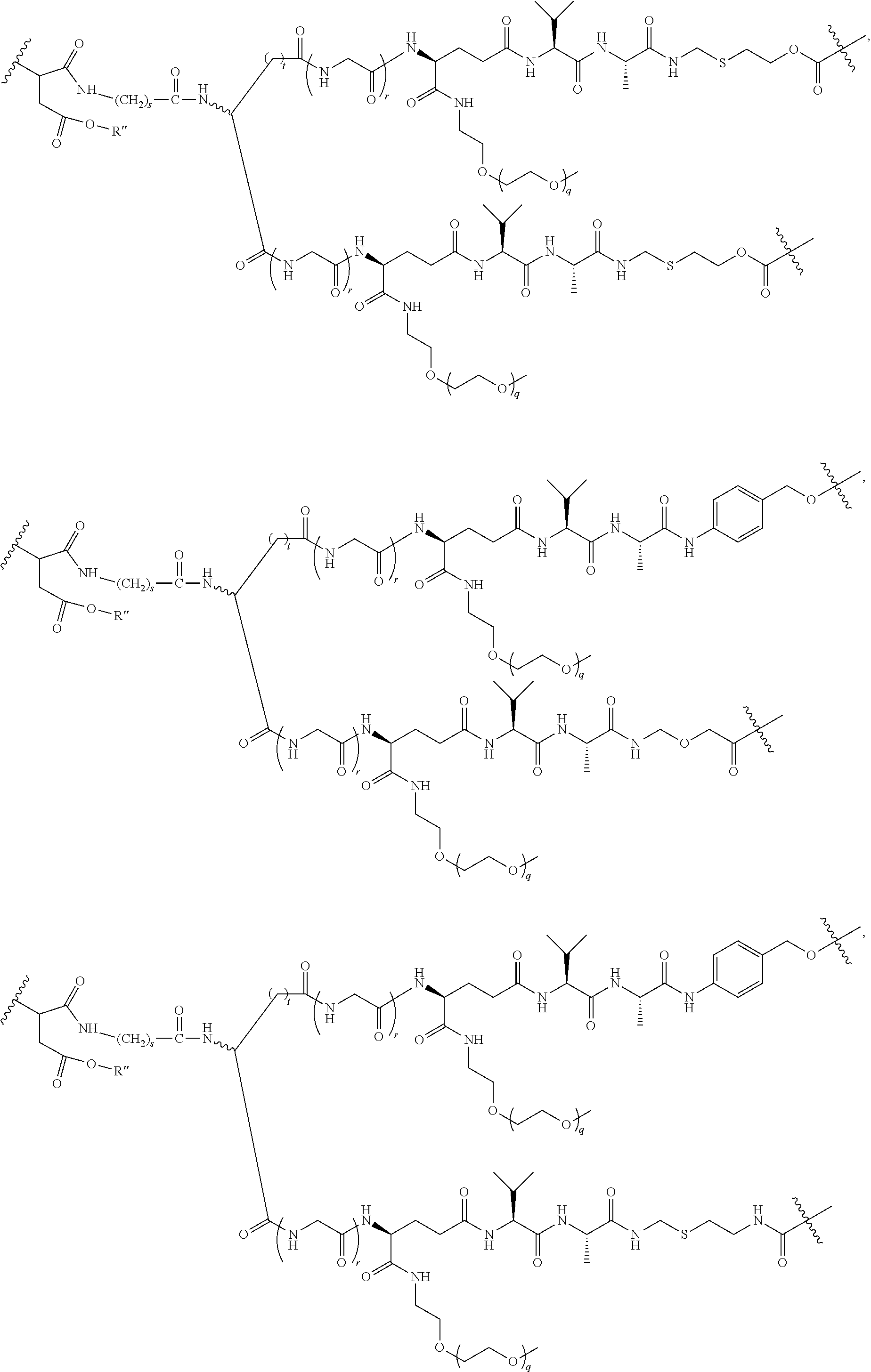

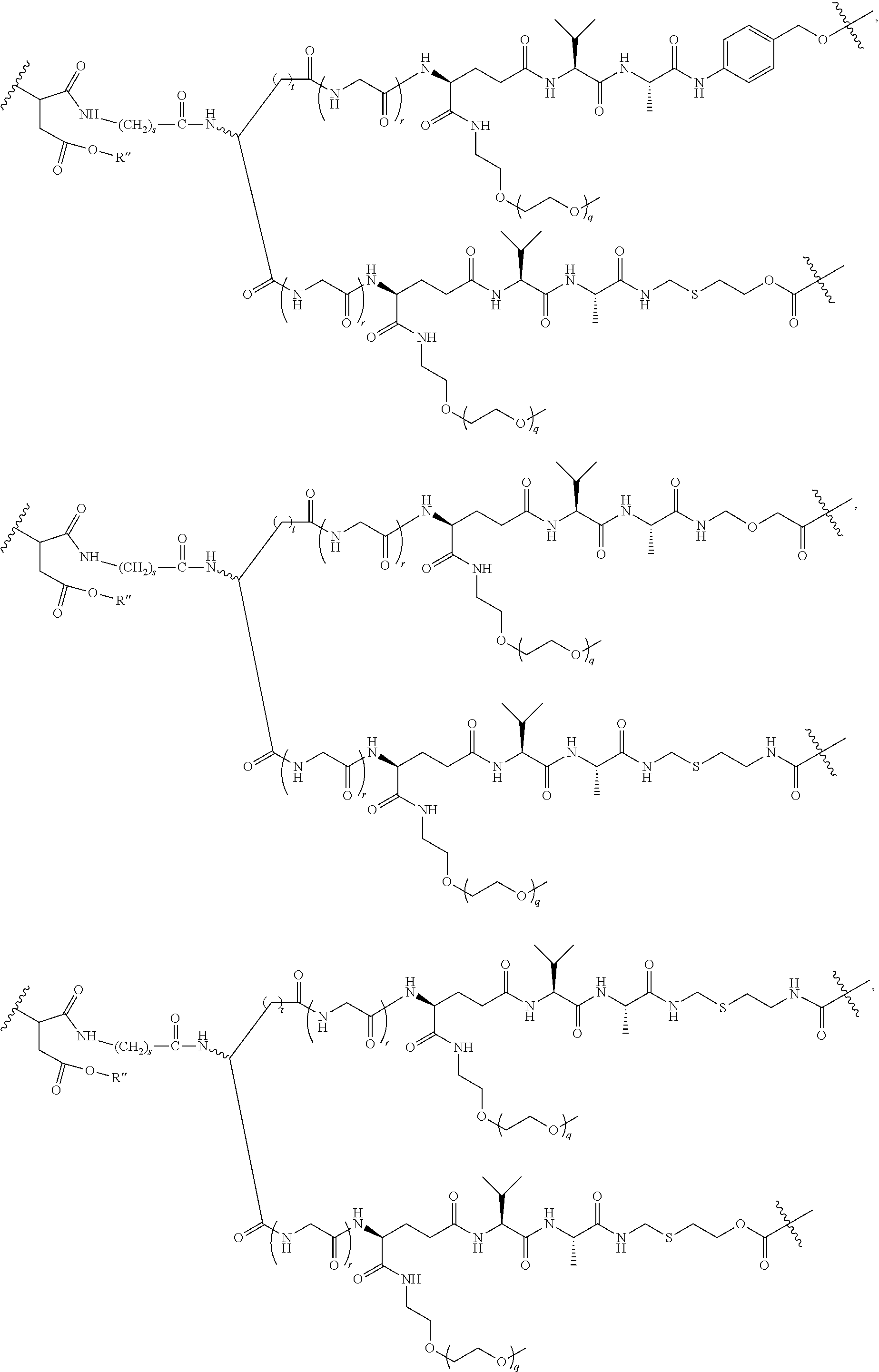

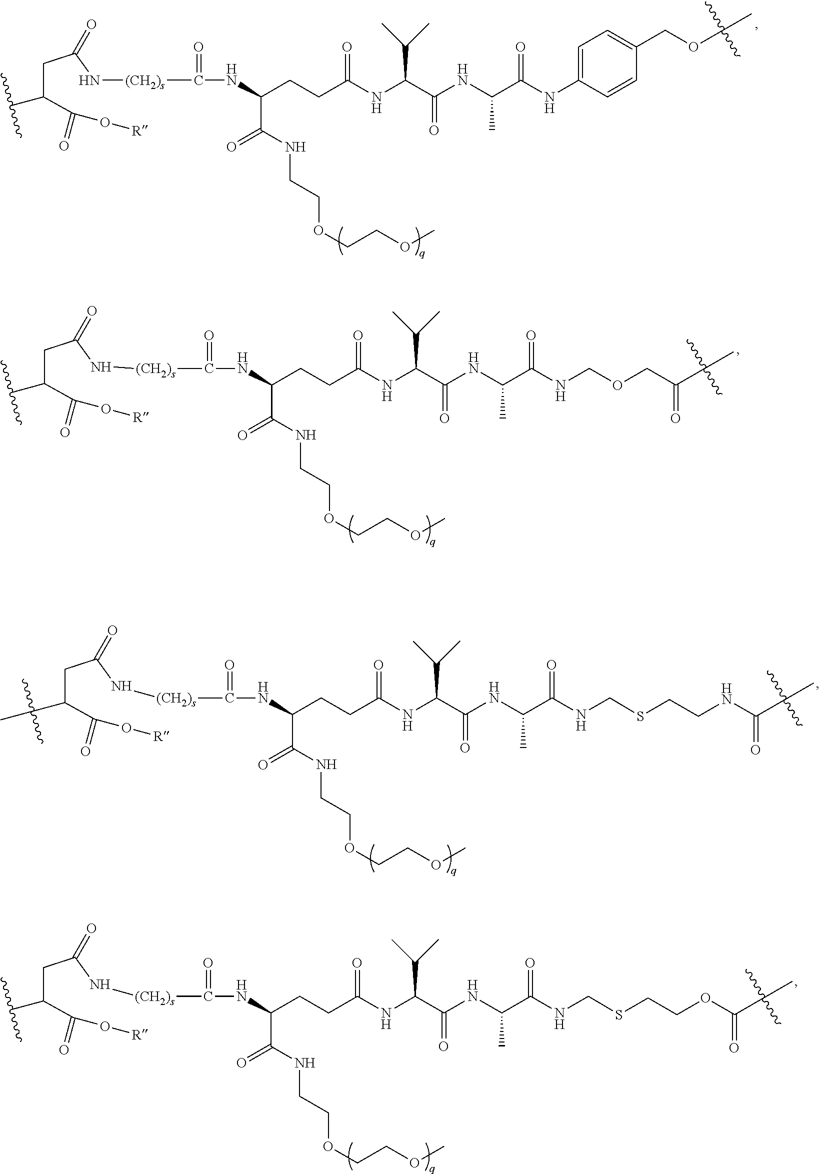

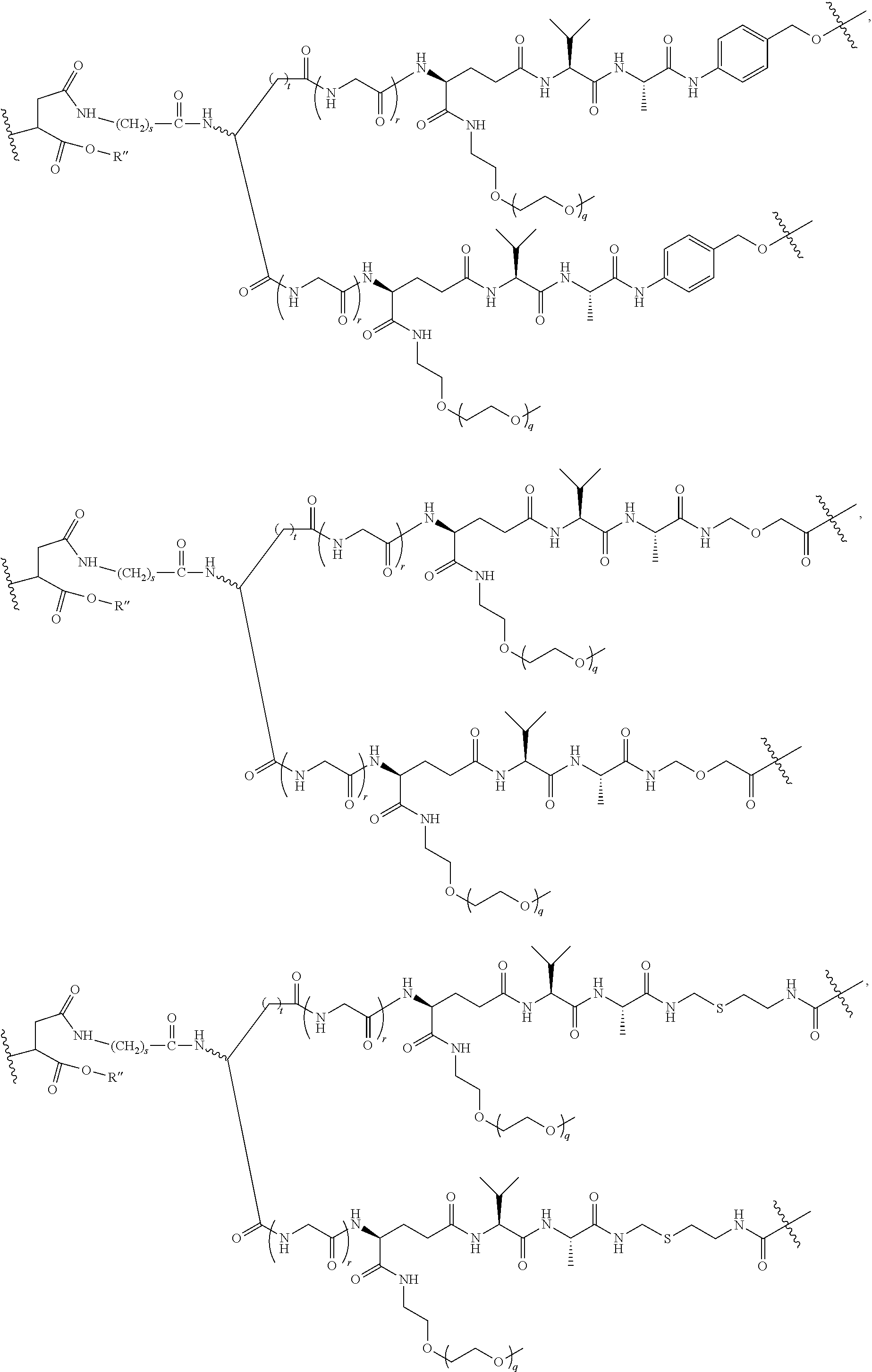

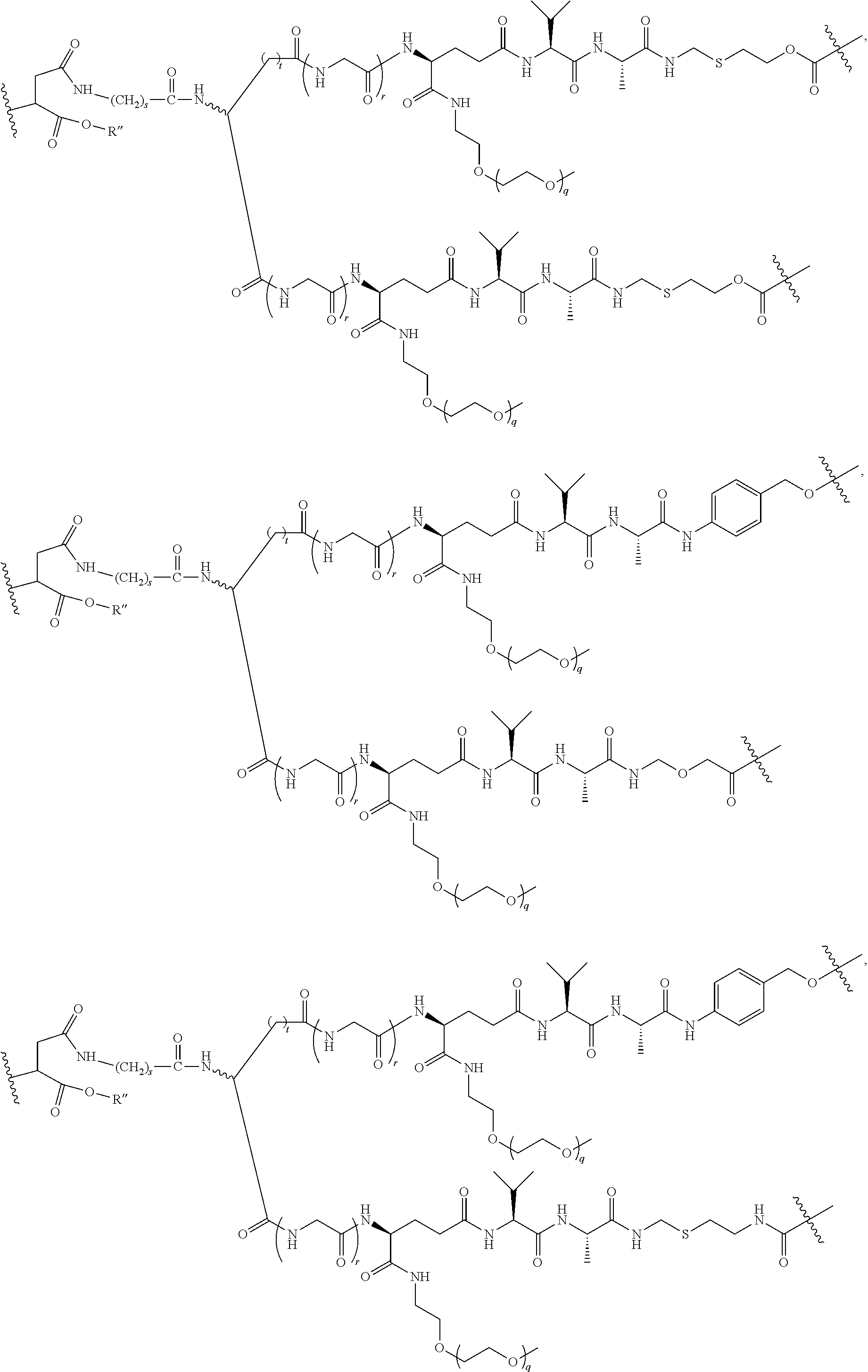

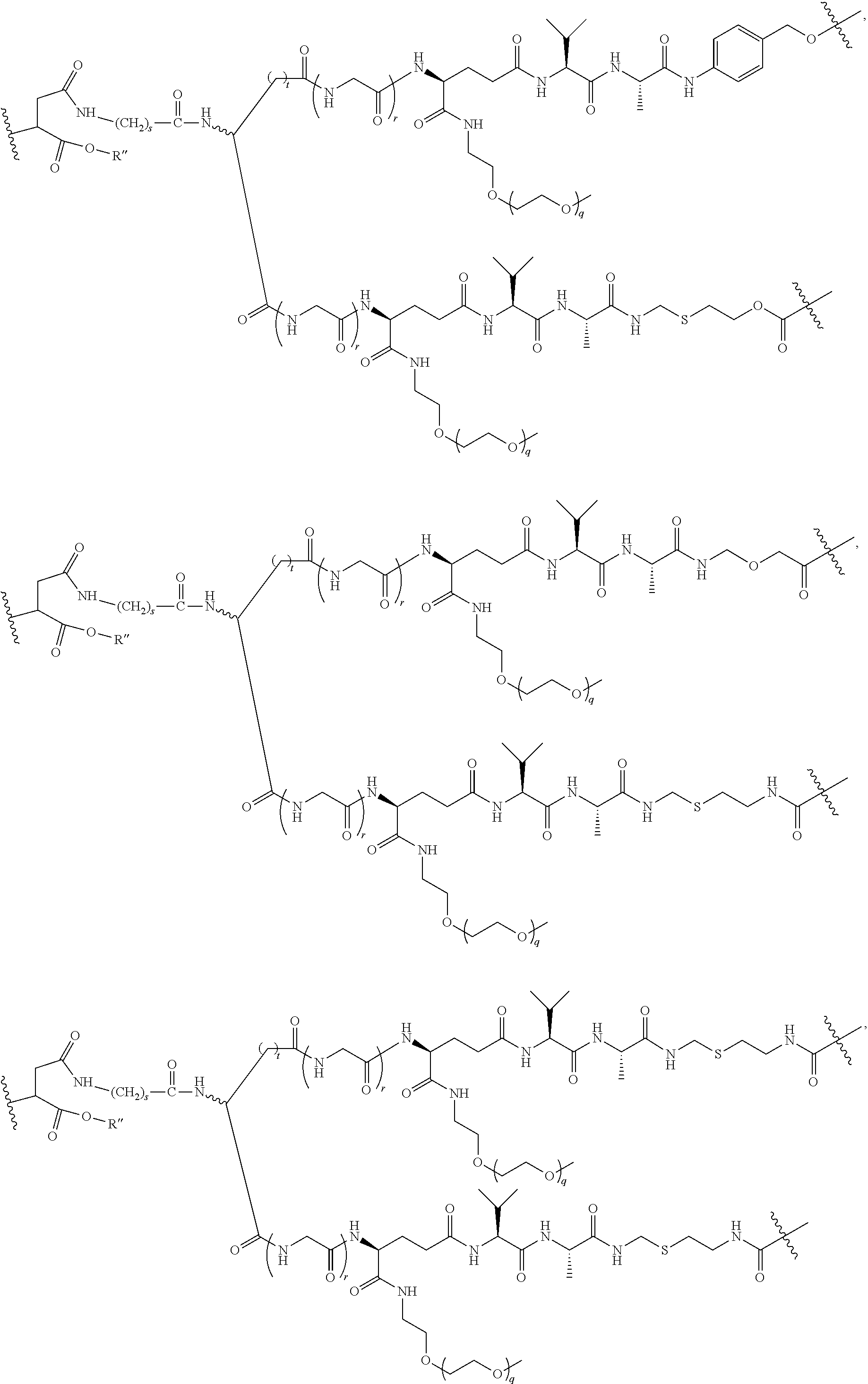

wherein R″ is selected from the group consisting of methyl, ethyl and propyl, each subscript s is any integer independently selected from 1-10, each subscript r is any integer independently selected from 1-4, each subscript t is any integer independently selected from 1-4, and each subscript q is any integer independently selected from 1-20, preferably 3-10, and most preferably 5-8.

In some embodiments, the linker is selected from the following structures:

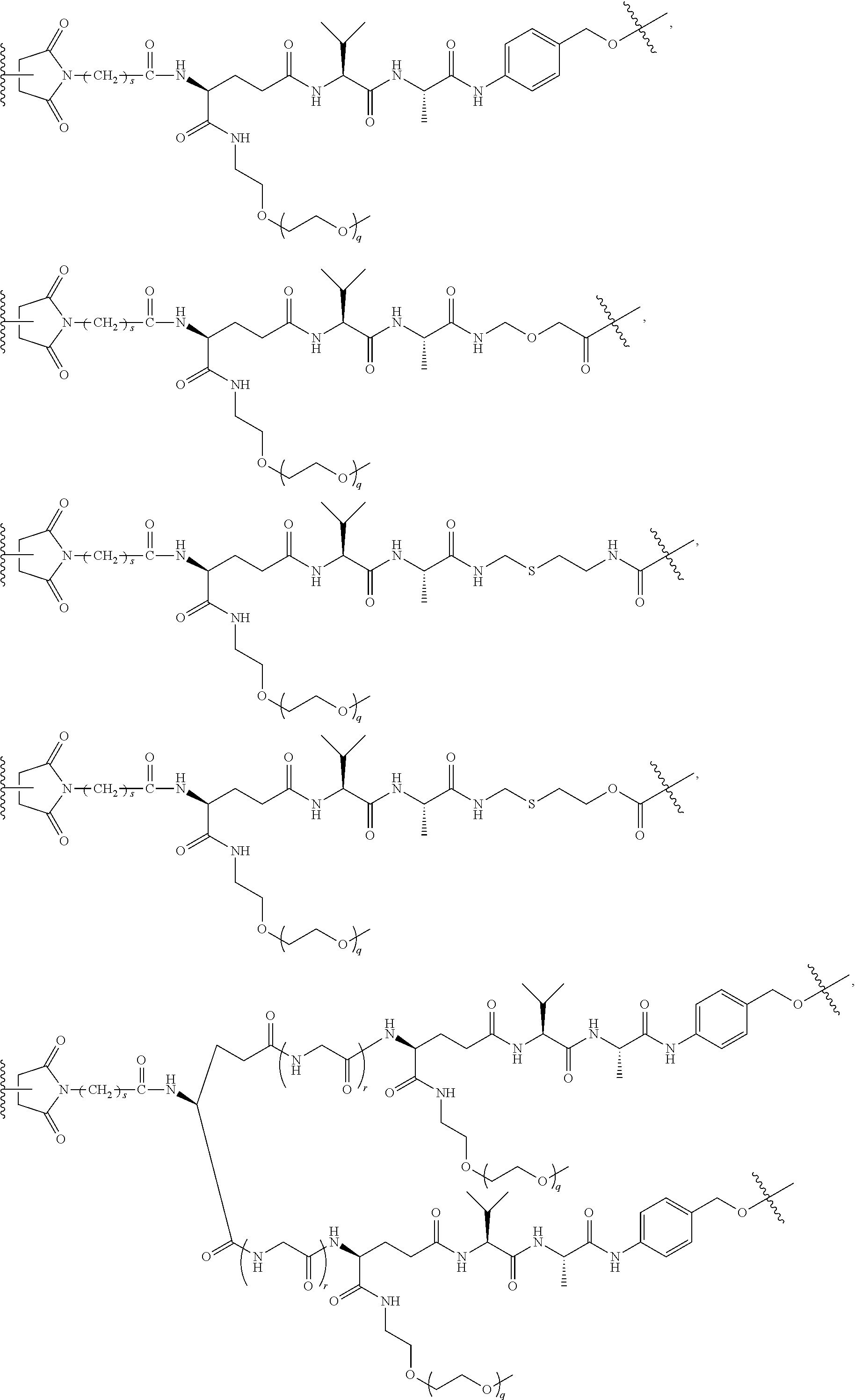

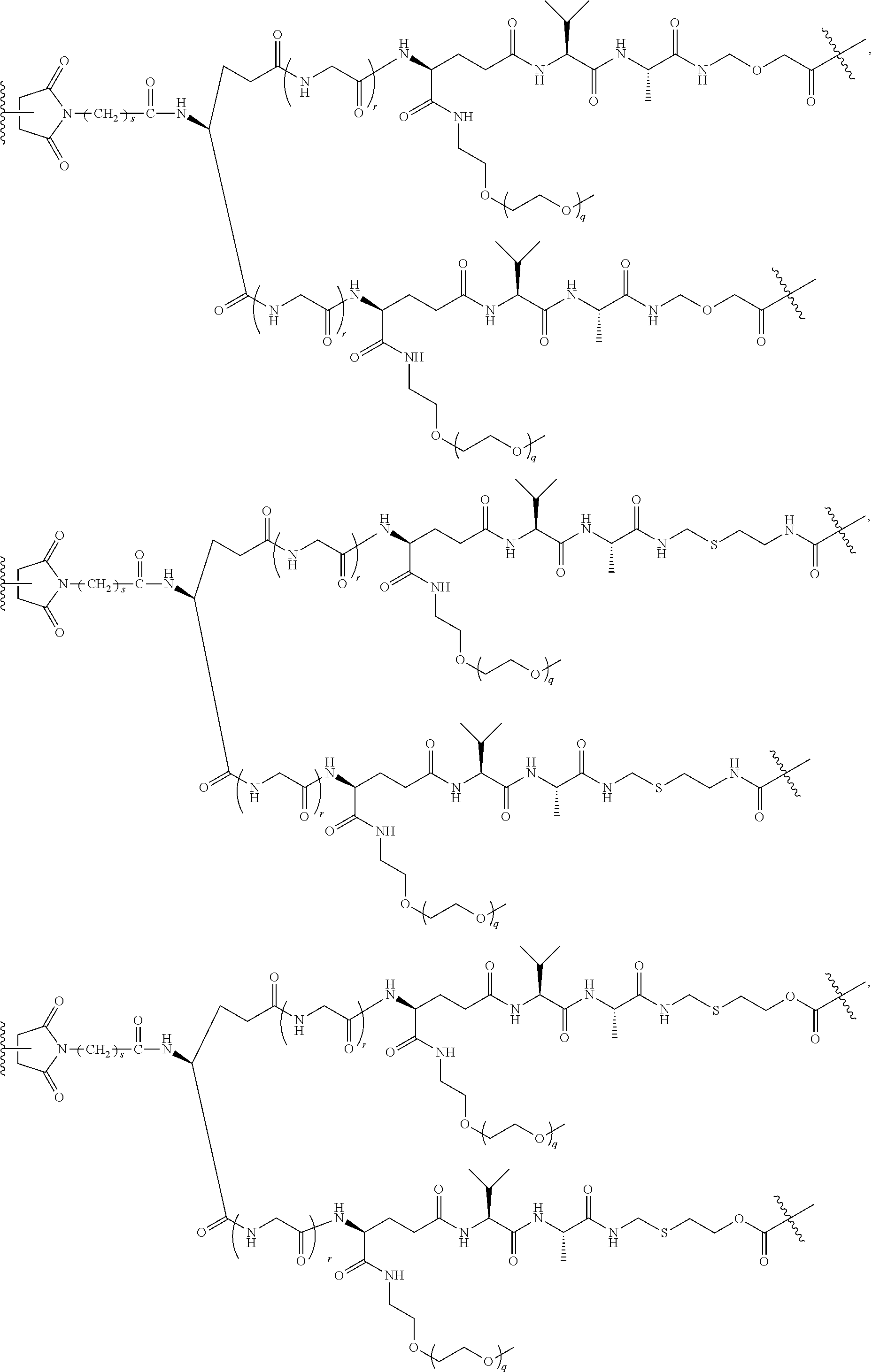

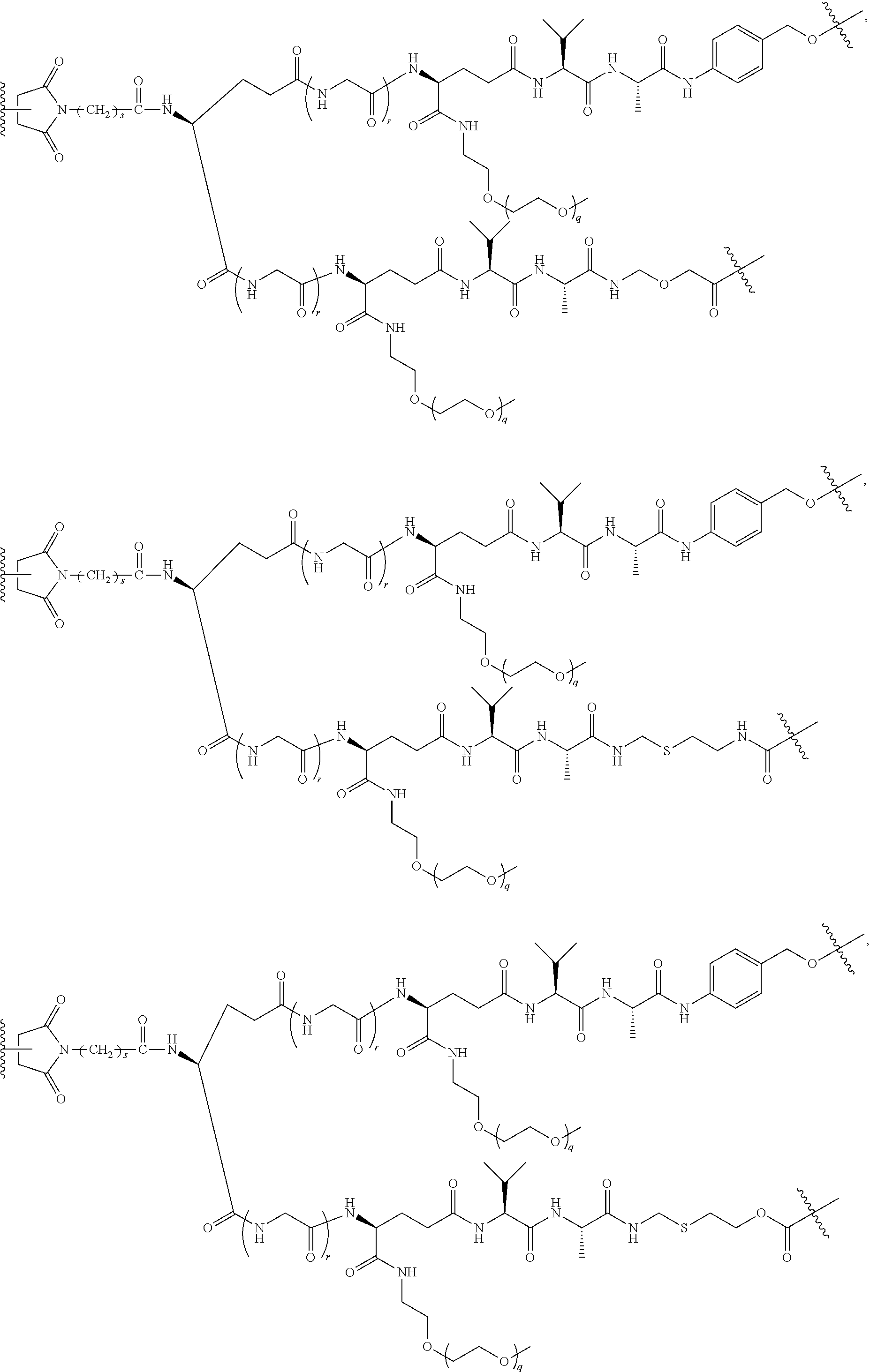

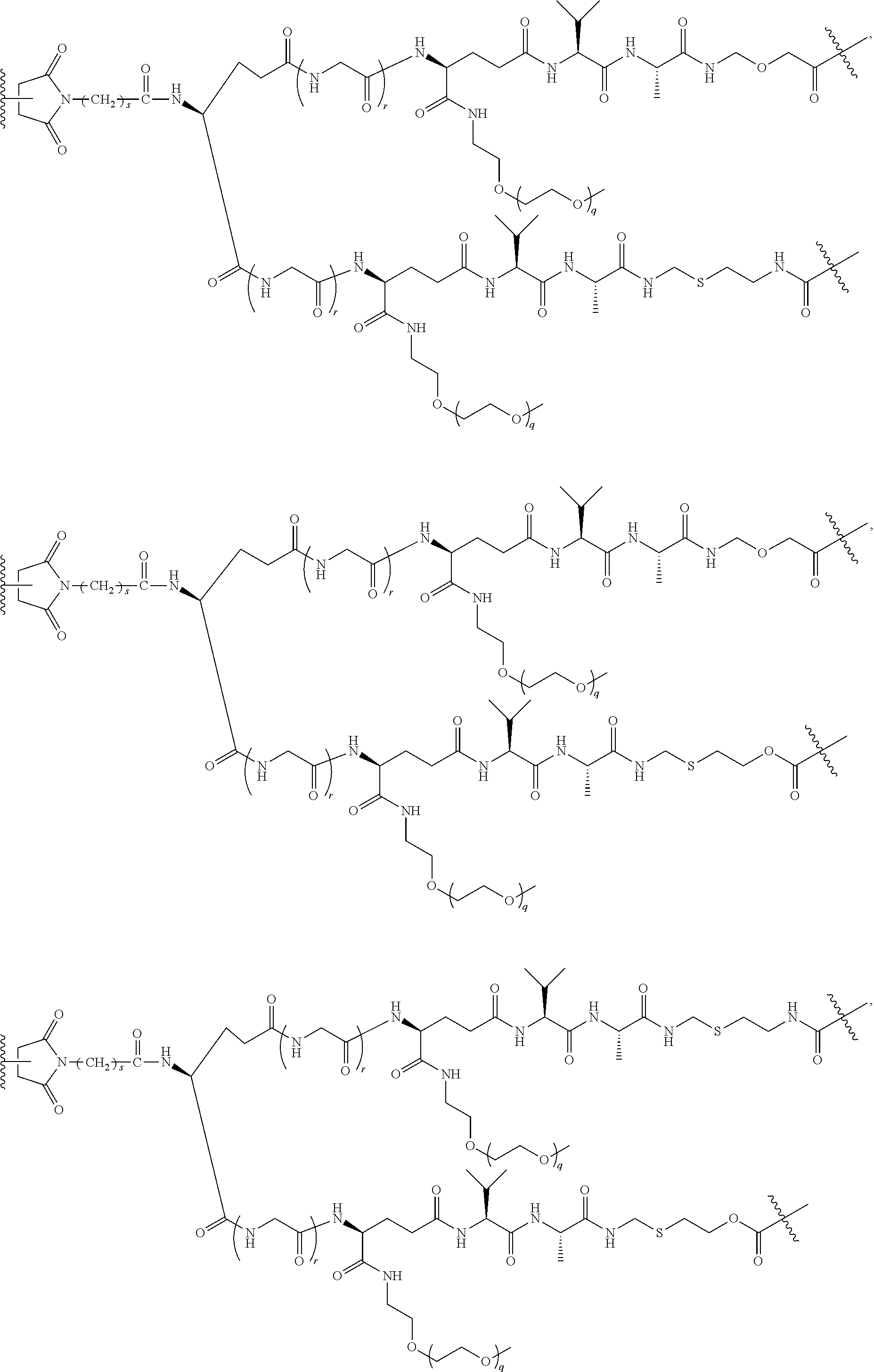

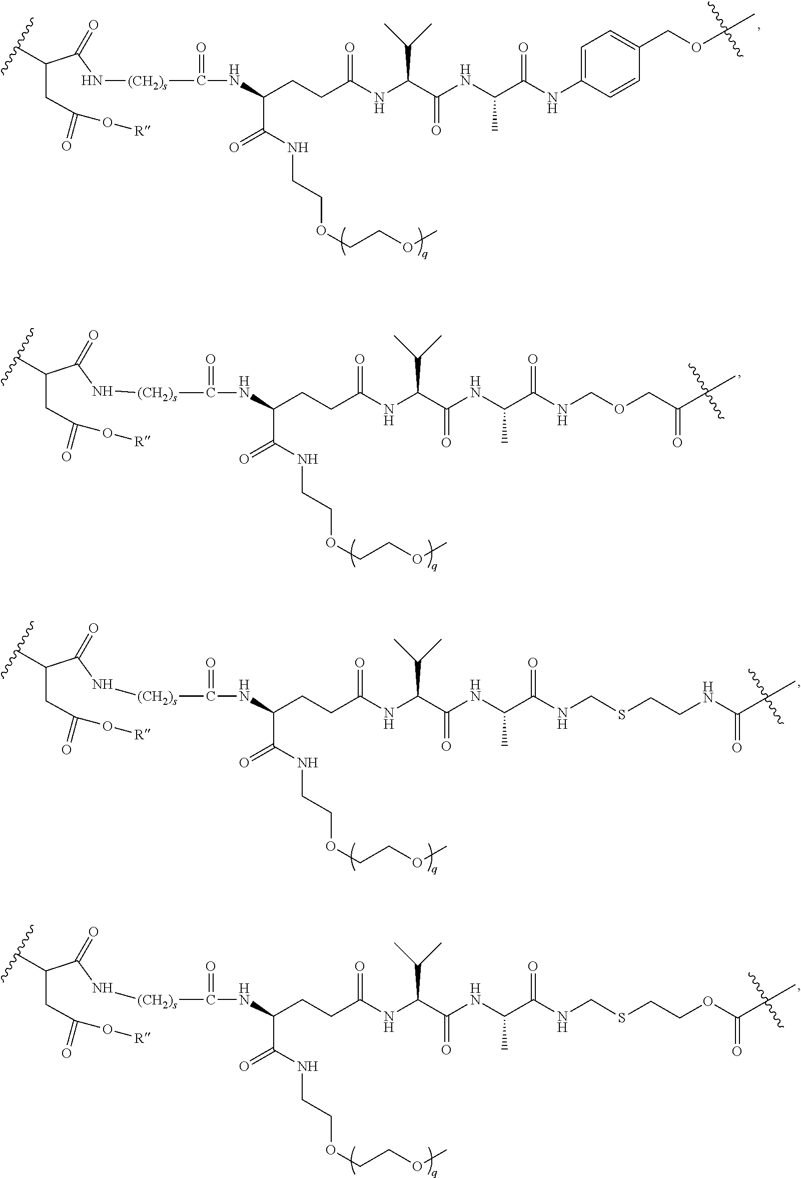

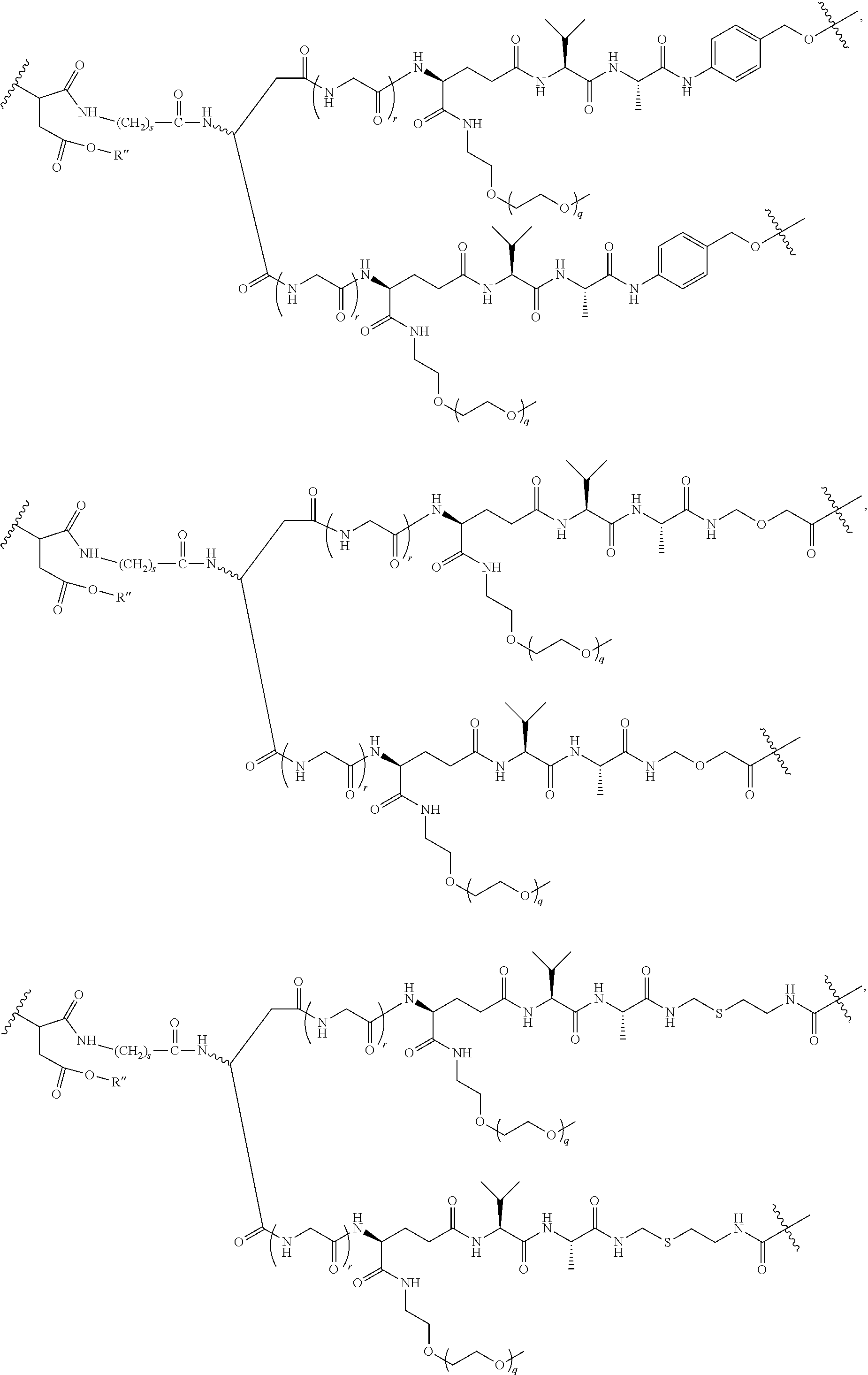

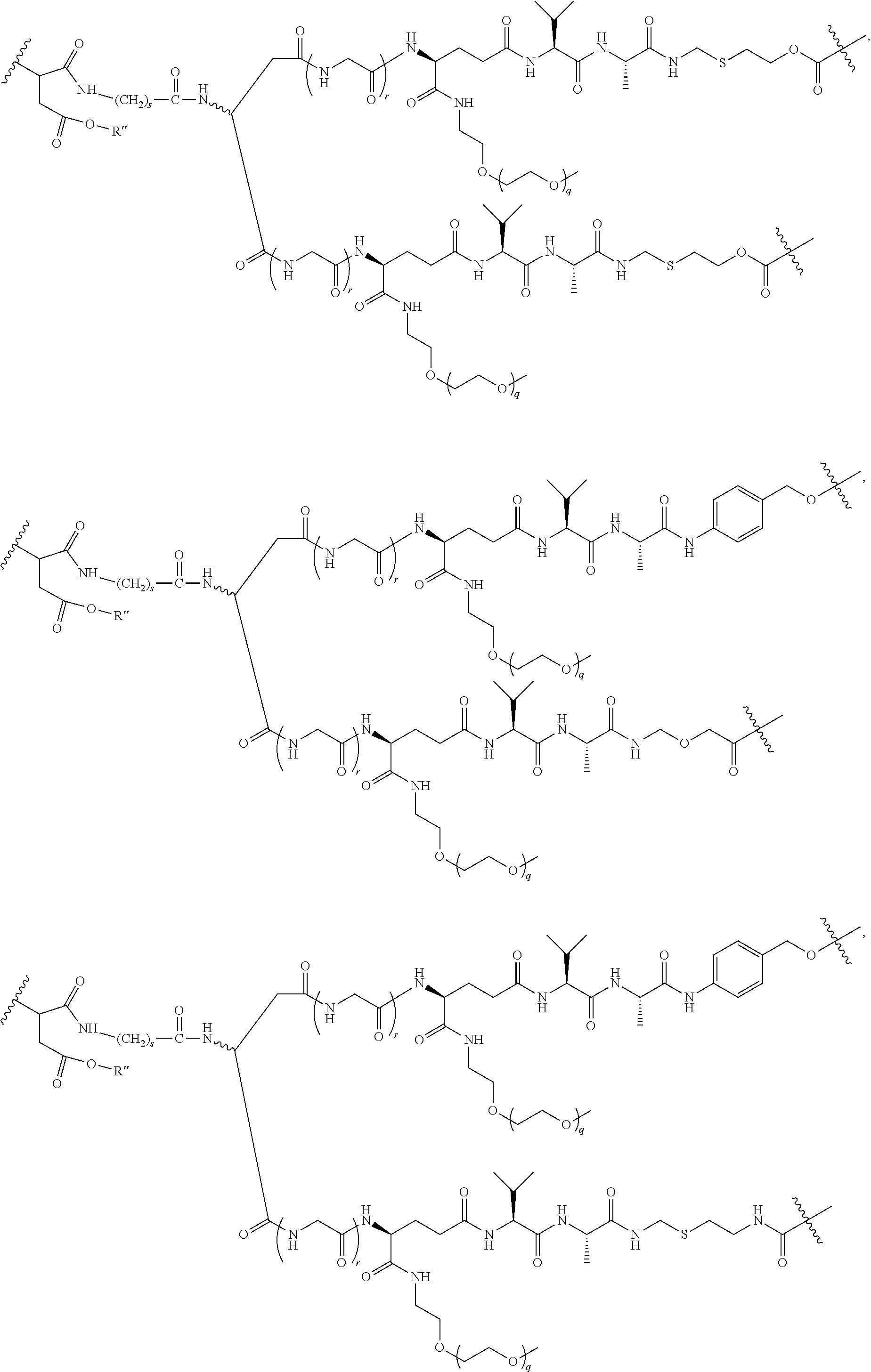

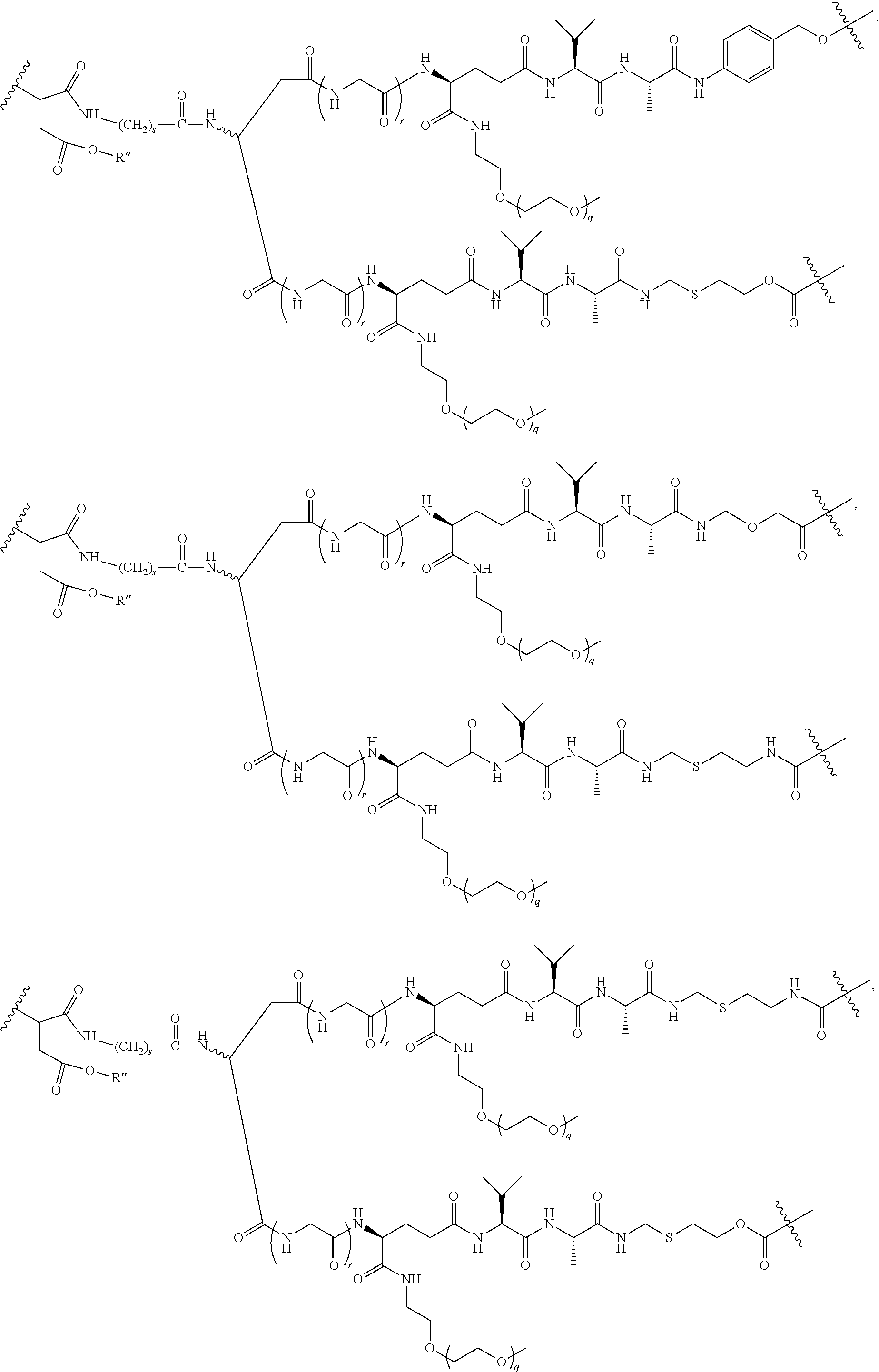

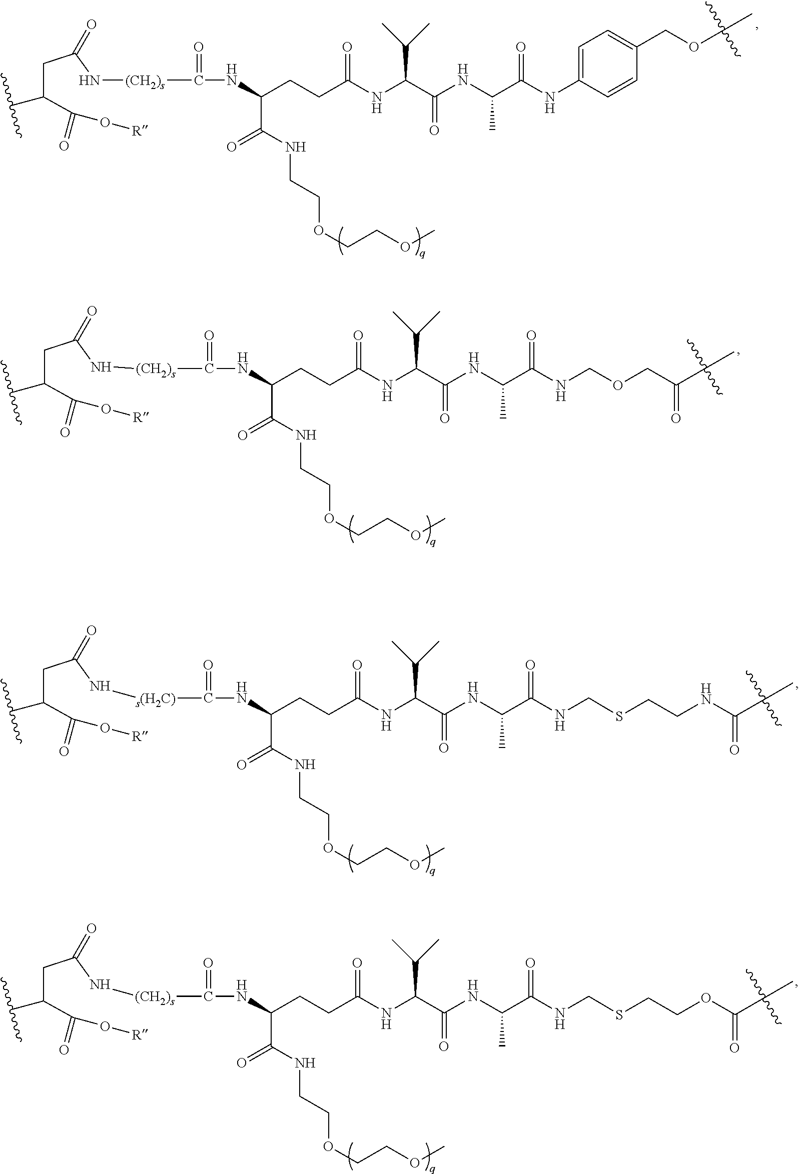

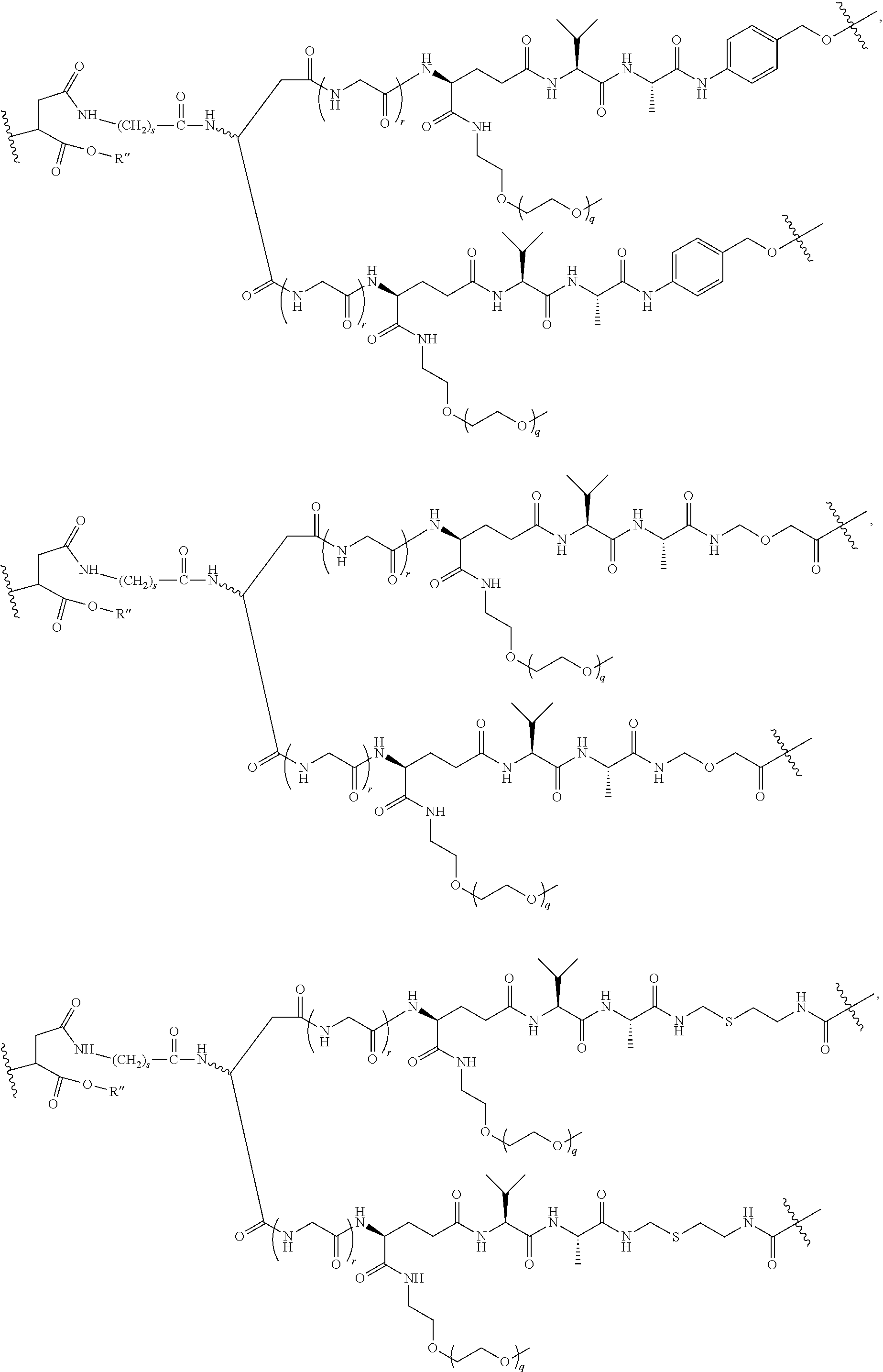

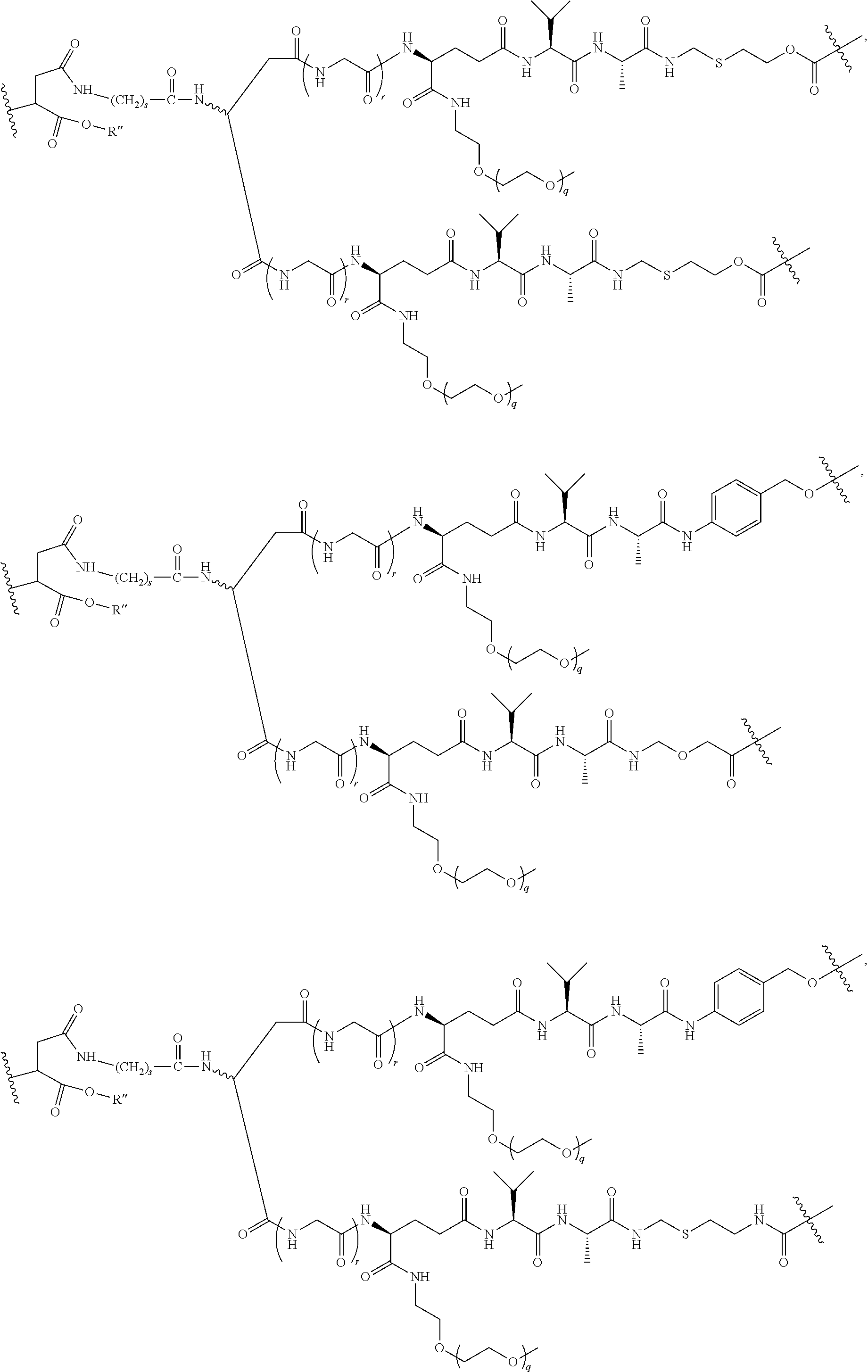

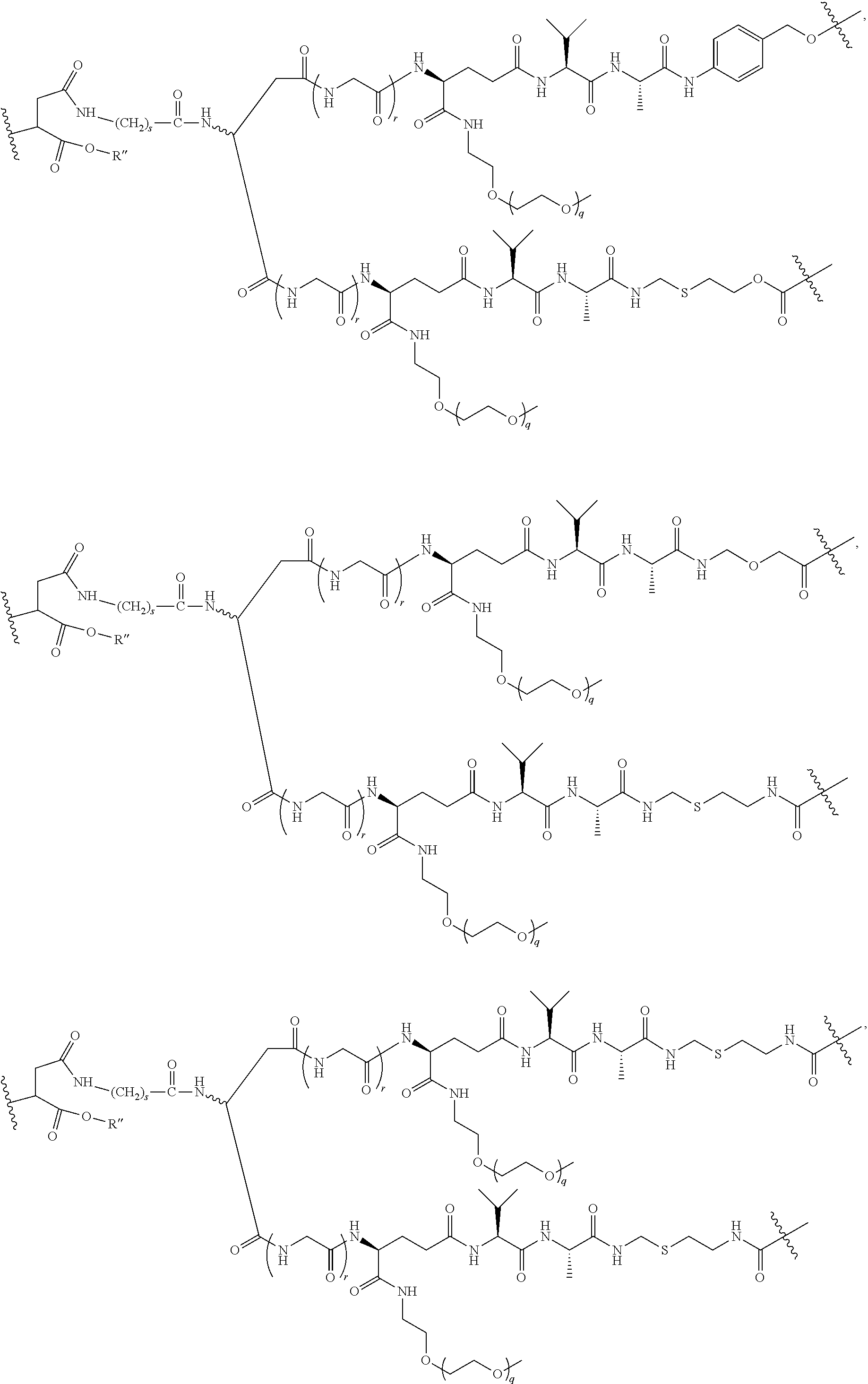

wherein R′ is selected from the group consisting of methyl, ethyl and propyl, each subscript s is any integer independently selected from 1-10, each subscript r is any integer independently selected from 1-4, and each subscript q is any integer independently selected from 1-20.

In another aspect, the present application provides use of the previously defined linker in preparation of a drug, wherein the drug comprises a linker-drug conjugate or a ligand-drug conjugate.

### Linker-Drug Conjugate

In another aspect, the present application provides a linker-drug conjugate comprising the previously defined linker and having a structure shown in Formula (II):

La—Bₚ [symbol] -Lb-Lc-D)ₘ Formula (II),
or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

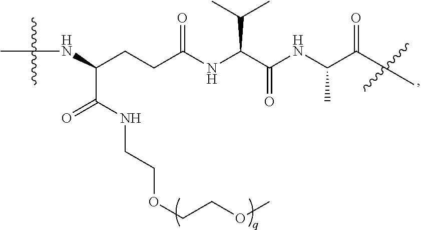

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the linker-drug conjugate has a structure shown in Formula (IIa), (IIb), (IIc), or (IId):

La-Lb-Lc-D, Formula (IIa),

La—B [symbol] Lb-Lc-D)₂, Formula (IIb),

La—B [symbol] Lb-Lc-D)₃, Formula (IIc),

La—B [symbol] Lb-Lc-D)₄, Formula (IId),
or a pharmaceutically acceptable salt thereof, wherein D is a drug unit.

In some embodiments, the linker-drug conjugate has a structure shown in Formula (IIa) or (IIb):

La-Lb-Lc-D, Formula (IIa),

La—B [symbol] Lb-Lc-D)₂, Formula (IIb),
or a pharmaceutically acceptable salt thereof, wherein,
La is an extension unit capable of linking a ligand unit;
B is a branching unit having a structure selected from the following formula:

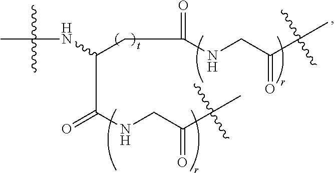

wherein a subscript t is any integer independently selected from 1-4, each subscript r is any integer independently selected from 1-4, and the B structure is linked to the La structure via amino and to the Lb structure fragment via carbonyl;
Lc is a releasable assembly unit; and
the Lb structure is selected from the following formula:

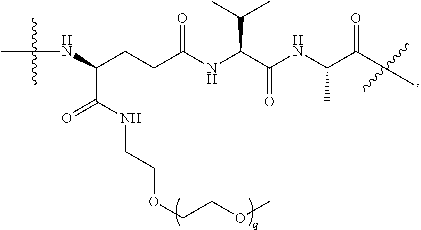

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.
For example, the linker-drug conjugate is selected from the following formula:

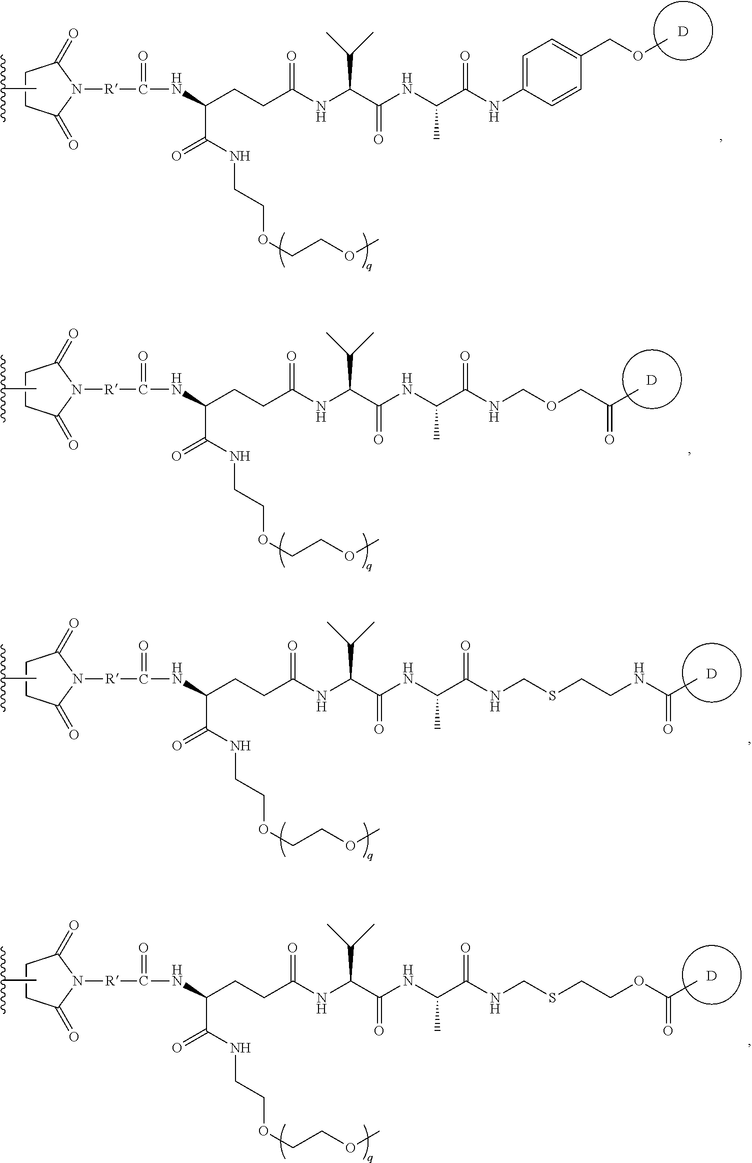

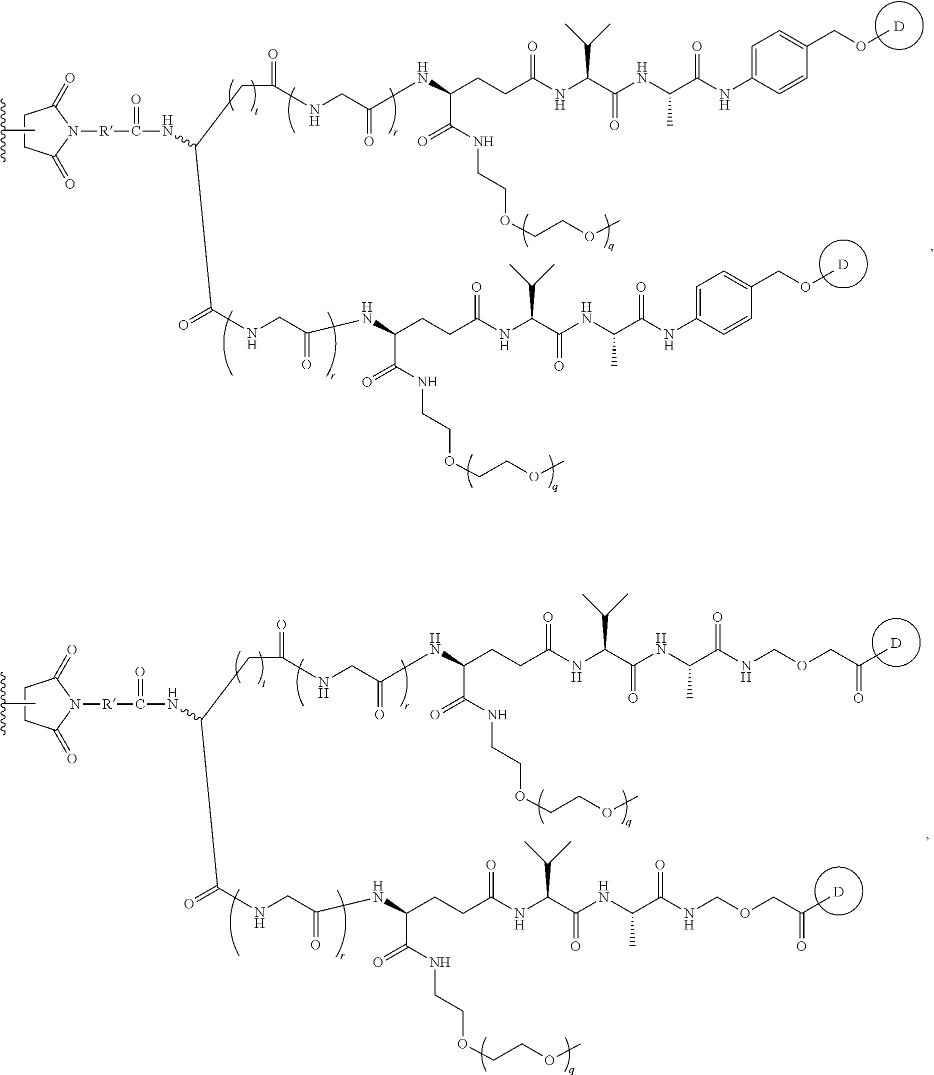

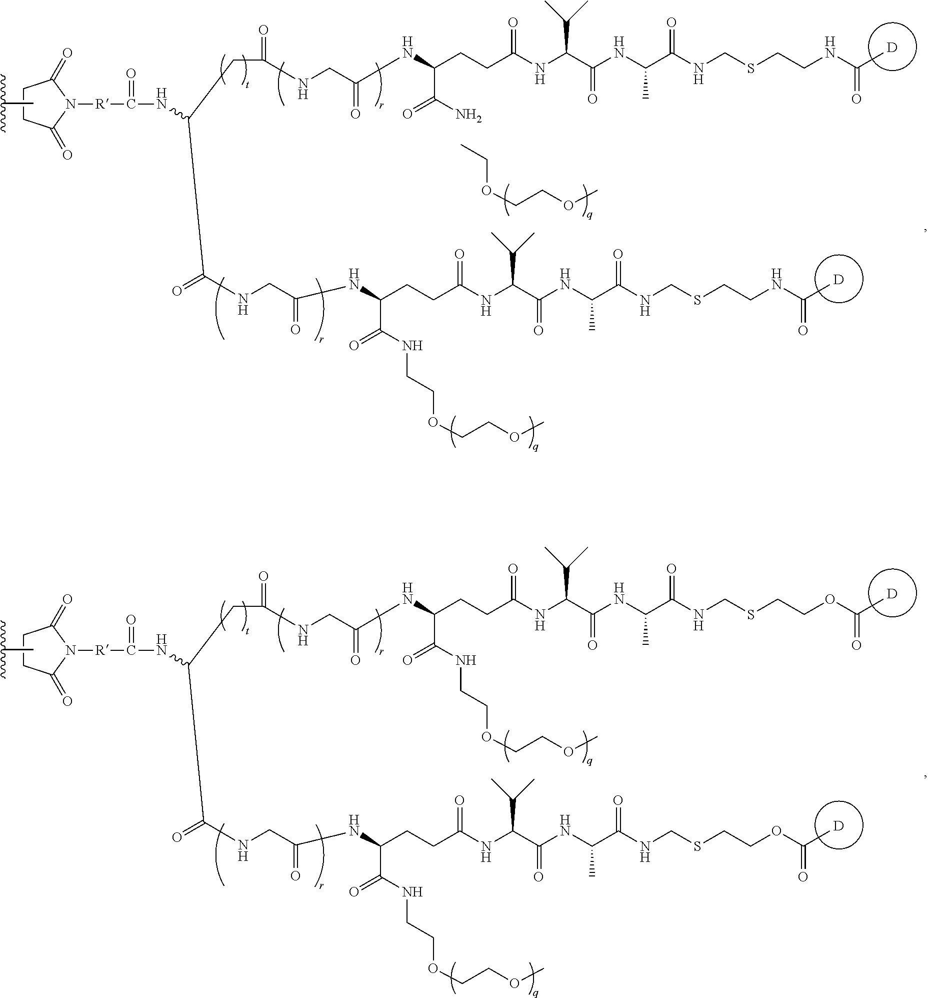

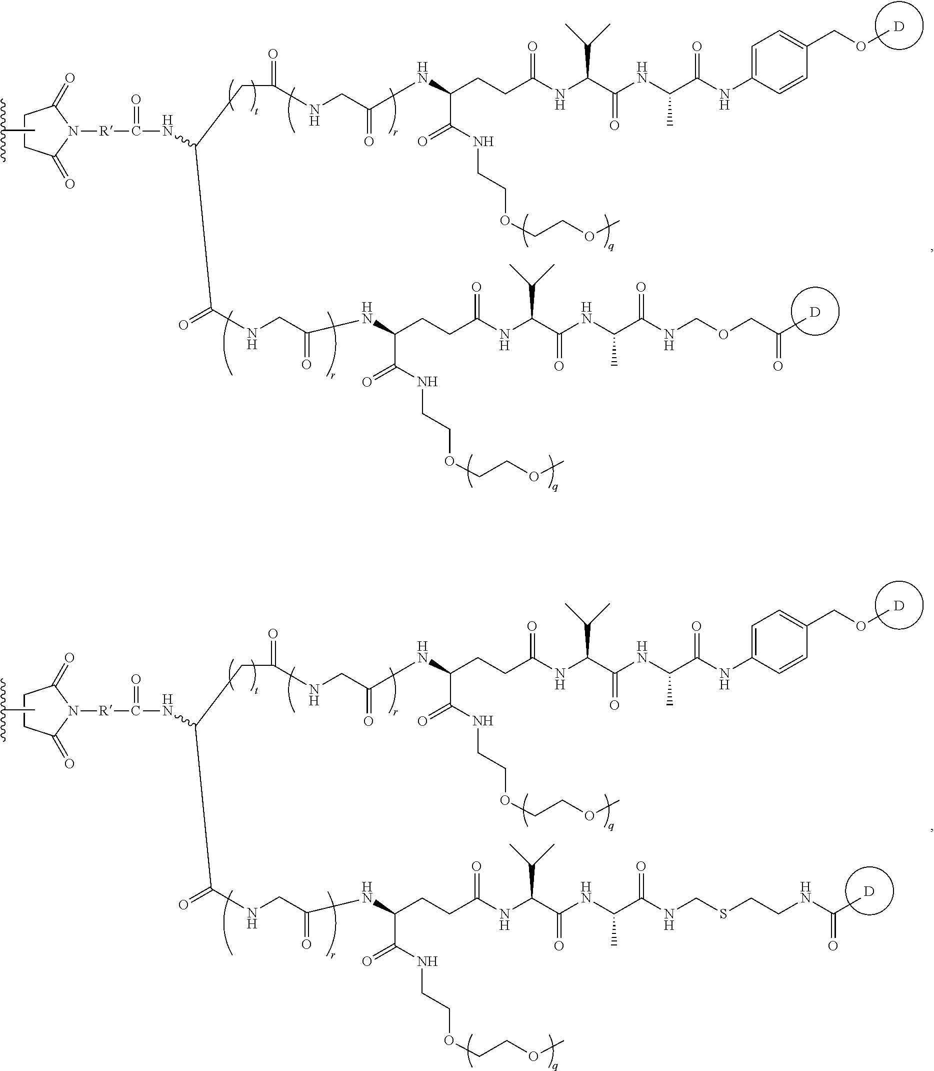

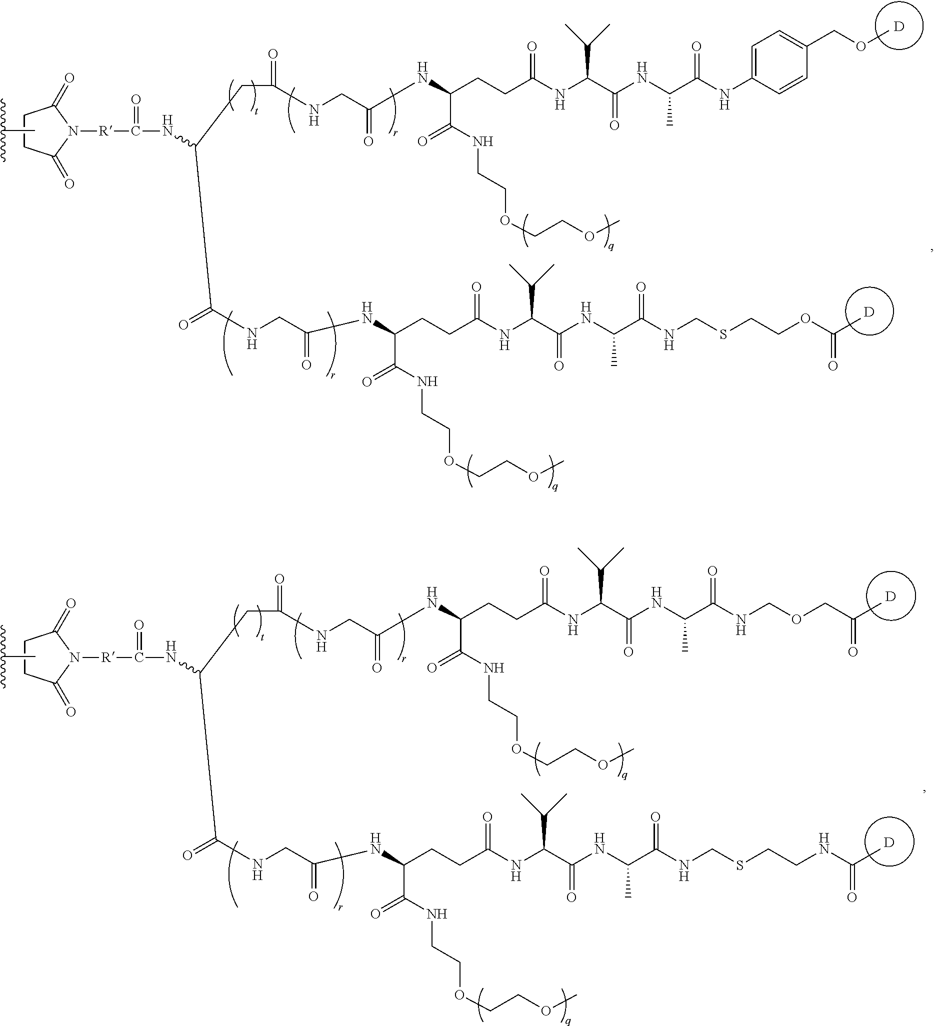

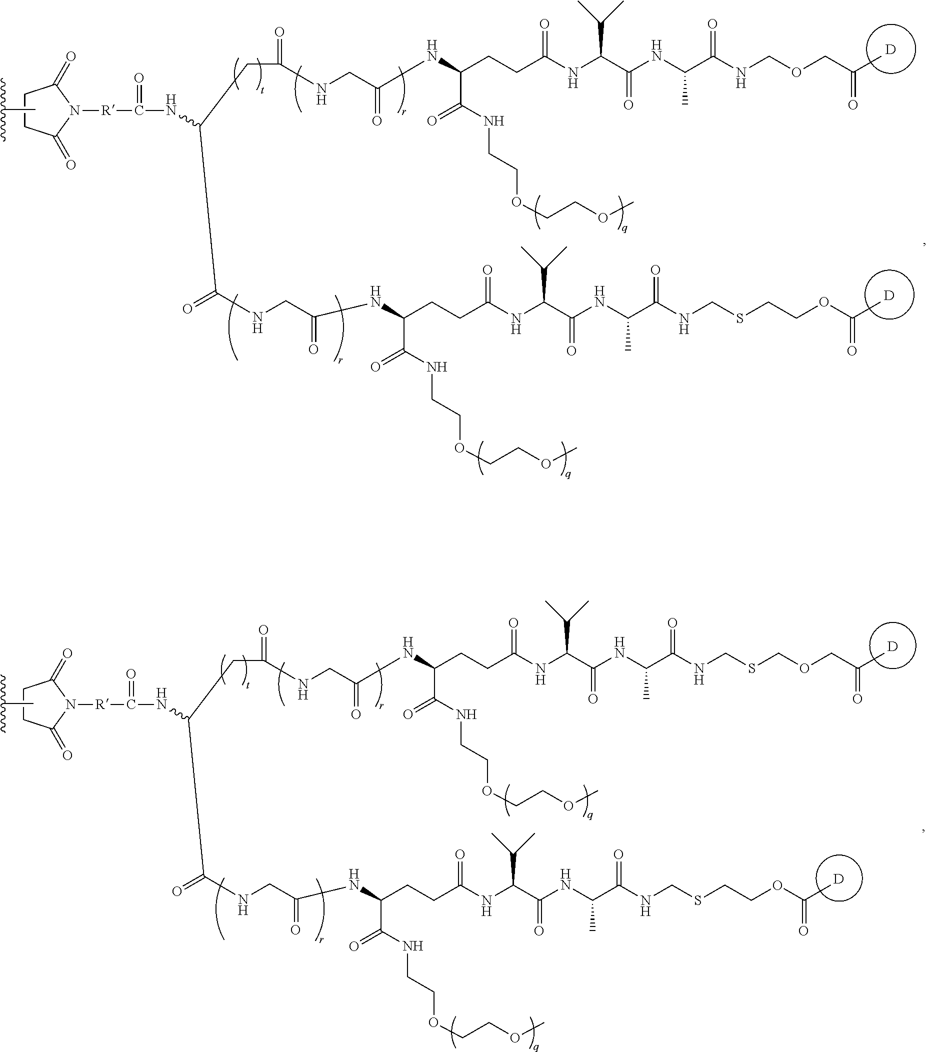

wherein,
R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl;
each subscript r is any integer independently selected from 1-4, each subscript t is any integer independently selected from 1-4, and each subscript q is any integer independently selected from 1-20, preferably 3-10, and most preferably 5-8.

In some embodiments, the drug unit D comprises a detectable label, a drug, a cytokine, an enzyme, or their combinations.

In some embodiments, the drug unit D comprises a cytotoxic agent and/or cytostatics.

In some embodiments, the drug unit D comprises a DNase topoisomerase I (topology I) inhibitor.

In some embodiments, the drug unit D comprises camptothecin and a derivative thereof.

In some embodiments, the linker-drug conjugate has a structure shown in Formula (III):

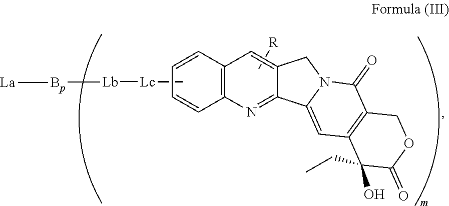

or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

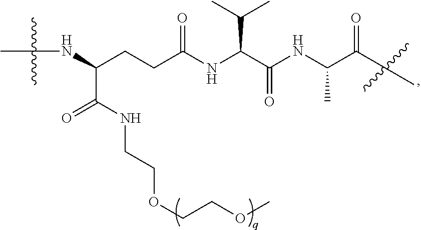

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

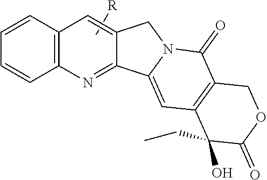

represents camptothecin or a derivative thereof.

In some embodiments, the linker-drug conjugate has a structure shown in Formula (IIIa), (IIIb), (IIIc) or (IIId):

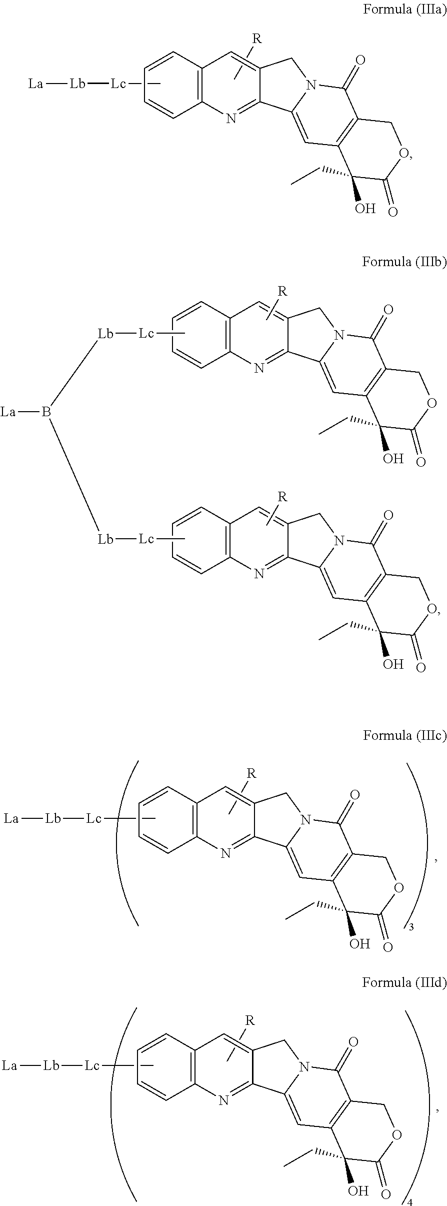

or a pharmaceutically acceptable salt thereof.

In some embodiments, the linker-drug conjugate has a structure shown in Formula (IIIa) or (IIIb):

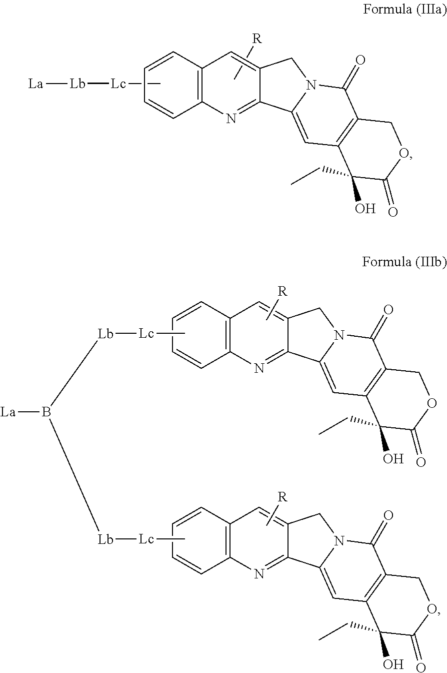

or a pharmaceutically acceptable salt thereof, wherein
La is an extension unit capable of linking a ligand unit;
B is a branching unit having a structure selected from the following formula:

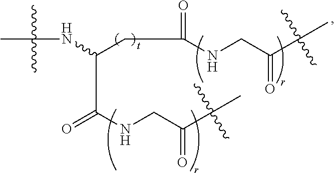

wherein a subscript t is any integer independently selected from 1-4, each subscript r is any integer independently selected from 1-4, and the B structure is linked to the La structure via amino and to the Lb structure fragment via carbonyl;

the Lb structure is selected from the following formula:

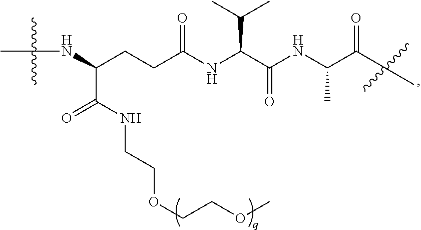

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

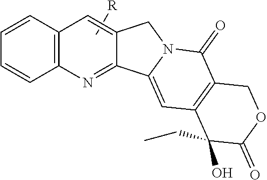

represents camptothecin or a derivative thereof.

For example, the linker-drug conjugate may be selected from the following structure:

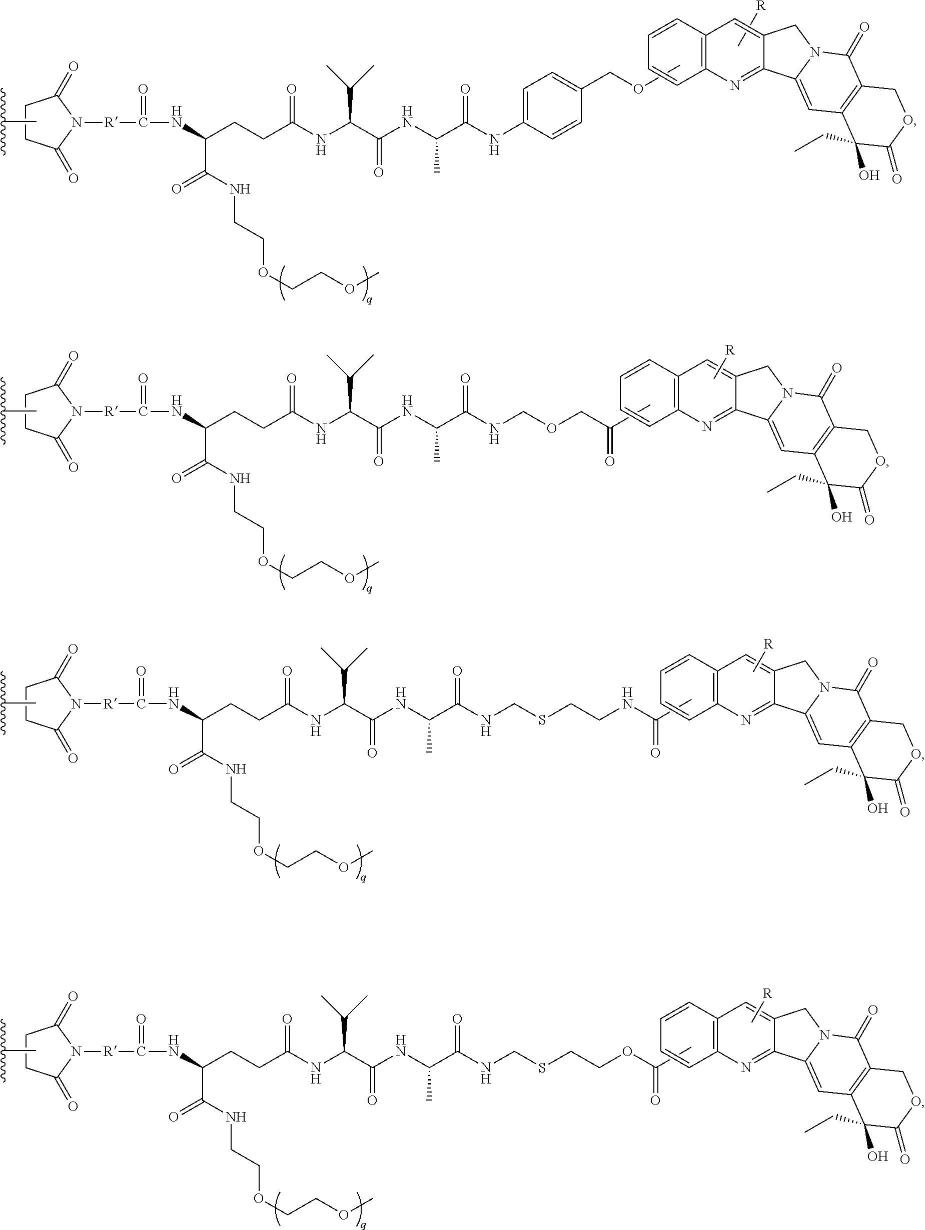

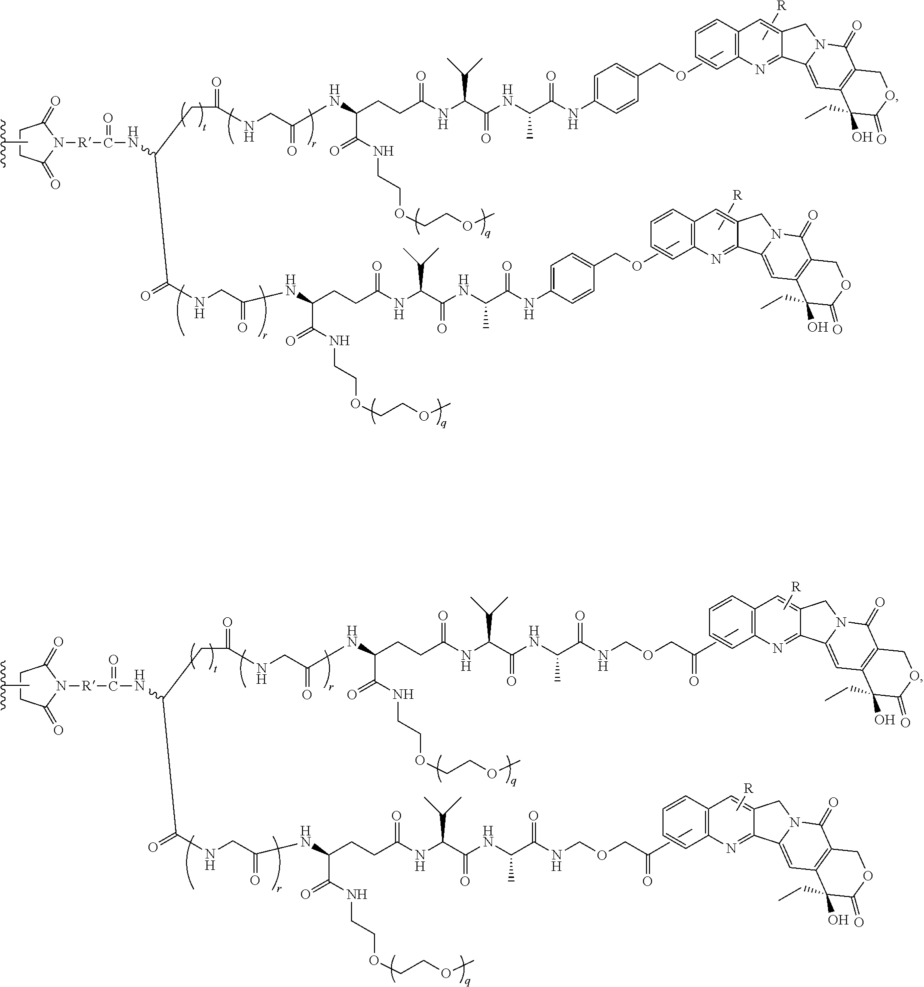

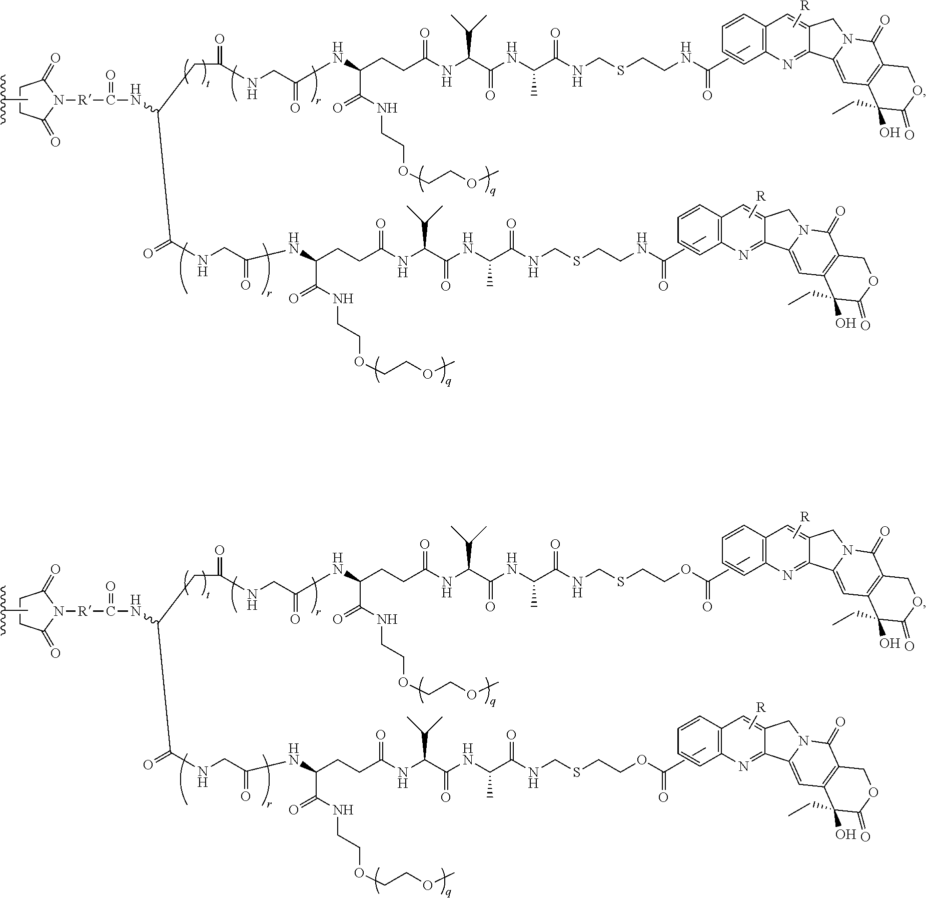

wherein,
R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl;
each subscript r is any integer independently selected from 1-4, each subscript t is any integer independently selected from 1-4, and each subscript q is any integer independently selected from 1-20, preferably 3-10, and most preferably 5-8.

In some embodiments, the camptothecin and the derivative thereof comprise 10-difluoromethyl camptothecin compound and a derivative thereof.

In some embodiments, the 10-difluoromethyl camptothecin compound has the following structure:

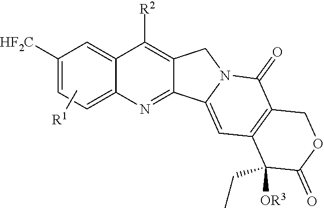

in which R¹ is selected from the group consisting of hydrogen, optionally substituted amino and optionally substituted C₁-C₆ alkyl, and a binding site of R¹ is any one of three unsubstituted sites on a benzene ring; R² is selected from the group consisting of hydrogen, optionally substituted amino and optionally substituted C₁-C₆ alkyl; and R³ is selected from the group consisting of hydrogen, acyl and optionally substituted C₁-C₆ alkyl.

In some embodiments, the 10-difluoromethyl camptothecin compound has the following structure:

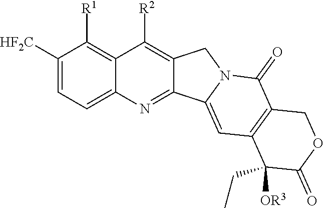

in which R¹ is selected from the group consisting of hydrogen, optionally substituted amino and optionally substituted C₁-C₆ alkyl; R² is selected from the group consisting of hydrogen, optionally substituted amino and optionally substituted C₁-C₆ alkyl; and R³ is selected from the group consisting of hydrogen, acyl and optionally substituted C₁-C₆ alkyl.

In some embodiments, R₁ is selected from the group consisting of hydrogen, optionally substituted amino and optionally substituted C₁-C₄ alkyl.

In some embodiments, R¹ is selected from the group consisting of hydrogen, amino and

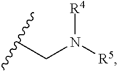

and R⁴ and R⁵ are each selected from the group consisting of hydrogen, optionally substituted C¹-C⁶ alkyl, and optionally substituted aryl.

In some embodiments, R² is selected from the group consisting of hydrogen, optionally substituted amino and optionally substituted C₁-C₄ alkyl.

In some embodiments, R² is selected from the group consisting of ethyl,

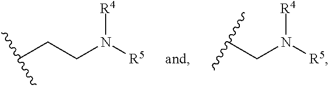

wherein R⁴ and R⁵ are each selected from the group consisting of hydrogen, optionally substituted C₁-C₆ alkyl, and optionally substituted aryl.

In some embodiments, R³ is selected from the group consisting of hydrogen, acyl and optionally substituted C₁-C₄ alkyl.

In some embodiments, R³ is selected from the group consisting of hydrogen,

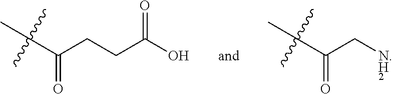

In some embodiments, the 10-difluoromethyl camptothecin compound has the following structure:

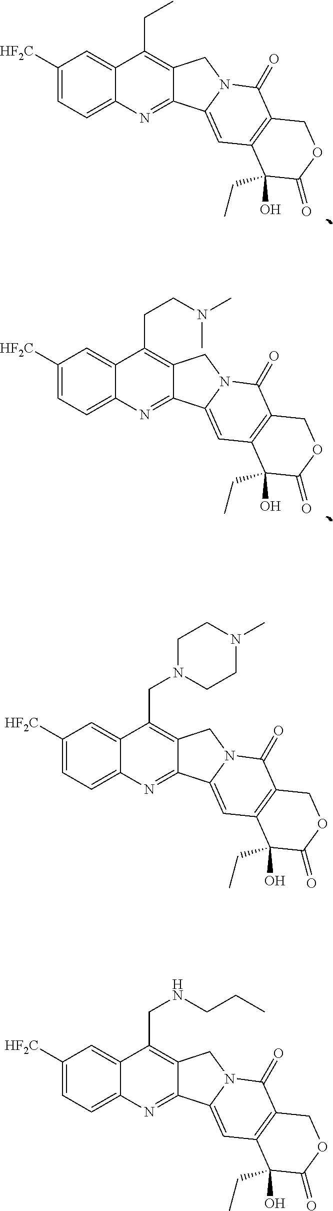

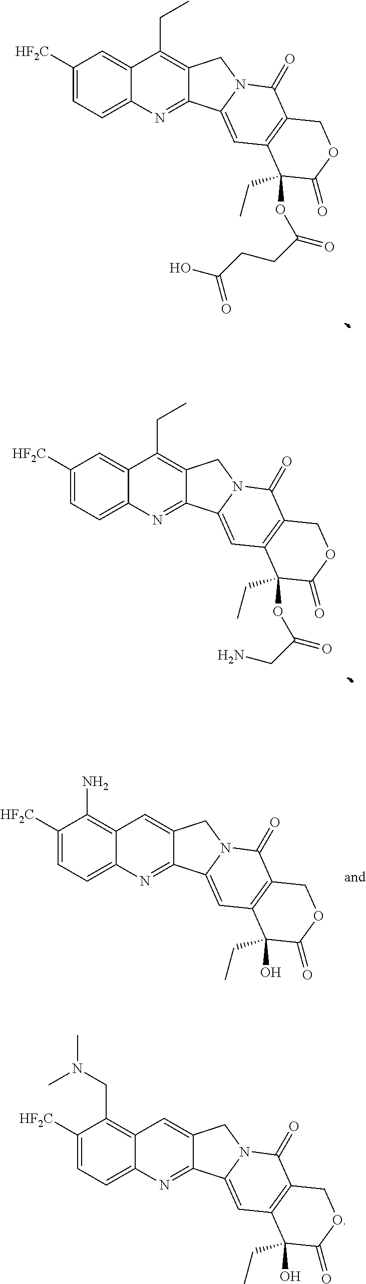

For example, the linker-drug conjugate may be selected from the following structure:

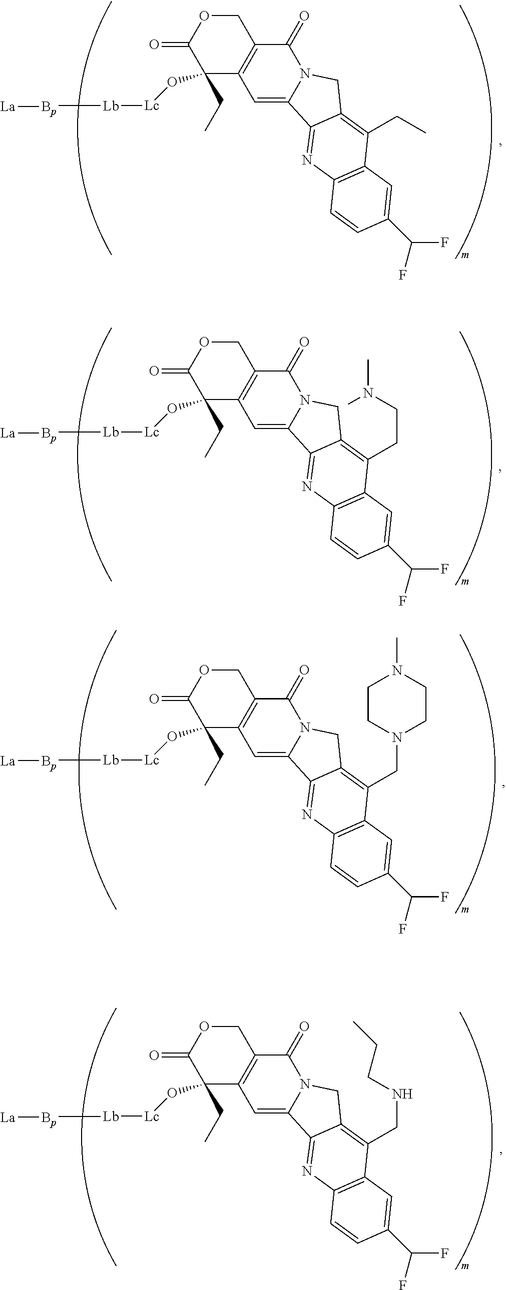

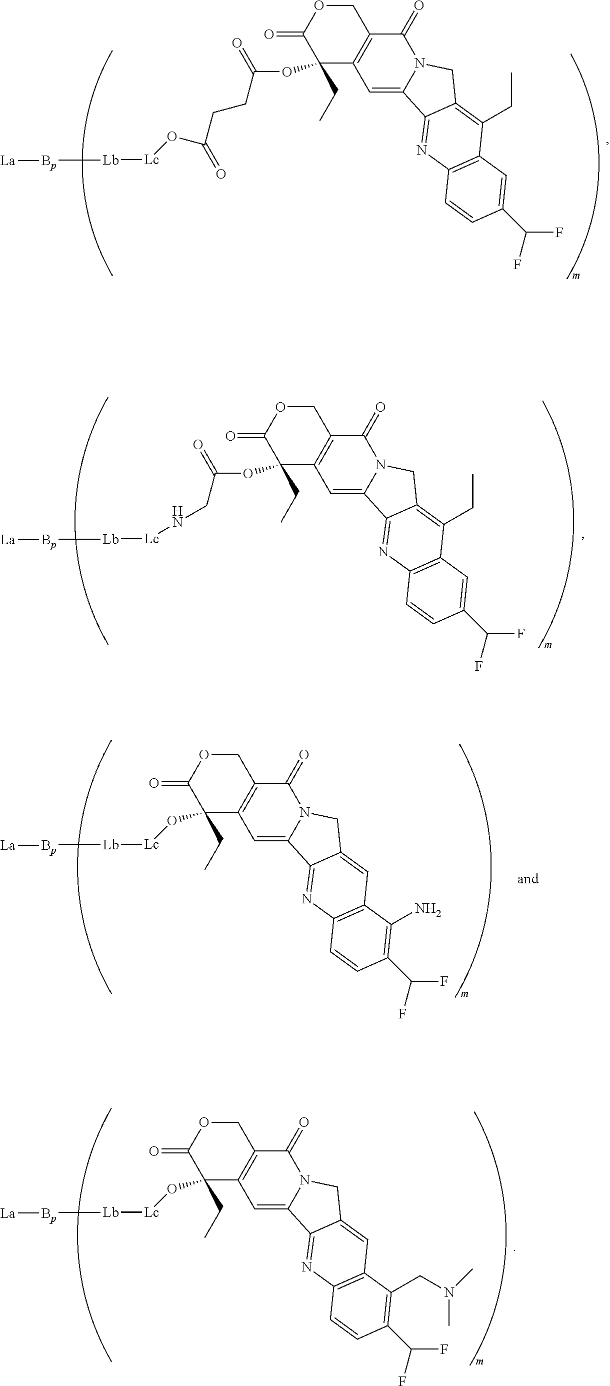

In some embodiments, the camptothecin and the derivative thereof are selected from the group consisting of: camptothecin, exatecan, topotecan, irinotecan, belotecan, lurtotecan GG-211, CKD-602, gimatecan ST1481, karenitecin BNP-1350, BN-80915, hydroxycamptothecin HCPT, 9-aminocamptothecin, 9-nitrocamptothecin, 7-ethyl-10-hydroxycamptothecin SN38, 7-ethyl-10-difluoromethylcamptothecin and their derivatives.

In some embodiments, the camptothecin and the derivative thereof are selected from the group consisting of camptothecin, exatecan, 7-ethyl-10-hydroxycamptothecin, 7-ethyl-10-difluoromethylcamptothecin, and their solvates, hydrates, stereoisomers, tautomers, isotope labels, metabolites, prodrugs, and pharmaceutically acceptable salts.

In some embodiments, the linker-drug conjugate has a structure shown in Formula (IVa), (IVb), (IVc), or (IVd):

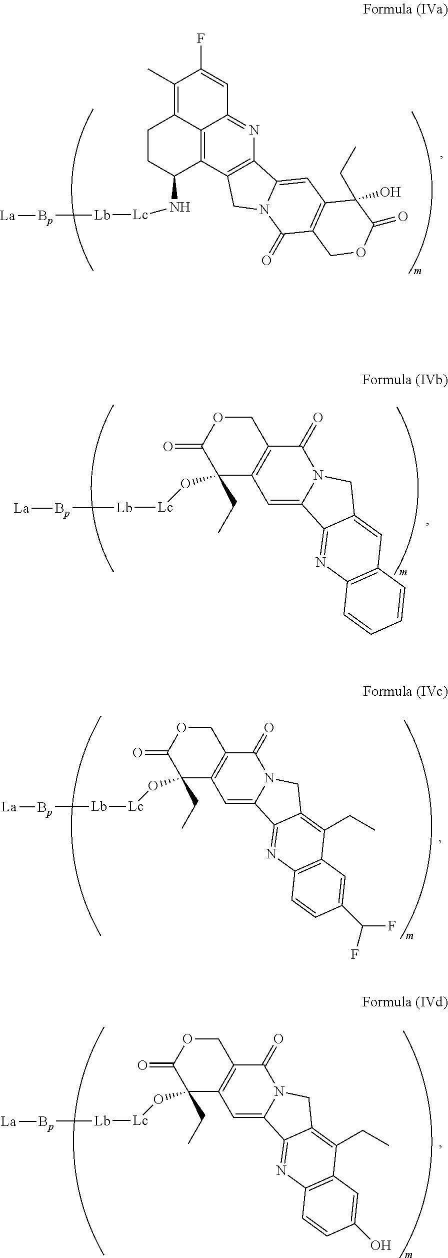

or a pharmaceutically acceptable salt thereof, wherein,
La is an extension unit capable of linking a ligand unit;
B is an optional branching unit, wherein a subscript p is 0 or 1, and wherein m is 1 when p is 0, and m is 2, 3, or 4 when p is 1;
the Lb structure is selected from the following formula:

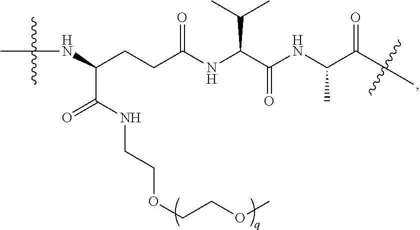

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the linker-drug conjugate is selected from the following structures:

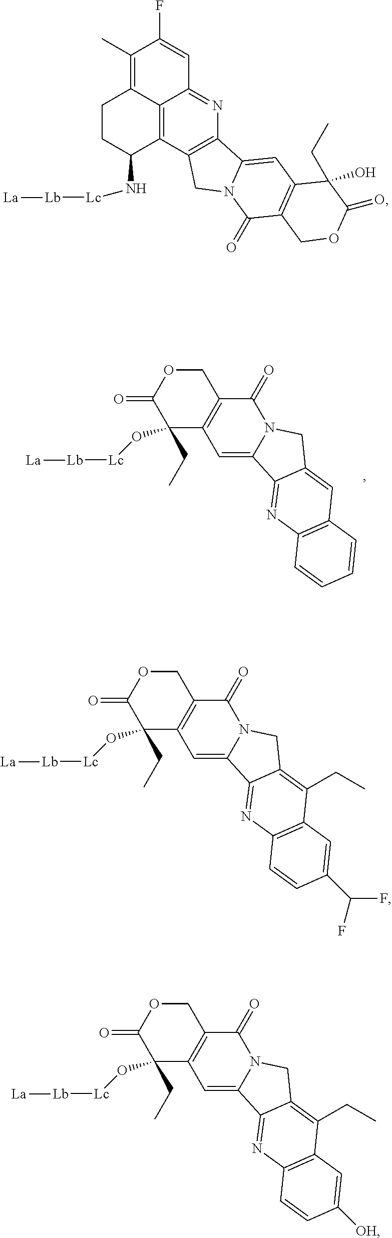

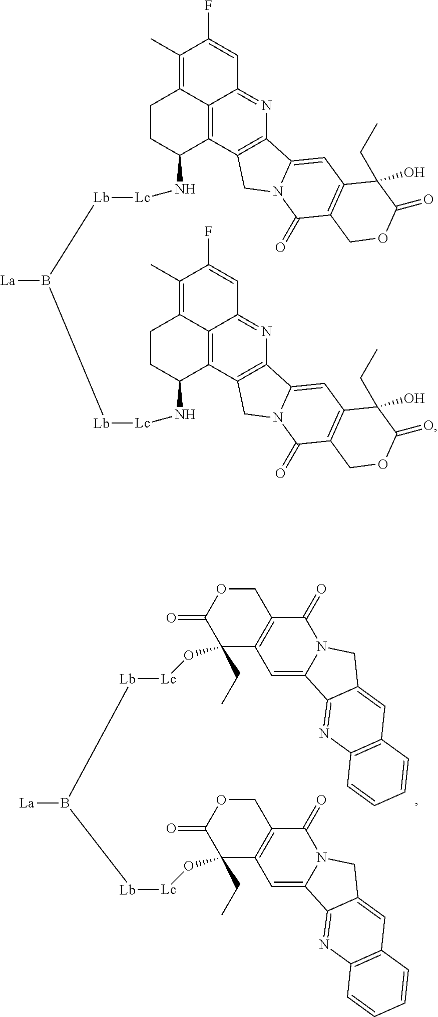

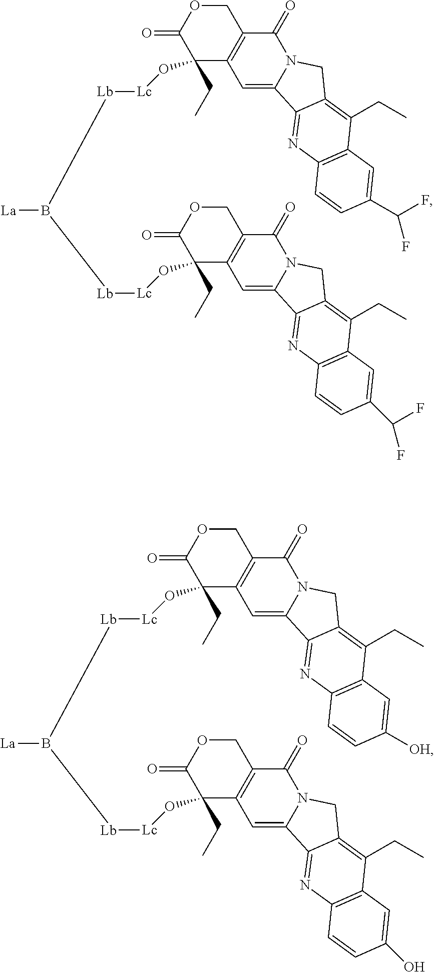

or a pharmaceutically acceptable salt thereof, wherein,
La is an extension unit;
B is a branching unit having a structure selected from the following formula:

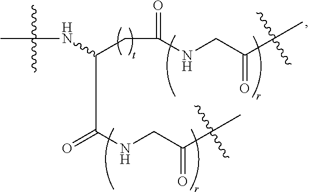

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

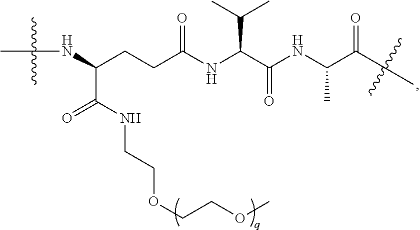

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

### Antibody-Drug Conjugate

In another aspect, the present application provides a ligand-drug conjugate comprising the previously defined linker or the previously defined linker-drug conjugate and having a structure shown in Formula (V):

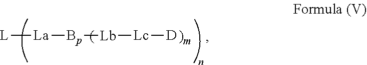

or a pharmaceutically acceptable salt thereof, wherein,
L is a ligand unit, wherein a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

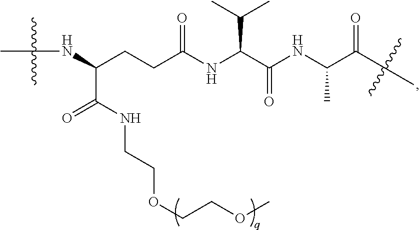

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (Va), (Vb), (Vc) or (Vd):

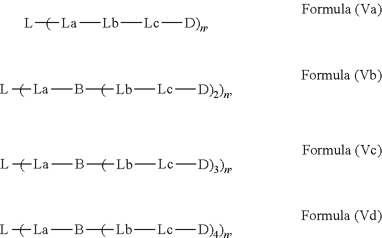

or a pharmaceutically acceptable salt thereof.

In some embodiments, the ligand-drug conjugate has a structure shown in Formula (Va) or (Vb):

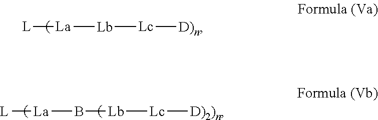

or a pharmaceutically acceptable salt thereof, wherein,
L is a ligand unit, wherein a subscript n is any integer selected from 1-8, and preferably 4-8;
La is an extension unit;
B is a branching unit having a structure selected from the following formula:

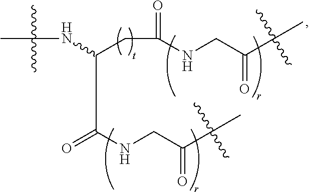

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

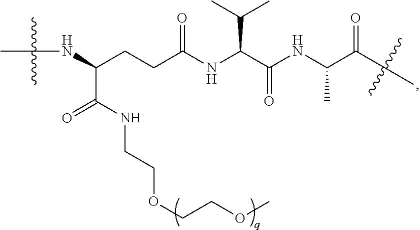

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the drug unit D comprises a cytotoxic agent and/or cytostatics.

In some embodiments, the drug unit D comprises a cytotoxic agent for cancer treatment.

In some embodiments, the drug unit D comprises a DNase topoisomerase I (topology I) inhibitor.

In some embodiments, the drug unit D comprises camptothecin and a derivative thereof.

In some embodiments, the ligand-drug conjugate has a structure shown in Formula (VI):

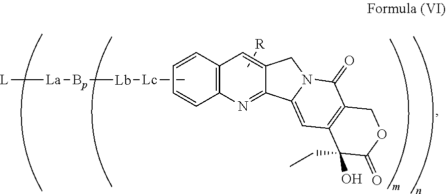

or a pharmaceutically acceptable salt thereof, wherein,
L is a ligand unit, and a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

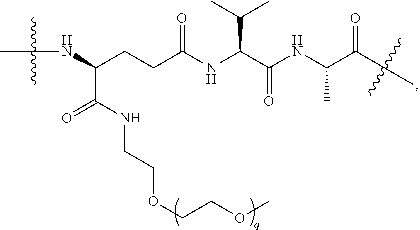

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

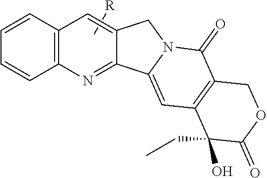

represents camptothecin or a derivative thereof.

In some embodiments, the ligand-drug conjugate has a structure shown in Formula (VIa), (VIb), (VIc) or (VId):

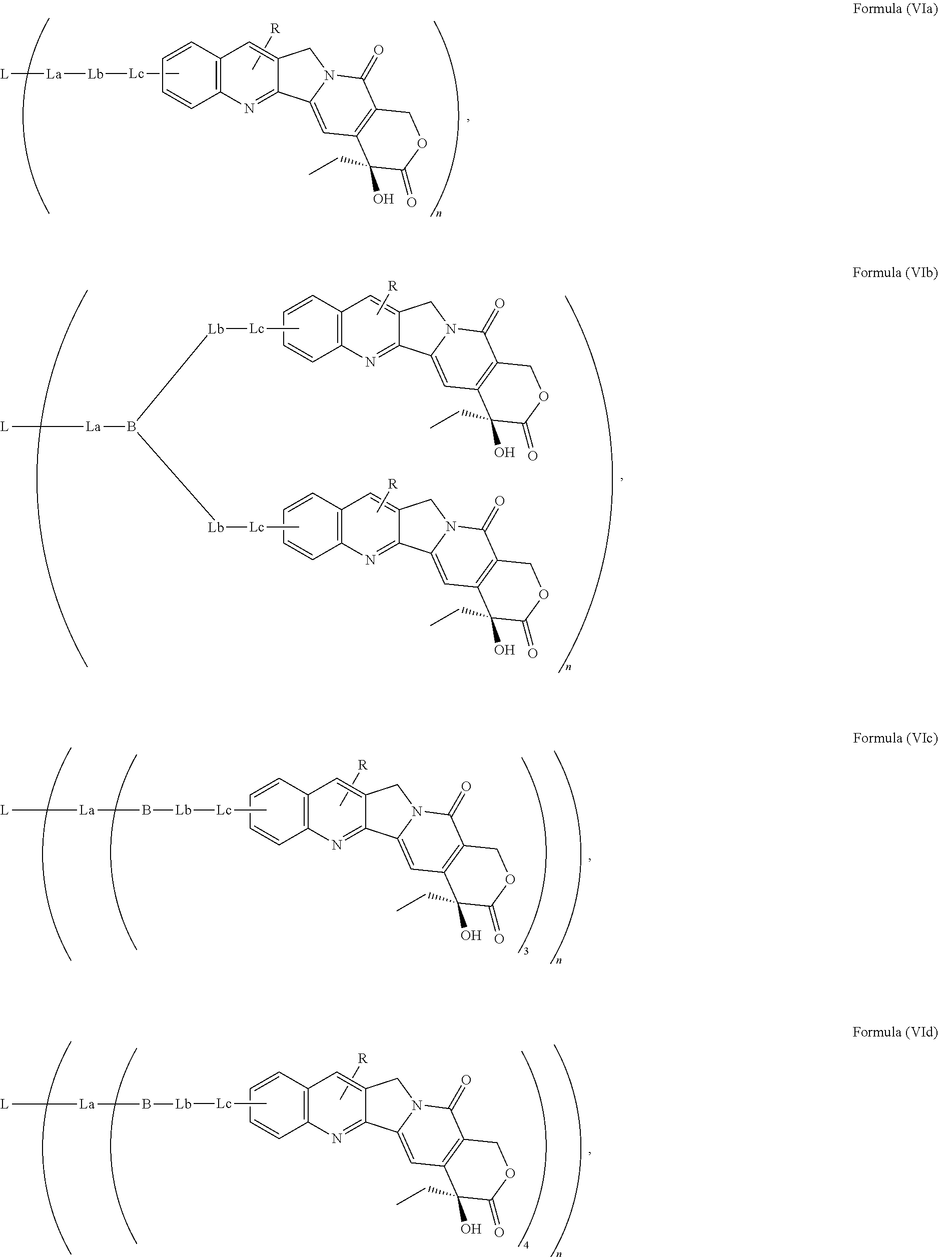

or a pharmaceutically acceptable salt thereof.

In some embodiments, the ligand-drug conjugate has a structure shown in Formula (VIa) or (VIb):

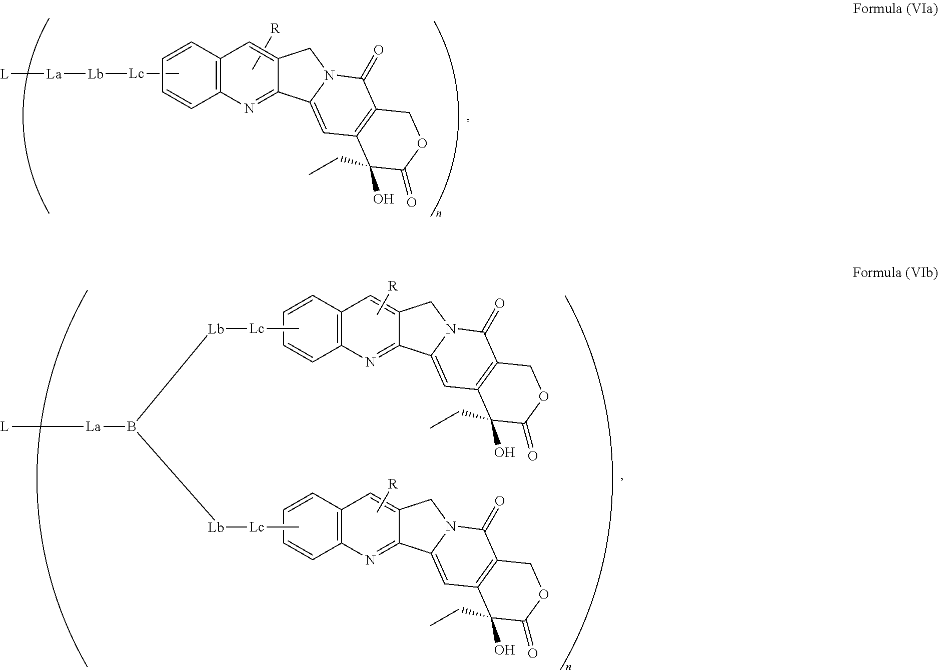

or a pharmaceutically acceptable salt thereof, wherein,
L is a ligand unit, wherein a subscript n is any integer selected from 1-8, and preferably 4-8;
La is an extension unit;
B is a branching unit having a structure selected from the following formula:

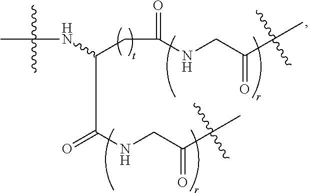

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

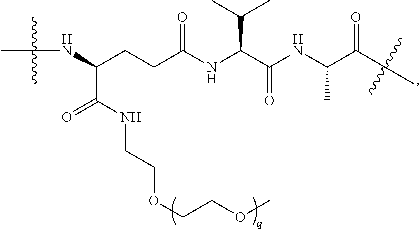

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

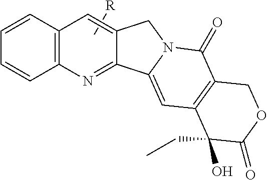

represents camptothecin or a derivative thereof.

In some embodiments, the camptothecin and the derivative thereof comprise 10-difluoromethyl camptothecin compound and a derivative thereof.

In some embodiments, the 10-difluoromethyl camptothecin compound has the following

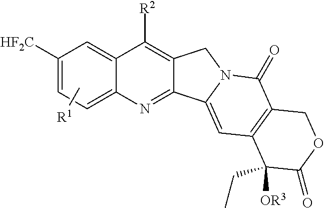

wherein,
R¹ is selected from the group consisting of hydrogen, optionally substituted amino and optionally substituted C₁-C₆ alkyl, and a binding site of R¹ is any one of three unsubstituted sites on a benzene ring;
R² is selected from the group consisting of hydrogen, optionally substituted amino and optionally substituted C₁-C₆ alkyl; and
R³ is selected from the group consisting of hydrogen, acyl and optionally substituted C₁-C₆ alkyl.

In some embodiments, the 10-difluoromethyl camptothecin compound has the following structure:

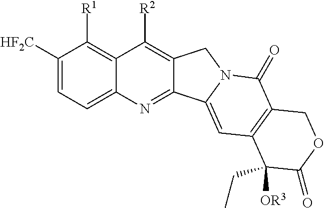

wherein,
R¹ is selected from the group consisting of hydrogen, optionally substituted amino and optionally substituted C₁-C₆ alkyl; and
R² is selected from the group consisting of hydrogen, optionally substituted amino and optionally substituted C₁-C₆ alkyl; and
R³ is selected from the group consisting of hydrogen, acyl and optionally substituted C₁-C₆ alkyl.

In some embodiments, R₁ is selected from the group consisting of hydrogen, optionally substituted amino and optionally substituted C₁-C₄ alkyl.

In some embodiments, R¹ is selected from the group consisting of hydrogen, amino and

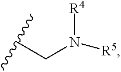

and R⁴ and R⁵ are each selected from the group consisting of hydrogen, optionally substituted C₁-C₆ alkyl, and optionally substituted aryl.

In some embodiments, R² is selected from the group consisting of hydrogen, optionally substituted amino and optionally substituted C₁-C₄ alkyl.

In some embodiments, R² is selected from the group consisting of ethyl,

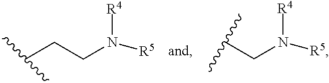

wherein R⁴ and R⁵ are each selected from the group consisting of hydrogen, optionally substituted C₁-C₆ alkyl, and optionally substituted aryl.

In some embodiments, R³ is selected from the group consisting of hydrogen, acyl and optionally substituted C₁-C₄ alkyl.

In some embodiments, R³ is selected from the group consisting of hydrogen,

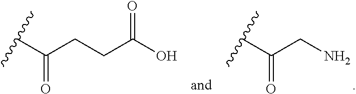 [SMILES: *C(=O)CCC(=O)O ; *C(=O)CN]

In some embodiments, the 10-difluoromethyl camptothecin compound has the following structure:

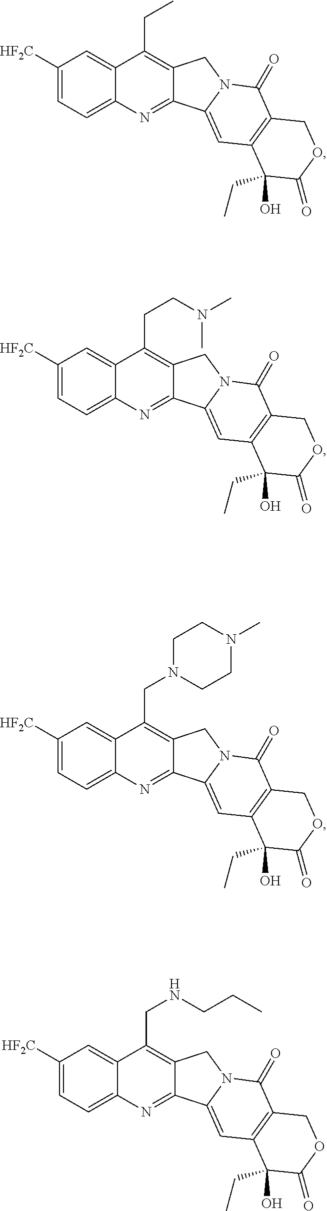

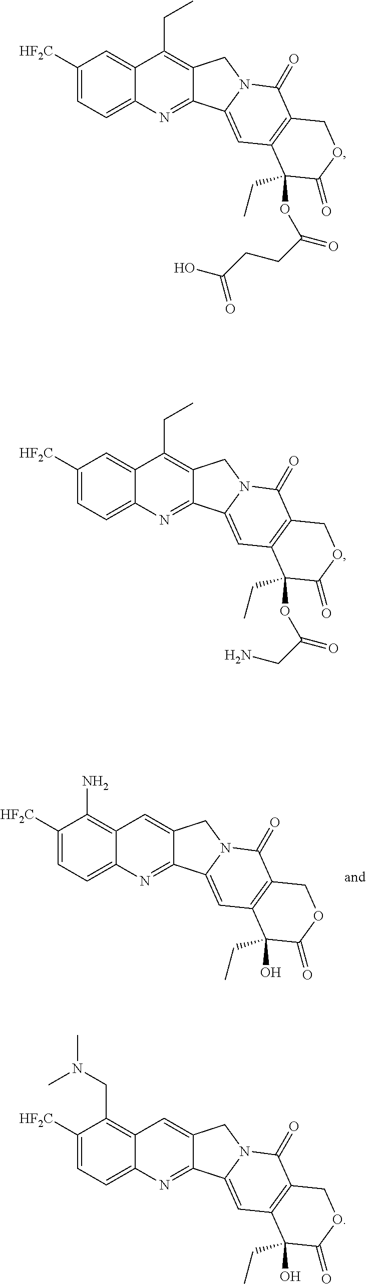

For example, the ligand-drug conjugate may be selected from the following structures:

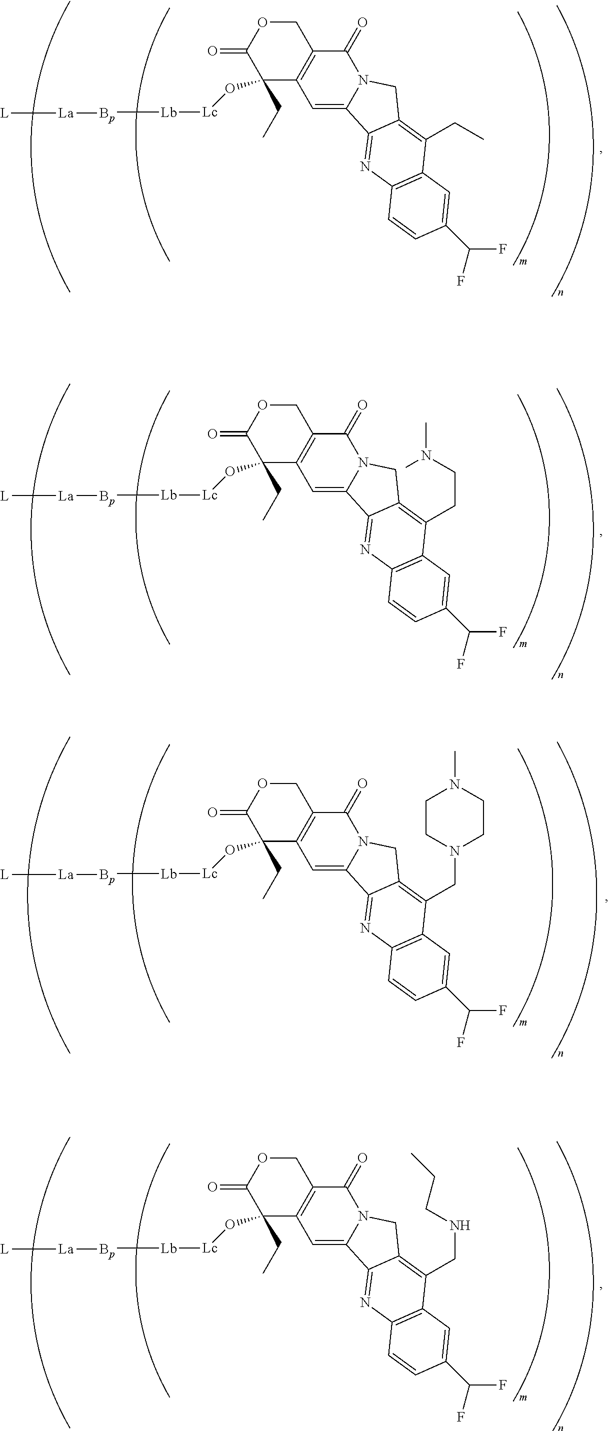

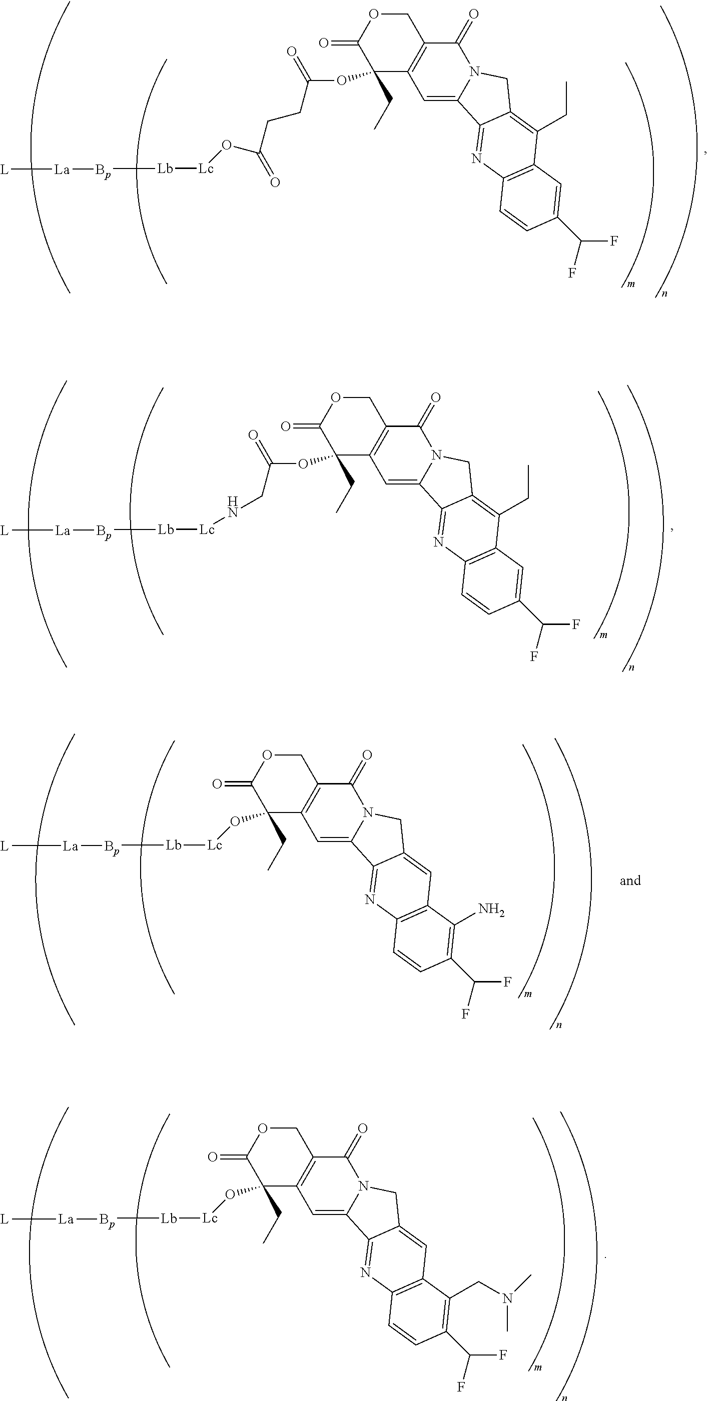

In some embodiments, the camptothecin and the derivative thereof are selected from the group consisting of: camptothecin, exatecan, topotecan, irinotecan, belotecan, lurtotecan GG-211, CKD-602, gimatecan ST1481, karenitecin BNP-1350, BN-80915, hydroxycamptothecin HCPT, 9-aminocamptothecin, 9-nitrocamptothecin, 7-ethyl-10-hydroxycamptothecin SN38, 7-ethyl-10-difluoromethylcamptothecin and their derivatives.

In some embodiments, the camptothecin and the derivative thereof are selected from the group consisting of camptothecin, exatecan, 7-ethyl-10-hydroxycamptothecin, 7-ethyl-10-difluoromethylcamptothecin, and their solvates, hydrates, stereoisomers, tautomers, isotope labels, metabolites, prodrugs, and pharmaceutically acceptable salts.

In some embodiments, the ligand-drug conjugate has a structure shown in Formula (VIIa), (VIIb), (VIIc) or (VIId):

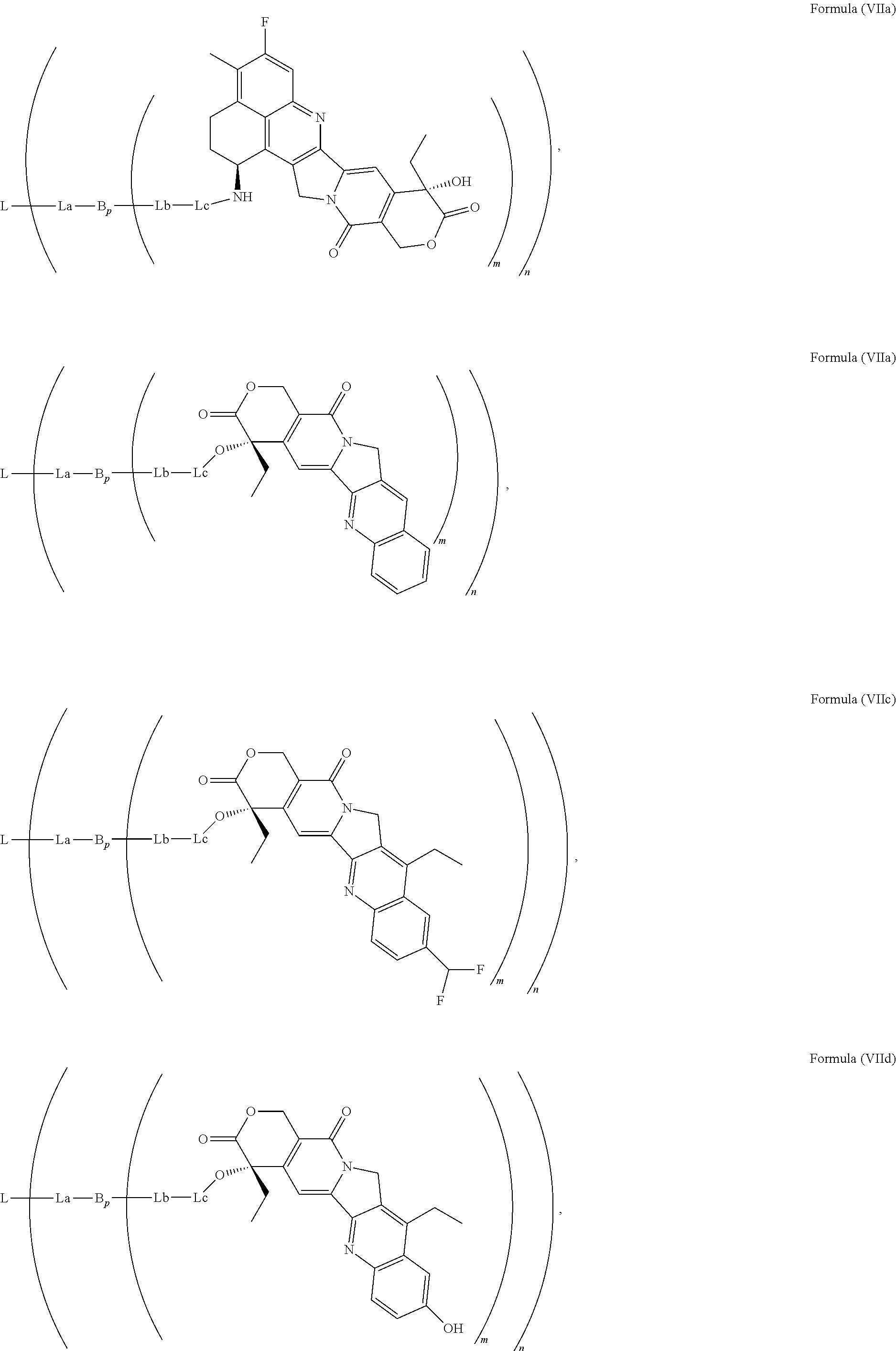

or a pharmaceutically acceptable salt thereof, wherein,
L is a ligand unit, and a subscript n is any integer selected from 1-8, and preferably 4-8;
La is an extension unit;
B is an optional branching unit, a subscript p is 0 or 1, and wherein m is 1 when p is 0, and m is 2, 3, or 4 when p is 1;
the Lb structure is selected from the following formula:

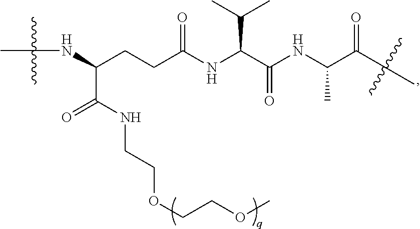

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

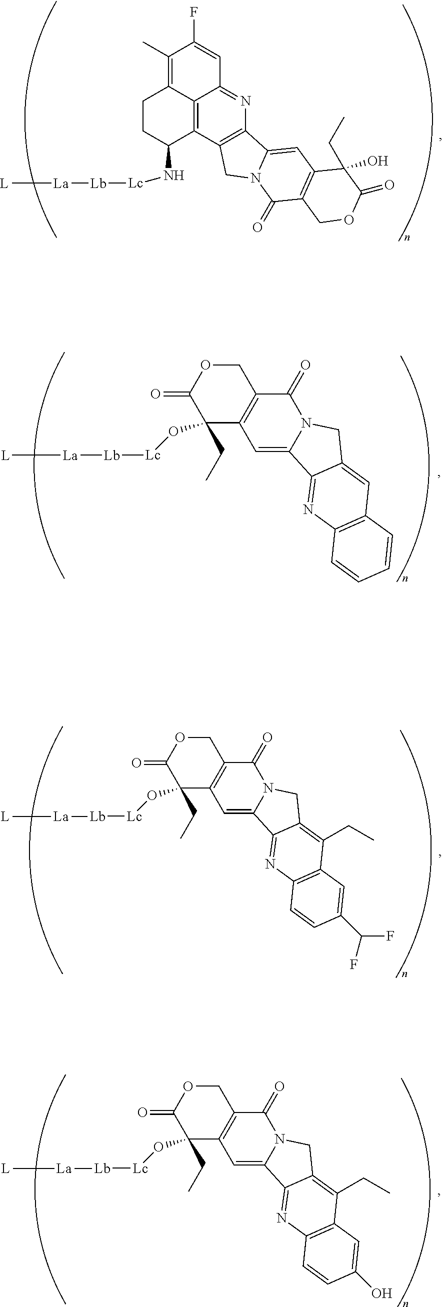

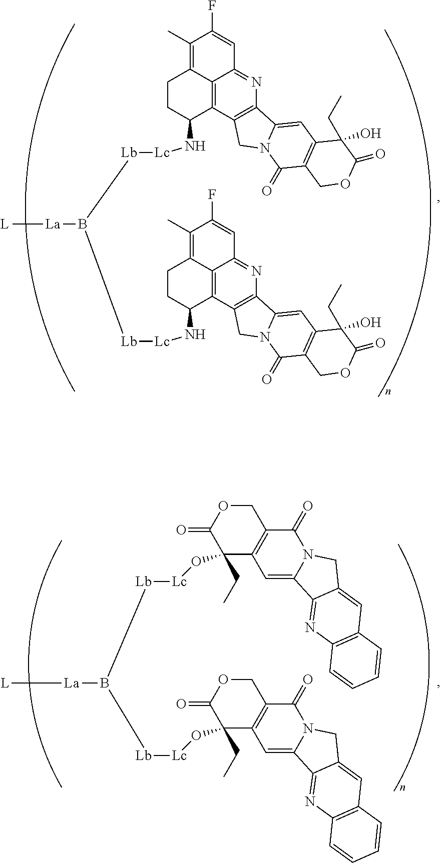

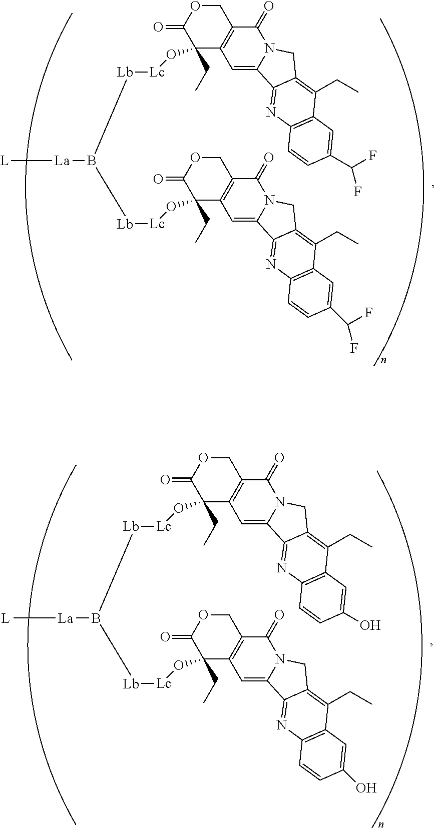

or a pharmaceutically acceptable salt thereof, wherein,
L is a ligand unit, and a subscript n is any integer selected from 1-8, and preferably 4-8;
La is an extension unit;
B is a branching unit having a structure selected from the following formula:

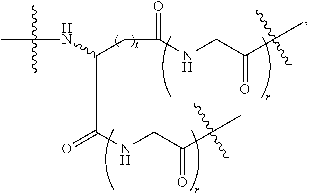

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

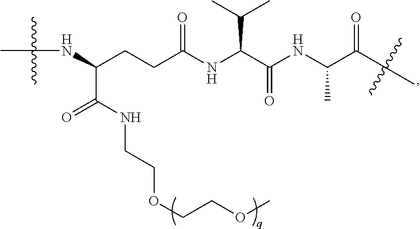

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand comprises an antibody.

In some embodiments, the antibody comprises a monoclonal antibody, a polyclonal antibody, a dimer, a multimer, a polyspecific antibody, a complete antibody, an antibody fragment, a human antibody, a humanized antibody, a chimeric antibody, or an antibody from another species.

In some embodiments, the antibody fragment comprises: Fab, Fab′, F(ab′)2, a Fv fragment, a scFv antibody fragment, a linear antibody, a single-domain antibody such as sdAB, a cameliaceae VHH domain, or a polyspecific antibody formed from an antibody fragment.

In some embodiments, the antibody comprises modified or unmodified analogues and derivatives, and the antibody is allowed to retain antigen-binding immune specificity thereof.

In some embodiments, the antibody or antibody fragment has a number of linkage points necessary for the drug-linker.

In some embodiments, the ligand-drug conjugate has a structure shown in Formula (VIII):

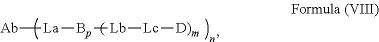

or a pharmaceutically acceptable salt thereof, wherein,
Ab represents an antibody, wherein a subscript n is any integer selected from 1-8, and preferably 4-8;
La is an extension unit;
B is an optional branching unit, wherein a subscript p is 0 or 1, and wherein m is 1 when p is 0, and m is 2, 3, or 4 when p is 1;
the Lb structure is selected from the following formula:

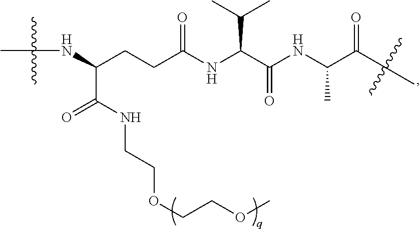

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (VIIIa) or (VIIIb):

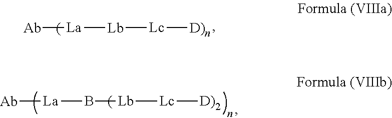

or a pharmaceutically acceptable salt thereof, wherein,
Ab represents an antibody, and a subscript n is any integer selected from 1-8, and preferably 4-8;
La is an extension unit; B is a branching unit having a structure selected from the following formula:

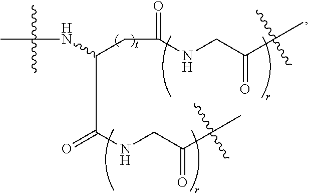

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

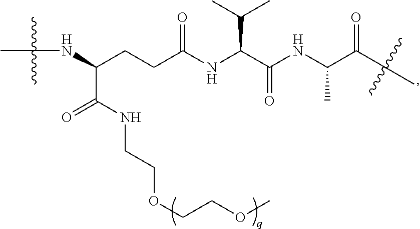

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and
D is a drug unit.

In some embodiments, the ligand-drug conjugate has a structure shown in Formula (IX):

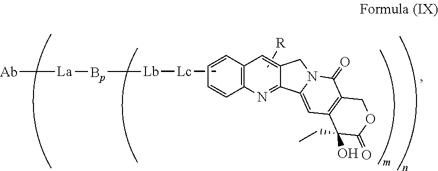

or a pharmaceutically acceptable salt thereof, wherein,
Ab represents an antibody, and a subscript n is any integer selected from 1-8, and preferably 4-8;
La is an extension unit,
B is an optional branching unit, a subscript p is 0 or 1, and wherein m is 1 when p is 0, and m is 2, 3, or 4 when p is 1;
the Lb structure is selected from the following formula:

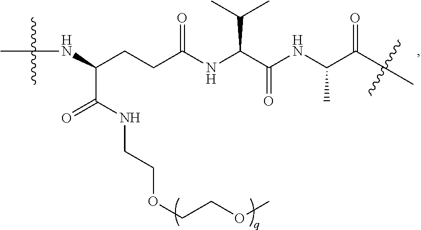

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

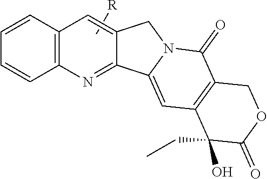

represents camptothecin or a derivative thereof.

In some embodiments, the ligand-drug conjugate has a structure shown in Formula (IXa), (IXb), (IXc) or (IXd):

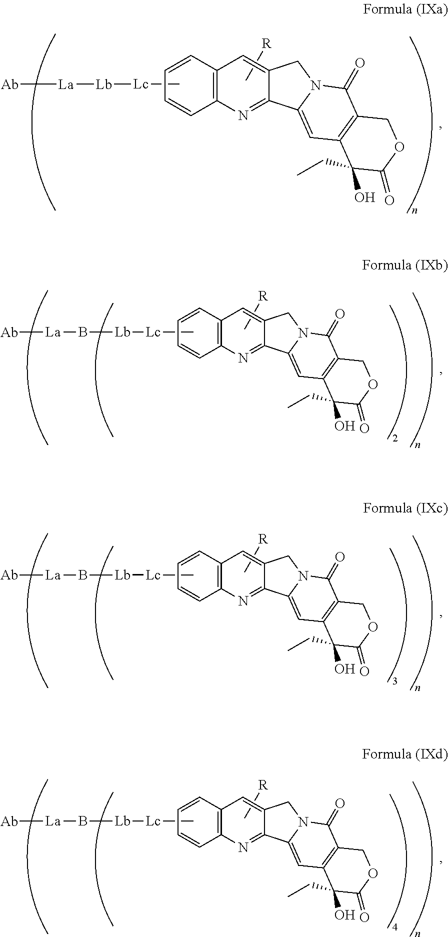

or a pharmaceutically acceptable salt thereof.

In some embodiments, the ligand-drug conjugate has a structure shown in Formula (IXa) or (IXb):

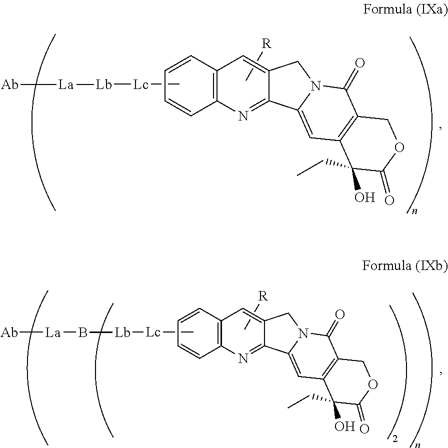

or a pharmaceutically acceptable salt thereof, wherein,
Ab represents an antibody, and a subscript n is any integer selected from 1-8, and preferably 4-8;
La is an extension unit;
B is a branching unit having a structure selected from the following formula:

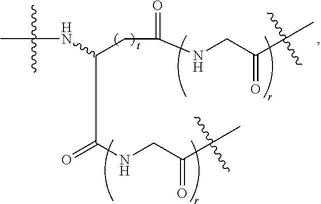

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

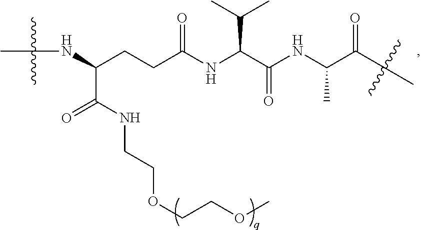

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit; and

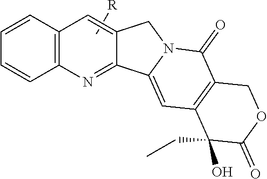

represents camptothecin or a derivative thereof.

In some embodiments, the camptothecin and the derivative thereof comprise 10-difluoromethyl camptothecin compound and a derivative thereof.

In some embodiments, the 10-difluoromethyl camptothecin compound has the following structure:

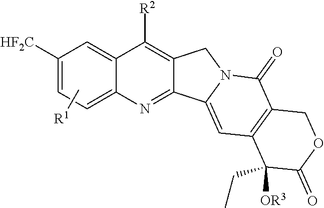

in which R¹ is selected from the group consisting of hydrogen, optionally substituted amino and optionally substituted C₁-C₆ alkyl, and a binding site of R¹ is any one of three unsubstituted sites on a benzene ring; R² is selected from the group consisting of hydrogen, optionally substituted amino and optionally substituted C₁-C₆ alkyl; and R³ is selected from the group consisting of hydrogen, acyl and optionally substituted C₁-C₆ alkyl.

In some embodiments, the 10-difluoromethyl camptothecin compound has the following structure:

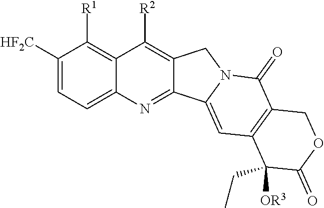

in which R¹ is selected from the group consisting of hydrogen, optionally substituted amino and optionally substituted C₁-C₆ alkyl; R² is selected from the group consisting of hydrogen, optionally substituted amino and optionally substituted C₁-C6 alkyl; and R³ is selected from the group consisting of hydrogen, acyl and optionally substituted C₁-C₆ alkyl.

In some embodiments, R¹ is selected from the group consisting of hydrogen, optionally substituted amino and optionally substituted C₁-C₄ alkyl.

In some embodiments, R¹ is selected from the group consisting of hydrogen, amino and

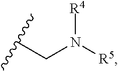

and R⁴ and R⁵ are each selected from the group consisting of hydrogen, optionally substituted C₁-C₆ alkyl, and optionally substituted aryl.

In some embodiments, R² is selected from the group consisting of hydrogen, optionally substituted amino and optionally substituted C₁-C₄ alkyl.

In some embodiments, R² is selected from the group consisting of ethyl,

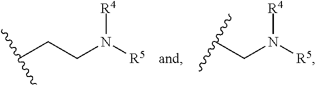

wherein R⁴ and R⁵ are each selected from the group consisting of hydrogen, optionally substituted C₁-C₆ alkyl, and optionally substituted aryl.

In some embodiments, R³ is selected from the group consisting of hydrogen, acyl and optionally substituted C₁-C₄ alkyl.

In some embodiments, R³ is selected from the group consisting of hydrogen,

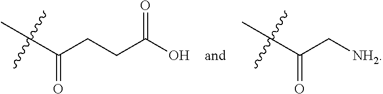 [SMILES: *C(=O)CCC(=O)O ; *C(=O)CN]

In some embodiments, the 10-difluoromethyl camptothecin compound has the following structure:

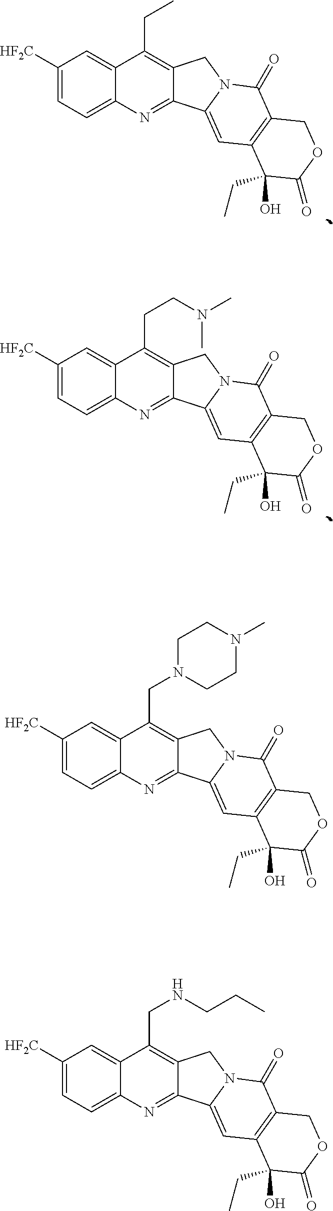

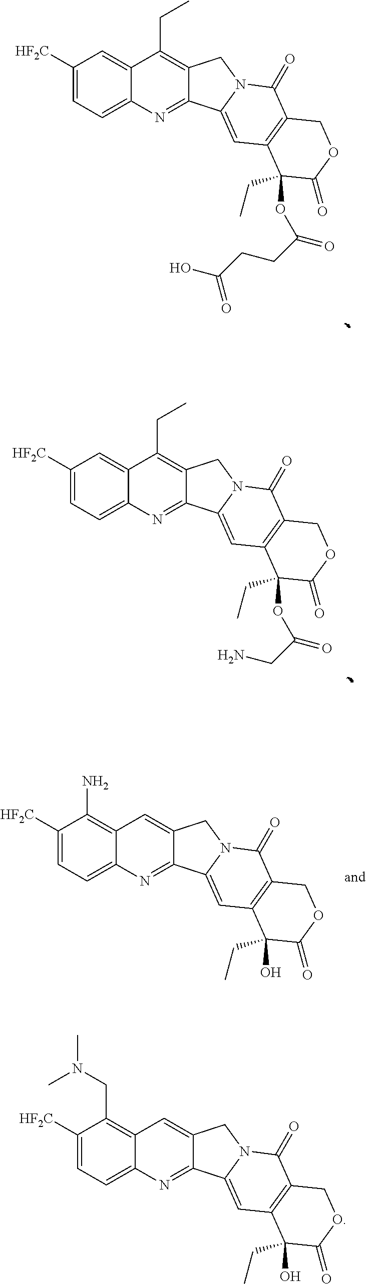

In some embodiments, the camptothecin and the derivative thereof are selected from the group consisting of: camptothecin, exatecan, topotecan, irinotecan, belotecan, lurtotecan GG-211, CKD-602, gimatecan ST1481, karenitecin BNP-1350, BN-80915, hydroxycamptothecin HCPT, 9-aminocamptothecin, 9-nitrocamptothecin, 7-ethyl-10-hydroxycamptothecin SN38, 7-ethyl-10-difluoromethylcamptothecin and their derivatives.

For example, the ligand-drug conjugate may be selected from the following structures:

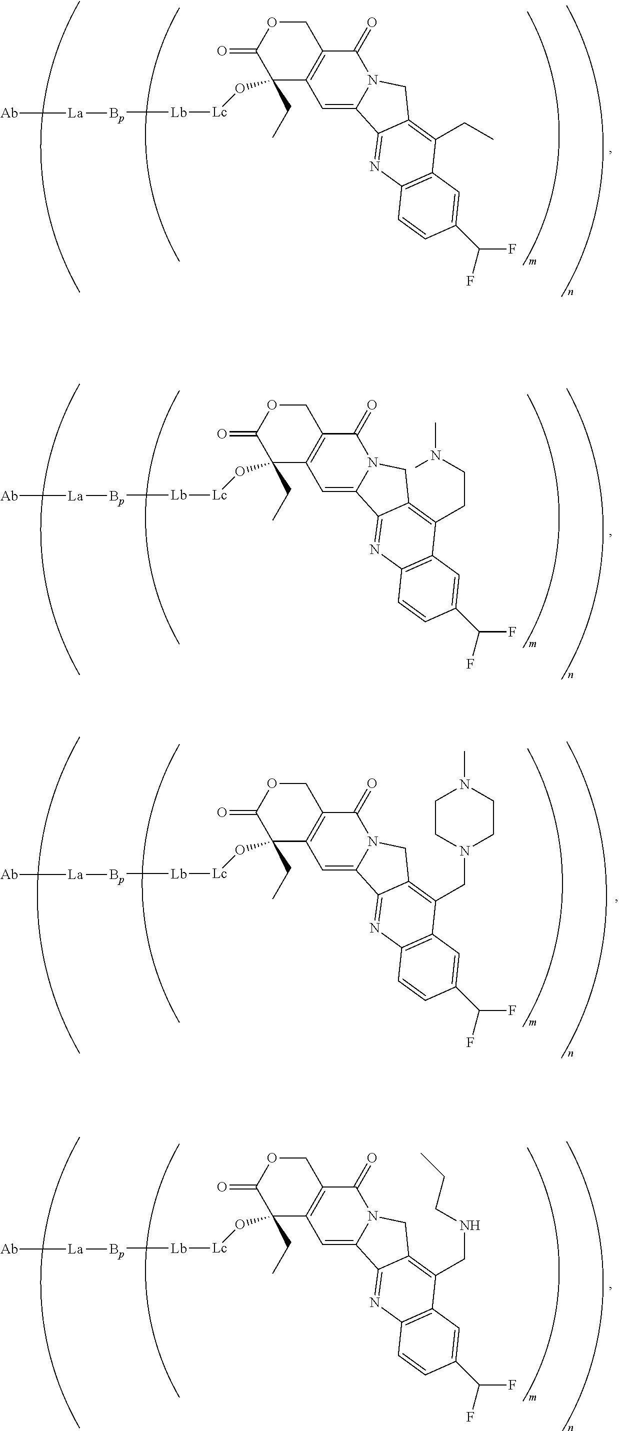

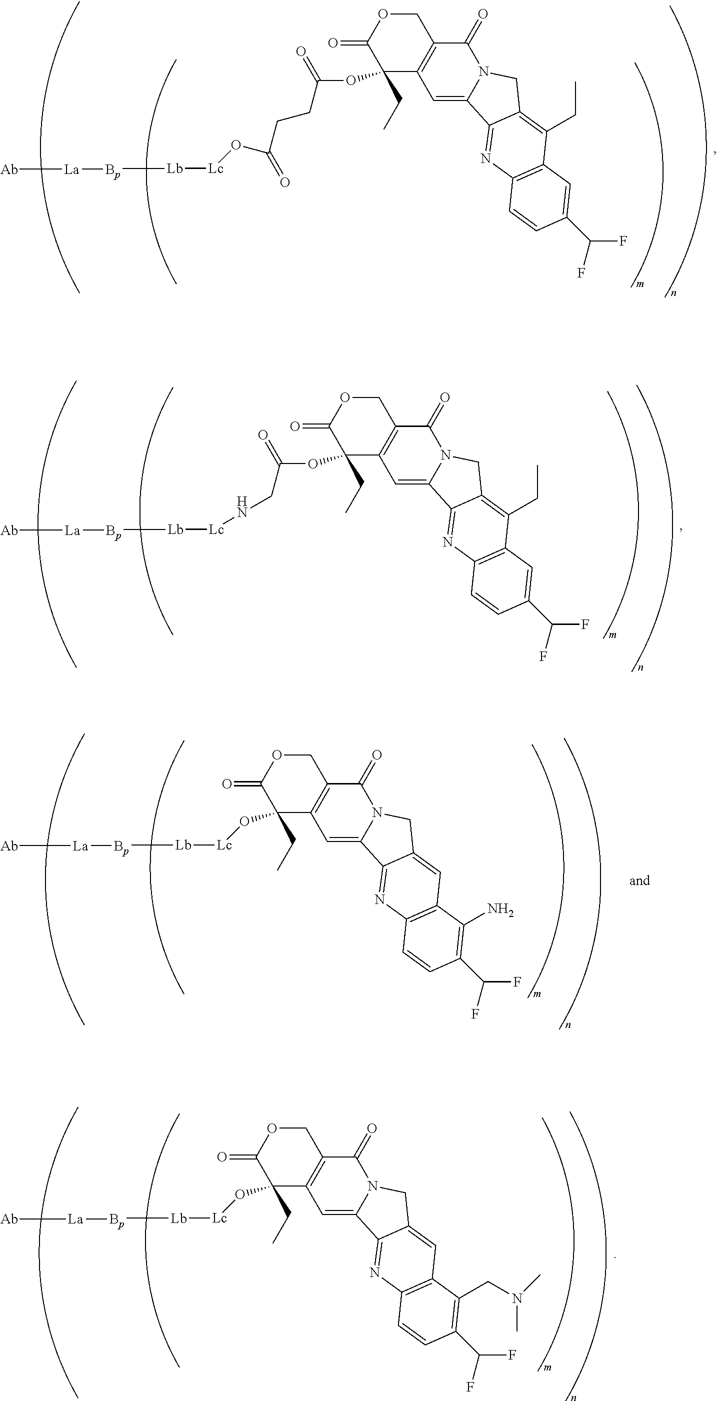

or a pharmaceutically acceptable salt thereof, wherein,
Ab represents an antibody, and a subscript n is any integer selected from 1-8, and preferably 4-8;
La is an extension unit;
B is an optional branching unit, a subscript p is 0 or 1, and wherein m is 1 when p is 0, and m is 2, 3, or 4 when p is 1; preferably, m is 2 when p is 1;
the Lb structure is selected from the following formula:

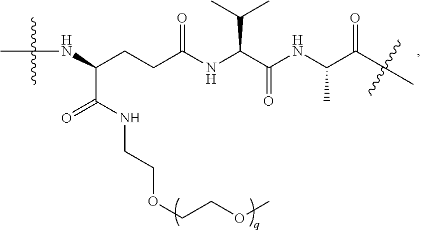

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

For example, the ligand-drug conjugate may be selected from the following structures:

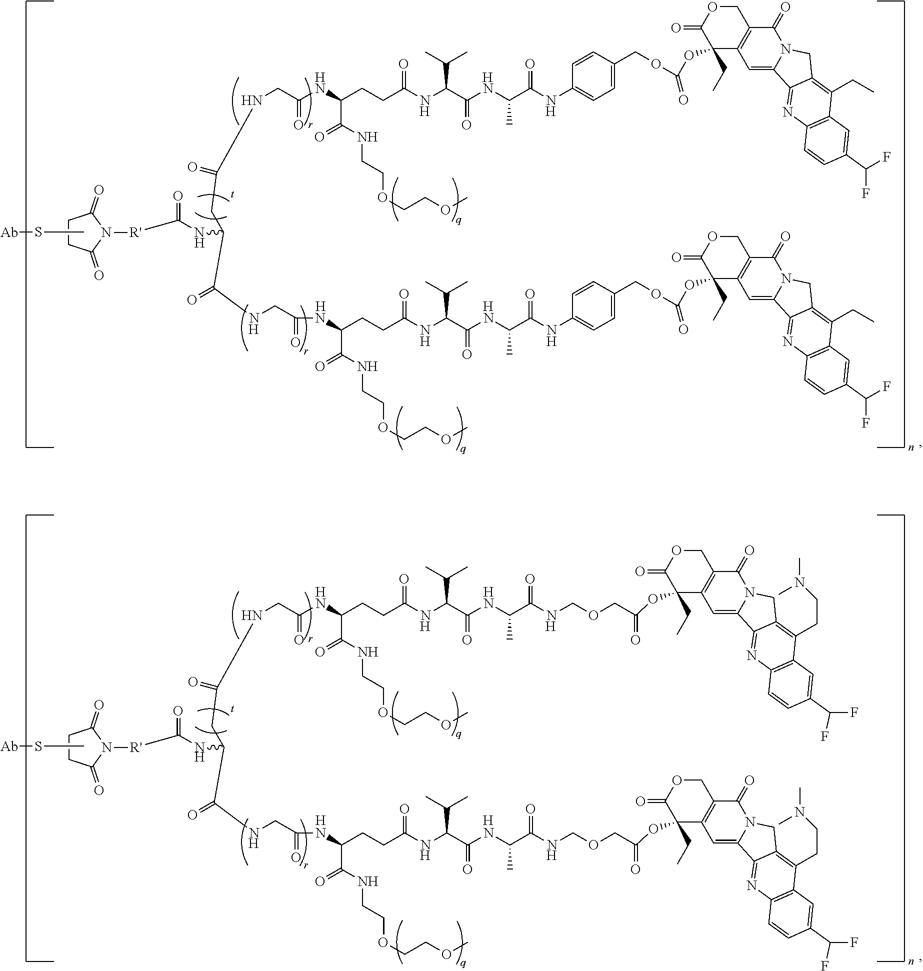

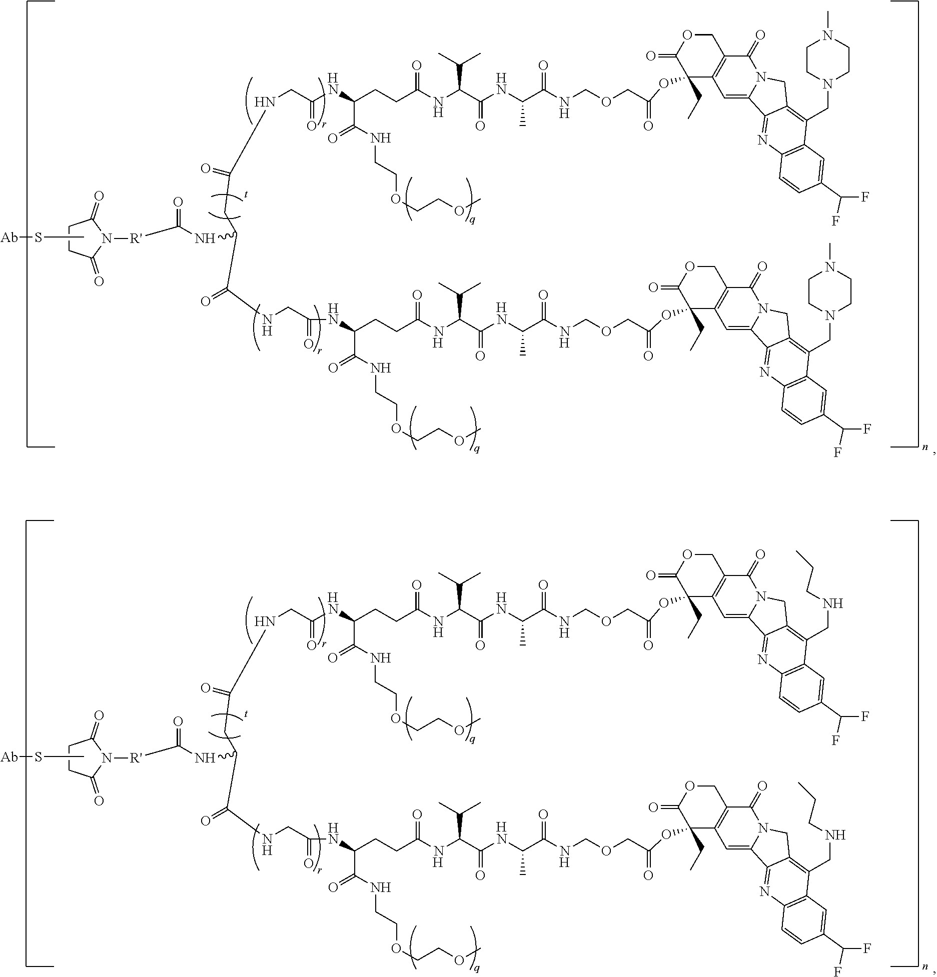

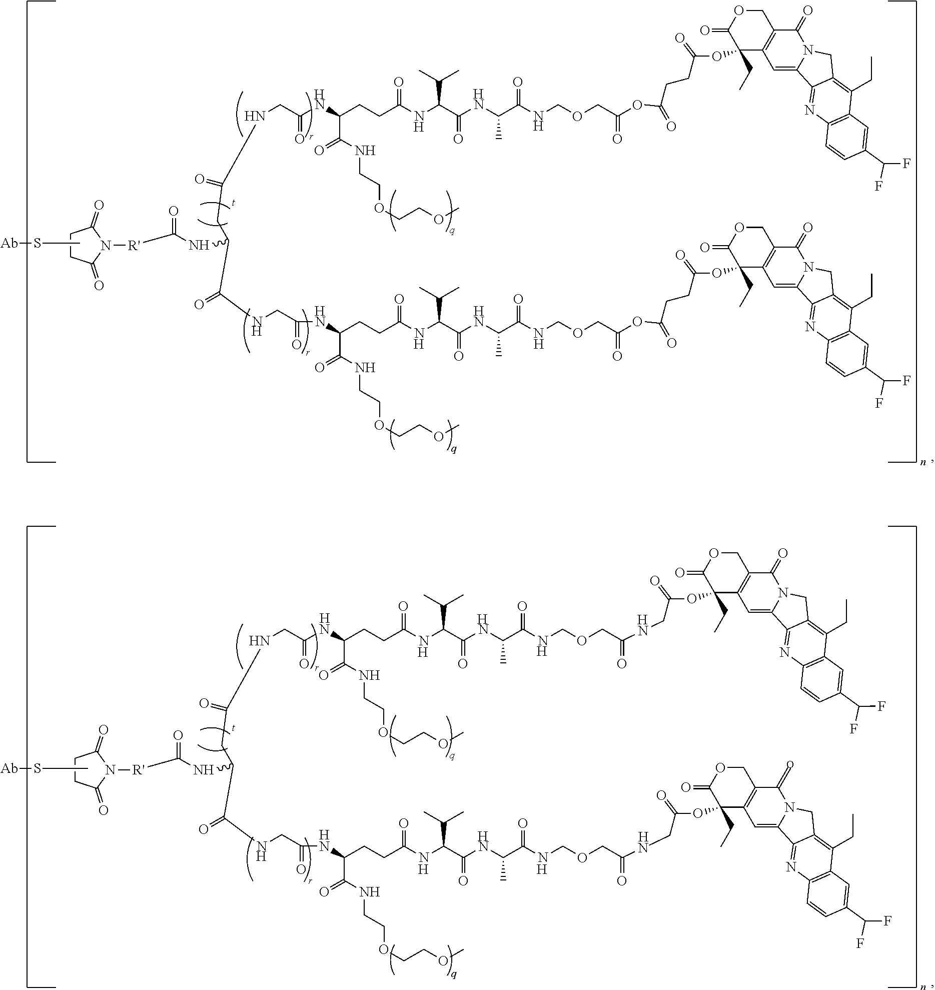

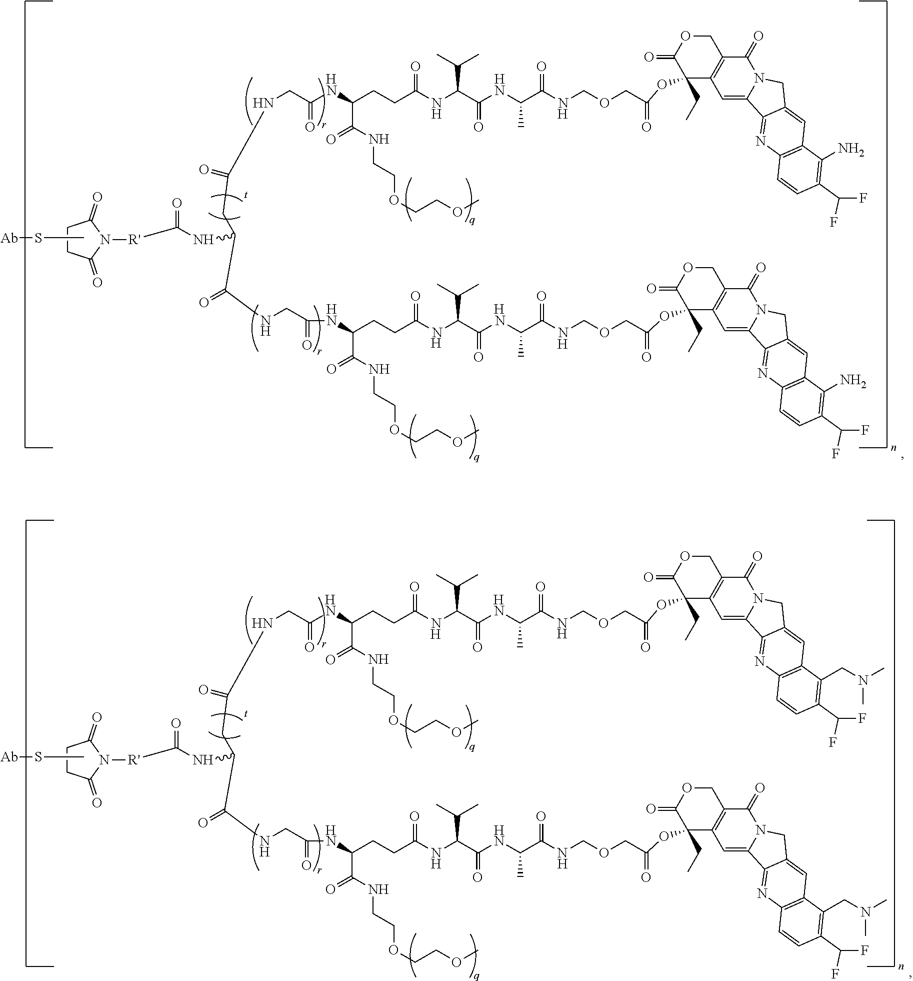

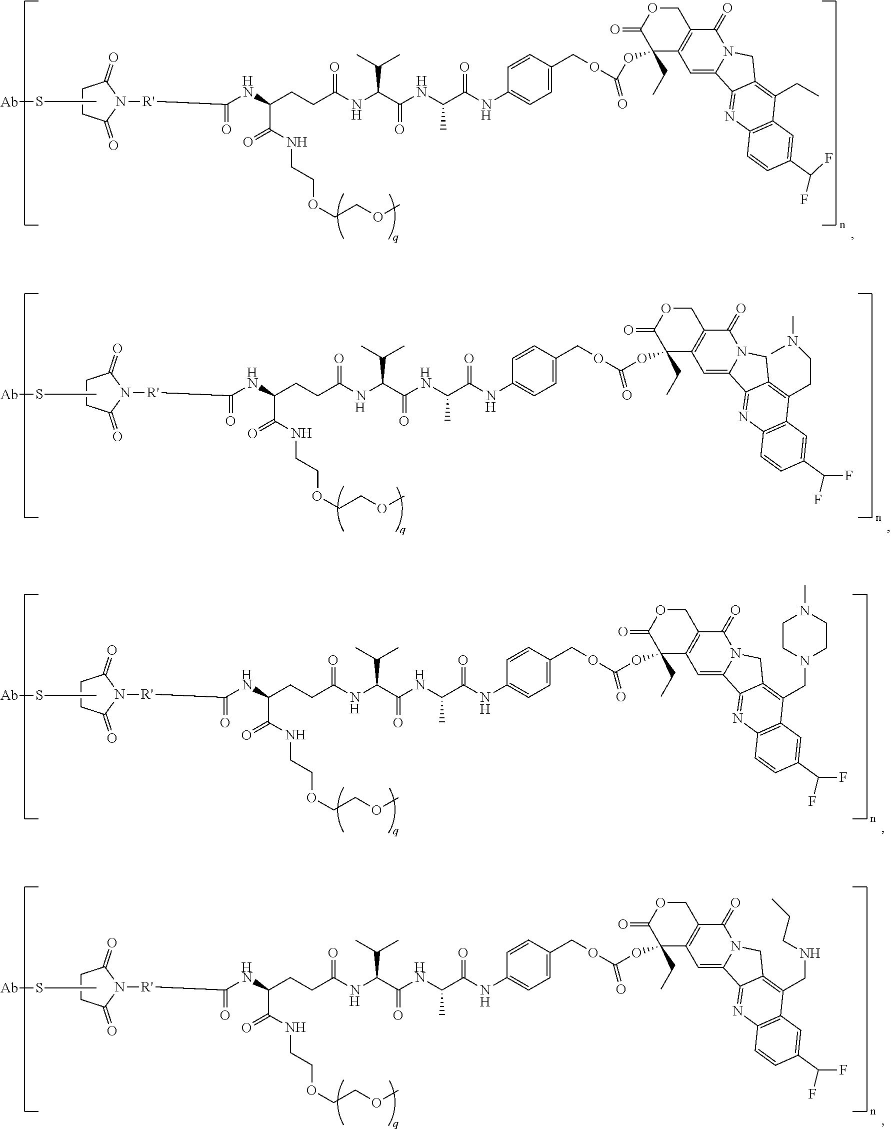

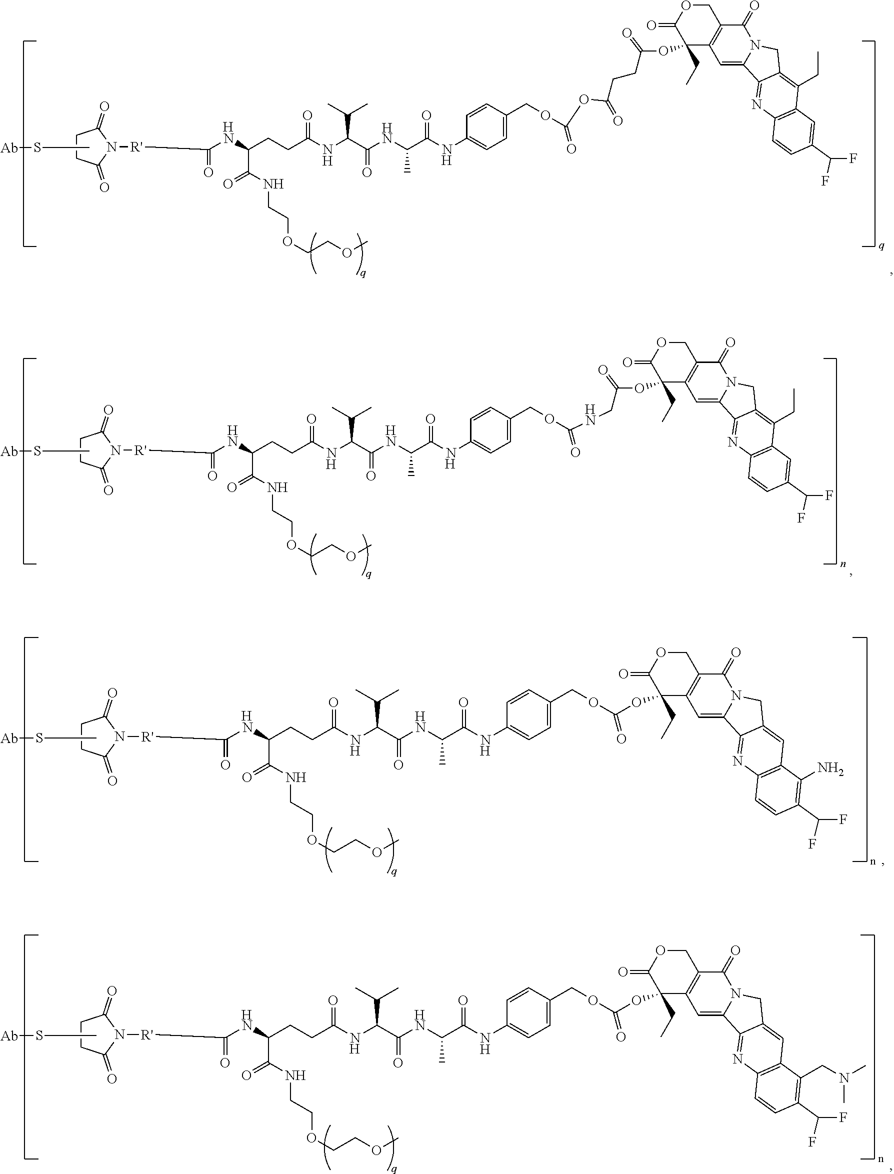

or a pharmaceutically acceptable salt thereof, wherein,
Ab represents an antibody,
R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl;
each subscript r is any integer independently selected from 1-4,
each subscript t is any integer independently selected from 1-4, and
each subscript q is any integer independently selected from 1-20, preferably 3-10, and most preferably 5-8;
each subscript n is any integer selected from 1-8, and preferably 4-8;

In some embodiments, the camptothecin and the derivative thereof are selected from the group consisting of camptothecin, exatecan, 7-ethyl-10-hydroxycamptothecin, 7-ethyl-10-difluoromethylcamptothecin, and their solvates, hydrates, stereoisomers, tautomers, isotope labels, metabolites, prodrugs, and pharmaceutically acceptable salts.

In some embodiments, the ligand-drug conjugate has a structure shown in Formula (Xa), (Xb), (Xc) or (Xd):

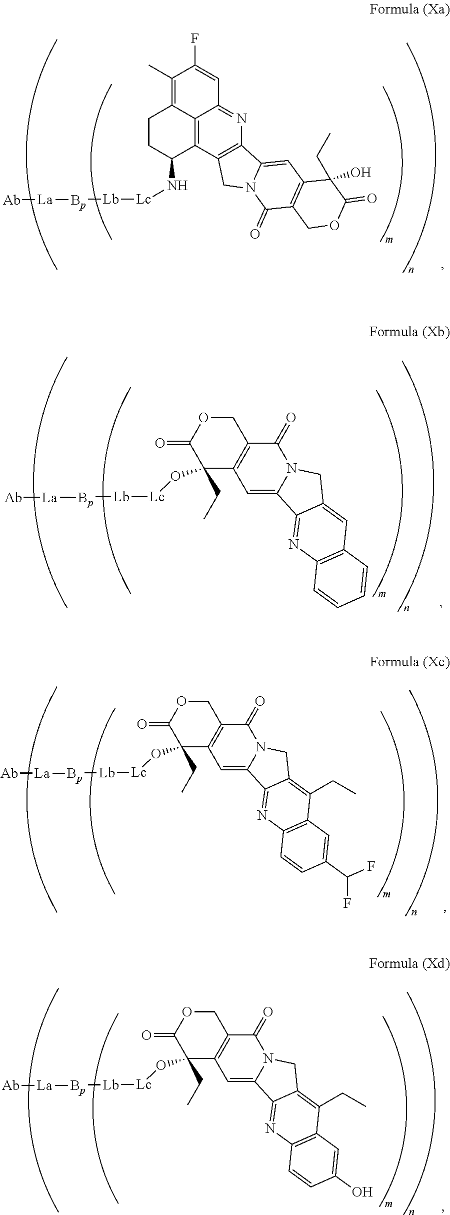

or a pharmaceutically acceptable salt thereof, wherein,
Ab represents an antibody, and a subscript n is any integer selected from 1-8, and preferably 4-8;
La is an extension unit;
B is an optional branching unit, a subscript p is 0 or 1, and wherein m is I when p is 0, and m is 2, 3, or 4 when p is 1;
the Lb structure is selected from the following formula:

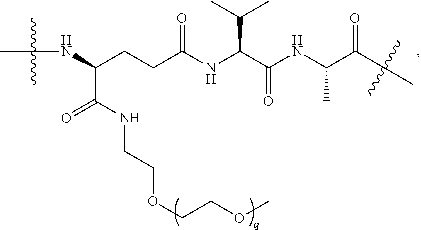

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

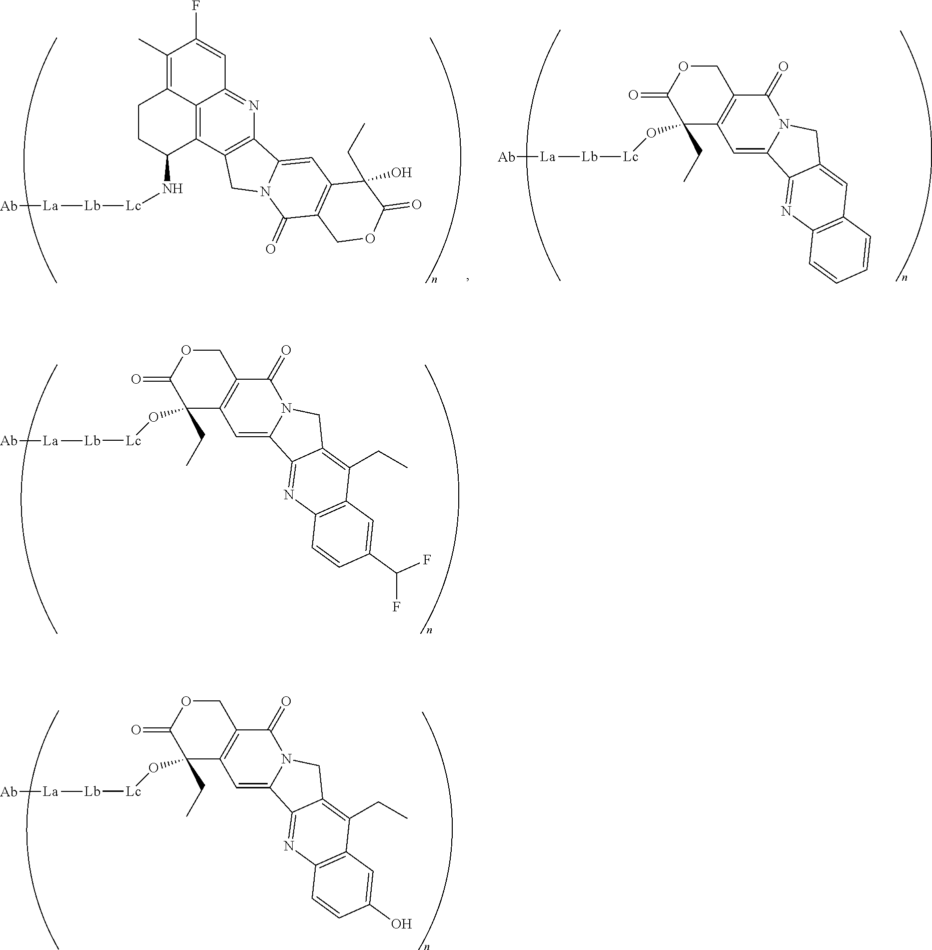

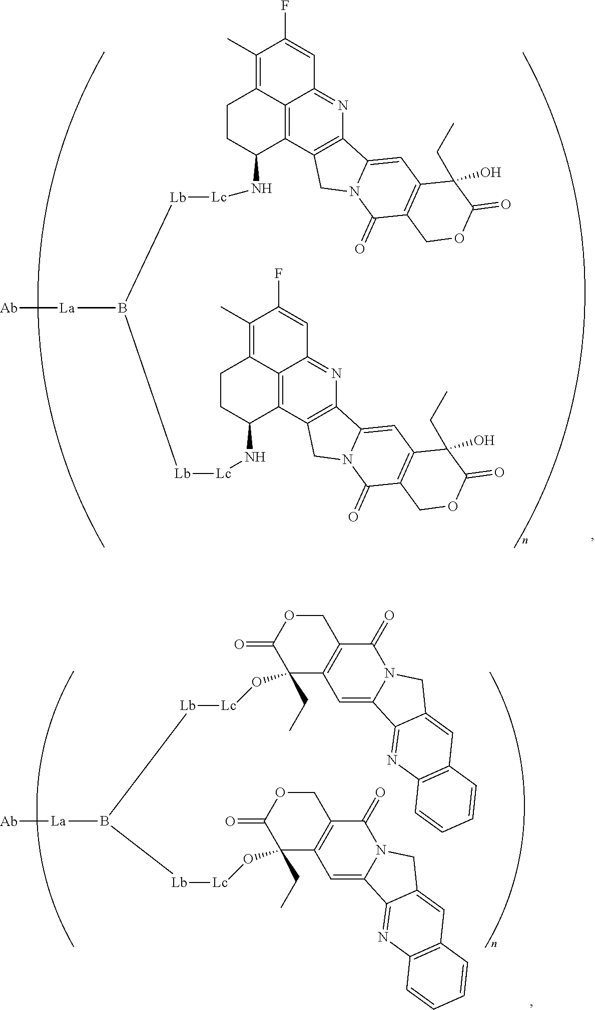

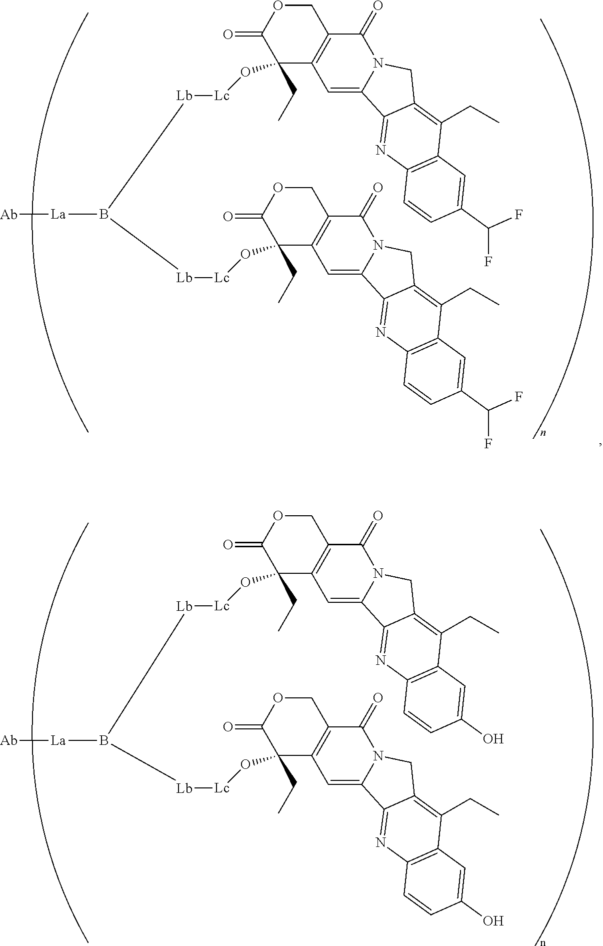

or a pharmaceutically acceptable salt thereof, wherein,
Ab represents an antibody, and a subscript n is any integer selected from 1-8, and preferably 4-8;
La is an extension unit;
B is a branching unit having a structure selected from the following formula:

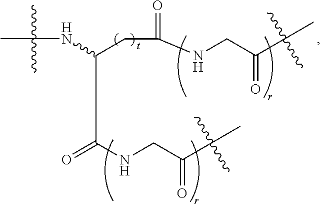

wherein a subscript t is any integer independently selected from 1-4, each subscript r is any integer independently selected from 1-4, and the B structure is linked to the La structure via amino and to the Lb structure fragment via carbonyl;
the Lb structure is selected from the following formula:

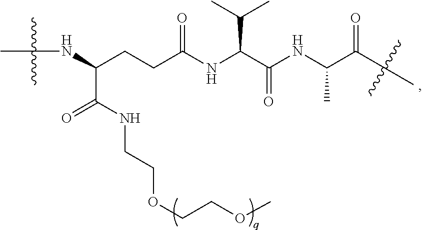

wherein a subscript q is any integer selected from 1-20, preferably 3-10, and most preferably 5-8, and the Lb structure is linked to an La or B structure via amino and to the Lc fragment via carbonyl; and
Lc is a releasable assembly unit.

In some embodiments, the ligand-drug conjugate is selected from the following structure:

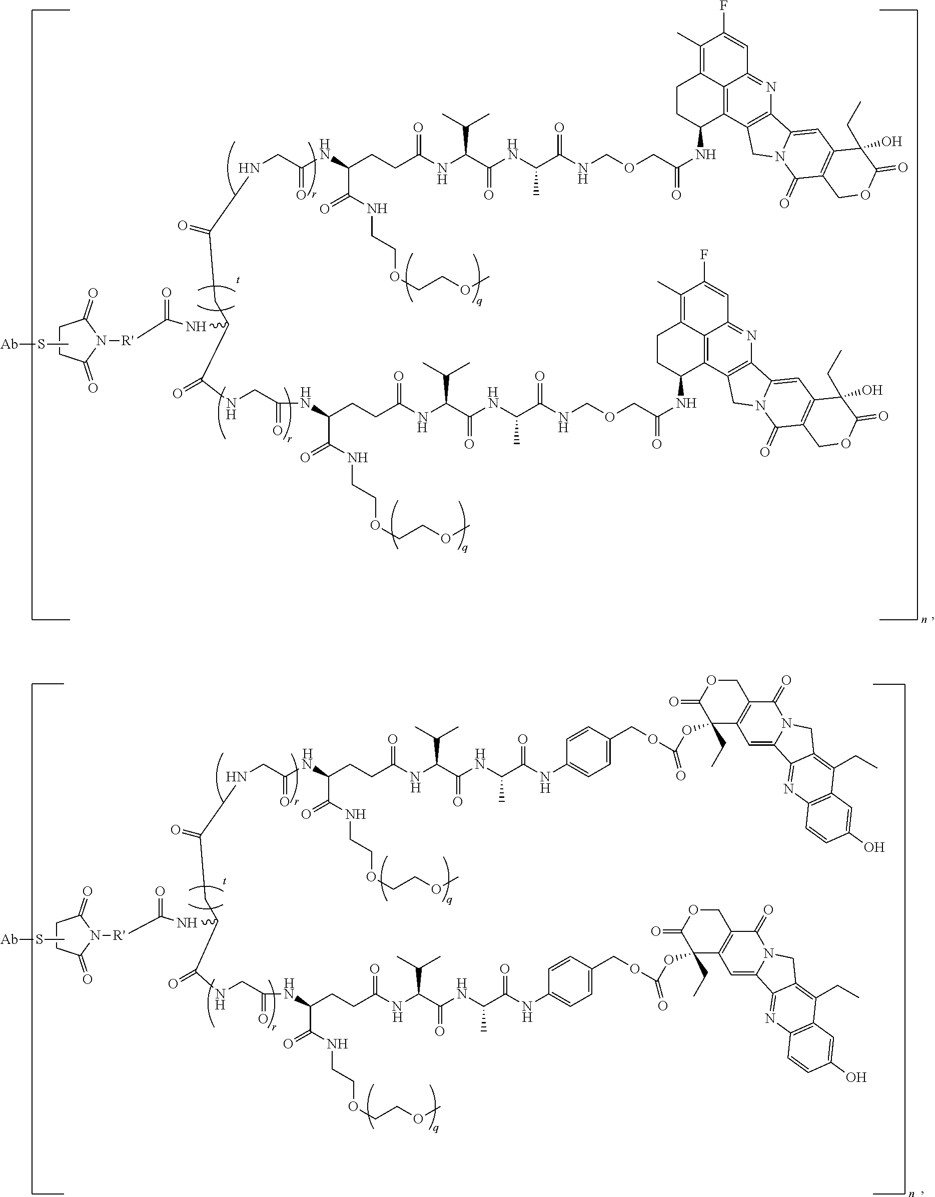

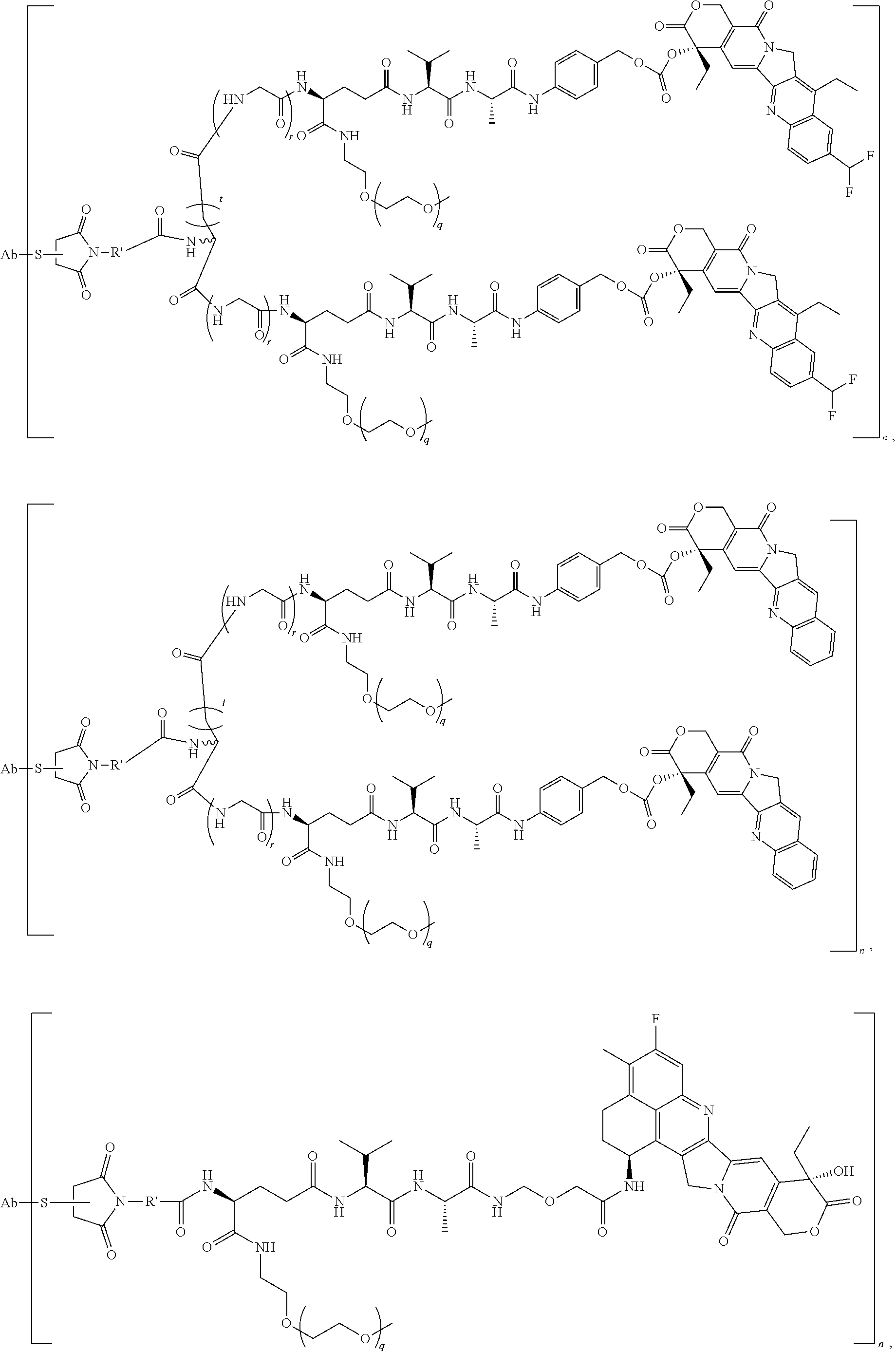

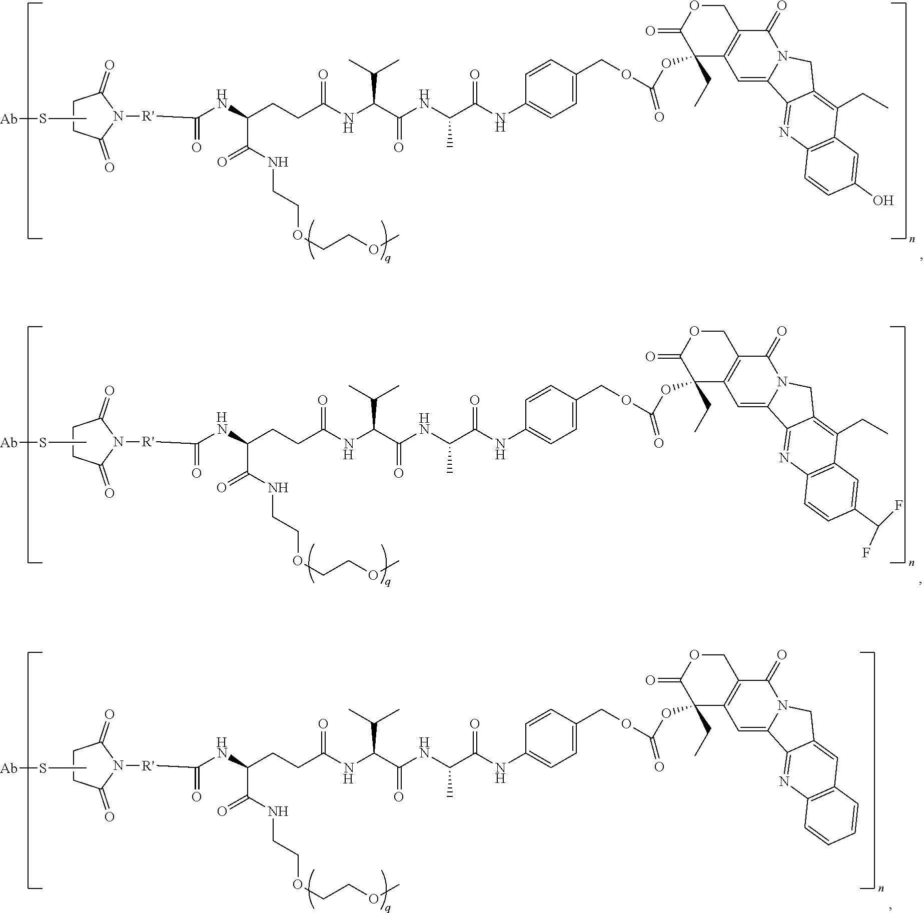

or a pharmaceutically acceptable salt thereof, wherein,
Ab represents an antibody,
R′ is selected from the group consisting of: optionally substituted C₁-C₁₀ alkyl, C₁-C₁₀ alkoxy, C₁-C₁₀ aminoalkyl and C₁-C₁₀ alkyl-aryl, wherein a substituent is selected from the group consisting of: amino, halogen, nitro, hydroxyl, acetyl, cyano, C₁-C₁₀ alkyl, C₁-C₁₀ alkoxy, C₁-C₁₀ aminoalkyl, C₁-C₁₀ haloalkyl, C₂-C₁₀ vinyl, C₂-C₁₀ alkynyl, amide group, C₃-C₈ cycloalkyl, and C₃-C₈ heterocycloalkyl;
each subscript r is any integer independently selected from 1-4,
each subscript t is any integer independently selected from 1-4, and
each subscript q is any integer independently selected from 1-20, preferably 3-10, and most preferably 5-8;
each subscript n is any integer selected from 1-8, and preferably 4-8;

In some embodiments, the ligand-drug conjugate is selected from the following structure:

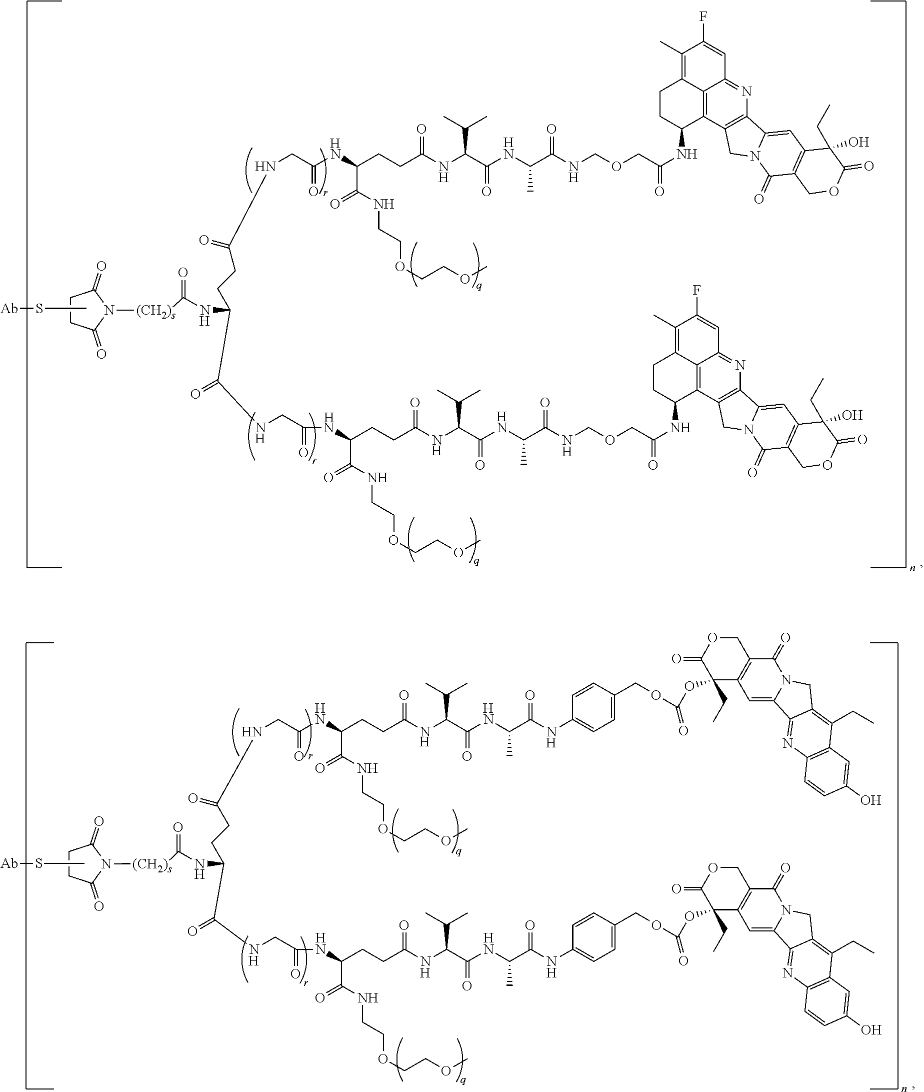

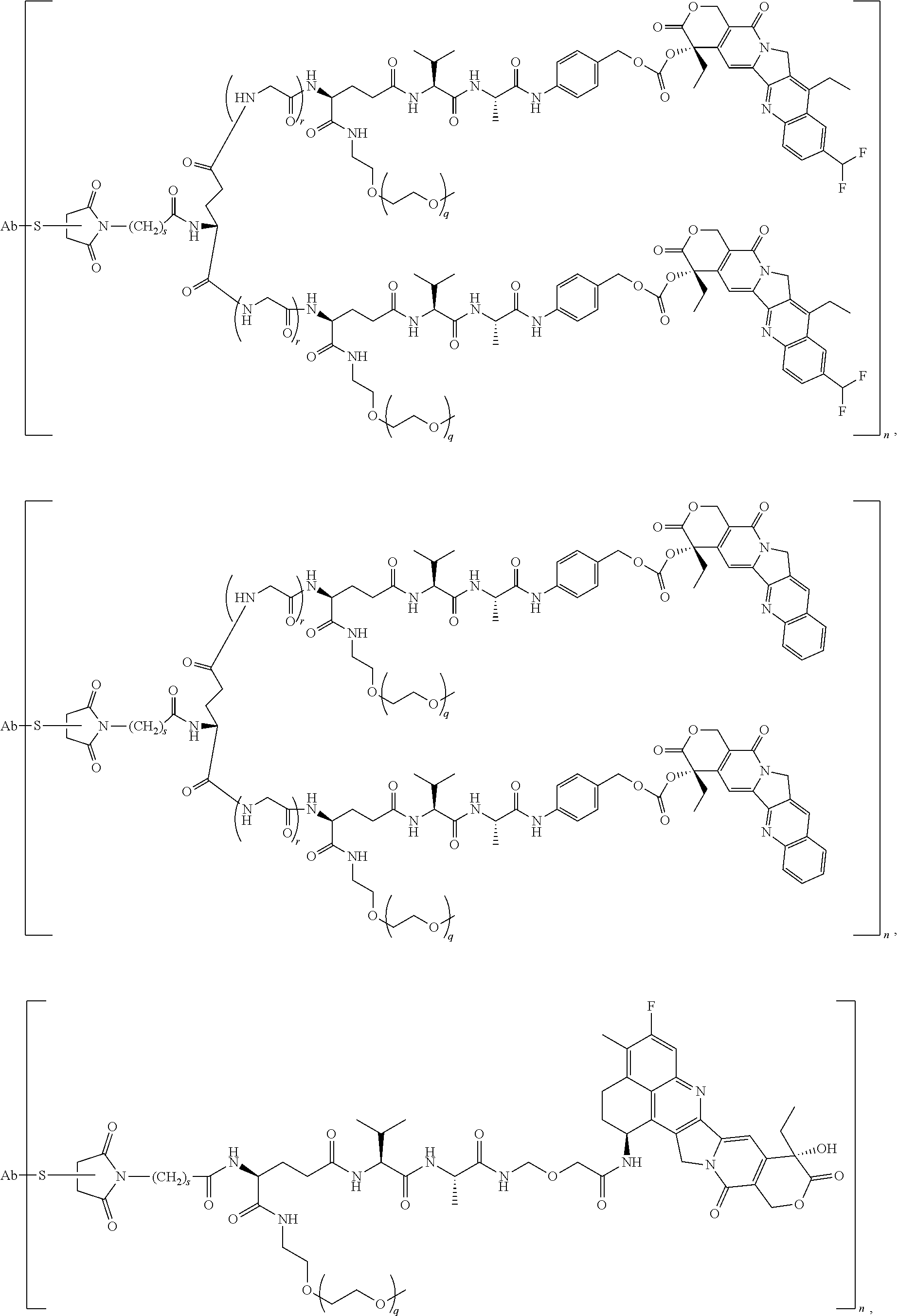

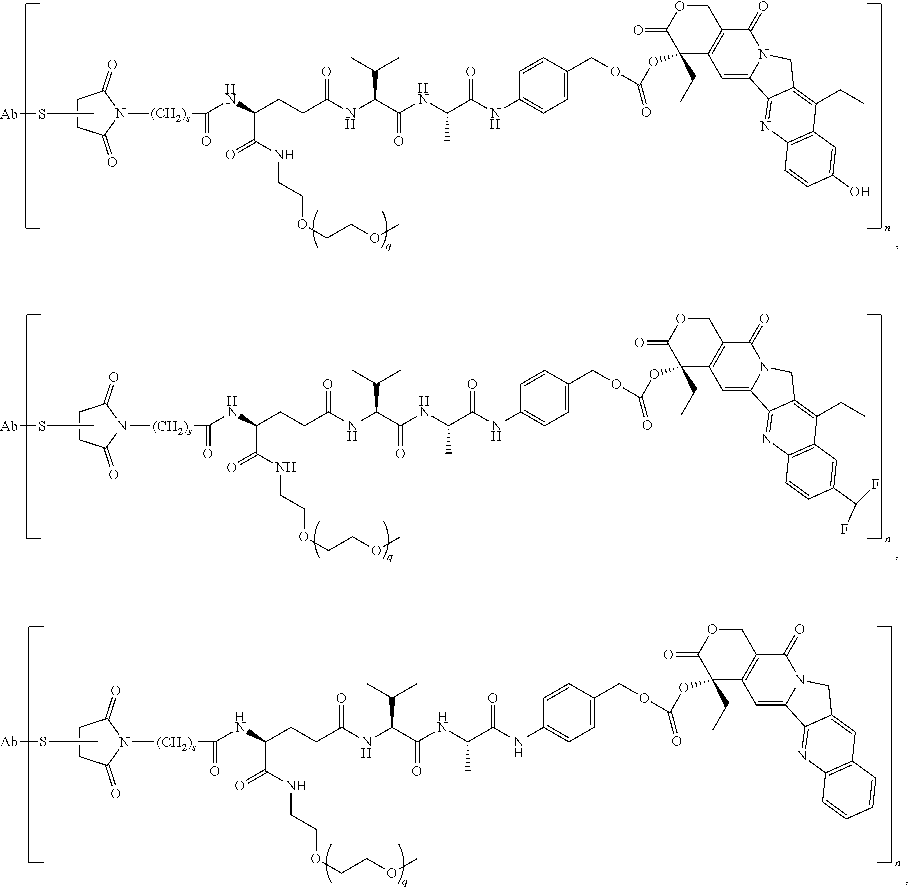

or a pharmaceutically acceptable salt thereof, wherein,
Ab represents an antibody;
each subscript s is any integer independently selected from 1-10, and preferably 1-5;
each subscript q is any integer selected from 1-20, preferably 3-10, and most preferably 5-8;
each subscript r is any integer selected from 1-4; and
each subscript n is any integer selected from 1-8, and preferably 4-8;

In some embodiments, the subscript s is 2.

In some embodiments, the subscript r is 2.

In some embodiments, the subscript q is 7.

In some embodiments, the La structure is linked to an antibody via a position 3 of succinimide.

In some embodiments, the ligand comprises a monoclonal antibody mAb or an antigen-binding fragment thereof.

In some embodiments, the ligand-drug conjugate is selected from the following structure:

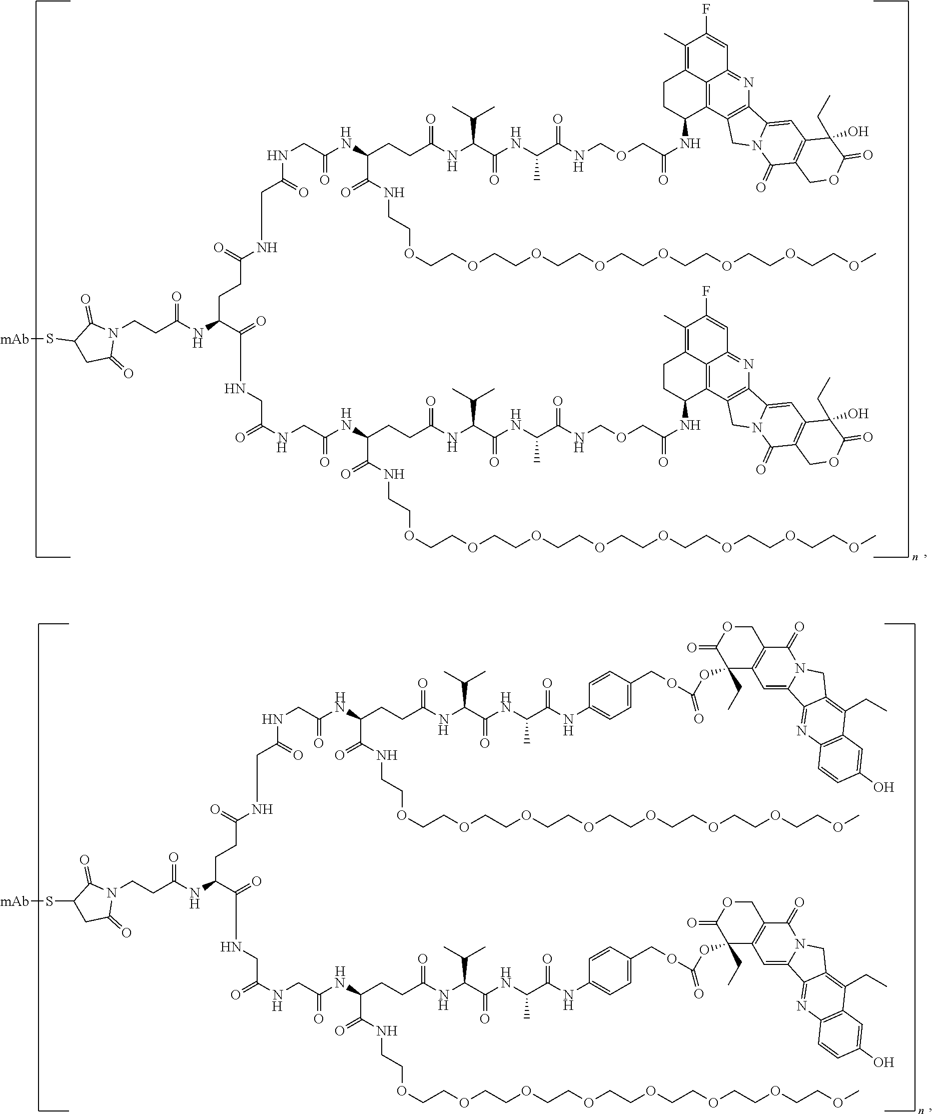

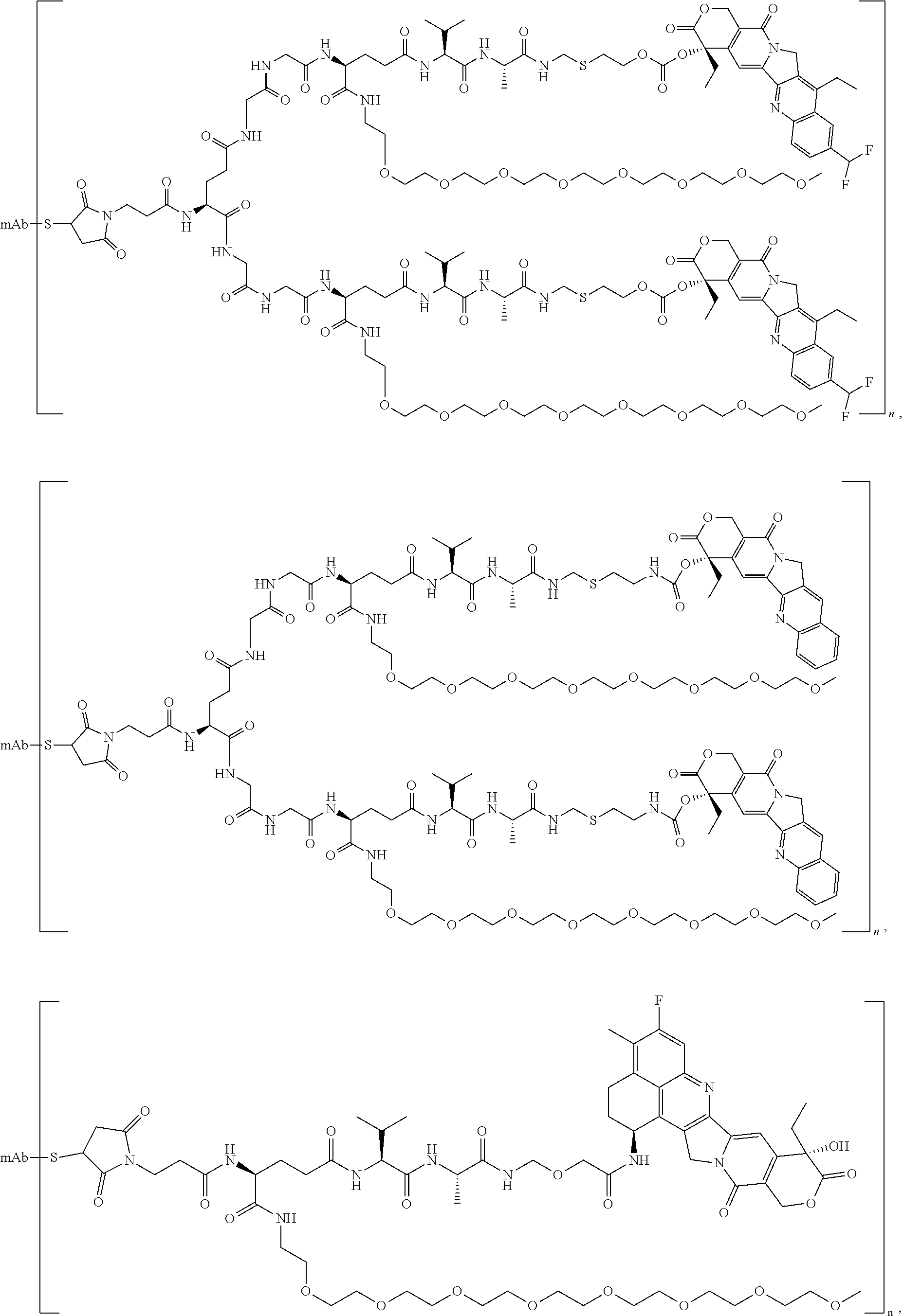

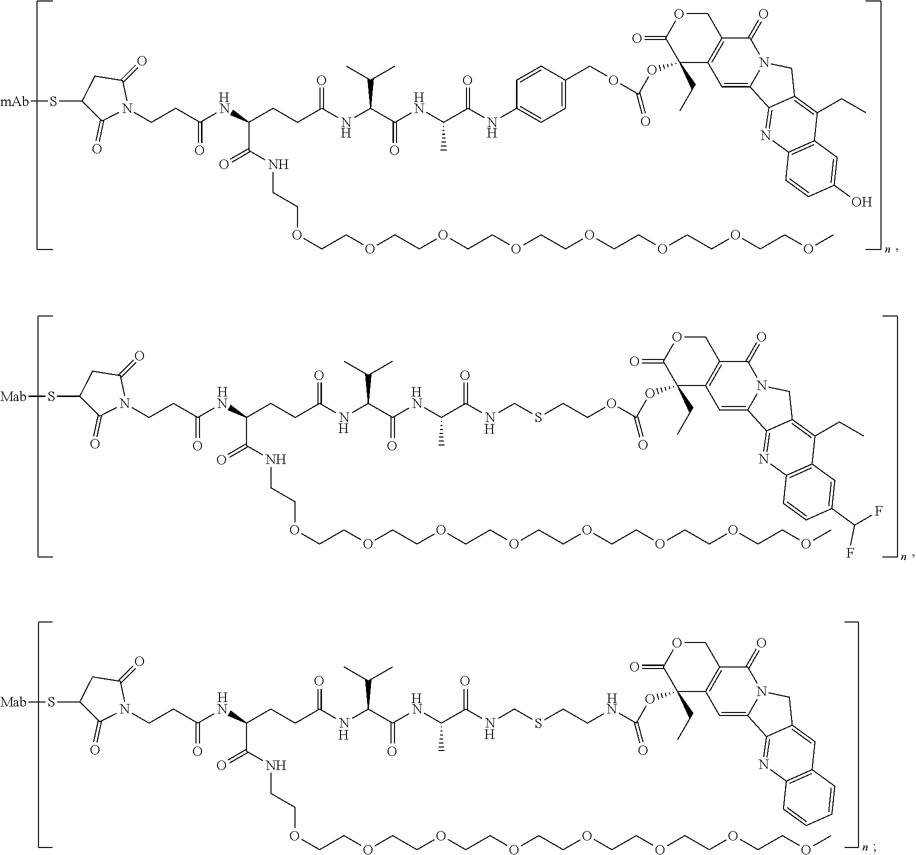

or a pharmaceutically acceptable salt thereof, wherein mAb represent a monoclonal antibody.

In some embodiments, the ligand targets and binds to a tumor antigen.

In some embodiments, in the ligand-drug conjugate, the ligand targets and binds to a receptor tyrosine-protein kinase ERBB2 (HER2), an epidermal growth factor receptor (EGFR), TROP2 and/or FGFR2b.

In some embodiments, the ligand comprises an anti-HER2 antibody, an anti-EGFR antibody, an anti-TROP2 antibody and an anti-FGFR2b antibody.

In some embodiments, the anti-HER2 antibody comprises trastuzumab, pertuzumab or inetetamab.

In some embodiments, the anti-TROP2 antibody comprises sacituzumab.

In some embodiments, the anti-FGFR2b antibody comprises bemarituzumab.

In some embodiments, the anti-EGFR antibody comprises cetuximab, panitumumab, necitumumab or nimotuzumab.

### Use

In another aspect, the present application provides use of the previously defined linker-drug conjugate or the previously defined ligand-drug conjugate in preparation of a drug for treatment of diseases with overexpressed tumor antigens.

In the present application, the term “tumor antigen” includes the meaning known in the art, including any molecule expressed on a tumor cell (or associated with tumor cell development), and known or recognized as having an effect on the tumorigenic properties of tumor cells. Many tumor antigens are known in the art. Whether a molecule is a tumor antigen may also be determined according to techniques and assays familiar to those skilled in the art, for example, clonogenic assay, transformation assay, in vitro or in vivo tumor formation assay, gel migration assay, gene knockout analysis or the like. Without limitation, the tumor antigen may include molecules such as EGFR, HER2/neu, HER3, HER4, TROP2, FGFR2b, EpCAM, CEA, TRAIL, a TRAIL receptor 1, a TRAIL receptor 2, a lymphotoxin β receptor, CCR4, CD19, CD20, CD22, CD28, CD33, CD40, CD80, CSF-1R, CTLA-4, a fibroblast activating protein (FAP), hepsin, melanoma-associated chondroitin sulfate proteoglycan (MCSP), a prostate-specific membrane antigen (PSMA), a VEGF receptor 1, a VEGF receptor 2, IGF1-R, TSLP-R, TIE-1, TIE-2, TNF-α, a TNF-like apoptosis weak inducer (TWEAK), or IL-1R.

In some embodiments, the tumor antigens comprise HER2, EGFR, TROP2 and/or FGFR2b.

In some embodiments, the diseases with overexpressed tumor antigens comprise tumors.

In some embodiments, the diseases with overexpressed tumor antigens comprise tumors expressing HER2, EGFR, TROP2 and/or FGFR2b. For example, the diseases with overexpressed tumor antigens may include HER2-positive tumors. For another example, the diseases with overexpressed tumor antigens may include TROP2-positive tumors. For another example, the diseases with overexpressed tumor antigens may include FGFR2b positive tumor.

In another aspect, the present application provides use of the previously defined linker-drug conjugate or the previously defined ligand-drug conjugate as a DNase topoisomerase I inhibitor.

In another aspect, the present application provides use of the previously defined linker-drug conjugate or the previously defined ligand-drug conjugate in preparation of a drug for cancers.

The ligand-drug conjugate (for example, antibody-camptothecin conjugate) of the present application may be used to inhibit the proliferation of tumor cells or cancer cells, induce apoptosis of tumor or cancer cells, or treat cancer in a patient. The ligand-drug conjugate is correspondingly for use in a variety of circumstances to treat cancer. The ligand-drug conjugate is intended to be used to deliver a drug to tumor cells or cancer cells. Unbound by the theory, in an embodiment, the ligand unit of the ligand-drug conjugate will bind or conjugate to cancer or tumor cell-associated antigens, and the ligand-drug conjugate is absorbed (internalized) within tumor or cancer cells by means of receptor-mediated endocytosis or other internalization mechanisms. In some embodiments, the antigens are attached to tumor or cancer cells, or may be extracellular matrix proteins associated with tumor or cancer cells. Once inside a cell, the drug is released inside the cell under the activation by an activation unit. In an alternative embodiment, a free drug is released from the ligand-drug conjugate outside a tumor or cancer cell, and subsequently penetrates the cell. In an embodiment, the ligand unit binds to the tumor or cancer cell. In another embodiment, the ligand unit binds to a tumor or cancer cell antigen on the surface of the tumor or cancer cell. In another embodiment, the ligand unit binds to a tumor or cancer cell antigen, which is an extracellular matrix protein associated with the tumor or cancer cell.

Cancers intended to be treated with the ligand-drug conjugate of the present application include, but are not limited to, hematopoietic system cancers, such as, for example, lymphoma (Hodgkin lymphoma and non-Hodgkin lymphoma) as well as leukemia and solid tumors. Examples of the cancers of the hematopoietic system include follicular lymphoma, anaplastic large cell lymphoma, mantle cell lymphoma, acute myeloblastic leukemia, chronic myeloblastic leukemia, chronic lymphocytic leukemia, diffuse large B-cell lymphoma, and multiple myeloma. Examples of the solid tumors include fibrosarcoma, myxosarcoma, liposarcoma, chondrosarcoma, osteosarcoma, chordoma, angiosarcoma, endothelial sarcoma, lymphangiosarcoma, lymphangioendothelial sarcoma, synovioma, mesothelioma, Ewing's sarcoma, leiomyosarcoma, rhabdomyosarcoma, colon cancer, colorectal cancer, kidney cancer, pancreatic cancer, bone cancer, breast cancer, ovarian cancer, prostate cancer, esophageal cancer, gastric cancer, oral cancer, nose cancer, throat cancer, squamous cell carcinoma, basal cell carcinoma, glandular cancer, sweat gland cancer, sebaceous gland cancer, papillary cancer, papillary adenocarcinoma, cystadenocarcinoma, medullary cancer, bronchial cancer, renal cell carcinoma, liver cancer, bile duct cancer, choriocarcinoma, seminoma, embryonal cancer, Wilms' tumor, cervical cancer, uterine cancer, testicular cancer, small cell lung cancer, bladder cancer, lung cancer, epithelial cancer, glioma, glioblastoma multiforme, astrocytoma, medulloblastoma, craniopharyngioma, ependymoma, pineal tumor, hemangioblastoma, acoustic neuroma, oligodendroglioma, meningiomas, neuroblastoma, and retinoblastoma.

In some embodiments, the cancers comprise cancers with upregulated expression and/or activity of HER2.

In some embodiments, the cancers comprise cancers with upregulated expression and/or activity of EGFR.

In some embodiments, the cancers comprise a lung cancer, a breast cancer, a gastric cancer, a colorectal cancer, an esophageal cancer, a salivary gland cancer, gastroesophageal junction adenocarcinoma, a biliary tract cancer, Paget's disease, a pancreatic cancer, an ovarian cancer or uterine carcinosarcoma.

In another aspect, the present application provides use of the previously defined linker-drug conjugate or the previously defined ligand-drug conjugate in treatment of cancers.

In another aspect, the present application provides a pharmaceutical composition, comprising the previously defined linker-drug conjugate or the previously defined ligand-drug conjugate, and a pharmaceutically acceptable carrier.

The present application provides a pharmaceutical composition comprising the ligand-drug conjugate described herein and at least one pharmaceutically acceptable carrier. The pharmaceutical composition exists in any form that allows the administration of the compound to a patient for the treatment of disorders associated with the expression of antigens bound to a ligand unit. For example, the conjugate is in the form of liquid or solid. The preferred route of administration is parenteral administration. The parenteral administration includes subcutaneous, intravenous, intramuscular, intrasternal injection or infusion techniques. In an embodiment, the composition is administered parenterally. In an embodiment, the conjugate is administered intravenously. The administration is carried out by any convenient route, for example, by infusion or bolus.

The pharmaceutical composition is formulated such that the ligand-drug conjugate is bioavailable after the administration of the composition to a patient. The composition is sometimes in the form of one or more dose units. Materials used in the preparation of the pharmaceutical composition are preferably non-toxic in the amount used. It is obvious for those of ordinary skills in the art that the optimal dose(s) of one or more active ingredients in the pharmaceutical composition will depend on a variety of factors. Relevant factors include, but are not limited to, the type of animal (for example, human), the particular form of the compound, the manner of administration, and the composition used. In some embodiments, the composition is in the form of liquid. In some embodiments, the liquid is used for delivery by injection. In some embodiments, in addition to the ligand-drug conjugate, the composition for administration by injection comprises one or more of the excipients selected from the group consisting of: surfactants, preservatives, humectants, dispersants, suspensions, buffers, stabilizers and isotonic agents. In some embodiments, a liquid composition, whether in the form of solution or suspension or in other similar forms, comprises one or more of the following: sterile diluents (such as water for injection), saline solutions (preferably normal saline), Ringer's solution, isotonic sodium chloride, non-volatile oils (such as synthetic monoglyceride or diglyceride) capable of acting as solvents or suspension media, polyethylene glycol, glycerin, cyclodextrin, propylene glycol or other solvents; antibacterial agents such as benzyl alcohol or methylparaben; antioxidants, such as ascorbic acid or sodium bisulfite; chelating agents, such as ethylenediaminetetraacetic acid; buffers such as amino acids, acetate, citrate or phosphate; detergents, such as nonionic surfactants, or polyols; and reagents for regulating tension, such as sodium chloride or dextrose. The parenteral composition is sometimes encapsulated in ampoules, disposable syringes or multi-dose vials made of glass, plastic or other materials. Saline is an exemplary adjuvant. An injectable composition is preferably sterile.

In another aspect, the present application provides a method for treating diseases or disorders, comprising administering an effective amount of the previously defined linker-drug conjugate, the previously defined ligand-drug conjugate, or the previously defined pharmaceutical composition to a subject in need thereof.

In some embodiments, the amount of conjugate effective in the treatment of a particular condition or disease will depend on the nature of the disease or condition and be determined by standard clinical techniques. In addition, optionally, in vitro or in vivo assays are used to help identify the optimal dose range. The precise dose to be used in the composition also depends on the route of administration and the severity of the disease or condition, and should be decided at the discretion of the practitioner and according to the situation of each patient.

In some embodiments, the diseases or disorders comprise cancers.

In another aspect, the present application provides a method for inhibiting cancer cell activity or killing cancer cells, comprising contacting cancer cells with an effective amount of the previously defined linker-drug conjugate, the previously defined ligand-drug conjugate, or the previously defined pharmaceutical composition.

In another aspect, the present application provides a drug cassette comprising the previously defined linker-drug conjugate, the previously defined ligand-drug conjugate, or the previously defined pharmaceutical composition, and optionally, an additional therapeutic agent.

Not wishing to be bound by any theory, the following examples are merely to illustrate the linker, linker-drug conjugate, ligand-drug conjugate, methods for preparing them, their uses and the like, and are not intended to limit the scope of the present invention.

### EXAMPLES

### Polymer/Lysate (SEC)

The measurement was conducted by referring to high performance liquid chromatography (Pharmacopoeia of the Peoples Republic of China 2020 Edition-General Rule 0512).

Test solution: It was used as the test solution.

Chromatographic conditions: filler: hydrophilic modified silica gel (Sepax Zenix SEC-300 (3 μm, 7.8 mm ID×300 mm), and mobile phase: 0.15 mol/L phosphate buffer (pH 7.0); flow rate: 1.0 ml per minute, and detection wavelength: 280 nm & 370 nm; column temperature: 25° C.; and injection volume: 10 μl.

Determination method: The test solution was accurately weighed out, and injected into a liquid chromatograph; and the chromatograms were recorded for 20 min.

### Drug-Antibody Conjugation Ratio (RPLC)

The measurement was conducted by referring to high performance liquid chromatography (Pharmacopoeia of the Peoples Republic of China 2020 Edition-General Rule 0512).

Test solution: It was prepared by fully mixing 60 μl of 1 mg/ml test solution and 15 μl of 100 mmol/L dithiothreitol (DTT) aqueous solution and holding the mixture in a water bath at 37° C. for 30 min.

Blank: The blank was prepared in the same way.

Chromatographic conditions: filler: octadecylsilane-bonded silica gel (PLRP-S, 1000 Å (8 μm, 4.6 mm ID×150 mm, Agilent technologies), mobile phase A: water/trifluoroacetic acid (1000:0.5, v/v), and mobile phase B: acetonitrile/trifluoroacetic acid (1000:0.4, v/v), with gradient elution conducted according to the table below; flow rate: 1.0 ml per minute; detection wavelength: 280 nm; column temperature: 80° C.; and injection volume: 10 μl.

| Time (min) | Mobile phase A (%) | Mobile phase B (%) |
|---|---|---|
| 0.01 | 70 | 30 |
| 24.00 | 54 | 46 |
| 25.00 | 70 | 30 |
| 35.00 | 70 | 30 |

Determination method: The test solution was accurately weighed out, and injected into a liquid chromatograph; and the chromatograms were recorded.

### Example 1: Synthesis of La—B Fragment

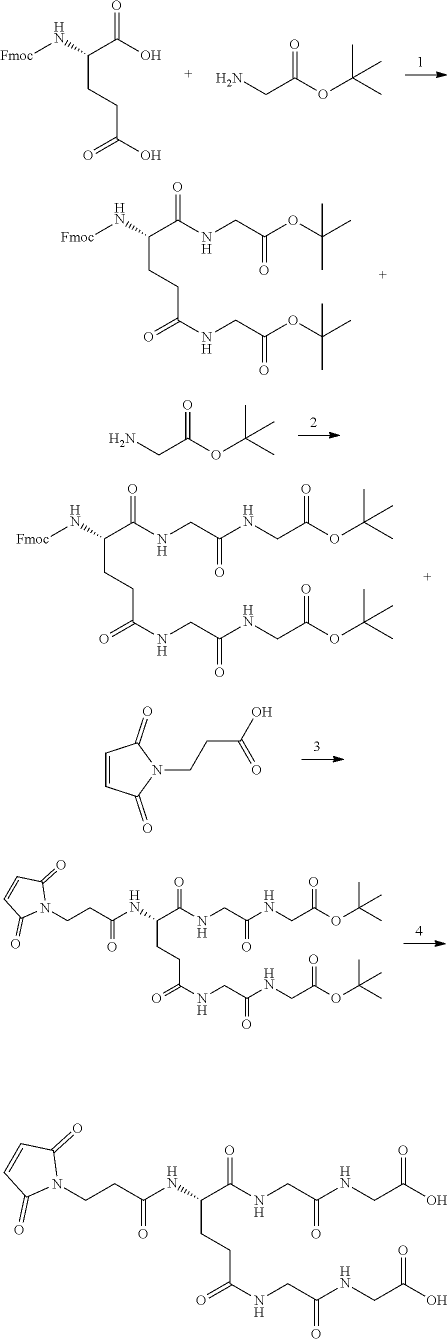

Reaction 1: 7.4 g of Fmoc-L-glutamic acid and 5.8 g of tert-butyl glycinate as well as 10 g of DIPEA, as reactants, were dissolved in a dichloromethane/DMF (150 mL/40 mL) mixture; 18 g of HATU was added; the resulting mixture was stirred at room temperature to allow reaction for 1 h; 150 mL of water was added to the reaction solution and stirred; and after stratification, an organic layer was washed with water, dried and evaporated to dryness to obtain a crude product. MS[ESI] m/z: 596.7[M+H]⁺.

Reaction 2: The above product was dissolved in 100 mL of dichloromethane; 30 mL of trifluoroacetic acid was added to allow reaction for 4 h at room temperature; 150 ml of water was added; and precipitated solids were filtered, washed with water, washed with ethyl acetate, and then dried to obtain 7.5 g of diacid. 8.9 g of diacid, 5.3 g of tert-butyl glycinate and 9.5 g of DIPEA were dissolved in the mixture of dichloromethane/DMF (180 mL/45 mL); 16.8 g of HATU was added; the resulting mixture was stirred at room temperature to allow reaction for 90 min; and 150 mL of water was added to the reaction solution; an organic layer was washed with water and dried, and then purified by column chromatography to obtain 11 g of product. MS[ESI] m/z: 710.6[M+H]+.

Reaction 3: 5 g of the above product was dissolved in 50 mL of DMF; 5 mL of morpholine was added; the resulting mixture was stirred at room temperature to allow reaction for 80 min; the reaction solution was evaporated to dryness under reduced pressure; the resulting product, together with 1.2 g of maleimidopropionic acid, was dissolved in the mixture of dichloromethane/DMF (50 mL/12.5 mL); 3.2 g of HATU was added to allow reaction for 80 min at room temperature; 50 mL of water was added to the reaction solution to quench the reaction; the mixture was extracted 3 times with 50 mL of dichloromethane; and organic phases were combined, washed with 10% sodium bicarbonate aqueous solution, water, 5% hydrochloric acid, and water in sequence, dried and evaporated to dryness, and pulped using ethyl acetate to obtain solids, which were dried to obtain 2.5 g of product. MS[ESI] m/z: 639.5[M+H]+.

Reaction 4: 3 g of the above product was dissolved in 30 mL of dried dichloromethane; 15 mL of trifluoroacetic acid was added to allow reaction for 2.5 h at room temperature; 1 mL of water was added to quench the reaction; the reaction solution was evaporated to dryness under reduced pressure to obtain a product; and the product was pulped for 2-3 h using 100 mL of ethyl acetate, and filtered to obtain solids, which were washed with ethyl acetate and dried to obtain 2.3 g of product. MS[ESI] m/z: 525.4[M−H]−, 1HNMR (400 MHZ, DMSO-d6): δ=13.78 (2H), 12.34 (1H), 8.77 (2H), 8.55 (2H), 7.51 (2H), 7.02 (4H), 3.62 (5H), 2.50 (8H).

### Example 2: Synthesis of Lb Fragment

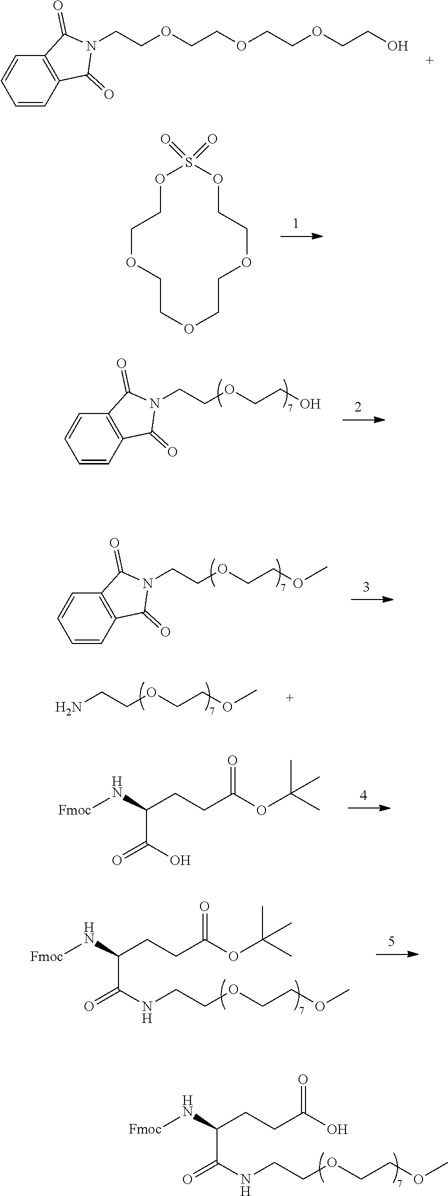

Reaction 1: 7.4 g of 60% NaH was added to 200 mL of anhydrous tetrahydrofuran, cooled to 0° C. over stirring, and stirred for 30 min; 46 g of a reactant phthalimide-substituted tetraethylene glycol was dissolved in 200 mL of tetrahydrofuran and added dropwise to the NaH mixture; after addition, the resulting mixture was heated naturally and stirred to react for 90 min; 34.46 g of a reactant cyclic disulfonate ester was dissolved in 200 mL of tetrahydrofuran and slowly added dropwise to the above reaction solution; after addition, the resulting mixture was stirred to react for 15 h; 18 mL of water and 25 mL of concentrated sulfuric acid were added to the reaction solution; the mixture was refluxed to react for 60 min, and cooled to room temperature; sodium bicarbonate was added to adjust the pH to 7-8; the mixture was evaporated to dryness under reduced pressure to obtain a product, which was then extracted with dichloromethane and separated by column chromatography to obtain 50 g of product. MS[ESI] m/z: 500.6[M+H]+.

Reaction 2: 4.9 g of 60% NaH was dispersed into 200 mL of anhydrous tetrahydrofuran and stirred for 10 min. 50 g of the above product was dissolved in 100 mL of anhydrous tetrahydrofuran, added dropwise to NaH, and stirred for 20 min at room temperature. 16.8 g of dimethyl sulfate was dissolved in anhydrous tetrahydrofuran, slowly add dropwise to the above reaction solution, and stirred at room temperature to react for 15 h; 50 mL of water was added to terminate the reaction; the reaction solution was evaporated to dryness; dichloromethane/water was added for stratification; the organic phase was washed with water, dried and evaporated to dryness, and purified column chromatography to obtain 35 g of product. MS[ESI] m/z: 514.6[M+H]+.

Reaction 3: 50 g of the above product was dissolved in 500 mL of methanol; 7 mL of hydrazine hydrate was added dropwise to react for 2 h at 70° C.; the reaction solution was cooled down and evaporated to dryness; 1 N dilute hydrochloric acid was added and stirred for 30 min; the resulting mixture was filtered by diatomite and washed with water; the aqueous phase was adjusted to the pH of 9-10 with sodium hydroxide, and evaporated to dryness under reduced pressure; the residues were dissolved in dichloromethane and filtered; the filtrate was dried and evaporated to dryness to obtain 25.5 g of crude product, which is to be used directly in the next reaction.

Reaction 4: 25.5 g of the above product and 23.6 g of Fmoc-mono-tert-butyl glutamate as well as 14.3 g of DIPEA were dissolved together in a dichloromethane/DMF (360 mL/80 mL) mixture; then, 25.28 g of HATU was added; the resulting mixture was stirred at room temperature to react for 40 min; the reaction solution was washed with water, washed 2 times with dilute hydrochloric acid, washed with water, then washed 2 times with sodium bicarbonate aqueous solution, washed with water, dried and evaporated to dryness to obtain 45 g of product. MS[ESI] m/z: 791.7[M+H]+.

Reaction 5: 45 g of the above product was dissolved in 330 mL of dichloromethane; 150 mL of trifluoroacetic acid was added to react for 1 h at room temperature; the reaction solution was washed four times with water; the organic phase was evaporated to dryness to obtain 39 g of product. MS[ESI] m/z: 718.5[M−H]−, 1HNMR (400 MHZ, DMSO-d6): δ=12.09 (OH), 7.93 (3H), 7.74 (1H), 7.39 (2H), 7.32 (2H), 4.25 (3H), 4.00 (1H), 3.48 (26H), 3.40 (5H), 3.26 (5H), 2.50 (1H), 2.24 (2H), 1.85 (1H), 1.79 (1H).

### Example 3: Synthesis of Lb-Lc Fragment 1

### Step 1: Synthesis of LC Fragment 1

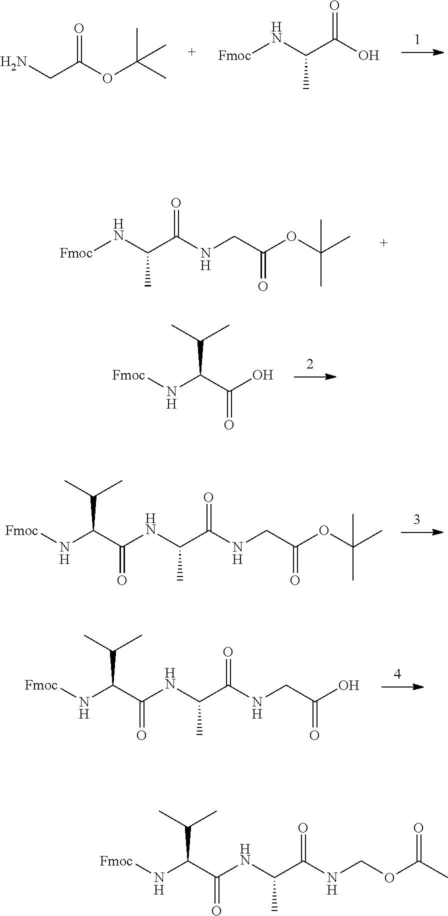

Reaction 1: 100 g of Fmoc-alanine and 65 g of HOBT as well as 92.36 g of EDCI were added to a dichloromethane/DMF (1.7 L/0.3 L) mixture, stirred and cooled to 0° C.; 104 g of DIPEA was slowly added to allow reaction for 30 min at 0° C.; 46.35 g of tert-butyl glycinate was added dropwise to allow reaction for 20 h at room temperature; the reaction solution was washed with water, washed twice with 2 N dilute hydrochloric acid, washed twice with potassium carbonate aqueous solution, washed twice with water; then the organic phase was dried and evaporated to obtain 144 g of crude product, which was to be used directly in next reaction. MS[ESI] m/z: 425.5[M+H]+.

Reaction 2: 110 g of the above product was dissolved in 1 L of DMF; then, 0.2 L of morpholine was added to allow reaction for 2 h at room temperature; and the reaction solution was evaporated to dryness under reduced pressure. 88 g of Fmoc-valine and 52.6 g of HOBT as well as 74.56 g of EDCI were dissolved in the dichloromethane/DMF (1 L/0.2 L) mixture; 100 g of DIPEA was added; the mixture was stirred for 30 min at room temperature; the above product with protected groups removed was dissolved in dichloromethane and added dropwise to the above reaction solution to allow reaction for 5 h reaction at room temperature; the reaction solution was washed with water and evaporated to dryness to obtain a crude product, which was separated by column chromatography to obtain 90 g of product. MS[ESI] m/z: 791.7[M+H]+.

Reaction 3: 70 g of the above product and 29 g of anisole were added to 0.5 L of dichloromethane and stirred; 100 mL of trifluoroacetic acid was slowed added dropwise and stirred to allow reaction for 5 h at room temperature; the reaction solution was evaporated to dryness under reduced pressure to obtain solids; 300 mL of ethyl acetate was added to the obtained solids and stirred for crystallization to obtain solids, which were filtered, washed with ethyl acetate and collected; and the mother solution was evaporated to dryness and then the above operation was repeated to obtain a total of 60 g of solids. MS[ESI] m/z: 466.5[M−H]−.

Reaction 4: 5 g of the above product, 7.4 mg of copper acetate, 5.5 g of lead tetraacetate, and 1.48 g of acetic acid were dissolved in 100 mL of DMF, and heated to 60° C. under the protection of nitrogen to react for 4 h; 1 L of ethyl acetate was added to the reaction solution, stirred for 10 min and filtered; the filtrate was washed twice with water, dried and evaporated to dryness to obtain solids; the obtained solids were pulped with a small amount of n-hexane, filtered, washed with a small amount of acetonitrile, and then dried to obtain 2.1 g of product. MS[ESI] m/z: 482.5[M+H]+, 1HNMR (400 MHZ, DMSO-d6): δ=8.89 (1H), 7.89 (2H), 7.89 (2H), 7.72 (1H), 7.41 (3H), 7.30 (2H), 5.08 (1H), 4.28 (2H), 4.22 (1H), 3.86 (3H, S), 1.98 (4H), 1.21 (3H, d), 0.85 (6H).

### Step 2: Synthesis of LB-LC Fragment 1

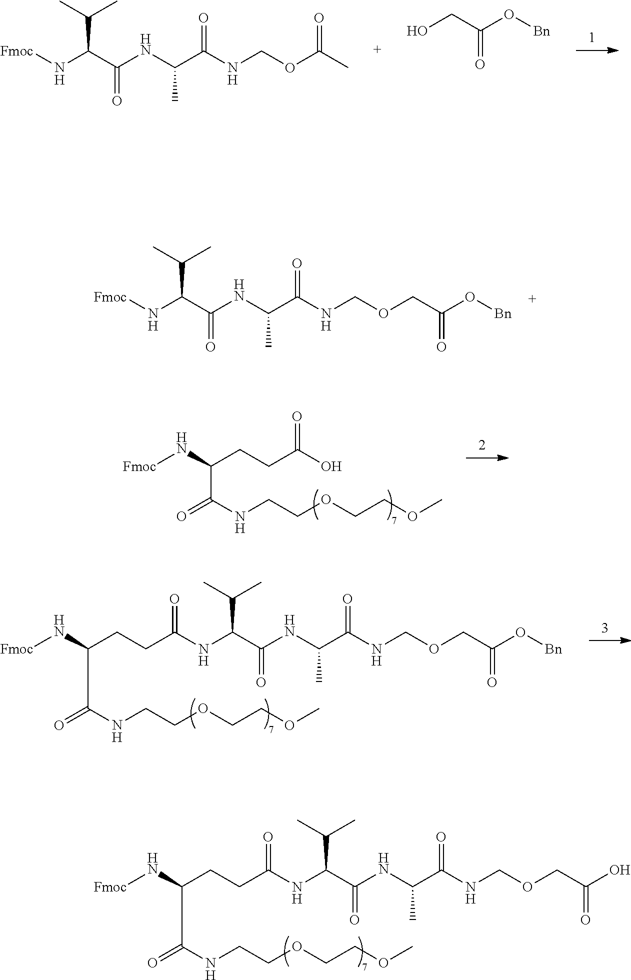

Reaction 1: 4.8 g of dipeptide compound (the product from step 1) and 0.25 g of PPTS were dissolved in 100 mL of dichloromethane; 17 g of benzyl glycolate was added; the resulting mixture was refluxed to allow reaction for 24 h at 40° C.; 50 mL of water was added to the reaction solution; and the organic layer was evaporated to dryness and then separated by column chromatography to obtain 5.2 g of product. MS[ESI] m/z: 588.6[M+H]+, 1HNMR (400 MHZ, DMSO-d6): δ=8.72 (1H), 8.08 (1H), 7.89 (2H), 7.74 (2H), 7.43 (10H), 5.13 (2H, S), 4.63 (2H, d), 4.25 (4H), 4.13 (1H, S), 3.32 (3H), 2.21 (3H, d), 0.84 (6H).

Reaction 2: 2 g of the above product was dissolved in 20 mL of DMF; 4 mL of morpholine was added to allow reaction for 1 h; the reaction solution was evaporated to dryness under reduced pressure; the resulting product and 2.4 g of the reactant Fmoc-L-monopolyethylene glycol glutamate (the product from Example 2) were dissolved in 25 mL of DMF; 0.43 g of DIPEA and 1.3 g of DMTMM were added; then, 2.5 mL of water was added to allow reaction for 80 min at room temperature; the reaction solution was introduced into 200 ml of water; and the resulting mixture was extracted twice with 50 mL of dichloromethane and then purified by column chromatography to obtain 3.5 g of product. MS[ESI] m/z: 1083.3[M+H]+.

Reaction 3: 2 g of the above product was dissolved in an ethanol/ethyl acetate (80/40 mL) mixture; 0.4 g of 10% Pd/C was added to allow hydrogenation for 20 h; the reaction solution was filtered and evaporated to dryness under reduced pressure; the resulting solids were pulped with ethyl acetate, filtered and dried to obtain 1.9 g of product. MS[ESI] m/z: 991.2[M−H]−.

### Example 4: Synthesis of Lb-LC Fragment 2

### Step 1: Synthesis of LC Fragment 2

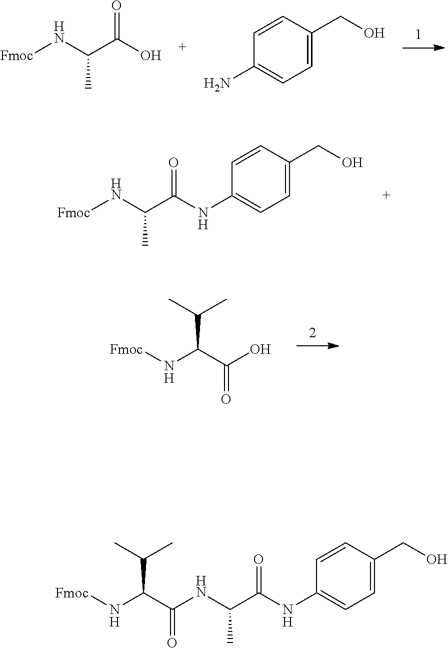

Reaction 1: 25 g of Fmoc-L-alanine and 14.8 g of p-aminobenzyl alcohol were dissolved in a DMF/water (300 mL/40 mL) mixture; 31.1 g of DMTMM was added over stirring at room temperature and then stirred to allow reaction for 30 min; the reaction solution was also introduced into 1.5 L of water, with pH adjusted to 2-3; the resulting mixture was extracted three times with ethyl acetate; the organic phase was washed with 1 N dilute hydrochloric acid, washed with water, dried and evaporated to dryness; the resulting solids were pulped with acetonitrile, filtered, washed with acetonitrile, and dried to obtain 20 g of crude product. MS[ESI] m/z: 417.5[M+H]+. 1HNMR (400 MHz, DMSO-d6): δ=9.94 (1H), 7.89 (2H), 7.75 (2H), 7.66 (1H), 7.56 (2H), 7.42 (2H), 7.34 (2H), 7.26 (2H), 5.11 (1H, d), 4.44 (2H, d), 4.19 (4H, d), 1.31 (3H).

Reaction 2: 15.5 g of the above product was dissolved in a DMF/morpholine (100 mL/50 mL) mixture, stirred at room temperature to react for 2 h, and then filtered; and the filtrate was evaporated to dryness under reduced pressure. 11.48 g of Fmoc-valine was added to this solid; the DMF/water (100 mL/15 mL) mixture was added and then stirred; 12.17 g of DMTMM was added at room temperature to allow reaction for 40 min; the reaction solution was introduced into 1.2 L of water to allow precipitation of solids, and then filtered; the solids were washed with 1 L of water; the filter cake was dissolved in 1 L of tetrahydrofuran; sodium chloride was added for stratification; the organic phase was dried and evaporated to dryness; and the resulting solids were pulped with acetonitrile at 40° C., cooled and then filtered to 14.5 g of product. MS[ESI] m/z: 516.6[M+H]+.

### Step 2: Synthesis of LB-LC Fragment 2

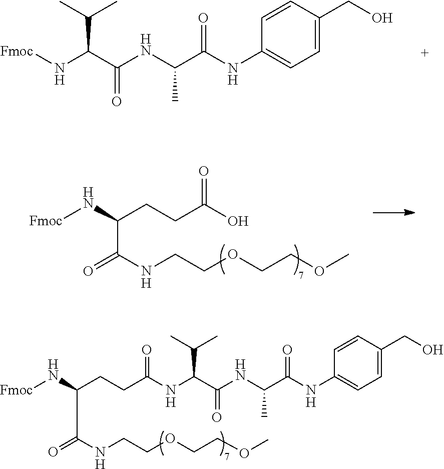

Reaction: 4.8 g of the reactant dipeptide p-aminobenzyl alcohol (the product from Step 1) was dissolved in 50 mL of DMF; 10 mL of morpholine was added to allow reaction for 1 h at room temperature; the reaction solution was evaporated to dryness under reduced pressure; the resulting product together with 6.2 g of the reactant Fmoc-L-monopolyethylene glycol glutamate (the product from Example 2) were dissolved in 100 mL of DMF; 14 mL of water was added and then stirred for 10 min; 3.048 g of DMTMM was added to the system to allow reaction for 90 min at room temperature; and dichloromethane and dilute hydrochloric acid were added to stratify the reaction solution; the organic phase was evaporated to dryness; the resulting product was stirred with the addition of a small amount of dilute hydrochloric acid; the product was precipitated, filtered, washed with water and dried to obtain 6.4 g of product. MS[ESI] m/z: 1011.2[M+H]+.

### Example 5: Synthesis of Lb-LC Fragment 3

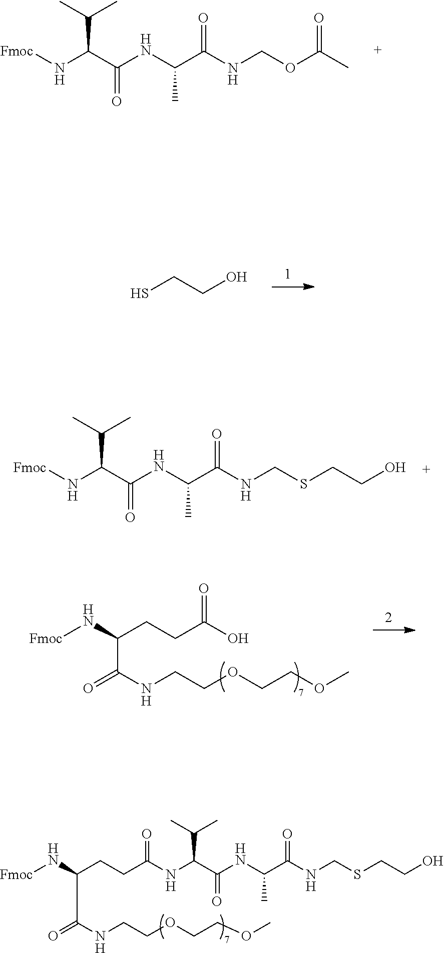

Reaction 1: 7.5 g of the dipeptide compound (the same as the product from Step 1 of Example 3) was dissolved in 300 mL of trifluoroacetic acid; 2.44 g of mercaptoethanol was added to allow reaction for 90 min at room temperature; 30 mL of water was added to quench the reaction; the reaction solution was evaporated under reduced pressure till about 30 mL of liquid was left; 150 mL of water was added and then stirred for 30 min for solid precipitation; the solids were filtered, washed with water to neutral, dried, and pulped with ethyl acetate to obtain 6.1 g of product. MS[ESI] m/z: 500.7[M+H]+.

Reaction 2: For 0.277 g of the above product, the protecting groups were removed with morpholine; the resultant was evaporated to dryness and then together with 0.735 g of the reactant Fmoc-L-monopolyethylene glycol glutamate (the product from Example 2), dissolved in 10 mL of DMF; 0.13 g of DIPEA and 0.388 g of DMTMM were added; 1 mL of water was then added to allow reaction for 40 min at room temperature; 50 mL of water was added; and the resulting mixture was extracted with 30 mL of dichloromethane; the organic layer was washed with water, dried and then purified by column chromatography to obtain 0.5 g of product. MS[ESI] m/z: 995.2[M+H]+. 1HNMR (400 MHz, DMSO-d6): δ=11.15 (OH), 7.89 (2H), 7.75 (2H), 7.42 (2H), 7.34 (2H), 4.25 (6H), 3.90 (1H), 3.49 (34H), 3.23 (3H), 2.19 (2H), 1.84 (2H), 1.28 (12H), 0.86 (6H).

### Example 6: Synthesis of Linker-Drug Conjugates 1a and 1b

### Step 1

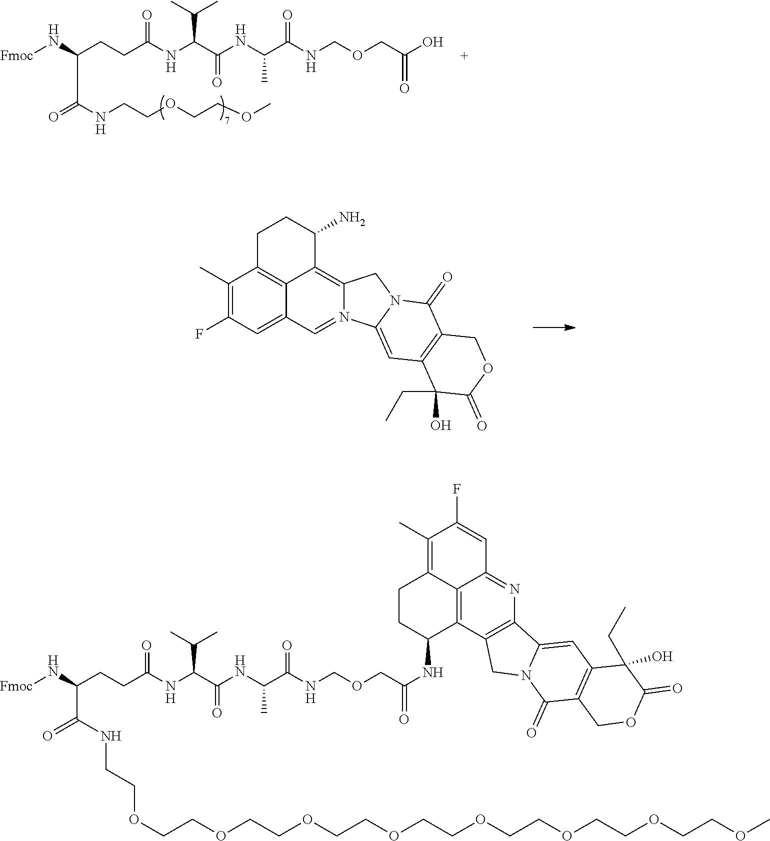

Reaction: 2.0 g of the reactant substituted glycolic acid and 0.85 g of exatecan mesylate were dissolved in 20 mL of DMF; 0.41 g of DIPEA was added; 0.62 g of DMTMM was then added; 2 mL of water was added to allow reaction for 1 h at room temperature; 200 mL of water was added to the reaction solution, which was then extracted three times with 100 mL of dichloromethane; and the organic phase was washed twice with 5% dilute hydrochloric acid, dried, evaporated to dryness and then purified by column chromatography to obtain 0.9 g of product. MS[ESI] m/z: 1410.6[M+H]+. 1HNMR (400 MHZ, DMSO-d6): δ=8.67 (OH), 7.75 (6H), 7.31 (6H), 4.62 (2H), 4.20 (5H), 3.93 (2H), 3.48 (36H), 3.23 (6H), 2.50 (2H), 2.38 (4H), 1.98 (8H), 1.17 (3H), 0.80 (12H).

### Step 2a: Synthesis of Linker-Drug Conjugate 1a

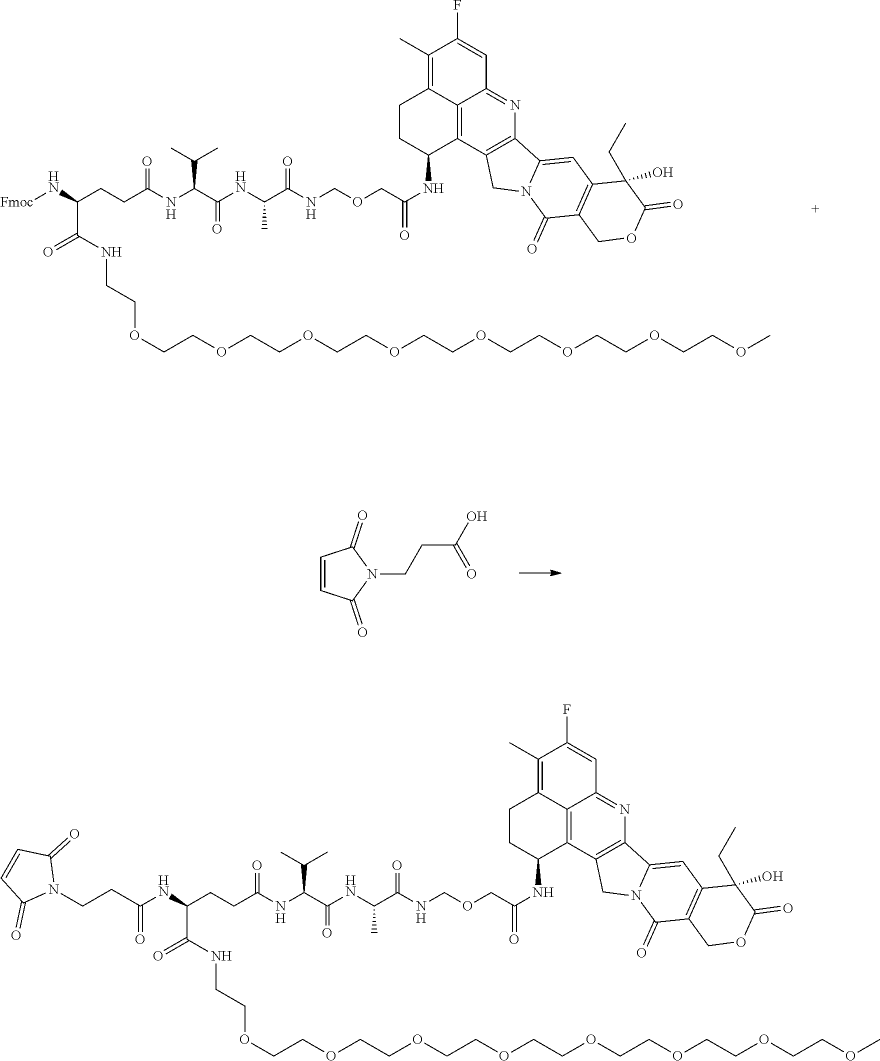

Reaction: 100 mg of the reactant linker-exatecan conjugate was dissolved in 6 mL of DMF; 0.5 mL of morpholine was added to allow reaction for 80 min at room temperature; the reaction solution was evaporated under reduced pressure; the resulting product together with 12 mg of maleimide propionic acid and 18 mg of DIPEA were dissolved in a dichloromethane/DMF (6/1 mL) mixture; 27 mg of HATU was then added to allow reaction for 1 h at room temperature; 20 mL of water was added for quenching; the reaction solution was extracted with dichloromethane; and the resulting product was purified by column chromatography to obtain 80 mg of product. MS[ESI] m/z: 1339.6[M+H]+. 1HNMR (400 MHz, DMSO-d6): δ=8.67 (1H), 8.48 (1H), 8.05 (1H), 7.88 (1H), 7.75 (2H), 7.31 (1H, s), 6.99 (2H), 6.51 (1H, s), 5.57 (1H), 5.42 (1H), 5.18 (1H), 4.62 (2H), 4.19 (4H), 3.90 (2H), 3.56 (2H), 3.50 (28H), 3.42 (4H), 3.23 (6H), 2.38 (4H), 2.19 (4H), 1.88 (4H), 1.27 (3H), 0.85 (6H), 0.75 (6H).

### Step 2b: Synthesis of Linker-Drug Conjugate 1b

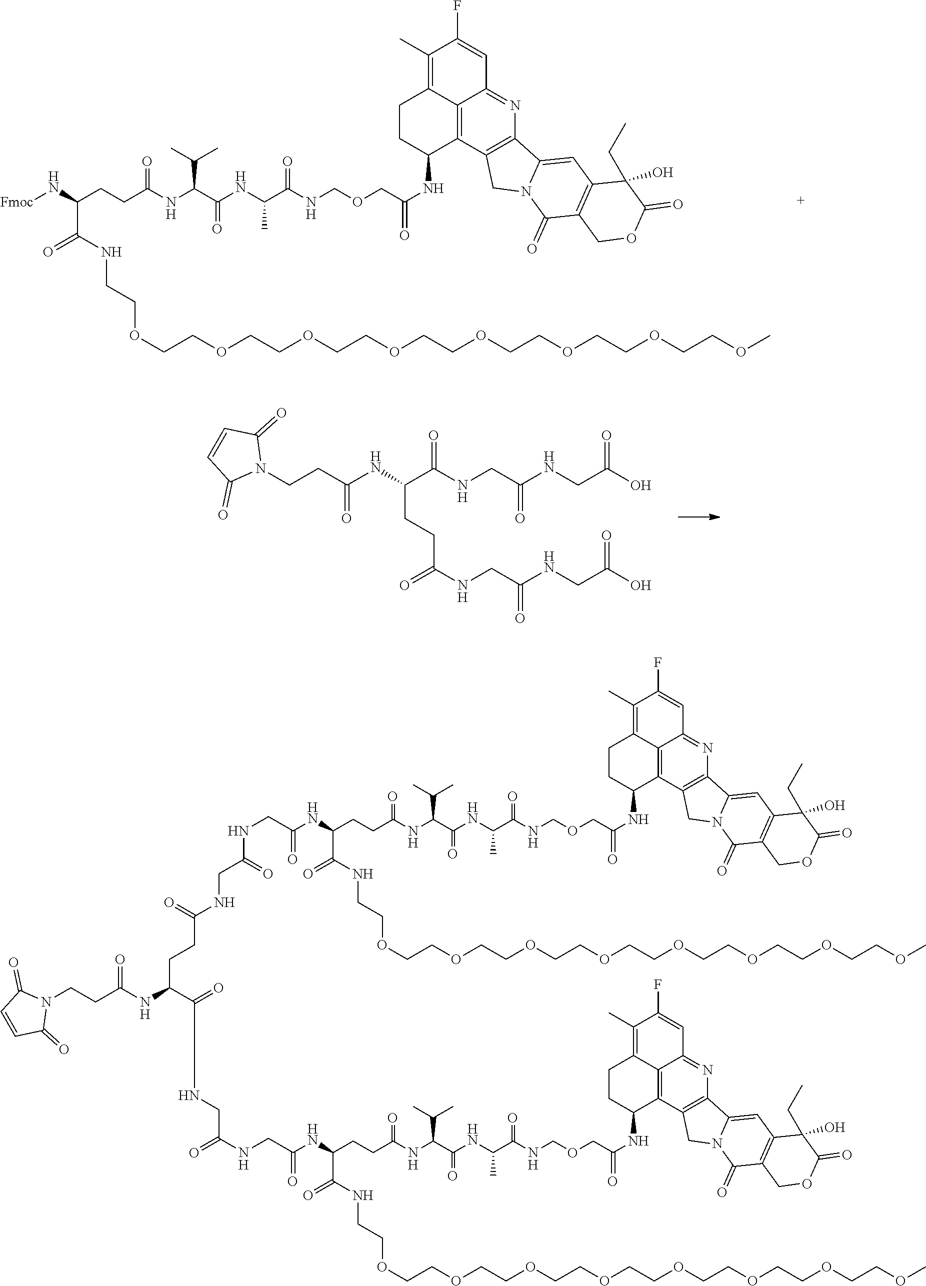

Reaction: 280 mg of the reactant linker-exatecan conjugate was dissolved in 10 mL of DMF; 1 mL of morpholine was added to allow reaction for 2 h at room temperature; the reaction solution was evaporated to dryness under reduced pressure; the resulting product together with 52 mg of diacid and 78 mg of DIPEA were dissolved in 30 mL of DMF, and stirred for 30 min; 95 mg of T3P was added in two portions to allow reaction for 1 h; and the reaction solution was evaporated to dryness and then pulped with ethyl acetate to obtain 100 mg of product. Then, the liquid phase was purified to obtain the pure product of linker-drug conjugate 1b (40 mg). MS[ESI] m/z: 1433.5[M+2H]2+.

1H NMR (400 MHZ, DMSO-d6): δ 8.68 (t, J=6.4 Hz, 2H), 8.48 (d, J=8.8 Hz, 2H), 8.24-8.21 (m, 2H), 8.11-8.07 (m, 5H), 7.99-7.93 (m, 4H), 7.78-7.75 (m, 4H), 6.98 (s, 2H), 6.51 (s, 2H), 5.62-5.57 (m, 2H), 5.42 (s, 4H), 5.17 (s, 4H), 4.66-4.57 (m, 4H), 4.21-4.18 (m, 5H), 4.10 (t, J=7.4 Hz, 2H), 3.98 (s, 4H), 3.77-3.69 (m, 8H), 3.61-3.58 (m, 2H), 3.51-3.47 (m, 60H), 3.43-3.38 (m, 8H), 3.23 (s, 6H), 3.21-3.18 (m, 4H), 2.43-2.38 (m, 8H), 2.20-2.14 (m, 10H), 1.91-1.80 (m, 10H), 1.18 (d, J=7.2 Hz, 6H), 0.86 (t, J=7.2 Hz, 6H), 0.78-0.74 (m, 12H).

### Example 7: Synthesis of Linker-Drug Conjugate 2

### Step 1

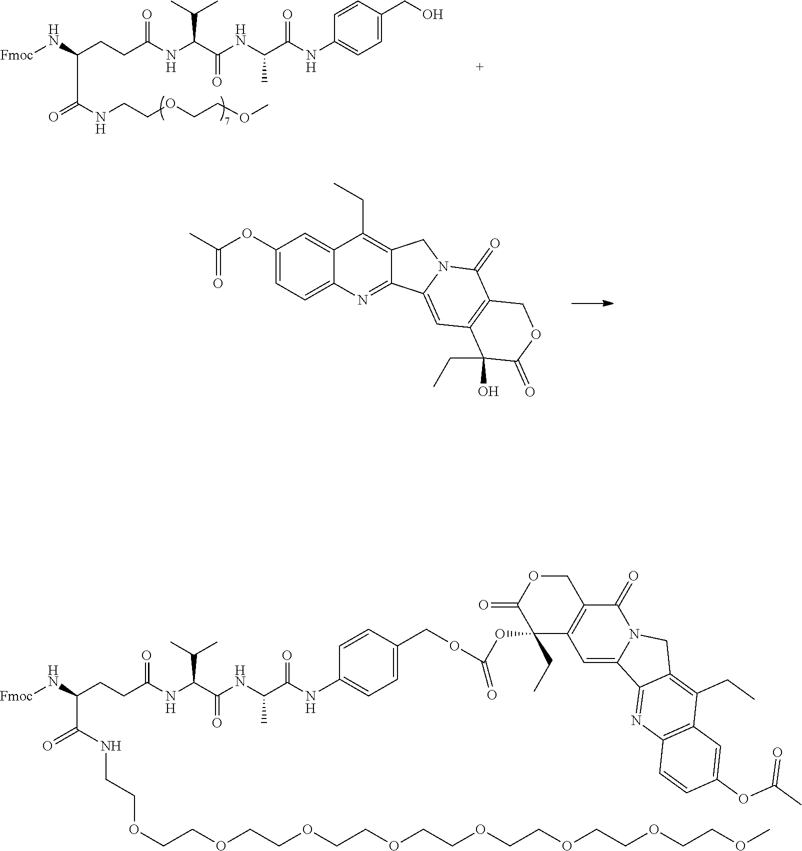

Reaction: 1.04 g of DMAP was dissolved in 80 mL of dried dichloromethane; 0.288 g of triphosgene was added; the mixture was stirred for 3 min; then 1.0 g acetylated SN38 was added and then stirred for 15 min. 2.8 g of the reactant substituted p-aminobenzyl alcohol was dissolved in 30 mL of DMF; the resulting mixture was added to the above reaction solution to allow reaction for 1 h at room temperature; water was added to quench the reaction; the reaction solution was extracted with dichloromethane and then purified by column chromatography obtain 2.55 g of product. MS[ESI] m/z: 1471.7[M+H]+. 1HNMR (400 MHZ, DMSO-d6): δ=9.97 (1H), 8.22 (2H), 7.85 (4H), 7.61 (2H), 7.51 (2H), 7.43 (1H), 7.39 (2H), 7.32 (3H), 7.03 (1H), 5.53 (2H), 5.31 (1H), 5.09 (2H), 4.32 (1H), 4.25 (5H), 3.90 (1H), 3.46 (34H), 3.23 (5H), 2.89 (2H), 2.73 (1H), 2.37 (3H, s), 2.21 (5H), 1.98 (3H), 1.88 (2H), 1.28 (4H), 0.84 (6H).

### Step 2a: Synthesis of Linker-Drug Conjugate 2a

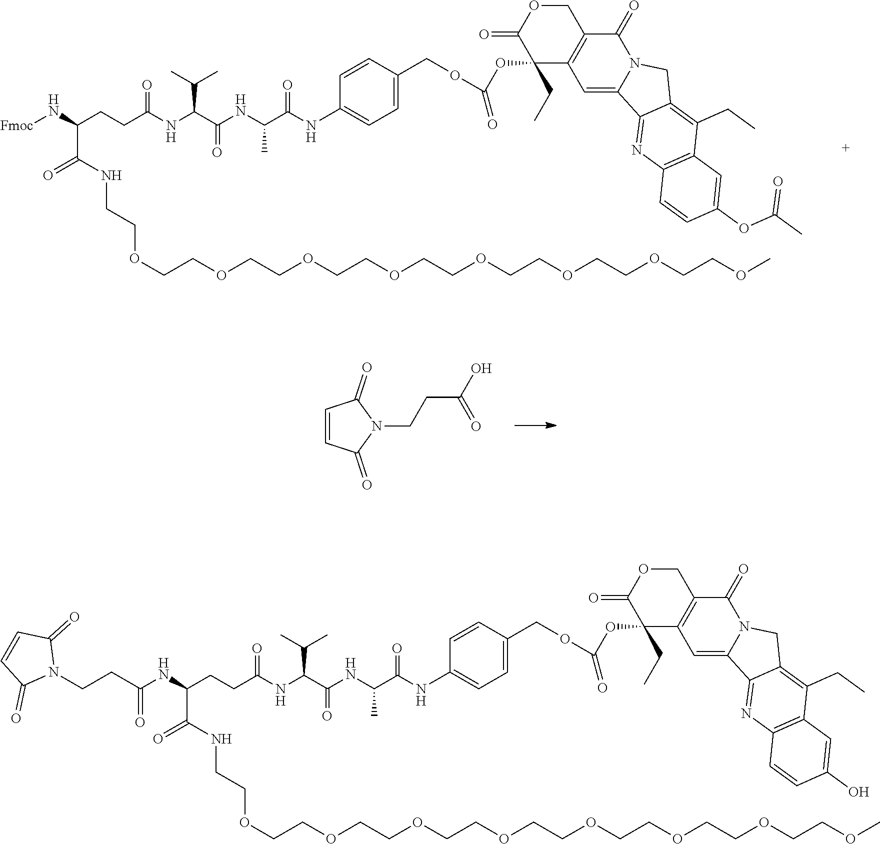

Reaction: 0.25 g of the reactant linker-acetylated SN38 conjugate was dissolved in 10 mL of DMF; 1 mL of morpholine was added to allow reaction for 1 h at room temperature; the reaction solution was evaporated under reduced pressure; the resulting product together with 30 mg of maleimide propionic acid and 45 mg of DIPEA were dissolved in 10 mL of DMF; 77 mg of HATU was added and then stirred for 40 min; and 10 ml of water was added to the reaction solution, which was then extracted with dichloromethane, evaporated to dryness, and then purified by column chromatography to obtain 120 mg of product. MS[ESI] m/z: 1358.5[M+H]+.

### Step 2b: Synthesis of Linker-Drug Conjugate 2b

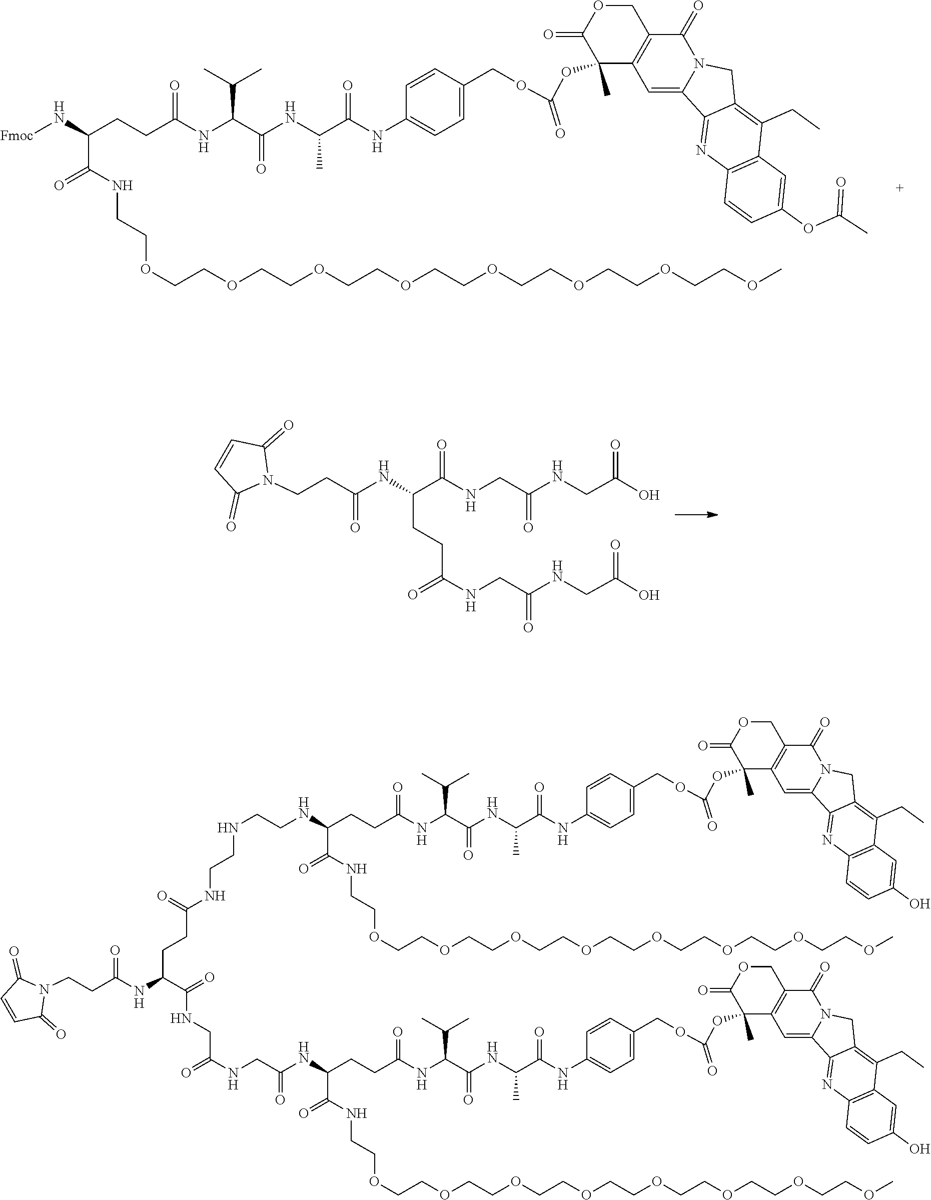

Reaction: 1.2 g of the reactant linker-acetylated SN38 conjugate was dissolved in 10 mL of DMF; 1 mL of morpholine was added to allow reaction for 3 h at room temperature; the reaction solution was evaporated under reduced pressure; the resulting product together with 280 mg of diacid and 113 mg of DIPEA as well as 340 mg of DMTMM were dissolved in 10 mL of DMF; 1 mL of water was added and then stirred for 80 min; and 50 mL of water was added to the reaction solution, which was then evaporated to dryness and purified by column chromatography to obtain 240 mg of product. MS[ESI] m/z: 1452.6[M+2H]2+.

### Example 8: Synthesis of Linker-Drug Conjugates 3a and 3b

### Step 1

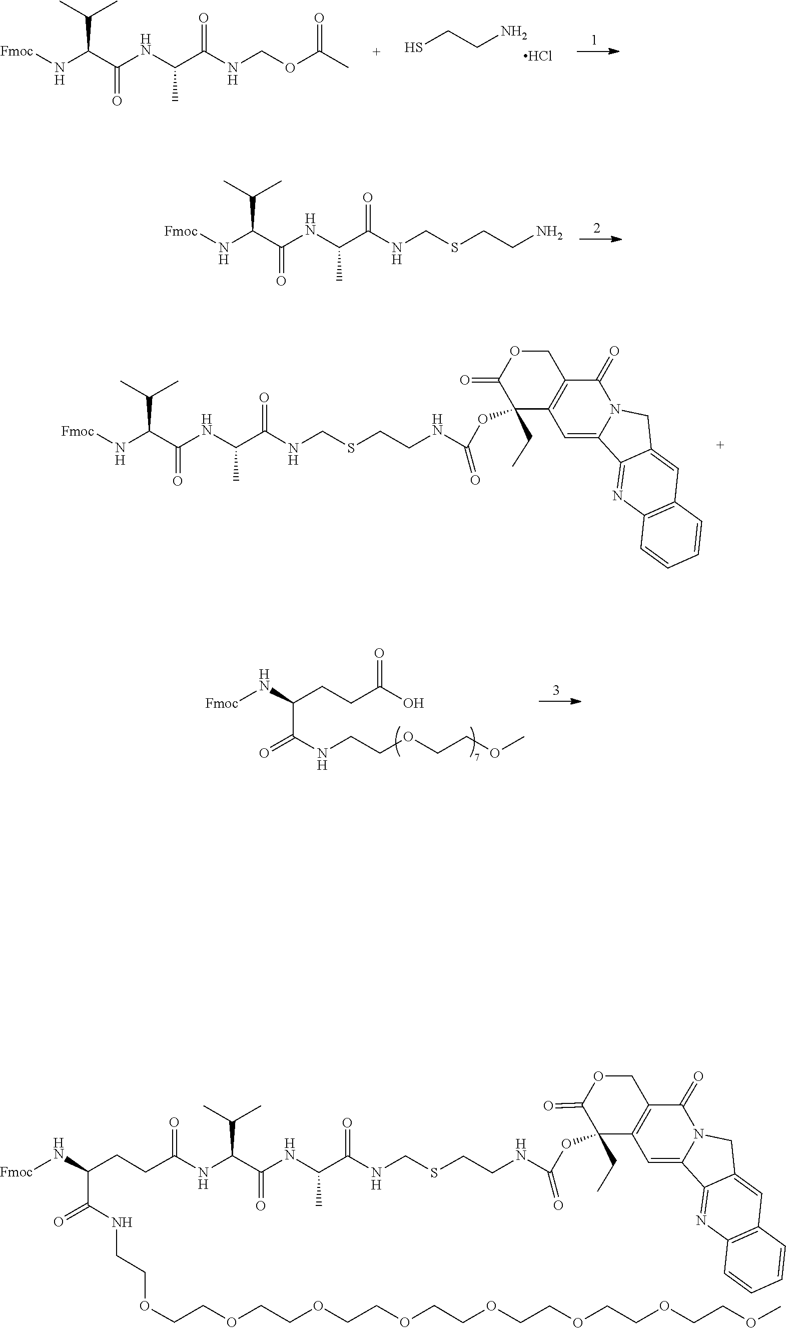

Reaction 1: 3.2 g of the reactant mercaptoethylamine hydrochloride was dissolved in 100 mL of trifluoroacetic acid and stirred for 30 min; then, 10 g of a dipeptide reactant was added to allow reaction for 3 h at room temperature; and the reaction solution was evaporated to dryness under reduced pressure to obtain a crude product, which was to be directly used for the next reaction. MS[ESI] m/z: 499.6[M+H]+.

Reaction 2: 4.5 g of DMAP was dissolved in 350 mL of dried dichloromethane; 1.1 g of triphosgene was added and then stirred to react for 3-5 min; and 3.5 g of camptothecin was added and stirred to react for 15 min, and the system was clarified. 7 g of the reactant substituted mercaptoethylamine was dissolved in 30 mL of DMF to react for 20 min; 200 mL of water was added to the reaction solution for quenching; the reaction solution was stratified; the organic phase was washed with water, washed with dilute hydrochloric acid, dried and evaporated to dryness, and then purified by column chromatography to obtain 6 g of product. MS[ESI] m/z: 874.1 [M+H]+. 1HNMR (400 MHz, DMSO-d6): δ=8.46 (NH), 8.04 (1H), 7.77 (3H), 7.73 (3H), 7.42 (4H), 7.31 (3H), 4.25 (8H), 4.07 (4H), 3.89 (2H), 2.67 (2H), 2.01 (2H), 1.22 (5H), 0.86 (9H).

Reaction 3: 5.9 g of the product from the above step was dissolved in 60 mL of DMF; 6 mL of morpholine was added to allow reaction for 3 h at room temperature; the reaction solution was evaporated to dryness under reduced pressure; the resulting product together with 5 g of the reactant Fmoc-L-monopolyethylene glycol glutamate were dissolved in 100 mL of DMF; 1.8 g of DIPEA and 1.3 g of DMTMM were added in sequence to allow reaction for 40 min at room temperature; 50 mL of water was added to the reaction solution for quenching; the reaction solution was extracted with dichloromethane; and the organic layer was washed with water, washed with dilute hydrochloric acid, washed with water to obtain a crude product, which was then purified by column chromatography to obtain 4.5 g of product. MS[ESI] m/z: 1368.6[M+H]+. 1HNMR (400 MHz, DMSO-d6): δ=8.69 (1H), 8.48 (1H), 8.13 (3H), 7.86 (4H), 7.77 (1H), 7.71 (2H), 7.48 (1H), 7.42 (3H), 7.31 (2H), 5.32 (2H), 4.89 (1H), 4.40 (2H), 4.36 (1H), 4.20 (6H), 4.16 (1H), 3.92 (1H), 3.85 (1H), 3.70 (1H), 3.48 (30H), 3.22 (4H, s), 3.16 (2H), 2.84 (1H), 2.77 (1H), 2.18 (2H), 1.98 (2H), 1.75 (2H), 1.19 (3H), 0.98 (3H), 0.79 (6H).

### Step 2a: Synthesis of Linker-Drug Conjugate 3a

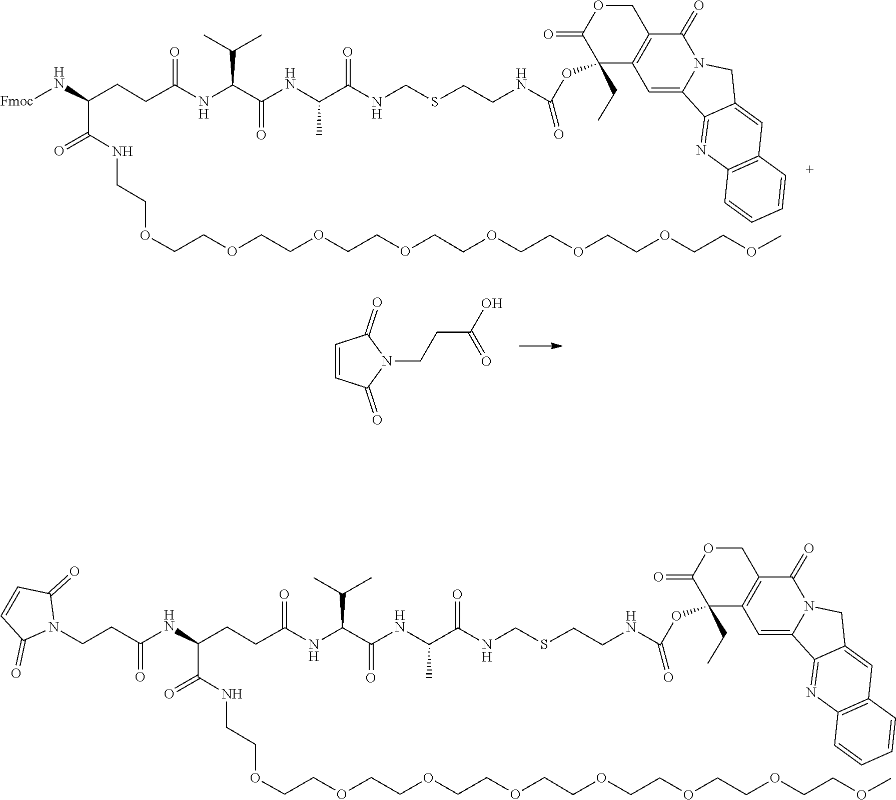

Reaction: 0.451 g of the reactant linker-camptothecin conjugate was dissolved in 4 mL of DMF; 0.5 mL of morpholine was added to allow reaction for 1.5 h at room temperature; the reaction solution was evaporated under reduced pressure; the resulting product together with 51 mg of maleimide propionic acid and 78 mg of DIPEA were dissolved in 5 mL of DMF; 137 mg of HATU was added and then stirred for 40 min; and 10 ml of water was added to the reaction solution, which was then extracted with dichloromethane, evaporated to dryness, and then purified by column chromatography to obtain 180 mg of product. MS[ESI] m/z: 1297.5[M+H]+.

### Step 2b: Synthesis of Linker-Drug Conjugate 3b

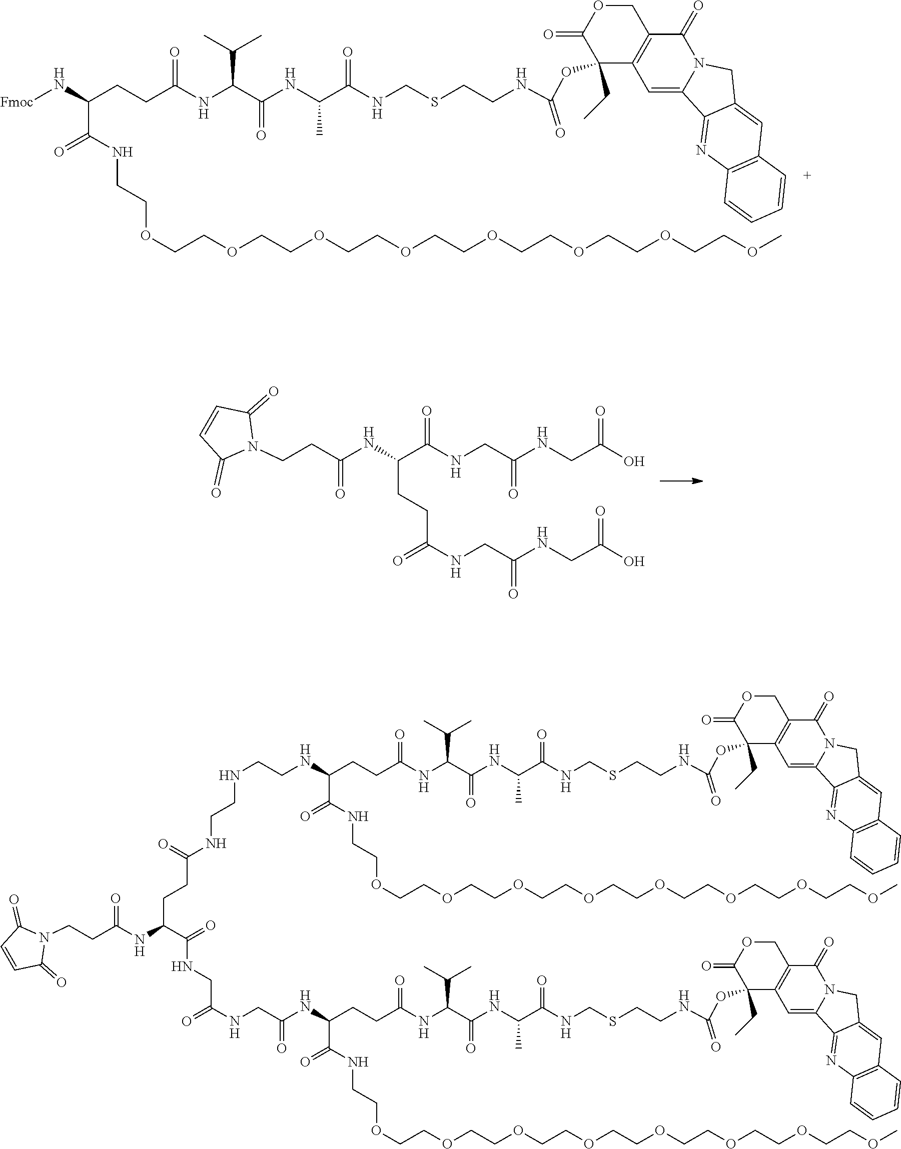

Reaction: 1.2 g of the reactant linker-camptothecin conjugate was dissolved in 10 mL of DMF; 1 mL of morpholine was added to allow reaction for 3 h at room temperature; the reaction solution was evaporated under reduced pressure; the resulting product together with 280 mg of diacid and 113 mg of DIPEA as well as 340 mg of DMTMM were dissolved in 10 mL of DMF; 1 mL of water was added and then stirred for 80 min; and 50 mL of water was added to the reaction solution, which was then evaporated to dryness and purified by column chromatography to obtain 240 mg of product. MS[ESI] m/z: 1391.6[M+2H]²⁺.

### Example 9: Synthesis of Linker-Drug Conjugate 4

### Step 1

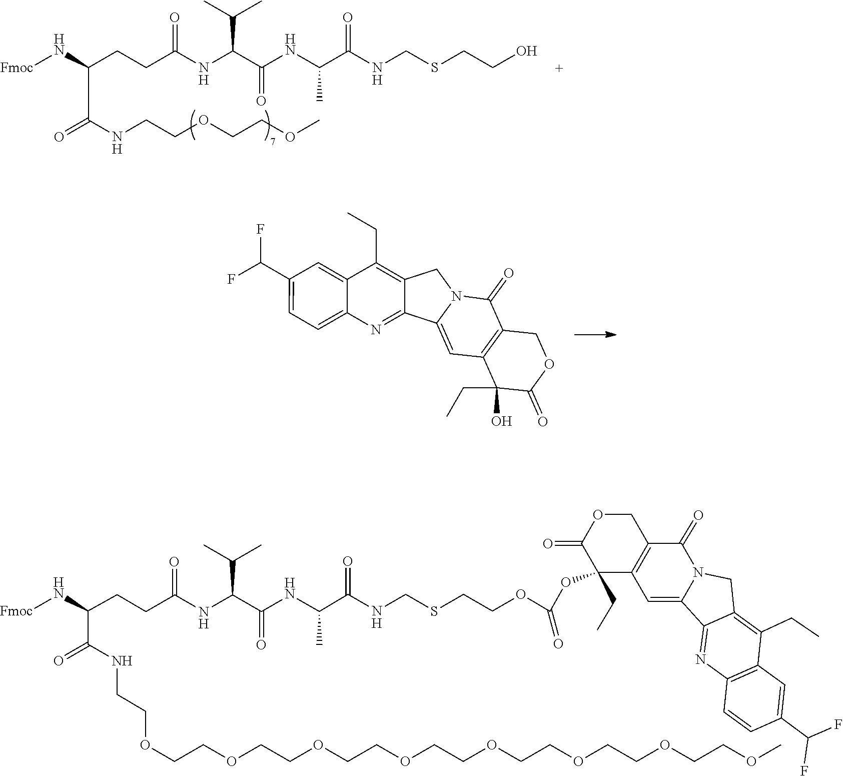

Reaction: 23 mg of DMAP was dissolved in 4 mL of dried dichloromethane; 5.5 mg of triphosgene was added; the mixture was stirred for 3 min; and then 21 mg of camptothecin derivative was added and then stirred for 10 min. 50 mg of the reactant substituted mercaptoethanol was dissolved in 0.5 mL of DMF; the resulting mixture was added to the above reaction solution to allow reaction for 4 h at room temperature; water was added to quench the reaction; and the reaction solution was extracted with dichloromethane and then purified by column chromatography obtain 40 mg of product. MS[ESI] m/z: 1447.6[M+H]+. 1HNMR (400 MHZ, DMSO-d6): δ=8.52 (1H), 8.29 (1H), 8.05 (2H), 7.88 (3H), 7.74 (2H), 7.41 (6H), 7.32 (1H), 5.53 (2H), 4.18 (10H), 3.48 (30H), 3.41 (6H), 3.23 (4H), 2.80 (2H), 2.18 (3H), 1.23 (8H), 0.80 (12H).

### Step 2: Synthesis of Linker-Drug Conjugate 4

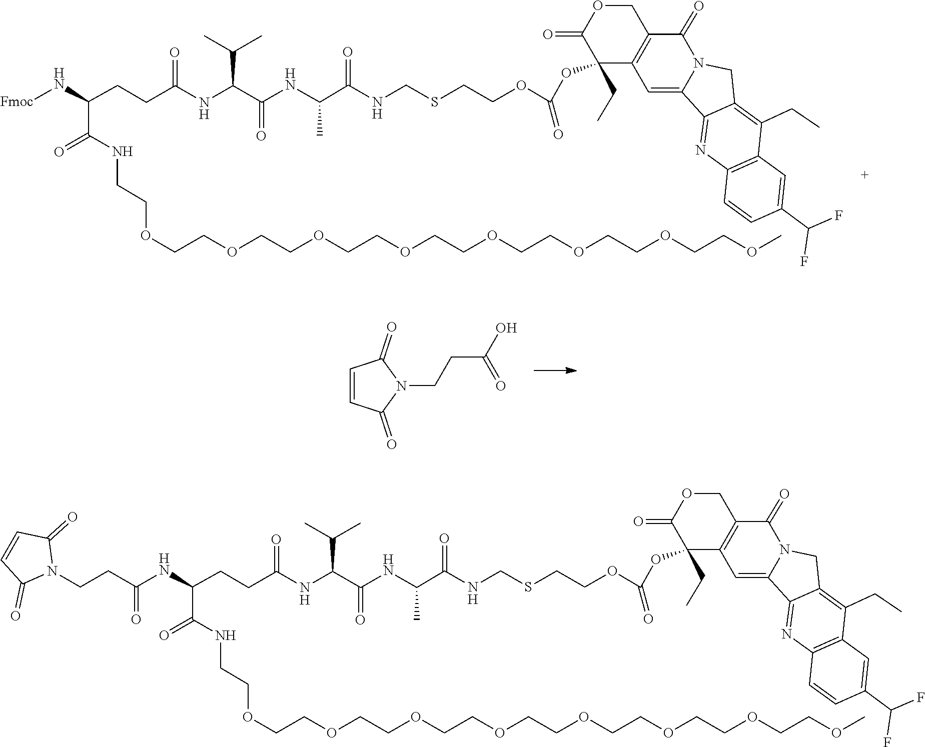

Reaction: 0.1 g of the reactant linker-camptothecin conjugate was dissolved in 4 mL of DMF; 0.5 mL of morpholine was added to allow reaction for 1.5 h at room temperature; the reaction solution was evaporated under reduced pressure; the resulting product together with 12 mg of maleimide propionic acid and 18 mg of DIPEA were dissolved in 5 mL of DMF; 32 mg of HATU was added and then stirred for 60 min; and 10 mL of water was added to the reaction solution, which was then extracted with dichloromethane, evaporated to dryness, and then purified by column chromatography to obtain 80 mg of product. MS[ESI] m/z: 1376.5[M+H]+.

### Example 10: Synthesis of Antibody-Drug Conjugate Tr-1a

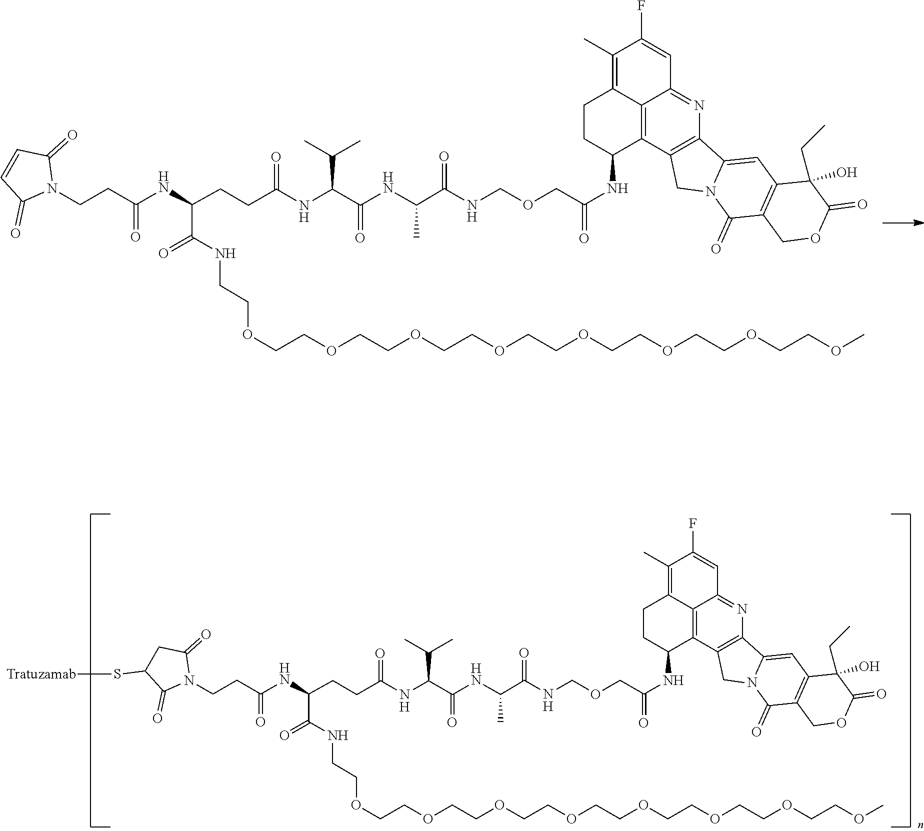

Reduction of antibodies: The concentration of a trastuzumab solution was adjusted to 10 mg/mL with an ABS buffer (pH 5.5); 60 μL of a TCEP solution (10 mM) was added to 1 mL of an antibody solution; then a small amount of Na₂HPO₄ solution (1 M) was added to adjust the pH of the reaction solution to 7.0; the reaction solution was incubated for 1 h in a water bath at 37° C., taken out and then cooled to room temperature for direct use in the next reaction.

Conjugation to antibodies: 8 mg small molecules (30 eq. with respect to the antibody) was dissolved with 200 uL of DMSO. The resultant was diluted with 500 uL of 10 mM ABS+5 mM EDTA buffer and added to a reaction tube. The tube was shaken by swirling for 0.5 h-1 h at room temperature (20-28° C.). 30 uL of 1M NAC solution was added to quench the reaction, and the tube was shaken by swirling for 20 min at room temperature (20-28° C.). Samples were collected and filtered with a sterile membrane (0.22 um), rinsed with a small amount of ABS+ EDTA buffer and then filtered.

Purification of drug-antibody conjugates: The above reactants were allowed to pass through a zeba desalted spin column (with the molecular weight cutoff of 10 KDa) to remove small molecules; the resulting target product solution was centrifuged and concentrated using an Amico Ultra (30,000 MWCO, Millipore Co.) vessel; the buffer was displaced; and the product solution was adjusted to ABS (pH 5.5) at a concentration of 4 mg/mL.

Evaluation of drug-antibody conjugate products: The product had a yield of 60% and was then measured in ultraviolet absorbance at 280 nm and at 370 nm, and with the molar extinction coefficients of the antibody and drug at 280 nm and 370 nm, respectively, it was calculated that the antibody of each molecule was linked to 7.88 drug molecules on average.

### Example 11: Synthesis of Antibody-Drug Conjugate Tr-1b

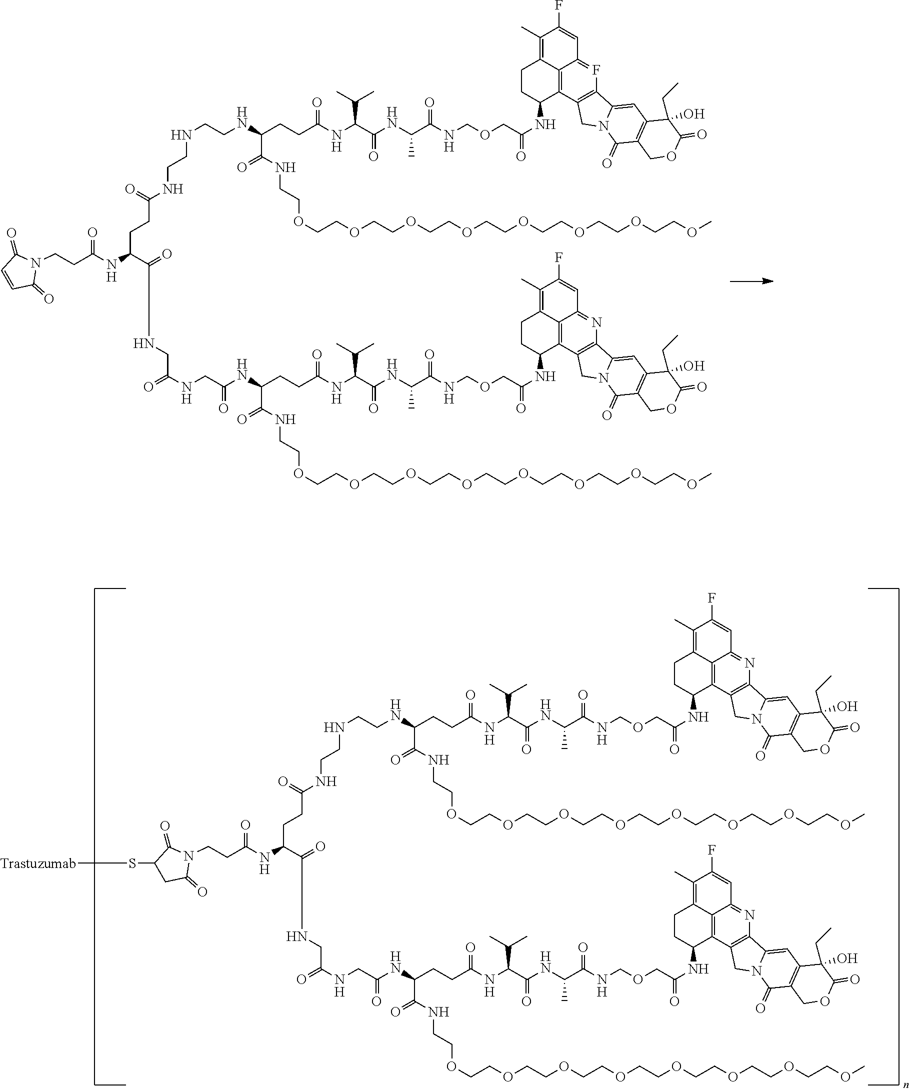

Reduction of antibodies: The concentration of a trastuzumab solution was adjusted to 10 mg/mL with an ABS buffer (pH 5.5); 60 μL of a TCEP solution (10 mM) was added to 1 mL of an antibody solution; then a small amount of Na₂HPO₄ solution (1 M) was added to adjust the pH of the reaction solution to 7.0; the reaction solution was incubated for 1 h in a water bath at 37° C., taken out and then cooled to room temperature for direct use in the next reaction.

Conjugation to antibodies: 8 mg small molecules (30 eq. with respect to the antibody) was dissolved with 200 uL of DMSO. The resultant was diluted with 500 uL of 10 mM ABS+5 mM EDTA buffer and added to a reaction tube. The tube was shaken by swirling for 0.5 h-1 h at room temperature (20-28° C.). 30 uL of 1M NAC solution was added to quench the reaction, and the tube was shaken by swirling for 20 min at room temperature (20-28° C.). Samples were collected and filtered with a sterile membrane (0.22 um), rinsed with a small amount of ABS+ EDTA buffer and then filtered.

Purification of drug-antibody conjugates: The above reactants were allowed to pass through a zeba desalted spin column (with the molecular weight cutoff of 10 KDa) to remove small molecules; the resulting target product solution was centrifuged and concentrated using an Amico Ultra (30,000 MWCO, Millipore Co.) vessel; the buffer was displaced; and the product solution was adjusted to ABS (pH 5.5) at a concentration of 4 mg/mL.

Evaluation of drug-antibody conjugate products: The product had a yield of 65% and was then measured in ultraviolet absorbance at 280 nm and at 370 nm, and with the molar extinction coefficients of the antibody and drug at 280 nm and 370 nm, respectively, it was calculated that the antibody of each molecule was linked to 15.88 drug molecules on average.

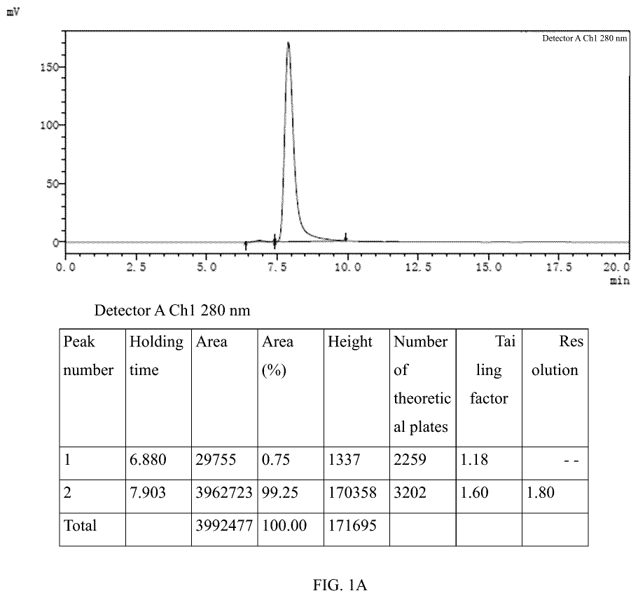

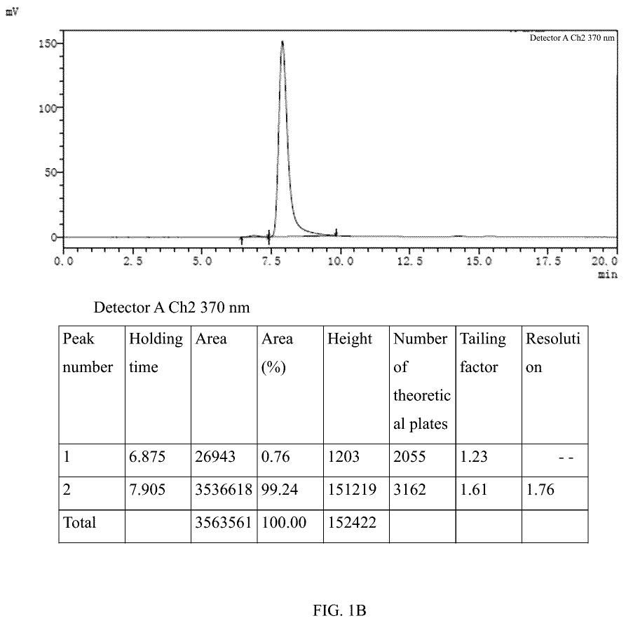

FIG. 1A to FIG. 1B show the polymer/lysate (SEC) charts of the of the antibody-drug conjugate Tr-1b of the present application.

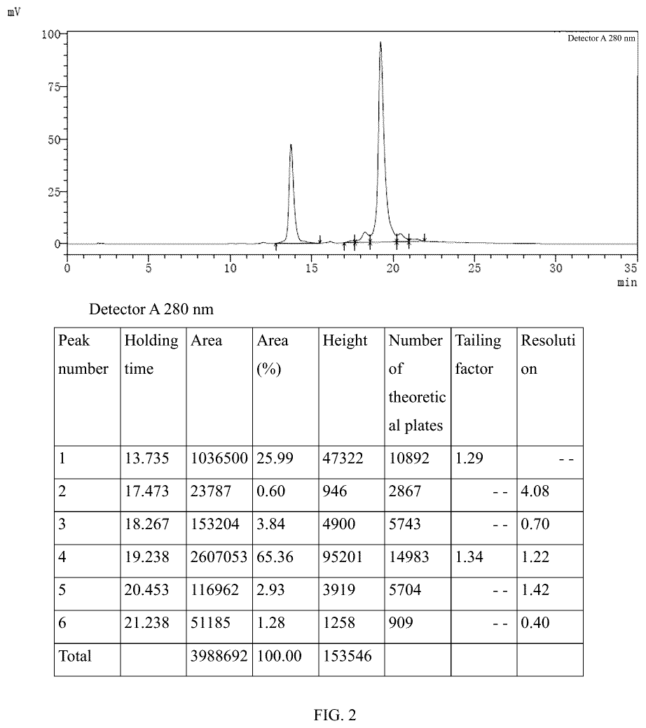

FIG. 2 shows the drug-antibody conjugation ratio (RPLC) chart of the antibody-drug conjugate Tr-1b of the present application.

### Example 12: Synthesis of Antibody-Drug Conjugate Tr-2a

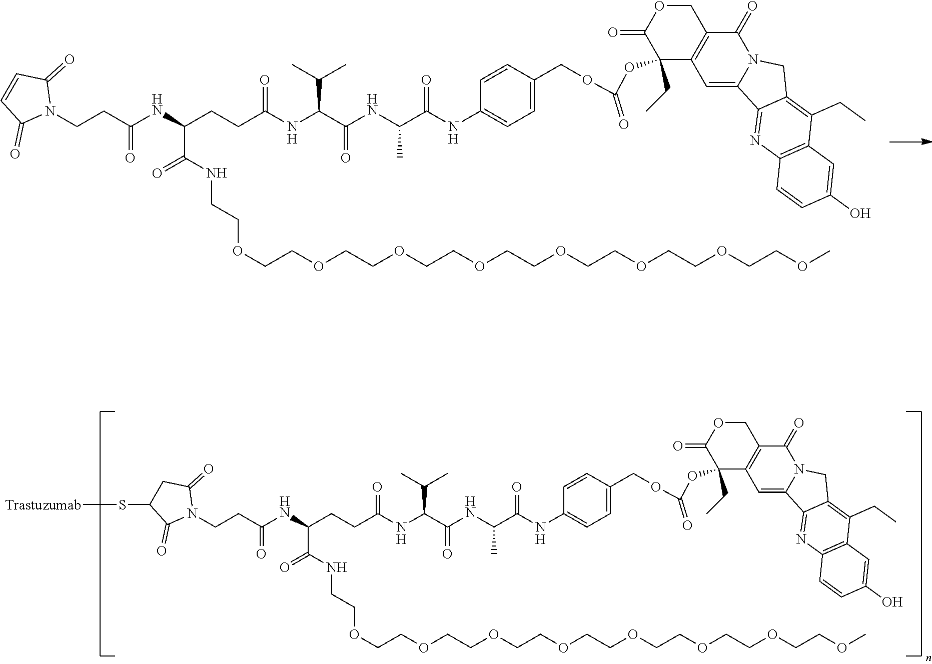

Reduction of antibodies: The concentration of a trastuzumab solution was adjusted to 10 mg/mL with an ABS buffer (pH 5.5); 60 μL of a TCEP solution (10 mM) was added to 1 mL of an antibody solution; then a small amount of Na₂HPO₄ solution (1 M) was added to adjust the pH of the reaction solution to 7.0; the reaction solution was incubated for 1 h in a water bath at 37° C., taken out and then cooled to room temperature for direct use in the next reaction.

Conjugation to antibodies: 10 mg small molecules (30 eq. with respect to the antibody) was dissolved with 200 uL of DMSO. The resultant was diluted with 500 uL of 10 mM ABS+5 mM EDTA buffer and added to a reaction tube. The tube was shaken by swirling for 0.5 h−1 h at room temperature (20-28° C.). 30 uL of 1M NAC solution was added to quench the reaction, and the tube was shaken by swirling for 20 min at room temperature (20-28° C.). Samples were collected and filtered with a sterile membrane (0.22 um), rinsed with a small amount of ABS+ EDTA buffer and then filtered.

Purification of drug-antibody conjugates: The above reactants were allowed to pass through a zeba desalted spin column (with the molecular weight cutoff of 10 KDa) to remove small molecules; the resulting target product solution was centrifuged and concentrated using an Amico Ultra (30,000 MWCO, Millipore Co.) vessel; the buffer was displaced; and the product solution was adjusted to ABS (pH 5.5) at a concentration of 4 mg/mL.

Evaluation of drug-antibody conjugate products: The product had a yield of 65% and was then measured in ultraviolet absorbance at 280 nm and at 370 nm, and with the molar extinction coefficients of the antibody and drug at 280 nm and 370 nm, respectively, it was calculated that the antibody of each molecule was linked to 7.86 drug molecules on average.

### Example 13: Synthesis of Antibody-Drug Conjugate Tr-2b

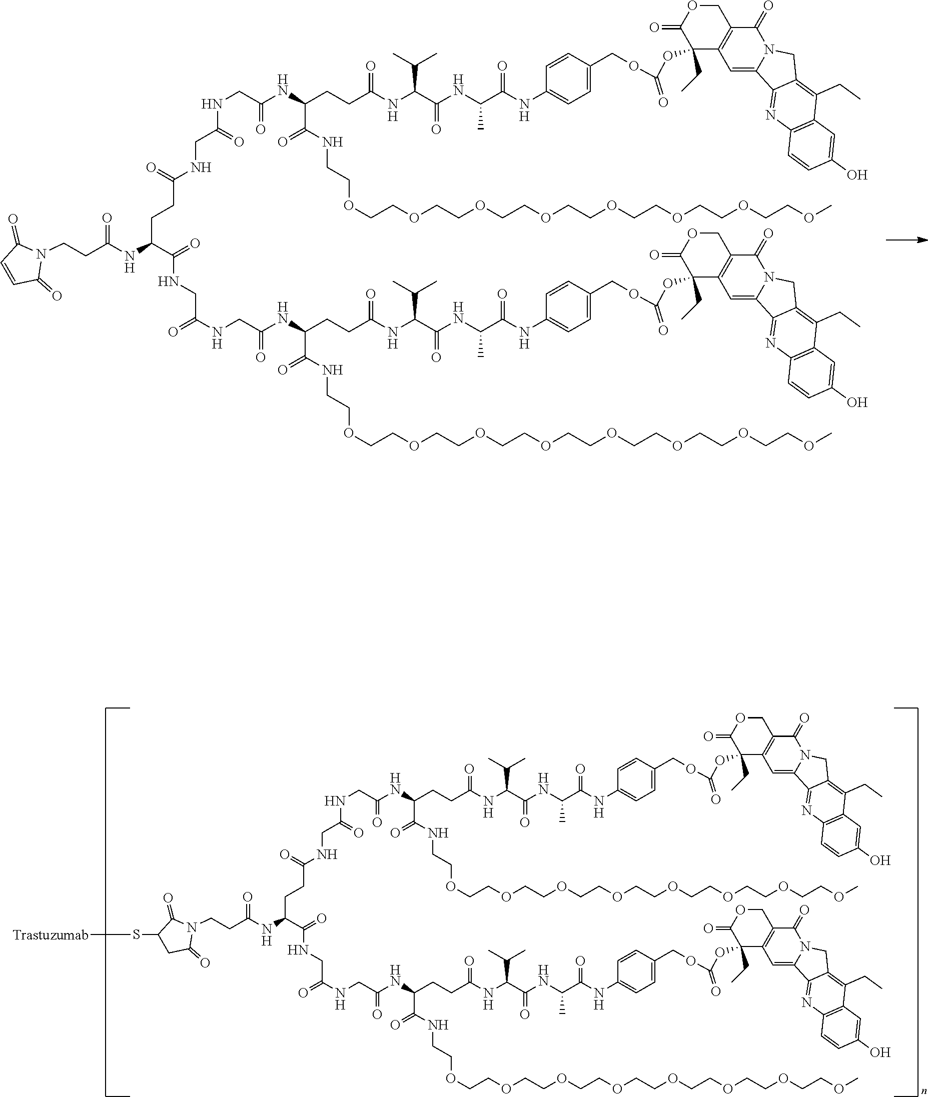

Reduction of antibodies: The concentration of a trastuzumab solution was adjusted to 10 mg/mL with an ABS buffer (pH 5.5); 60 μL of a TCEP solution (10 mM) was added to 1 mL of an antibody solution; then a small amount of Na₂HPO₄ solution (1 M) was added to adjust the pH of the reaction solution to 7.0; the reaction solution was incubated for 1 h in a water bath at 37° C., taken out and then cooled to room temperature for direct use in the next reaction.

Conjugation to antibodies: 10 mg small molecules (30 eq. with respect to the antibody) was dissolved with 200 uL of DMSO. The resultant was diluted with 500 uL of 10 mM ABS+5 mM EDTA buffer and added to a reaction tube. The tube was shaken by swirling for 0.5 h-1 h at room temperature (20-28° C.). 30 uL of 1M NAC solution was added to quench the reaction, and the tube was shaken by swirling for 20 min at room temperature (20-28° C.). Samples were collected and filtered with a sterile membrane (0.22 um), rinsed with a small amount of ABS+ EDTA buffer and then filtered.

Purification of drug-antibody conjugates: The above reactants were allowed to pass through a zeba desalted spin column (with the molecular weight cutoff of 10 KDa) to remove small molecules; the resulting target product solution was centrifuged and concentrated using an Amico Ultra (30,000 MWCO, Millipore Co.) vessel; the buffer was displaced; and the product solution was adjusted to ABS (pH 5.5) at a concentration of 4 mg/mL.

Evaluation of drug-antibody conjugate products: The product had a yield of 60% and was then measured in ultraviolet absorbance at 280 nm and at 370 nm, and with the molar extinction coefficients of the antibody and drug at 280 nm and 370 nm, respectively, it was calculated that the antibody of each molecule was linked to 15.82 drug molecules on average.

### Example 14: Synthesis of Antibody-Drug Conjugate Tr-3a

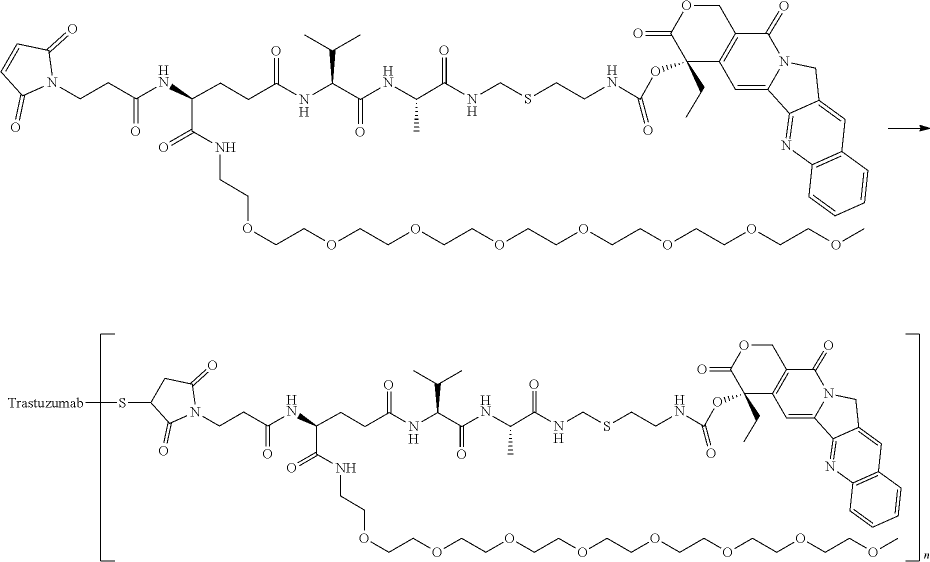

Reduction of antibodies: The reduction conditions for the antibodies were the same as those in Example 11.

Conjugation to antibodies: The conjugation conditions to the antibodies were the same as those in Example 11.

Purification of drug-antibody conjugates: The purification conditions were the same as those in Example 11.

Evaluation of drug-antibody conjugate products: The product had a yield of 60% and was then measured in ultraviolet absorbance at 280 nm and at 370 nm, and with the molar extinction coefficients of the antibody and drug at 280 nm and 370 nm, respectively, it was calculated that the antibody of each molecule was linked to 7.86 drug molecules on average.

### Example 15: Synthesis of Antibody-Drug Conjugate Tr-3b

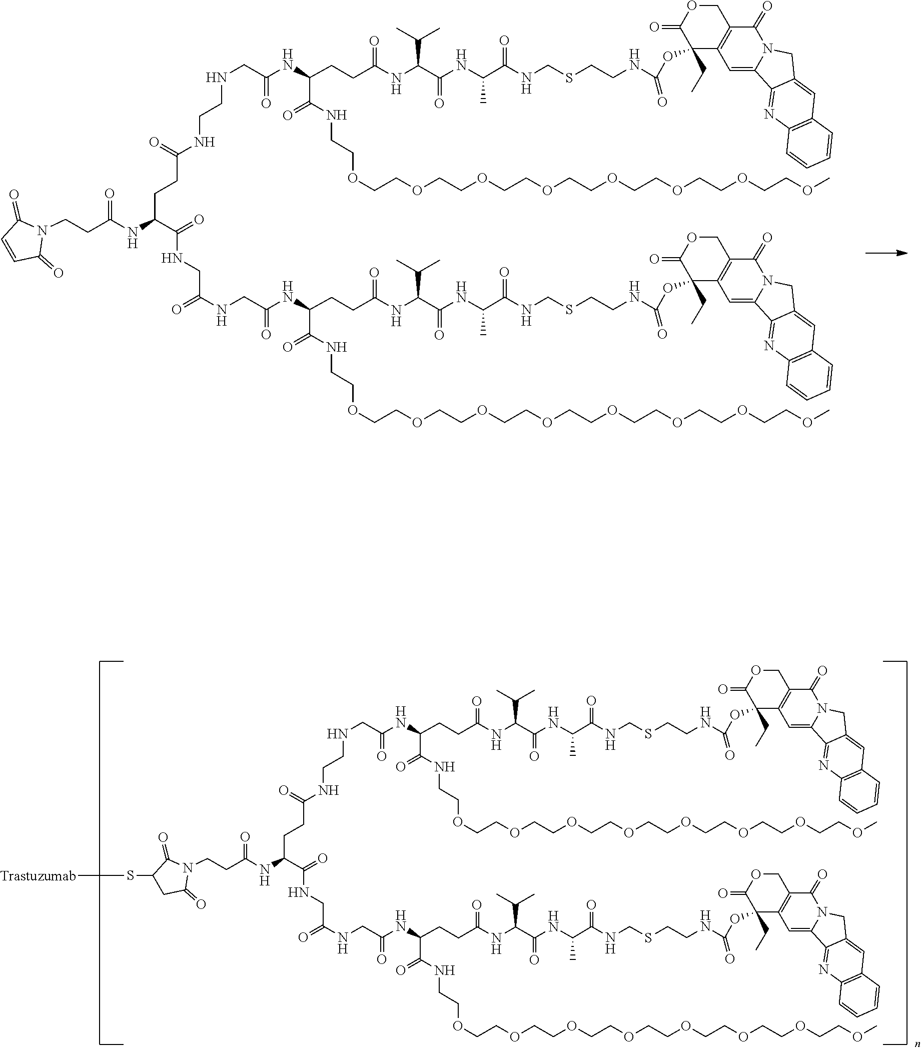

Reduction of antibodies: The reduction conditions for the antibodies were the same as those in Example 11.

Conjugation to antibodies: The conjugation conditions to the antibodies were the same as those in Example 11.

Purification of drug-antibody conjugates: The purification conditions were the same as those in Example 11.

Evaluation of drug-antibody conjugate products: The product had a yield of 72% and was then measured in ultraviolet absorbance at 280 nm and at 370 nm, and with the molar extinction coefficients of the antibody and drug at 280 nm and 370 nm, respectively, it was calculated that the antibody of each molecule was linked to 15.86 drug molecules on average.

### Example 16: Synthesis of Antibody-Drug Conjugate Tr-4

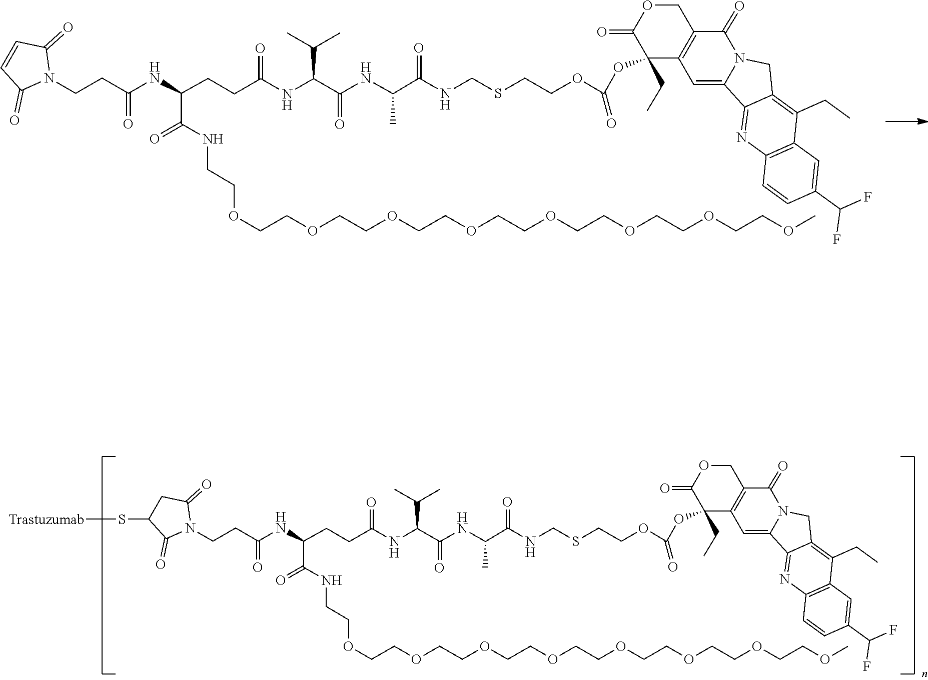

Reduction of antibodies: The reduction conditions for the antibodies were the same as those in Example 11.

Conjugation to antibodies: The conjugation conditions to the antibodies were the same as those in Example 11.

Purification of drug-antibody conjugates: The purification conditions were the same as those in Example 11.

Evaluation of drug-antibody conjugate products: The product had a yield of 50% and was then measured in ultraviolet absorbance at 280 nm and at 370 nm, and with the molar extinction coefficients of the antibody and drug at 280 nm and 370 nm, respectively, it was calculated that the antibody of each molecule was linked to 7.64 drug molecules on average.

### Example 17: Synthesis of Antibody-Drug Conjugate Sa-1b

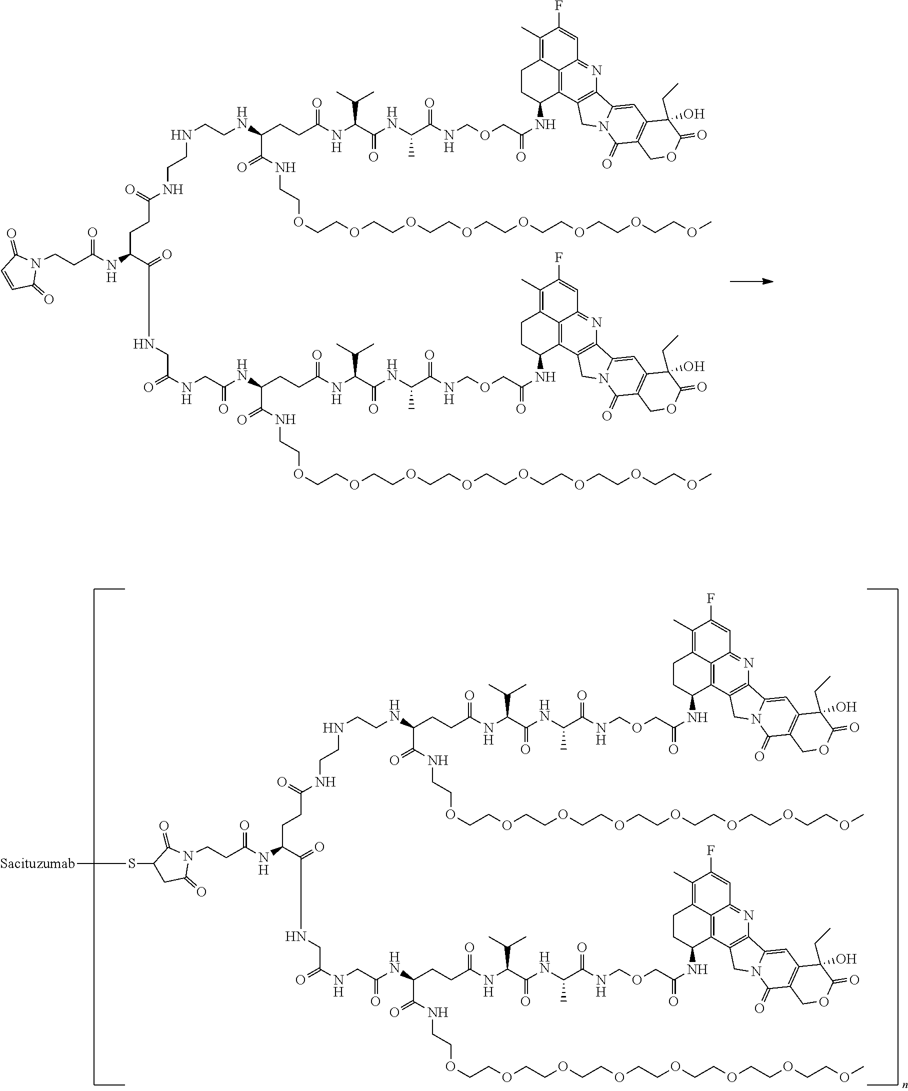

Reduction of antibodies: Sacituzumabs (anti-TROP2 antibodies) were used, and the reduction conditions for the antibodies were the same as those in Example 11.

Conjugation to antibodies: The conjugation conditions to the antibodies were the same as those in Example 11.

Purification of drug-antibody conjugates: The purification conditions were the same as those in Example 11.

Evaluation of drug-antibody conjugate products: The product had a yield of 61% and was then measured in ultraviolet absorbance at 280 nm and at 370 nm, and with the molar extinction coefficients of the antibody and drug at 280 nm and 370 nm, respectively, it was calculated that the antibody of each molecule was linked to 15.88 drug molecules on average.

### Example 18: Synthesis of Antibody-Drug Conjugate Be-1b

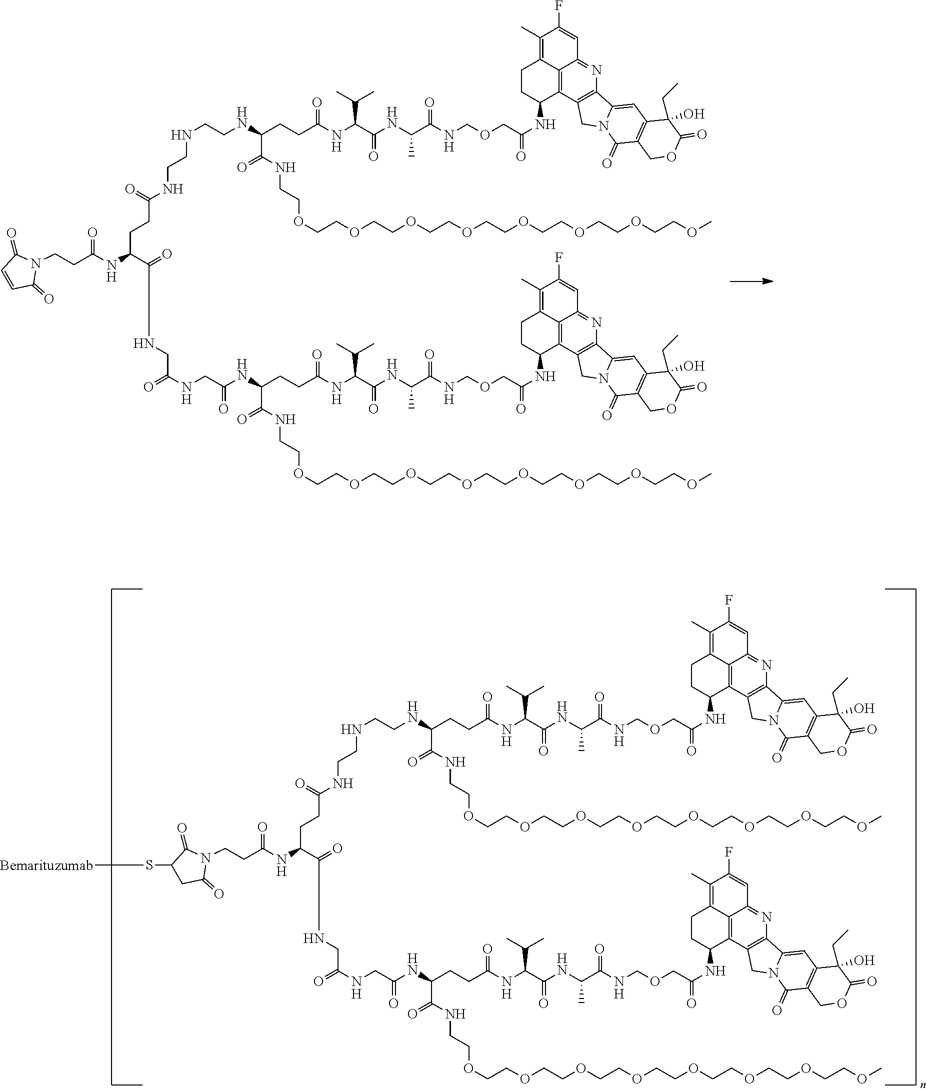

Reduction of antibodies: Bemarituzumabs (anti-FGFR2b antibodies) were used, and the reduction conditions for the antibodies were the same as those in Example 11.

Conjugation to antibodies: The conjugation conditions to the antibodies were the same as those in Example 11.

Purification of drug-antibody conjugates: The purification conditions were the same as those in Example 11.

Evaluation of drug-antibody conjugate products: The product had a yield of 61% and was then measured in ultraviolet absorbance at 280 nm and at 370 nm, and with the molar extinction coefficients of the antibody and drug at 280 nm and 370 nm, respectively, it was calculated that the antibody of each molecule was linked to 15.92 drug molecules on average.

### Example 19: Detection of Endocytosis and Activity of Antibody-Drug Conjugate Tr-1b

The tumor strains used in this test were as follows:

| | | Growth | |
|---|---|---|---|
| Cell line | Cell line type | characteristics | Complete medium |
| NCI-N87 | Gastric cancer | Adherent | RPMI-1640 + 10% |
| | cells | | FBS |
| SK-BR-3 | Breast cancer | Adherent | RPMI-1640 + 10% |
| | cells | | FBS |

Test samples: Ab1: DS-8201 (Enhertu, fam-trastuzumab deruxtecan-nxki); Ab2: antibody-drug conjugate Tr-1b.

### 19.1 Endocytosis Test

In the endocytosis test, resuscitated cells were detected for the fluorescence density at time points 0 h, 0.5 h, 1 h, and 2 h after dosing, and the endocytosis rate was calculated. The results were as follows:

### 1) SK-BR-3 Cells

Detected values of HB0321a fluorescence density for endocytosis at each time point

| | Binding | 37° C. | 37° C. | 37° C. | 37° C. |
|---|---|---|---|---|---|
| Group | Control Group | 0 h | 0.5 h | 1 h | 2 h |
| Ab1 | 353 | 12.8 | 31.5 | 41.6 | 64.8 |
| Ab2 | 315 | 11.5 | 22.8 | 28.3 | 48.6 |
| Herceptin | 384 | 12.7 | 26.3 | 35.9 | 99 |
| IgG1 | 15.8 | 6.02 | 6.23 | 6.45 | 6.36 |

TABLE 1
Endocytosis rate of SK-BR-3 cells at each time point

| Endocytosis rate (%) | 37° C. 0.5 h | 37° C. 1 h | 37° C. 2 h |
|---|---|---|---|
| Ab1 | 5.62 | 8.65 | 15.62 |
| Ab2 | 3.83 | 5.69 | 12.58 |
| Herceptin | 3.74 | 6.37 | 23.71 |

### 2) NCI-N87 Cells

Detected values of HB0321a fluorescence density for endocytosis at each time point

| | Binding | 37° C. | 37° C. | 37° C. | 37° C. |
|---|---|---|---|---|---|
| Group | Control Group | 0 h | 0.5 h | 1 h | 2 h |
| Ab1 | 588 | 22.4 | 47.1 | 57.7 | 127 |
| Ab2 | 579 | 22.3 | 32.9 | 62.3 | 85 |
| Herceptin | 763 | 23.7 | 41.3 | 86.8 | 94.0 |
| IgG1 | 34.0 | 8.09 | 8.53 | 8.89 | 8.35 |

TABLE 2
Endocytosis rate of NCI-N87 cells at each time point

| Endocytosis rate (%) | 37° C. 0.5 h | 37° C. 1 h | 37° C. 2 h |
|---|---|---|---|
| Ab1 | 4.28 | 6.11 | 18.12 |
| Ab2 | 1.87 | 7.04 | 11.03 |
| Herceptin | 2.34 | 8.39 | 9.34 |

According to the results shown in Tables 1-2, both SK-BR-3 cells and NCI-N87 cells show good endocytosis effects on the antibody-drug conjugate Tr-1b of the present application.

### 19.2 Cell Proliferation Inhibition Test

Viability assay: The resuscitated cells were cultured for 6 days after dosing, cell proliferation results were detected, and IC50 values were calculated. The details were as follows:
a) all cell lines were cultured in the complete medium at 37° C. with 5% CO₂;
b) cells in the logarithmic growth phase were harvested and then counted using a platelet counter, and the cell viability was tested by the trypan blue rejection method to ensure that the cell viability was above 90%;
c) the cell density was adjusted using the complete medium and then seeded on a 96-well cell culture plate, with 90 μL per well for a total of 3000 cells;
d) the cells in the 96-well plate were cultured at 37° C. with 5% CO₂;
e) a 10-fold drug solution was prepared, the test samples HB01, HB02 and HB03 had the working solution of 20,000 ng/ml and diluted 3.16-fold for 9 concentrations, the test samples HB04 and HB05 had the working solution of 10 μM and diluted 3.16-fold for 9 concentrations, and then the serial diluted products (10 μL for each) were transferred to the corresponding experimental wells of the 96-well cell plate, with three duplicate wells for each drug concentration;
f) the dosed cells in the 96-well plate were cultured at 37° C. with 5% CO₂ for another 6 days, and then subjected to CTG analysis; and
g) the CTG reagent was thawed, and the cell plate was equilibrated to room temperature for 30 min.
h) An equal volume of CTG solution was added per well.
i) The cells were shaken on an orbital shaker for 5 min for lysis.
j) The cell plate was treated at room temperature for 20 min to stabilize the cold light signal.
k) The cold light values were read to collect data.

The data were analyzed using software GraphPad Prism 7.0, and fitted by the nonlinear S-curve regression to obtain a dose-effect curve, based on which the IC50 value was calculated.

$$Cell survival rate ( \% ) = ( Lum test drug - Lum culture medium control ) / ( Lum cell control - Lum culture medium control ) \times 100 \% .$$

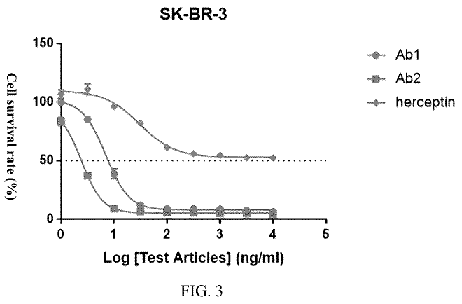

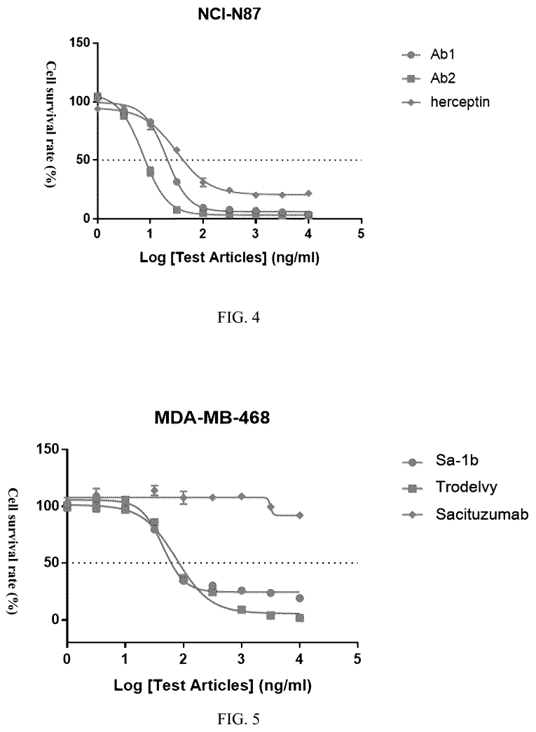

According to the results shown in Table 3 and FIG. 3 to FIG. 4, that the antibody-drug conjugate Tr-1b of the present application show a good killing effect on both SK-BR-3 and NCI-N87 cancer cells, and can effectively inhibit the proliferation of cancer cells.

### Example 3: IC50s of Antibody-Drug Conjugate Tr-1b for Cells

IC50 (ng/ml)

| No. | Sample name | SK-BR-3 | NCI-N87 |
|---|---|---|---|
| 1 | Ab1 | 6.95 | 20.11 |
| 2 | Ab2 | 2.39 | 7.23 |
| 3 | Herceptin | 30.51 | 30.96 |

In summary, the antibody-drug conjugate Tr-1b of the present application has a significant proliferation inhibition effect on both the humanized breast cancer SK-BR-3 and the humanized gastric cancer NCI-N87. Moreover, its effect is better than that of DS-8201 and has a tumor suppression activity about 3 times that of DS-8201.

### Example 20: Detection of Activity of Antibody-Drug Conjugate Sa-1b

MDA-MB-468 cells (human triple-negative breast cancer cells), SNU216 cells (human gastric cancer cells), NCI-H1650 cells (human non-small cell lung adenocarcinoma cells), and NCI-H596 cells (human lung cancer cells), purchased from ATTC, were used to evaluate the inhibitory activity of Sa-1b against tumor proliferation. All the cells were those with high expression of Trop2 antigens. Trodelvy was the tradename of sacituzumab govitecan injector. Sacituzumab was an outsourced anti-Trop2 antibody.

All cell lines were cultured in the complete medium at 37° C. with 5% CO₂. Cells in the logarithmic growth phase were harvested and then counted using a platelet counter, and the cell viability was tested by the trypan blue rejection method to ensure that the cell viability was above 90%. The cell density was adjusted using the complete medium and then seeded on a 96-well cell culture plate, with 180 μL per well for a total of 1,000 cells. The cells in the 96-well plate were cultured at 37° C. with 5% CO2. A 10-fold drug solution was prepared; a test sample with a working concentration of 10,000 ng/ml was diluted 3.16-fold for 9 concentrations; and then, the serial diluted products (20 μL for each) were transferred to the corresponding experimental wells of the 96-well cell plate, with three duplicate wells for each drug concentration. The cells from the dosed 96-well plate were cultured at 37° C. with 5% CO2 for another 6 days, followed by CTG analysis (CTG, a CellTiter-Glo kit, was a homogeneous cell viability assay for quantifying ATP to determine the viability of cultured cells).

The CTG reagent was thawed, and the cell plate was equilibrated to room temperature for 30 min. 100 μL of CTG solution was added per well. The cells were shaken on an orbital shaker for 5 min for lysis. The cell plate was treated at room temperature for 20 min to stabilize the cold light signal. The cold light values were read to collect data.

The data were analyzed using software GraphPad Prism 7.0, and fitted by the nonlinear S-curve regression to obtain a dose-effect curve, based on which the IC50 value was calculated.

$$Cell survival rate ( \% ) = ( Lum test drug - Lum culture medium control ) / ( Lum cell control - Lum culture medium control ) \times 100 \%$$

FIG. 5 shows that, for MDA-MB-468 cells, Sa-1b has an efficacy slightly better than that of Trodelvy, its IC50 is about 1/7 times that of Trodelvy, and both Sa-1b and Trodelvy are superior to Sacituzumab.

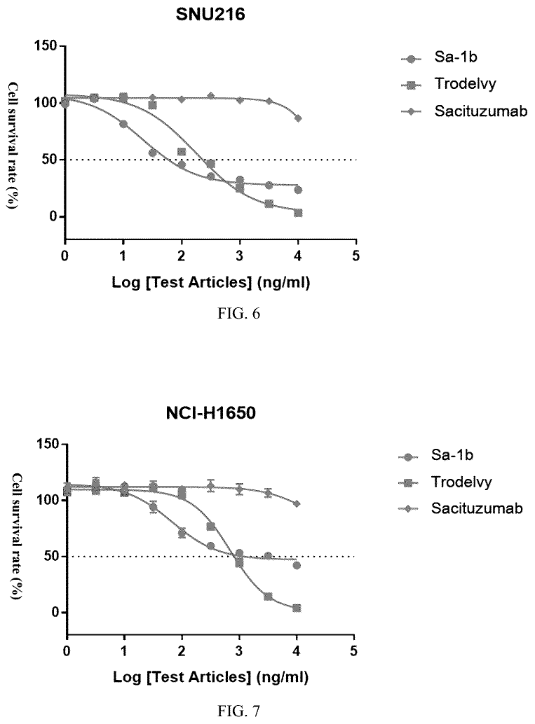

FIG. 6 shows that, for SNU216 cells, Sa-1b has an efficacy significantly better than that of Trodelvy, its IC50 is about 1/8.2 times that of Trodelvy, and both Sa-1b and Trodelvy are superior to Sacituzumab.

FIG. 7 shows that, for NCI-H1650 cells, Sa-1b has an efficacy significantly better than that of Trodelvy, its IC50 is about ⅙ times that of Trodelvy, and both Sa-1b and Trodelvy are superior to Sacituzumab.

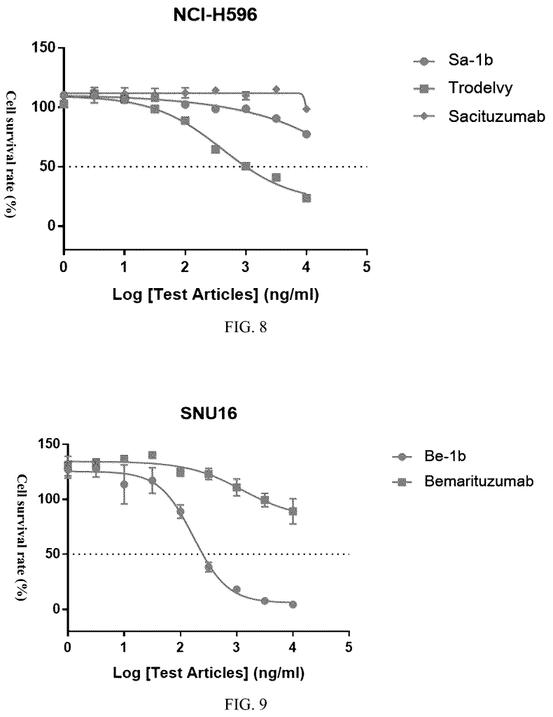

FIG. 8 shows that, for NCI-H596 cells, Sa-1b has an efficacy significantly weaker than that of Trodelvy and is almost ineffective and similar to that of Sacituzumab, and Trodelvy has certain efficacy.

### Example 21: Detection of Activity of Antibody-Drug Conjugate Be-1b

The activity of BE-1b to inhibit tumor proliferation was evaluated by using SNU16 cells (human gastric cancer cells) and OCUM-2M cells (human gastric cancer cells), purchased from ATTC, with high expression FGFR2b antigens were used to evaluate the inhibitory activity of Be-ab against tumor proliferation. Bemarituzumab was an outsourced anti-FGFR2b antibody.

All cell lines were cultured in the complete medium at 37° C. with 5% CO₂. Cells in the logarithmic growth phase were harvested and then counted using a platelet counter, and the cell viability was tested by the trypan blue rejection method to ensure that the cell viability was above 90%. The cell density was adjusted using the complete medium and then seeded on a 96-well cell culture plate, with 180 μL per well for a total of 1,000 cells. The cells in the 96-well plate were cultured at 37° C. with 5% CO₂. A 10-fold drug solution was prepared; a test sample with a working concentration of 10,000 ng/ml was diluted 3.16-fold for 9 concentrations; and then, the serial diluted products (20 μL for each) were transferred to the corresponding experimental wells of the 96-well cell plate, with three duplicate wells for each drug concentration. The cells from the dosed 96-well plate were cultured at 37° C. with 5% CO2 for another 6 days, followed by CTG analysis (CTG, a CellTiter-Glo kit, was a homogeneous cell viability assay for quantifying ATP to determine the viability of cultured cells).

The CTG reagent was thawed, and the cell plate was equilibrated to room temperature for 30 min. 100 μL of CTG solution was added per well. The cells were shaken on an orbital shaker for 5 min for lysis. The cell plate was treated at room temperature for 20 min to stabilize the cold light signal. The cold light values were read to collect data.

The data were analyzed using software GraphPad Prism 7.0, and fitted by the nonlinear S-curve regression to obtain a dose-effect curve, based on which the IC50 value was calculated.

$$Cell survival rate ( \% ) = ( Lum test drug - Lum culture medium control ) / ( Lum cell control - Lum culture medium control ) \times 100 \%$$

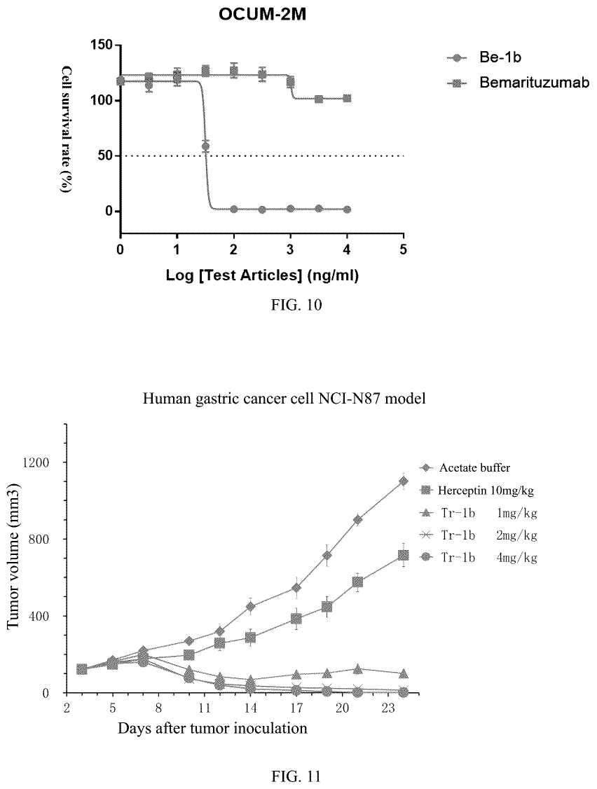

According to the results shown in FIG. 9 and FIG. 10, for SNU16 cells and OCUM-2M cells, Be-1b has an effect significantly better than that of Bemarituzumab, which is almost ineffective, indicating that the antibody-drug conjugate of the present application has a good tumor-killing effect on targeted cells.

### Example 22: Detection of In Vivo Activity of Antibody-Drug Conjugate Tr-1b

### 22.1 Human Gastric Cancer Cell NCI-N87 Model

Mice: female nude mice of 6-8 weeks old (species: Mus Musculus, BALB/c nude; SpePharm (Beijing) Biotechnology Co., Ltd.) for experiments.

The human gastric cancer strain NCI-N87 cells purchased from ATCC were suspended in normal saline, and inoculated subcutaneously with 1×10⁷ cells (human gastric cancer cell NCI-N87) to the right body side of female nude mice (Day 0); at the time point when the tumor volume reached 80-120 mm³, the mice were randomly divided into 5 groups according to the tumor volume, namely, a control group, a Herceptin (the tradename of trastuzumab) group, and Tr-1b low-, medium- and high-dose groups; and the mice were administrated once via the tail vein at an injection volume of 10 mL/kg. The dose administered in each group was as follows: acetic acid buffer administrated to the blank control group; 10 mg/kg dosed to the Herceptin group; and 1 mg/kg, 2 mg/kg, and 4 mg/kg for the low-, medium- and high-dose groups of TR-1b, respectively.

Measurement and calculation of tumor volume:

The tumor volume (mm³) was calculated by measuring the long diameter and short diameter of the tumor twice a week with calipers.

$$Calculation formula : tumor volume ( {mm}^{3} ) = 0.52 \times length diameter ( mm ) \times {[ short diameter ( mm ) ]}^{2}$$

As shown in FIG. 11, for the human gastric cancer cell NCI-N87 model, Hercepin and Tr-1b at each dose show an inhibitory effect against tumor proliferation when compared with the control group, and the tumor inhibition effect of Tr-1b is significantly better than that of Herceptin. The tumor inhibition effect of TR-1b at the high dose was stronger than that of the medium dose, which was stronger than that of the low dose, showing a significant dose-effect relationship.

### 22.2 Human Breast Cancer BT474 Model:

Mice: female nude mice of 6-9 weeks old (species: Mus Musculus, CB-17 SCID; SpePharm (Beijing) Biotechnology Co., Ltd.) for experiments.

The human breast cancer BT474 cells purchased from ATCC were suspended in normal saline, and inoculated subcutaneously with 5×10⁶ cells (human breast cancer BT474 cells) to the right body side of female nude mice (Day 0); at the time point when the tumor volume reached 80-120 mm³, the mice were randomly divided into 5 groups according to the tumor volume, namely, a control group, a Herceptin (the tradename of trastuzumab) group, and Tr-1b low-, medium- and high-dose groups; and the mice were administrated once via the tail vein at an injection volume of 10 mL/kg. The dose administered in each group was as follows: acetic acid buffer administrated to the blank control group; 10 mg/kg dosed to the Herceptin group; and 5 mg/kg, 10 mg/kg, and 15 mg/kg for the low-, medium- and high-dose groups of TR-1b, respectively.

Measurement and calculation of tumor volume:

The tumor volume (mm³) was calculated by measuring the long diameter and short diameter of the tumor twice a week with calipers.

Calculation formula: tumor volume (mm³)=0.52×length diameter (mm)×[short diameter (mm)]²

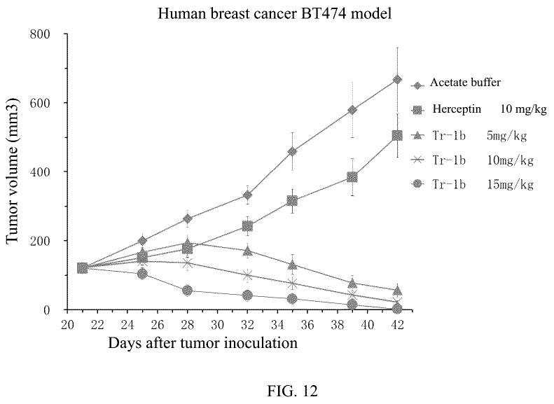

As shown in FIG. 12, for the model of human breast cancer NCI-N87, Hercepin and Tr-1b at each dose show an inhibitory effect against tumor proliferation when compared with the control group, and the tumor inhibition effect of Tr-1b is significantly better than that of Herceptin. The tumor inhibition effect of TR-1b at the high dose was stronger than that of the medium dose, which was stronger than that of the low dose, showing a significant dose-effect relationship.

1. A linker comprising an Lb structure fragment,
wherein the Lb structure fragment is selected from the following formula: 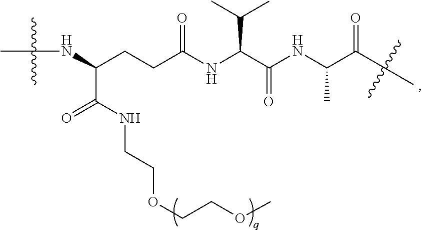
and
wherein q is any integer selected from 1-20.

2. The linker according to claim 1, further comprising
an Lb-Lc structure fragment,
wherein the Lb structure fragment is linked to an Lc fragment via a carbonyl group,
wherein the Lc fragment is a releasable assembly unit and is capable of linking a drug unit.

3. The linker according to claim 2, wherein the Lc fragment is selected from the following formulas:

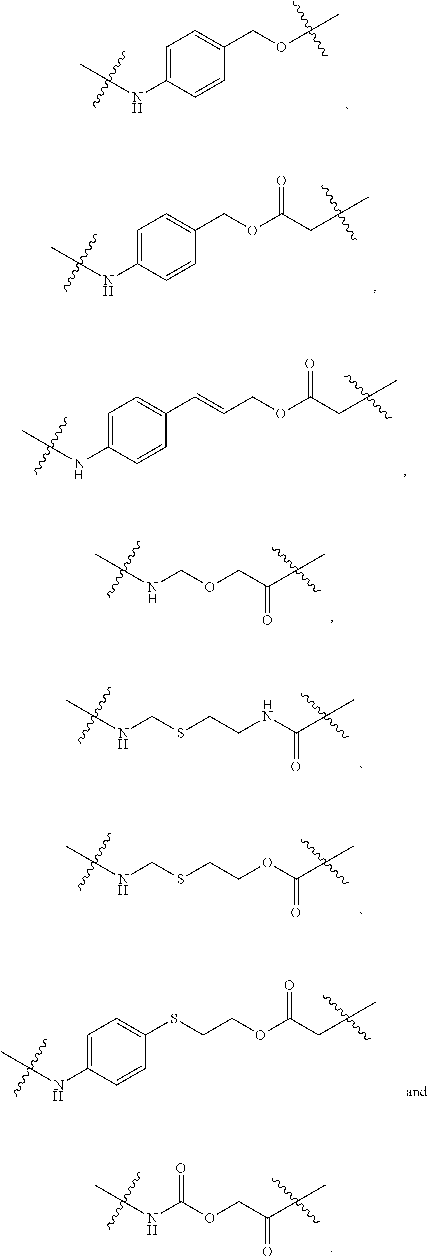

4. The linker according to claim 2, wherein the linker has a structure as shown in Formula (I):
La—Bₚ [symbol] Lb-Lc)ₘ Formula (I),
wherein La is an extension unit, being capable of linking to a ligand unit,
wherein B is an optional branching unit,
wherein p is 0 or 1,
wherein m is 1 when p is 0,
wherein m is 2, 3, or 4 when p is 1,
wherein the Lb structure fragment is selected from the following formulas: 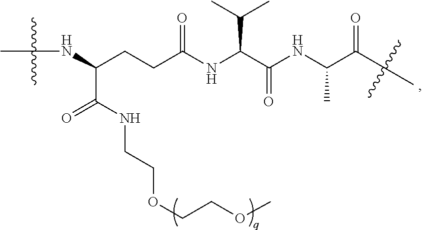
wherein q is any integer selected from 1-20,
wherein the Lb structure fragment is linked to La or B via an amino group and to the Lc fragment via a carbonyl group,
wherein the Lc fragment is a releasable assembly unit,
wherein the Lc fragment is linked to the carbonyl group of the Lb structure fragment via an amino group, and
wherein the Lc fragment is capable of linking the drug unit via a carbonyl group.

5. The linker according to claim 4, wherein B is selected from the following formula: 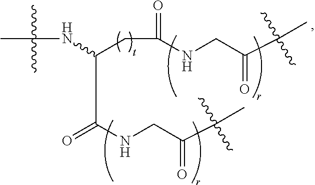
wherein t is any integer independently selected from 1-4,
wherein r is any integer independently selected from 1-4, and
wherein B is linked to La via an amino group and to the Lb structure fragment via a carbonyl group.

6. (canceled)

7. The linker according to claim 4, wherein La comprises a maleimide type linker fragment.

8. The linker according to claim 7, wherein La has the following structure: 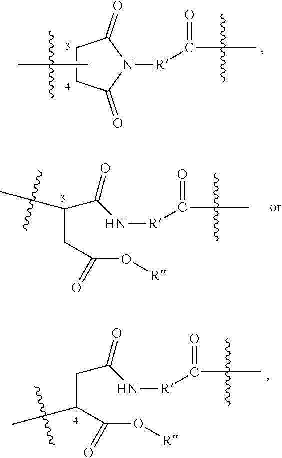
wherein La is linked to the Lb structure fragment or B via a carbonyl group, and is capable of linking to the ligand unit via position(s) 3 and/or 4 of succinimide/succinimide,
wherein R′ is selected from the group consisting of an optionally substituted C₁-C₁₀ alkyl, a C₁-C₁₀ alkoxy, a C₁-C₁₀ aminoalkyl and C₁-C₁₀ alkyl-aryl, and a 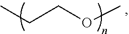
wherein R″ is selected from the group consisting of an optionally substituted C₁-C₁₀ alkyl,
wherein a substituent is selected from the group consisting of an amino group, a halogen, a nitro group, a hydroxyl group, an acetyl group, a cyano group, a C₁-C₁₀ alkyl, a C₁-C₁₀ alkoxy, a C₁-C₁₀ aminoalkyl, a C₁-C₁₀ haloalkyl, a C₂-C₁₀ vinyl, a C₂-C₁₀ alkynyl, an amide group, a C₃-C₈ cycloalkyl, and a C₃-C₈ heterocycloalkyl, and
wherein n is any integer selected from 1-10.

9. The linker according to claim 4, wherein La is selected from the following formulas: 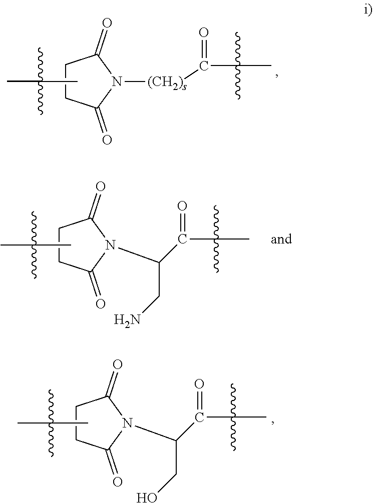
wherein La is linked to the Lb structure fragment or B via a carbonyl group, and is capable of linking the ligand unit via position(s) 3 and/or 4 of succinimide; 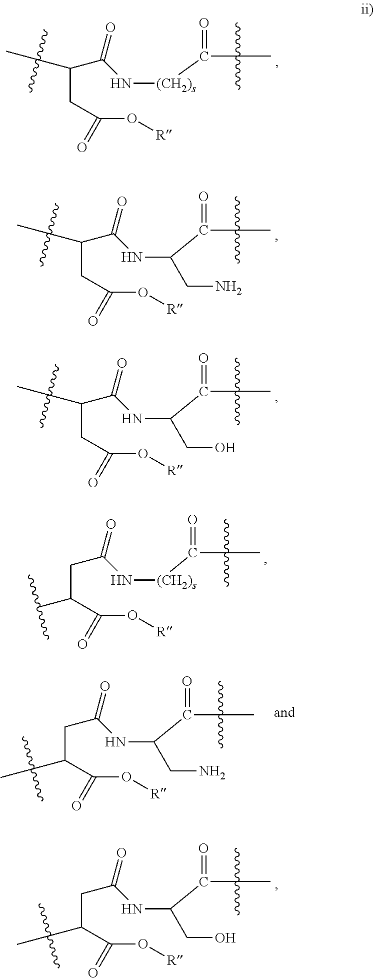
wherein La is linked to the Lb structure fragment or B via a carbonyl group, and is capable of linking the ligand unit via position(s) 3 and/or 4 of succinamide;
wherein R″ is selected from the group consisting of a methyl group, an ethyl group, and a propyl group;
wherein s is any integer independently selected from 1-10.

10. The linker according to claim 4, wherein La is selected from the following formula: 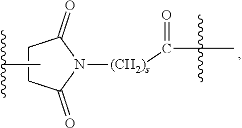
wherein La is linked to the Lb structure fragment or B via a carbonyl group, and is capable of linking the ligand unit via position(s) 3 and/or 4 of succinimide, and
wherein s is any integer selected from 1-10.

11. The linker according to claim 4, wherein La is selected from the following formulas: 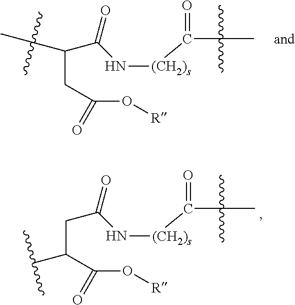
wherein R″ is selected from the group consisting of a methyl group, an ethyl group, and a propyl group,
wherein s is any integer selected from 1-10, and
wherein La is linked to B or the Lb structure fragment via a carbonyl group.

12. (canceled)

13. The linker according to claim 1, wherein the linker is selected from the following structures: 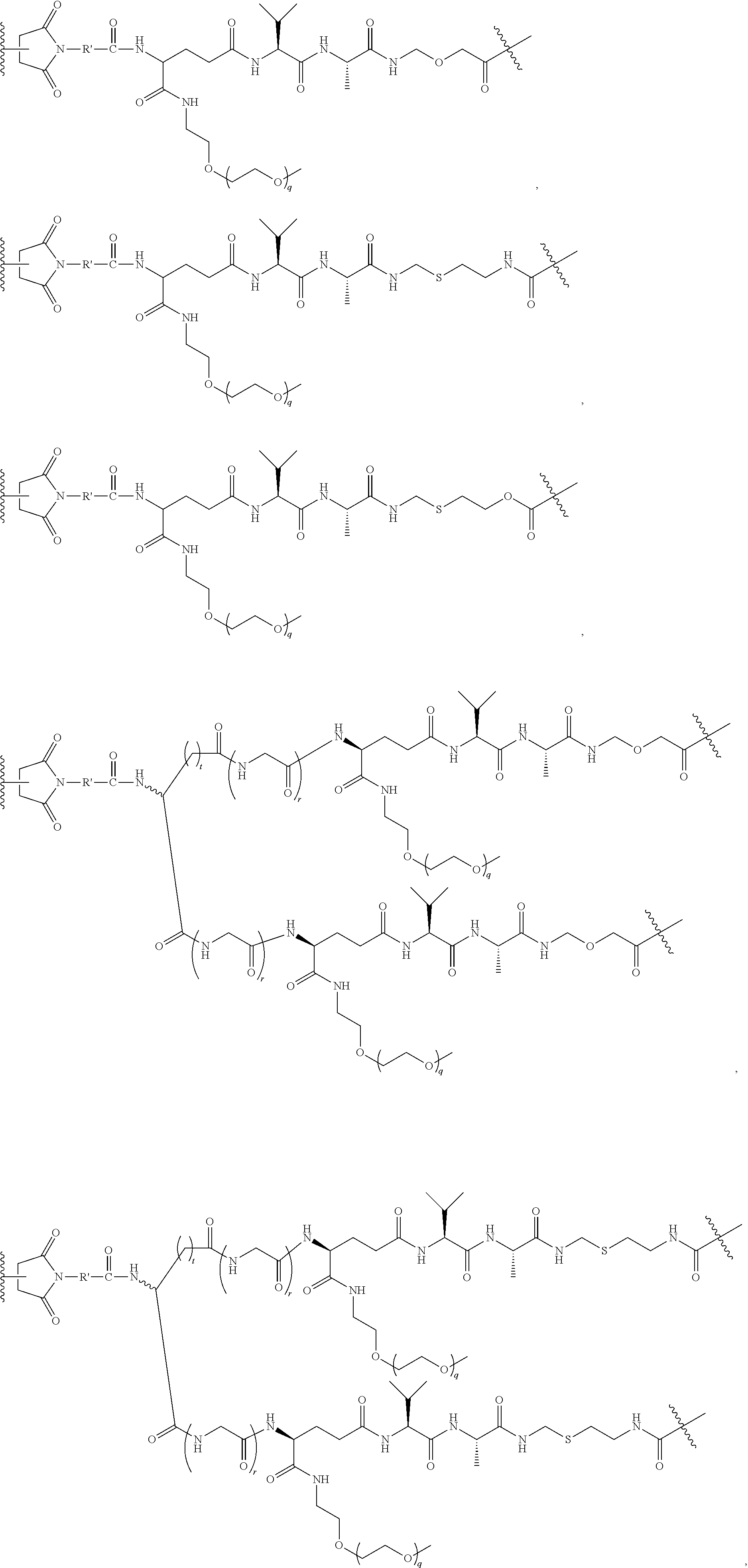 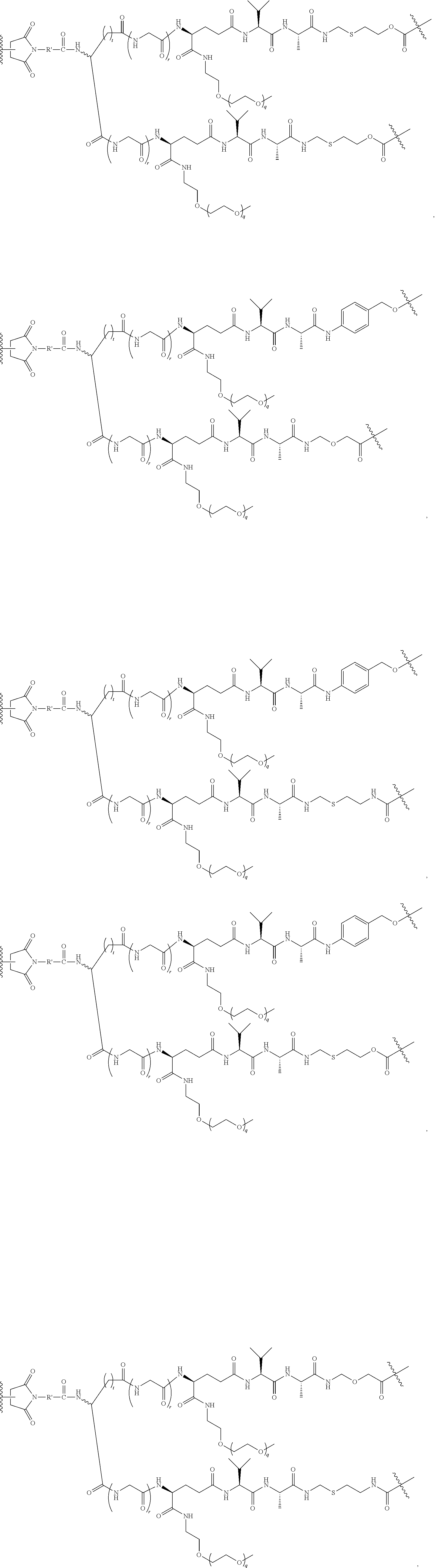 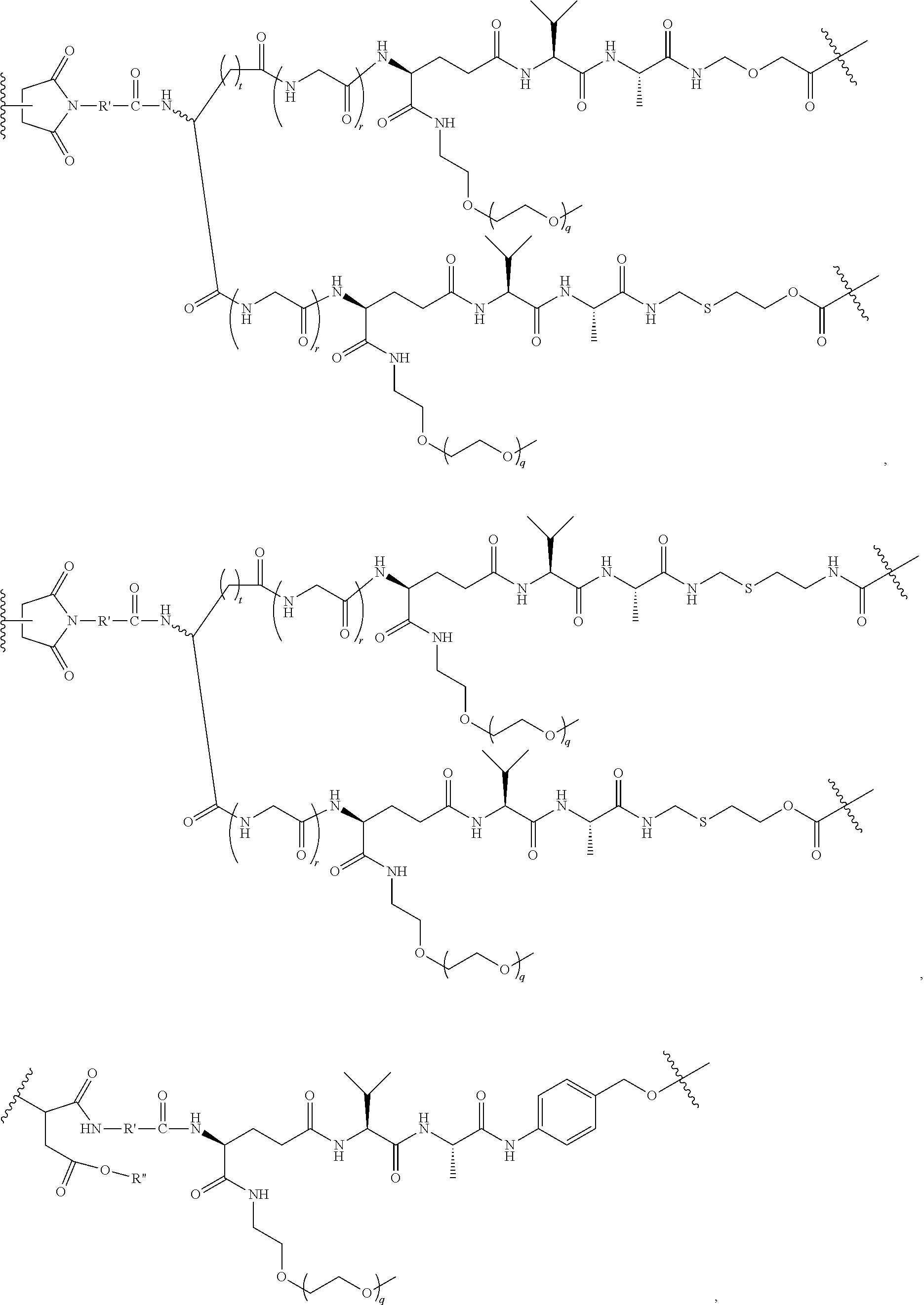 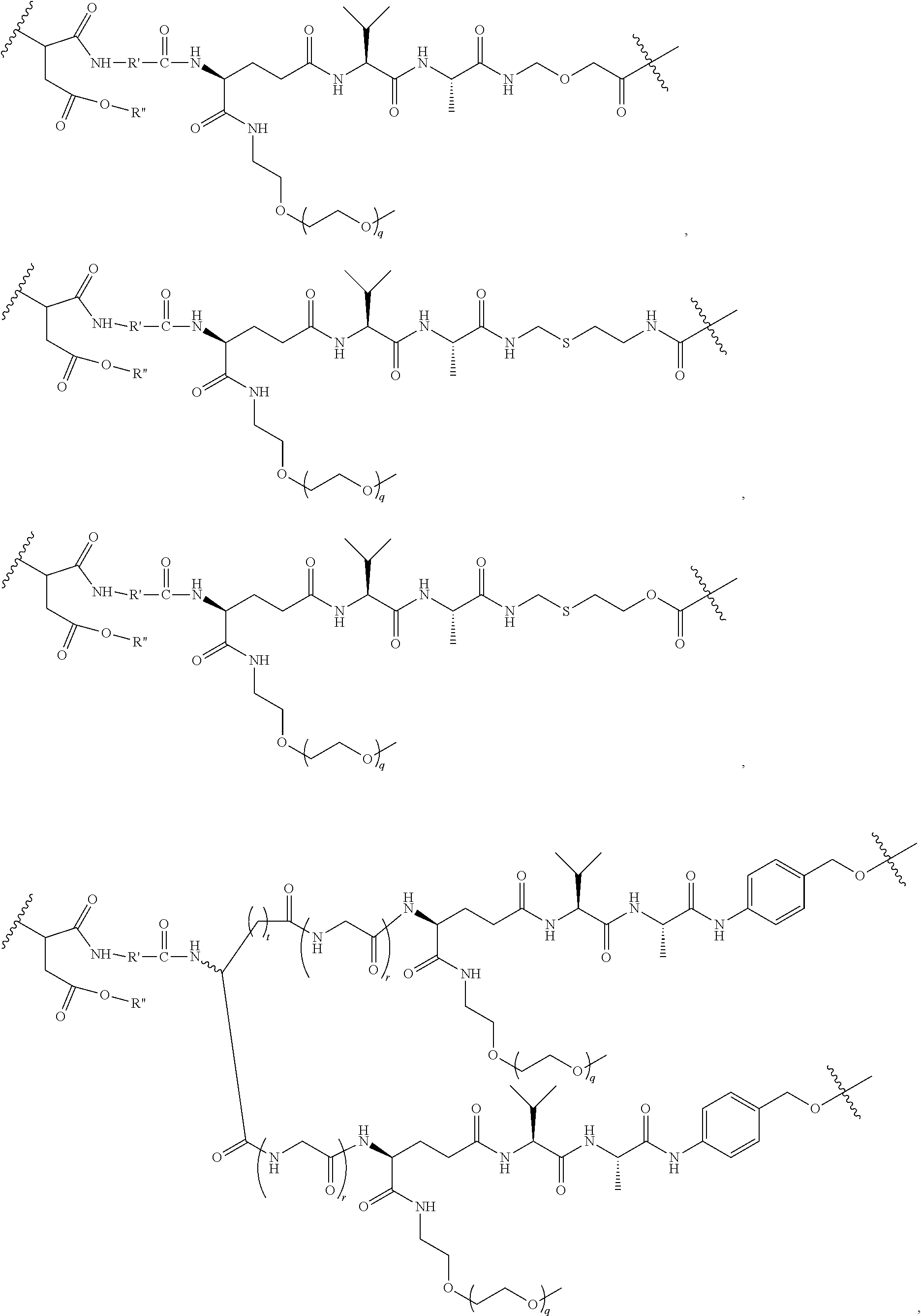 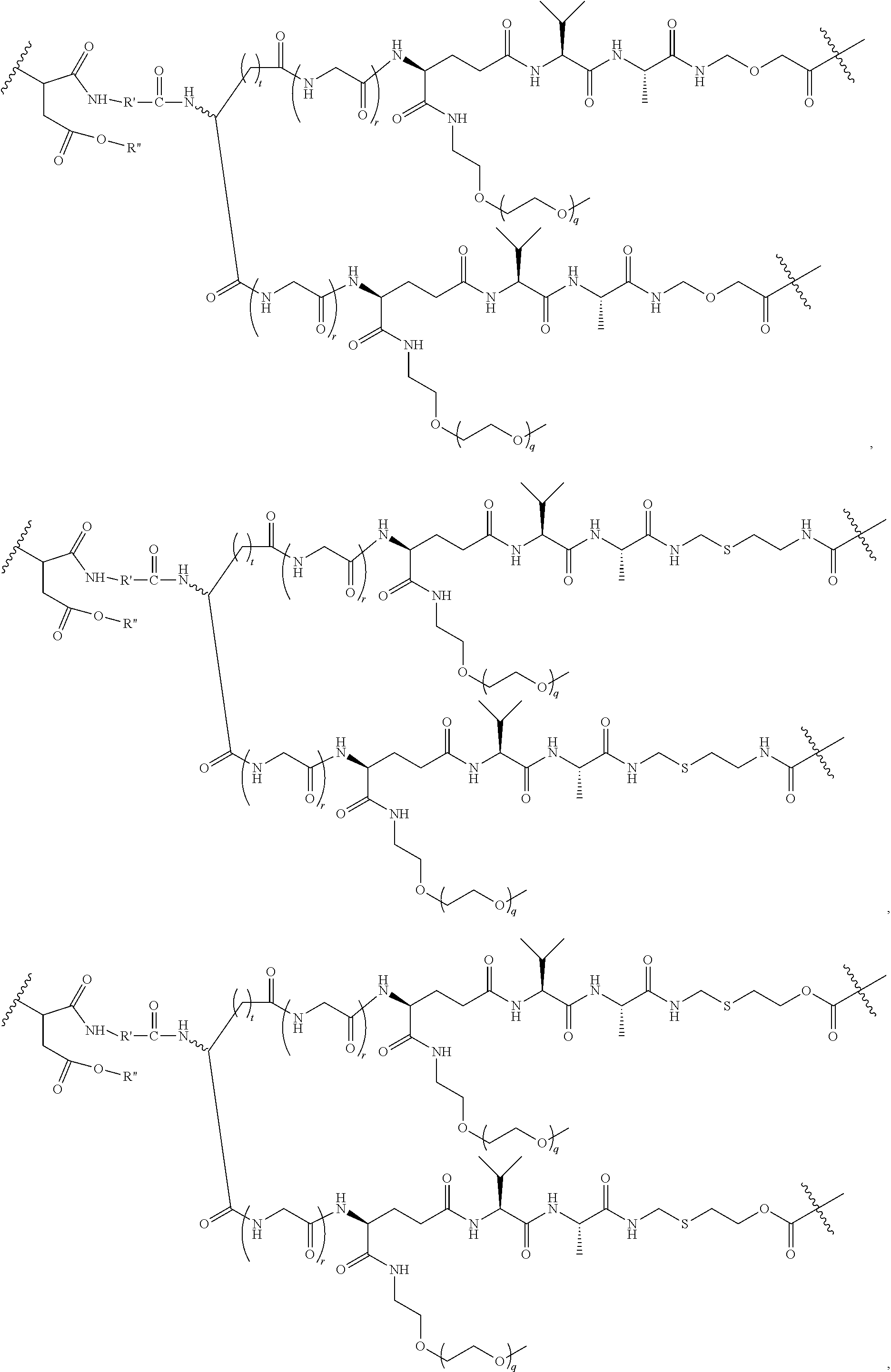 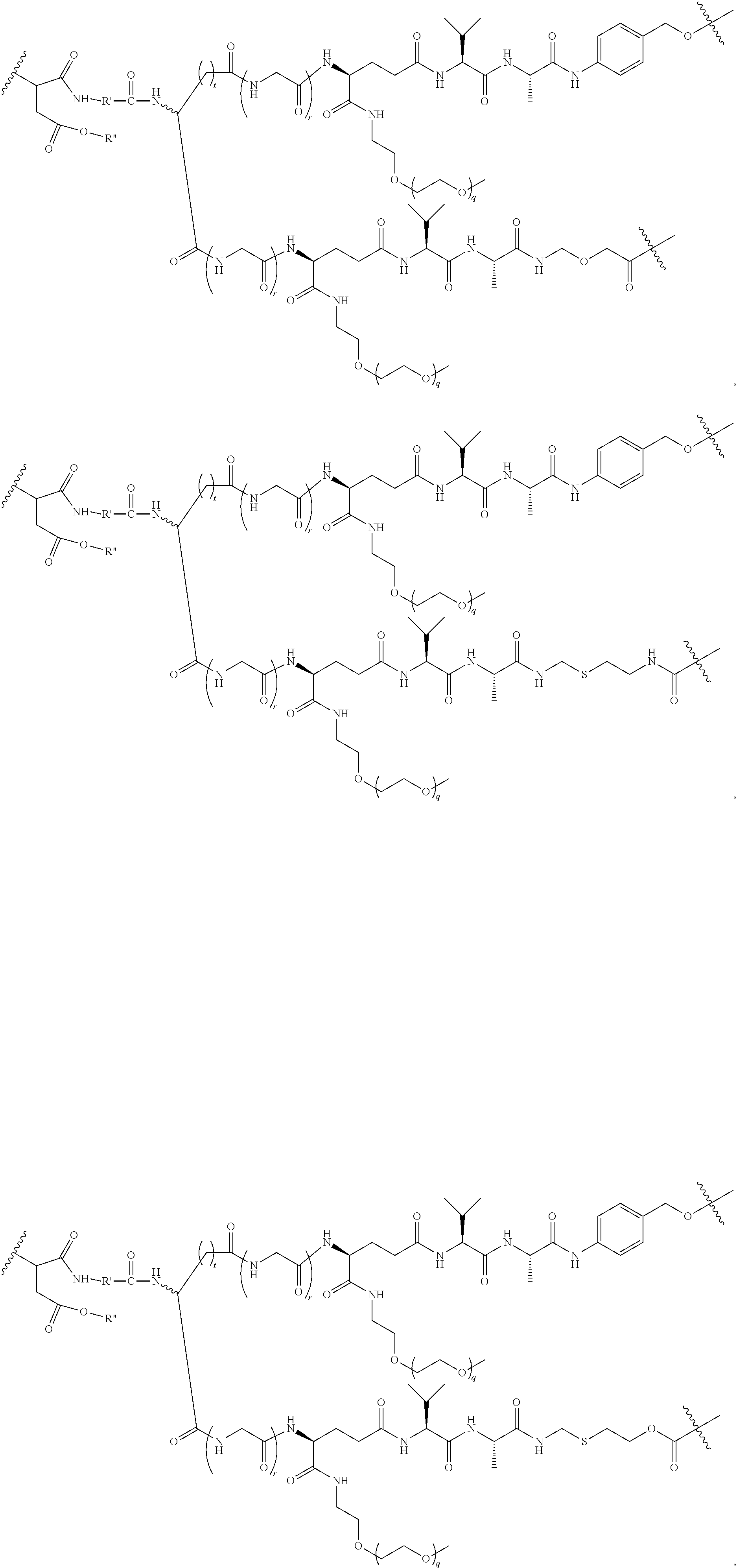 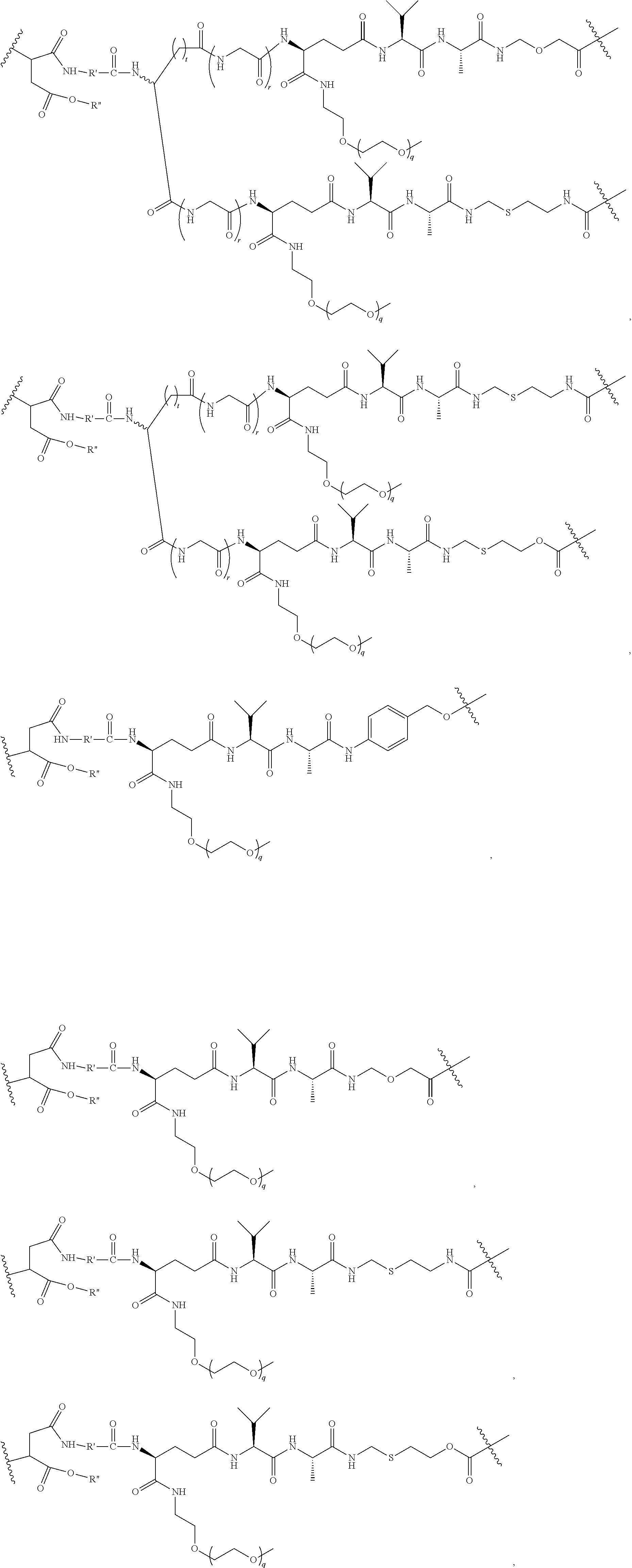 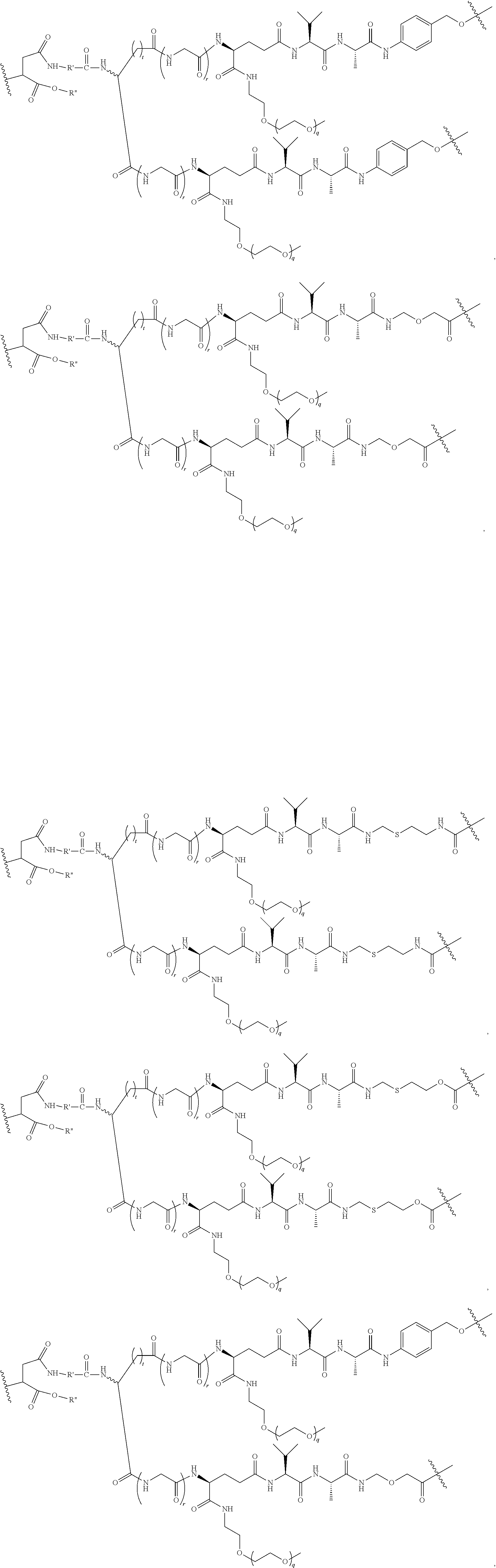 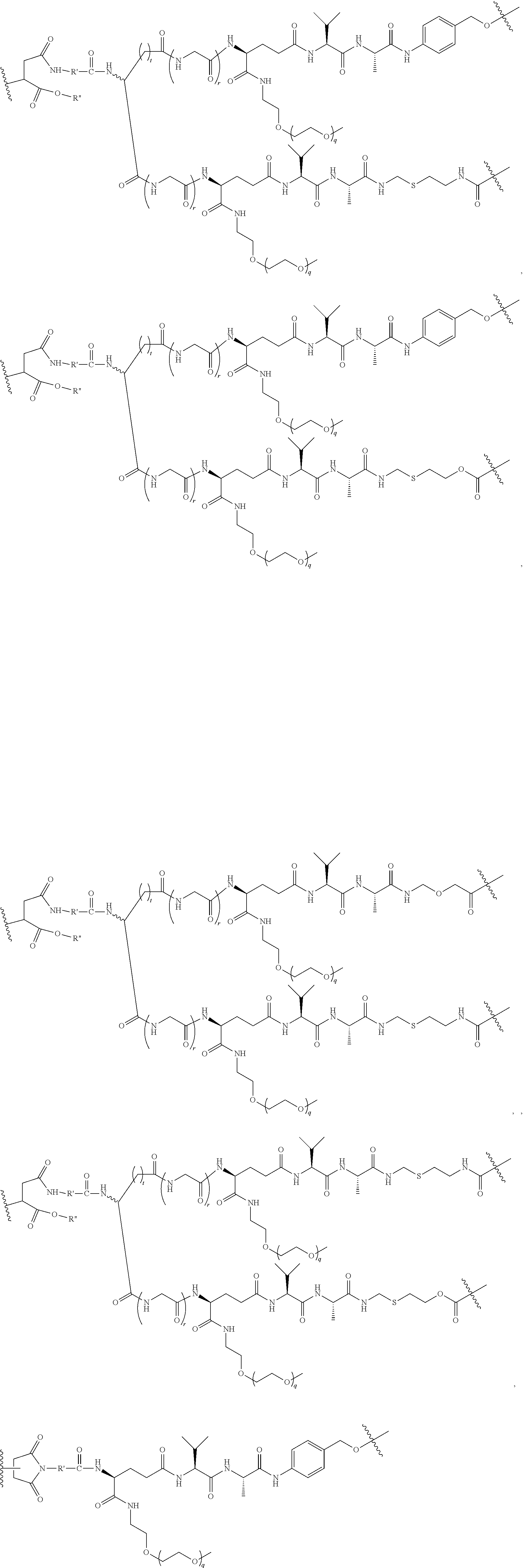
wherein R′ is selected from the group consisting of an optionally substituted C₁-C₁₀ alkyl, a C₁-C₁₀ alkoxy, a C₁-C₁₀ aminoalkyl and a C₁-C₁₀ alkyl-aryl, 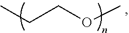
wherein R″ is an optionally substituted C₁-C₁₀ alkyl,
wherein a substituent is selected from the group consisting of an amino group, a halogen, a nitro group, a hydroxyl group, an acetyl group, a cyano group, a C₁-C₁₀ alkyl, a C₁-C₁₀ alkoxy, a C₁-C₁₀ aminoalkyl, a C₁-C₁₀ haloalkyl, a C₂-C₁₀ vinyl, a C₂-C₁₀ alkynyl, an amide group, a C₃-C₈ cycloalkyl, and a C₃-C₈ heterocycloalkyl,
wherein n is any integer selected from 1-10,
wherein r is any integer independently selected from 1-4,
wherein t is any integer independently selected from 1-4, and
wherein q is any integer independently selected from 1-20.

14-16. (canceled)

17. A linker-drug conjugate comprising,
the linker according to claim 1, wherein the linker has a structure as shown in Formula (II):
La—Bₚ [symbol] Lb-Lc-D)ₘ Formula (II)
or a pharmaceutically acceptable salt thereof,
wherein La is an extension unit capable of linking a ligand unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 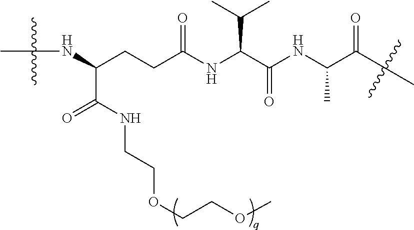
wherein q is any integer selected from 1-20,
wherein the Lb structure fragment is linked to La or B via an amino group and to the Lc fragment via a carbonyl group,
wherein the Lc fragment is a releasable assembly unit, and
wherein D is a drug unit.

18-22. (canceled)

23. The linker-drug conjugate according to claim 17, wherein the linker-drug conjugate has a structure as shown in Formula (III): 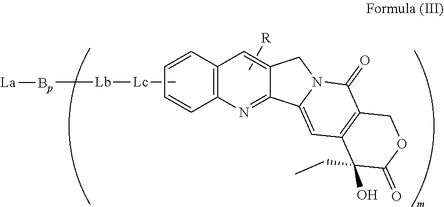
or a pharmaceutically acceptable salt thereof,
wherein La is an extension unit capable of linking a ligand unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 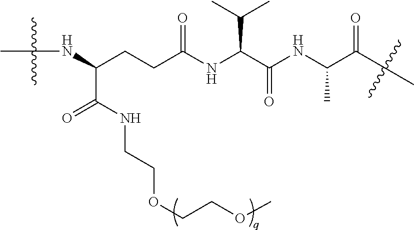
wherein q is any integer selected from 1-20,
wherein the Lb structure fragment is linked to La or B via an amino group and to the Lc fragment via a carbonyl group,
wherein the Lc fragment is a releasable assembly unit, and
wherein 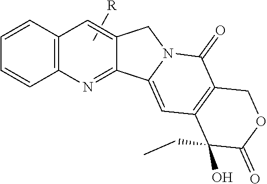
represents a camptothecin or a derivative thereof.

24-25. (canceled)

26. The linker-drug conjugate according to claim 23, wherein the camptothecin or the derivative thereof comprises a 10-difluoromethyl having the following structure: 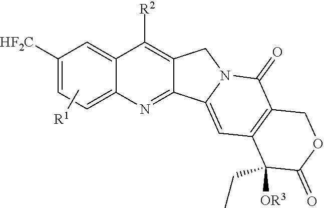
wherein R¹ is selected from the group consisting of a hydrogen, an optionally substituted amino group, and an optionally substituted C₁-C₆ alkyl,
wherein a binding site of R¹ is any one of three unsubstituted sites on a benzene ring,
wherein R² is selected from the group consisting of a hydrogen, an optionally substituted amino group, and an optionally substituted C₁-C₆ alkyl,
wherein R³ is selected from the group consisting of a hydrogen, an acyl group, and an optionally substituted C₁-C₆ alkyl.

27-29. (canceled)

30. The linker-drug conjugate according to claim 17, wherein the linker-drug conjugate has a structure as shown in Formula (IVa), (IVb), (IVc), or (IVd): 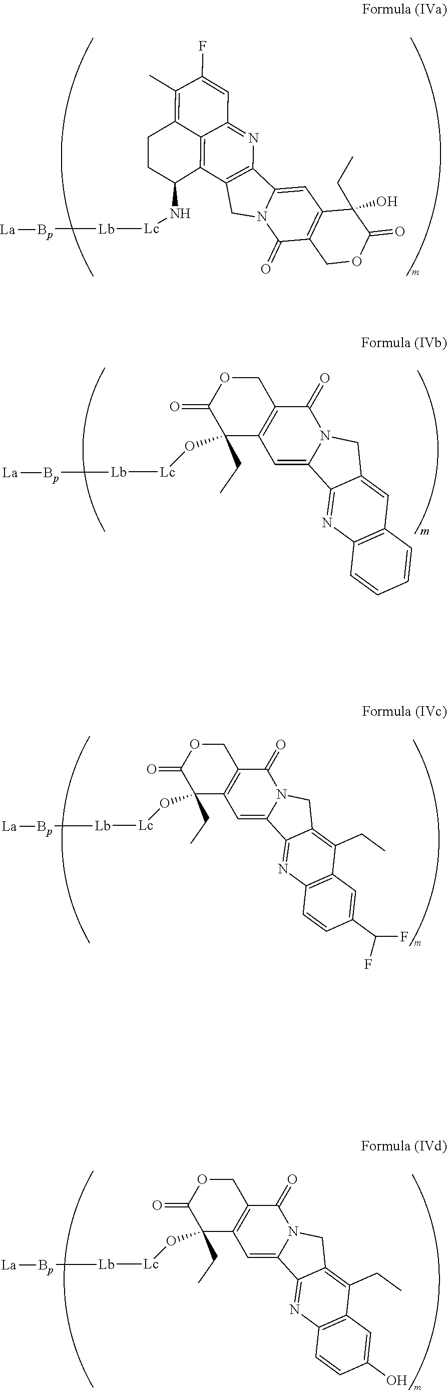
or a pharmaceutically acceptable salt thereof,
wherein La is an extension unit capable of linking a ligand unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 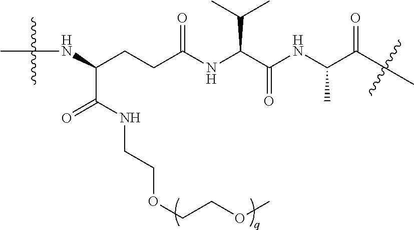
wherein q is any integer selected from 1-20,
wherein the Lb structure fragment is linked to La or B via an amino group and to the Lc fragment via a carbonyl group, and
wherein the Lc fragment is a releasable assembly unit.

31. (canceled)

32. A ligand-drug having a structure shown in Formula (V): 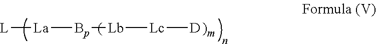
or a pharmaceutically acceptable salt thereof,
wherein L is a ligand unit,
wherein the subscript n is any integer selected from 1-8,
wherein La is an extension unit,
wherein B is an optional branching unit,
wherein the subscript p is 0 or 1,
wherein m is 1 when p is 0,
wherein m is 2, 3, or 4 when p is 1,
wherein the Lb structure fragment is selected from the following formula: 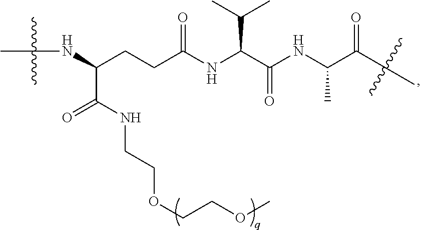
wherein q is any integer selected from 1-20,
wherein the Lb structure is linked to La or B via an amino group and to the Lc fragment via a carbonyl group,
wherein the Lc fragment is a releasable assembly unit, and
wherein D is a drug unit.

33-37. (canceled)

38. The ligand-drug conjugate according to claim 32, wherein the ligand-drug conjugate has a structure as shown in Formula (VI): 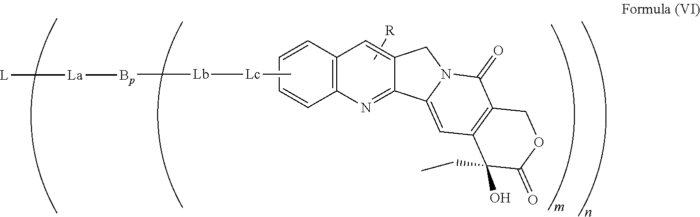
or a pharmaceutically acceptable salt thereof,
wherein L is a ligand unit,
wherein n is any integer selected from 1-8,
wherein La is an extension unit,
wherein B is an optional branching unit,
wherein a subscript p is 0 or 1,
wherein m is 1 when p is 0,
wherein m is 2, 3, or 4 when p is 1,
wherein the Lb structure fragment is selected from the following formula: 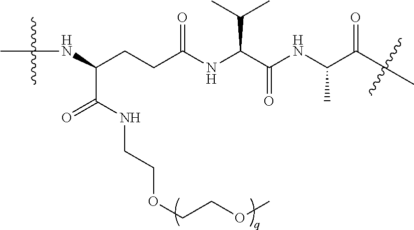
wherein q is any integer selected from 1-20,
wherein the Lb structure fragment is linked to La or B via an amino group and to the Lc fragment via a carbonyl group
wherein the Lc fragment is a releasable assembly unit, and
wherein 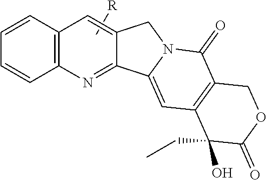
represents a camptothecin or a derivative thereof.

39-44. (canceled)

45. The ligand-drug conjugate according to claim 32, wherein the ligand-drug conjugate has a structure as shown in Formula (VIIa), (VIIb), (VIIc) or (VIId): 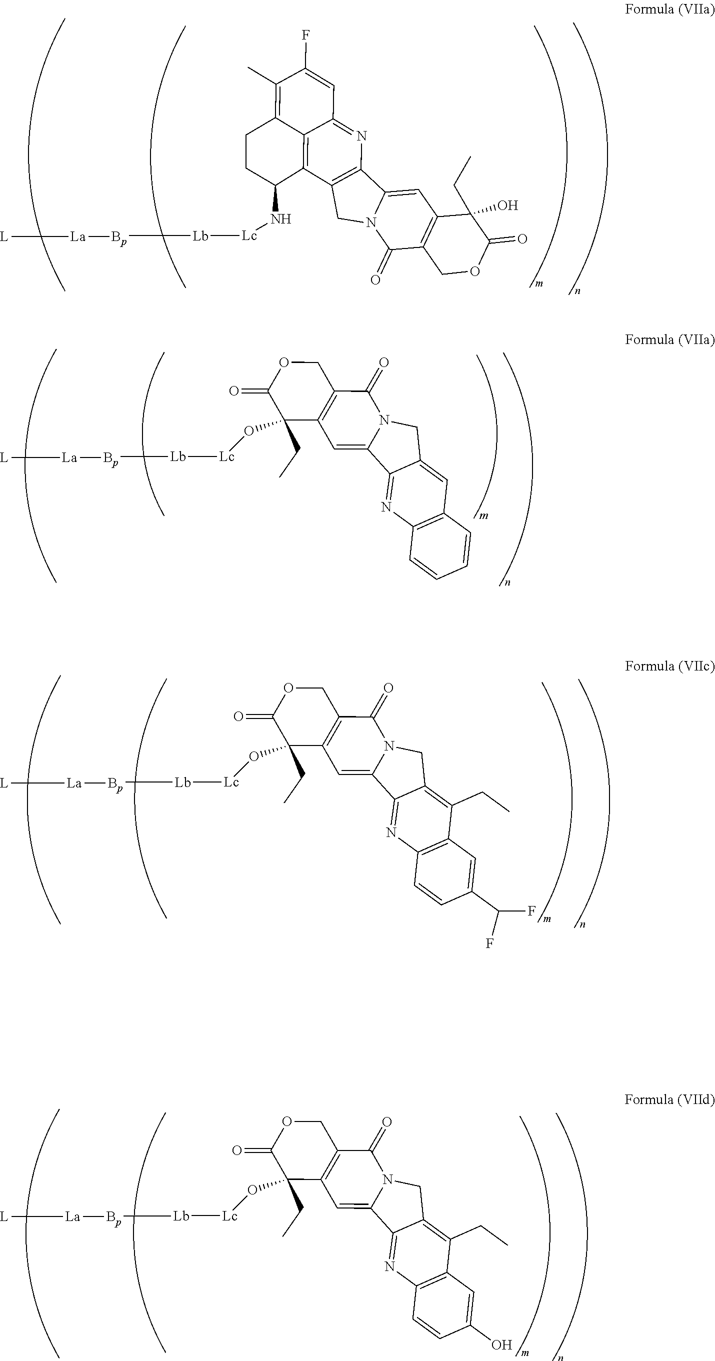
or a pharmaceutically acceptable salt thereof,
wherein L is a ligand unit,
wherein n is any integer selected from 1-8,
wherein La is an extension unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 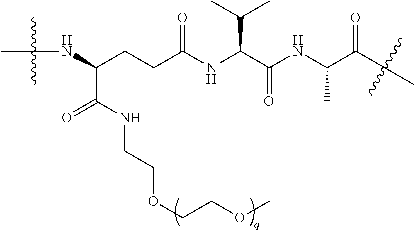
wherein q is any integer selected from 1-20,
wherein the Lb structure fragment is linked to La or B via an amino group and to the Lc fragment via a carbonyl group, and
wherein the Lc fragment is a releasable assembly unit.

46-50. (canceled)

51. The ligand-drug conjugate according to claim 32, the ligand-drug conjugate has a structure as shown in Formula (VIII): 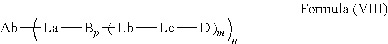
or a pharmaceutically acceptable salt thereof,
wherein Ab represents an antibody,
wherein n is any integer selected from 1-8,
wherein La is an extension unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 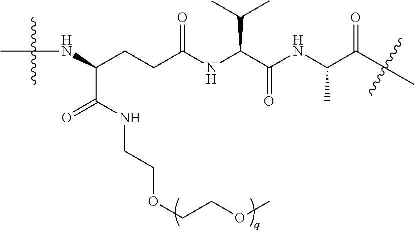
wherein q is any integer selected from 1-20,
wherein the Lb structure fragment is linked to La or B via an amino group and to the Lc fragment via a carbonyl group,
wherein the Lc fragment is a releasable assembly unit, and
wherein D is a drug unit.

52. (canceled)

53. The ligand-drug conjugate according to claim 32, wherein the ligand-drug conjugate has a structure as shown in Formula (IX): 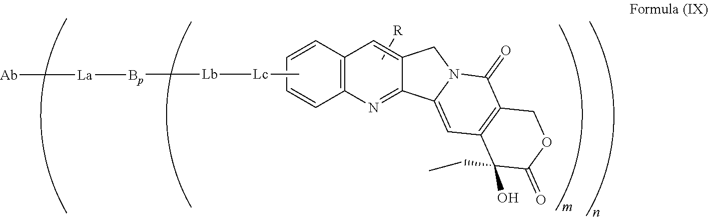
or a pharmaceutically acceptable salt thereof,
wherein Ab represents an antibody,
wherein n is any integer selected from 1-8,
wherein La is an extension unit,
wherein B is an optional branching unit,
wherein p is 0 or 1,
wherein m is 1 when p is 0,
wherein m is 2, 3, or 4 when p is 1,
wherein the Lb structure fragment is selected from the following formula: 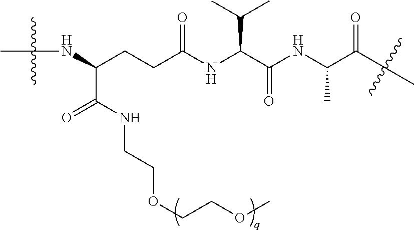
wherein q is any integer selected from 1-20,
wherein the Lb structure fragment is linked to La or B via an amino group and to the Lc fragment via a carbonyl group,
wherein the Lc fragment is a releasable assembly unit, and
wherein 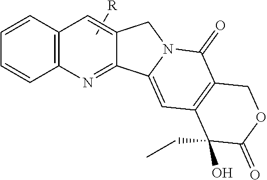
represents a camptothecin or a derivative thereof.

54-88. (canceled)
